(19) United States
(12) Patent Application Publication
(10) Pub. No.: US 2023/0227428 A1
(43) Pub. Date: Jul. 20, 2023

(54) AMIDO COMPOUNDS

(71) Applicant: Anaxis Pharma Pty Ltd, Parkville, Victoria (AU)

(72) Inventors: Jean-Marc Daniel Garnier, Parkville (AU); Martin Brzozowski, Parkville (AU); John Thomas Feutrill, Parkville (AU); Guillaume Laurent Lessene, Parkville (AU); Christopher Gardner, Parkville (AU); Peter Edward Czabotar, Parkville (AU); Angus Cowan, Parkville (AU); Pooja Sharma, Parkville (AU); Carole Annie Schuster-Klein, Parkville, Victoria (AU); Christophe Poitevin, Parkville (AU)

(21) Appl. No.: 18/011,408

(22) PCT Filed: Jun. 21, 2021
(86) PCT No.: PCT/AU2021/050644

§ 371 Date: Dec. 19, 2022

(30) Foreign Application Priority Data
Jun. 19, 2020 (AU) 2020902035
Jun. 19, 2020 (AU) 2020902036
Jun. 18, 2021 (WO) PCT/AU2021/050638

(51) Int. Cl. C07D 401/12; C07D 401/14; C07D 409/14; C07D 403/12; C07D 413/14; C07D 231/38; A61P 29/00

(52) U.S. Cl. CPC C07D 401/12; C07D 401/14; C07D 409/14; C07D 403/12; C07D 413/14; C07D 231/38; A61P 29/00

(57) ABSTRACT

This invention relates to compounds of formula (I) and salts, solvates, tautomers, N-oxides, stereoisomers, polymorphs and/or prodrugs thereof. Also disclosed is the use of the compound of formula (I) to treat necroptosis and/or inhibit RIP1 and/or MLKL.

This application claims priority to Australian provisional patent application nos. 2020902035 and 2020902036 (both filed on 19 Jun. 2020) and to international application no. PCT/AU2021/050638 (filed on 18 Jun. 2021 which in turn claims priority to AU2020902035); the entire contents of each of which is hereby incorporated by reference.

### FIELD

The present disclosure relates to compounds that treat necroptosis and/or inhibit RIP1 and/or MLKL, and methods for their use.

### BACKGROUND

In many diseases, cell death is mediated through apoptotic and/or necrotic pathways. While much is known about the mechanisms of action that control apoptosis, control of necrosis is not as well understood. Understanding the mechanisms in respect of both necrosis and apoptosis in cells is essential to being able to treat conditions, such as neurodegenerative diseases, stroke, coronary heart disease, kidney disease, liver disease, AIDS and the conditions associated with AIDS.

Cell death has traditionally been categorized as either apoptotic or necrotic based on morphological characteristics (Wyllie et al., Int. Rev. Cytol. 68: 251 (1980)). These two modes of cell death were also initially thought to occur via regulated (caspase-dependent) and non-regulated processes, respectively. More recent studies, however, demonstrate that the underlying cell death mechanisms resulting in these two phenotypes are much more complicated and under some circumstances interrelated. Furthermore, conditions that lead to necrosis can occur by either regulated caspase-independent or non-regulated processes.

One regulated caspase-independent cell death pathway with morphological features resembling necrosis, called necroptosis, has been described (Degterev et al., Nat. Chem. Biol. 1:112, 2005). This manner of cell death can be initiated with various stimuli (e.g., TNF-[alpha] and Fas ligand) and in an array of cell types (e.g., monocytes, fibroblasts, lymphocytes, macrophages, epithelial cells and neurons). Necroptosis may represent a significant contributor to and in some cases predominant mode of cellular demise under pathological conditions involving excessive cell stress, rapid energy loss and massive oxidative species generation, where the highly energy-dependent apoptosis process is not operative.

In WO2015/172203, we reported that particular compounds described in US2005/0085637 have been found to be suitable for inhibiting necroptosis. We also discussed particularly suitable compounds for inhibiting necroptosis in WO2016/127213.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

As discussed above, certain compounds described in WO2016/127213, US2005/0085637 and WO2015/172203 have been found to be suitable for treating necroptosis. Surprisingly, the inventors have now discovered that other types of compounds are also suitable for treating necroptosis. Further, and equally surprising, the compounds described in this invention target two key effectors of the necroptotic pathway, namely receptor-interacting serine/threonine protein kinase 1 (RIP1 or RIPK1) and mixed lineage kinase domain-like protein (MLKL). RIP1 is the switch by which cell death is either directed towards an apoptotic or necroptotic phenotype. MLKL is the last known effector of the necroptotic cascade. Thus, inhibition of these two proteins may represent an added benefit over compounds that inhibit only one of these proteins.

In one aspect, there is provided a compound according to Formula (I)

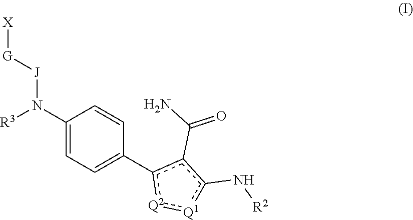

wherein
Q¹ and Q² are selected from N and NR¹, wherein when Q² is N, Q¹ is NR¹ and when Q¹ is N, Q² is NR¹;
R¹ and R³ are independently selected from H and an optionally substituted C₁₋₆-alkyl;
R² is H, an optionally substituted C₁-C₆-alkyl, an optionally substituted aryl or an optionally substituted heterocyclyl;
X is selected from optionally substituted C₁₋₆alkyl, optionally substituted haloC₁₋₆alkyl, optionally substituted —C₁₋₆alkylamino, optionally substituted C₂₋₆alkynyl, optionally substituted cycloalkyl, optionally substituted halocycloalkyl, optionally substituted aryl, optionally substituted alkylaryl, optionally substituted heterocyclyl, optionally substituted C₁₋₆ alkylheterocyclyl;
J is selected from carbonyl and

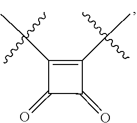 [SMILES: *c1c(*)c(=O)c1=O]

and
G is selected from a single bond, NR³, CR⁴R⁵ and optionally substituted heterocyclyl,
R⁴ and R⁵ are independently selected from H, optionally substituted C₁₋₆alkyl, optionally substituted aryl and optionally substituted amino;
or a pharmaceutically acceptable salt, solvate, tautomer, stereoisomer, N-oxide and/or prodrug thereof.

The inventors have found that compounds of formula (I) are inhibitors of RIP1 and MLKL.

In some embodiments, the compound of formula (I) is selected from any of compounds 1-130 described herein, or a pharmaceutically acceptable salt, solvate, tautomer, stereoisomer, N-oxide and/or prodrug thereof, preferably any of compounds 1-127 or a pharmaceutically acceptable salt, solvate, tautomer, stereoisomer, N-oxide and/or prodrug thereof, more preferably a compound selected from compounds 1, 3, 7-8, 13, 21-25, 27, 30, 32, 39, 44, 47-48, 58-59, 60-61, 64, 66-67, 78, 80-83, 87-98, 103, 105, 107, 109-111, and 115, or a pharmaceutically acceptable salt, solvate, tautomer, stereoisomer, N-oxide and/or prodrug thereof.

In another aspect, there is provided a medicament comprising a compound of the invention.

In another aspect, there is provided a pharmaceutical composition comprising a compound of the invention and a pharmaceutically acceptable excipient.

In another aspect, there is provided a method of treating necroptosis, comprising administering to a subject in need thereof an effective amount of a compound of the invention.

In another aspect, there is provided a method of inhibiting RIP1 and/or MLKL, comprising contacting a cell with a compound of the invention.

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### Definitions

Unless otherwise herein defined, the following terms will be understood to have the general meanings which follow.

The term “C₁₋₆alkyl” refers to optionally substituted straight chain or branched chain hydrocarbon groups having from 1 to 6 carbon atoms. Examples include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl and the like. Unless the context requires otherwise, the term “C₁₋₆alkyl” also encompasses alkyl groups containing one less hydrogen atom such that the group is attached via two positions i.e. divalent. “C₁₋₄alkyl” and “C₁₋₃alkyl” including methyl, ethyl, propyl, isopropyl, n-butyl, iso-butyl, sec-butyl and tert-butyl are preferred with methyl being particularly preferred.

The term “C₂₋₆alkenyl” refers to optionally substituted straight chain or branched chain hydrocarbon groups having at least one double bond of either E or Z stereochemistry where applicable and 2 to 6 carbon atoms. Examples include vinyl, 1-propenyl, 1- and 2-butenyl and 2-methyl-2-propenyl. Unless the context requires otherwise, the term “C₂₋₆alkenyl” also encompasses alkenyl groups containing one less hydrogen atom such that the group is attached via two positions i.e. divalent. “C₂₋₄alkenyl” and “C₂₋₃alkenyl” including ethenyl, propenyl and butenyl are preferred with ethenyl being particularly preferred.

The term “C₂₋₆alkynyl” refers to optionally substituted straight chain or branched chain hydrocarbon groups having at least one triple bond and 2 to 6 carbon atoms. Examples include ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl and the like. Unless the context indicates otherwise, the term “C₂₋₆alkynyl” also encompasses alkynyl groups containing one less hydrogen atom such that the group is attached via two positions i.e. divalent. C₂₋₃alkynyl is preferred.

The term “C₃₋₈cycloalkyl” refers to non-aromatic cyclic groups having from 3 to 8 carbon atoms, including cyclopropyl, cyclobutyl, cyclopentyl, cyclohexyl, cycloheptyl and cyclooctyl. It will be understood that cycloalkyl groups may be saturated such as cyclohexyl or unsaturated such as cyclohexenyl. C₃₋₆cycloalkyl such as cyclopropyl, cyclobutyl, cyclopentyl and cyclohexyl are preferred.

The terms “hydroxy” and “hydroxyl” refer to the group —OH.

The term “oxo” refers to the group ═O.

The term “C₁₋₆alkoxy” refers to an alkyl group as defined above covalently bound via an O linkage containing 1 to 6 carbon atoms, such as methoxy, ethoxy, propoxy, isoproxy, butoxy, tert-butoxy and pentoxy. “C₁₋₄alkoxy” and “C₁₋₃alkoxy” including methoxy, ethoxy, propoxy and butoxy are preferred with methoxy being particularly preferred.

The terms “haloC₁₋₆alkyl” and “C₁₋₆alkylhalo” refer to a C₁₋₆alkyl which is substituted with one or more halogens. HaloC₁₋₃alkyl groups are preferred, such as for example, —CH₂CF₃, and —CF₃.

The terms “haloC₁₋₆alkoxy” and “C₁₋₆alkoxyhalo” refer to a C₁₋₆alkoxy which is substituted with one or more halogens. C₁₋₃alkoxyhalo groups are preferred, such as for example, —OCF₃.

The term “carboxylate” or “carboxyl” refers to the group —COO— or —COOH.

The term “ester” refers to a carboxyl group having the hydrogen replaced with, for example a C₁₋₆alkyl group (“carboxylC₁₋₆alkyl” or “alkylester”), an aryl or aralkyl group (“arylester” or “aralkylester”) and so on. CO₂C₁₋₃alkyl groups are preferred, such as for example, methylester (CO₂Me), ethylester (CO₂Et) and propylester (CO₂Pr) and includes reverse esters thereof (e.g. —OC(O)Me, —OC(O)Et and —OC(O)Pr).

The terms “cyano” and “nitrile” refer to the group —CN.

The term “nitro” refers to the group —NO₂.

The term “amino” refers to the group —NH₂.

The term “substituted amino” or “secondary amino” refers to an amino group having a hydrogen replaced with, for example a C₁₋₆alkyl group (“C₁₋₆alkylamino”), an aryl or aralkyl group (“arylamino”, “aralkylamino”) and so on. C₁₋₃alkylamino groups are preferred, such as for example, methylamino (NHMe), ethylamino (NHEt) and propylamino (NHPr).

The term “disubstituted amino” or “tertiary amino” refers to an amino group having the two hydrogens replaced with, for example a C₁₋₆alkyl group, which may be the same or different (“dialkylamino”), an aryl and alkyl group (“aryl(alkyl)amino”) and so on. Di(C₁₋₃alkyl)amino groups are preferred, such as for example, dimethylamino (NMe₂), diethylamino (NEt₂), dipropylamino (NPr₂) and variations thereof (e.g. N(Me)(Et) and so on).

The term “aldehyde” refers to the group —C(═O)H.

The term “acyl” refers to the group —C(O)CH₃.

The term “ketone” refers to a carbonyl group which may be represented by —C(O)—.

The term “substituted ketone” refers to a ketone group covalently linked to at least one further group, for example, a C₁₋₆alkyl group (“C₁₋₆alkylacyl” or “alkylketone” or “ketoalkyl”), an aryl group (“arylketone”), an aralkyl group (“aralkylketone) and so on. C₁₋₃alkylacyl groups are preferred.

The term “amido” or “amide” refers to the group —C(O)NH₂.

The term “substituted amido” or “substituted amide” refers to an amido group having a hydrogen replaced with, for example a C₁₋₆alkyl group (“C₁₋₆alkylamido” or “C₁₋₆alkylamide”), an aryl (“arylamido”), aralkyl group (“aralkylamido”) and so on. C₁₋₃alkylamide groups are preferred, such as for example, methylamide (—C(O)NHMe), ethylamide (—C(O)NHEt) and propylamide (—C(O)NHPr) and includes reverse amides thereof (e.g. —NHMeC(O)—, —NHEtC(O)— and —NHPrC(O)—).

The term “disubstituted amido” or “disubstituted amide” refers to an amido group having the two hydrogens replaced with, for example a C₁₋₆alkyl group (“di(C₁₋₆alkyl)amido” or “di(C₁₋₆ alkyl)amide”), an aralkyl and alkyl group (“alkyl(aralkyl)amido”) and so on.

Di(C₁₋₃alkyl)amide groups are preferred, such as for example, dimethylamide (—C(O)NMe₂), diethylamide (—C(O)NEt₂) and dipropylamide ((—C(O)NPr₂) and variations thereof (e.g. —C(O)N(Me)Et and so on) and includes reverse amides thereof.

The term “thiol” refers to the group —SH.

The term “C₁₋₆alkylthio” refers to a thiol group having the hydrogen replaced with a C₁₋₆alkyl group. C₁₋₃alkylthio groups are preferred, such as for example, thiolmethyl, thiolethyl and thiolpropyl.

The terms “thioxo” refer to the group ═S.

The term “sulfinyl” refers to the group —S(═O)H.

The term “substituted sulfinyl” or “sulfoxide” refers to a sulfinyl group having the hydrogen replaced with, for example a C₁₋₆alkyl group (“C₁₋₆alkylsulfinyl” or “C₁₋₆alkylsulfoxide”), an aryl (“arylsulfinyl”), an aralkyl (“aralkyl sulfinyl”) and so on. C1-3alkylsulfinyl groups are preferred, such as for example, —SOmethyl, —SOethyl and —SOpropyl.

The term “sulfonyl” refers to the group —SO₂H.

The term “substituted sulfonyl” refers to a sulfonyl group having the hydrogen replaced with, for example a C₁₋₆alkyl group (“sulfonylC₁₋₆alkyl”), an aryl (“arylsulfonyl”), an aralkyl (“aralkylsulfonyl”) and so on. SulfonylC₁₋₃alkyl groups are preferred, such as for example, —SO₂Me, —SO₂Et and —SO₂Pr.

The terms “sulfonylamido”, “sulfonamide”, “sulphonylamido” or “sulphonamide” refer to the group —SO₂NH₂.

The terms “substituted sulfonamido”, “substituted sulphonamido”, “substituted sulfonamide” or “substituted sulphonamide” refer to an sulfonylamido group having a hydrogen replaced with, for example a C₁₋₆alkyl group (“sulfonylamidoC₁₋₆alkyl”), an aryl (“arylsulfonamide”), aralkyl (“aralkylsulfonamide”) and so on. SulfonylamidoC₁₋₃alkyl groups are preferred, such as for example, —SO₂NHMe, —SO₂NHEt and —SO₂NHPr and includes reverse sulfonamides thereof (e.g. —NHSO₂Me, —NHSO₂Et and —NHSO₂Pr).

The terms “disubstituted sulfonamido”, “disubstituted sulphonamido”, “disubstituted sulfonamide” or “disubstituted sulphonamide” refer to an sulfonylamido group having the two hydrogens replaced with, for example a C₁₋₆alkyl group, which may be the same or different (“sulfonylamidodi(C₁₋₆alkyl)”), an aralkyl and alkyl group (“sulfonamido(aralkyl)alkyl”) and so on. Sulfonylamidodi(C₁₋₃alkyl) groups are preferred, such as for example, —SO₂NMe₂, —SO₂NEt₂ and —SO₂NPr₂ and variations thereof (e.g. —SO₂N(Me)Et and so on) and includes reserve sulfonamides thereof (e.g. —N(Me)SO₂Me and so on).

The term “sulfate” refers to the group OS(O)₂OH and includes groups having the hydrogen replaced with, for example a C₁₋₆alkyl group (“alkylsulfates”), an aryl (“arylsulfate”), an aralkyl (“aralkylsulfate”) and so on. C₁₋₃sulfates are preferred, such as for example, OS(O)₂OMe, OS(O)₂OEt and OS(O)₂OPr.

The term “sulfonate” refers to the group SO₃H and includes groups having the hydrogen replaced with, for example a C₁₋₆alkyl group (“alkylsulfonate”), an aryl (“arylsulfonate”), an aralkyl (“aralkylsulfonate”) and so on. C₁₋₃sulfonates are preferred, such as for example, SO₃Me, SO₃Et and SO₃Pr.

The term “aryl” refers to a carbocyclic (non-heterocyclic) aromatic ring or mono-, bi- or tri-cyclic ring system. The aromatic ring or ring system is generally composed of 6 to 10 carbon atoms. Examples of aryl groups include but are not limited to phenyl, biphenyl, naphthyl and tetrahydronaphthyl. 6-membered aryls such as phenyl are preferred. The term “alkylaryl” refers to C₁₋₆alkylaryl such as benzyl.

The term “alkoxyaryl” refers to C₁₋₆alkyloxyaryl such as benzyloxy.

The term “heterocyclyl” refers to a moiety obtained by removing a hydrogen atom from a ring atom of a heterocyclic compound which moiety has from 3 to 10 ring atoms (unless otherwise specified), of which 1, 2, 3 or 4 are ring heteroatoms each heteroatom being independently selected from O, S and N.

In this context, the prefixes 3-, 4-, 5-, 6-, 7-, 8-, 9- and 10-membered denote the number of ring atoms, or range of ring atoms, whether carbon atoms or heteroatoms. For example, the term “3-10 membered heterocylyl”, as used herein, pertains to a heterocyclyl group having 3, 4, 5, 6, 7, 8, 9 or 10 ring atoms. Examples of heterocylyl groups include 5-6-membered monocyclic heterocyclyls and 9-10 membered fused bicyclic heterocyclyls.

Examples of monocyclic heterocyclyl groups include, but are not limited to, those containing one nitrogen atom such as aziridine (3-membered ring), azetidine (4-membered ring), pyrrolidine (tetrahydropyrrole), pyrroline (e.g., 3-pyrroline, 2,5-dihydropyrrole), 2H-pyrrole or 3H-pyrrole (isopyrrole, isoazole) or pyrrolidinone (5-membered rings), piperidine, dihydropyridine, tetrahydropyridine (6-membered rings), and azepine (7-membered ring); those containing two nitrogen atoms such as imidazoline, pyrazolidine (diazolidine), imidazoline, pyrazoline (dihydropyrazole) (5-membered rings), piperazine (6-membered ring); those containing one oxygen atom such as oxirane (3-membered ring), oxetane (4-membered ring), oxolane (tetrahydrofuran), oxole (dihydrofuran) (5-membered rings), oxane (tetrahydropyran), dihydropyran, pyran (6-membered rings), oxepin (7-membered ring); those containing two oxygen atoms such as dioxolane (5-membered ring), dioxane (6-membered ring), and dioxepane (7-membered ring); those containing three oxygen atoms such as trioxane (6-membered ring); those containing one sulfur atom such as thiirane (3-membered ring), thietane (4-membered ring), thiolane (tetrahydrothiophene) (5-membered ring), thiane (tetrahydrothiopyran) (6-membered ring), thiepane (7-membered ring); those containing one nitrogen and one oxygen atom such as tetrahydrooxazole, dihydrooxazole, tetrahydroisoxazole, dihydroisoxazole (5-membered rings), morpholine, tetrahydrooxazine, dihydrooxazine, oxazine (6-membered rings); those containing one nitrogen and one sulfur atom such as thiazoline, thiazolidine (5-membered rings), thiomorpholine (6-membered ring); those containing two nitrogen and one oxygen atom such as oxadiazine (6-membered ring); those containing one oxygen and one sulfur such as: oxathiole (5-membered ring) and oxathiane (thioxane) (6-membered ring); and those containing one nitrogen, one oxygen and one sulfur atom such as oxathiazine (6-membered ring).

Heterocyclyls also encompass aromatic heterocyclyls and non-aromatic heterocyclyls. Such groups may be substituted or unsubstituted.

The term “aromatic heterocyclyl” may be used interchangeably with the term “heteroaromatic” or the term “heteroaryl” or “hetaryl”. The heteroatoms in the aromatic heterocyclyl group may be independently selected from N, S and O. The aromatic heterocyclyl groups may comprise 1, 2, 3, 4 or more ring heteroatoms. In the case of fused aromatic heterocyclyl groups, only one of the rings must contain a heteroatom and not all rings must be aromatic.

“Heteroaryl” is used herein to denote a heterocyclic group having aromatic character and embraces aromatic monocyclic ring systems and polycyclic (e.g. bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to about four heteroatoms typically selected from nitrogen, sulphur and oxygen. The heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Aromatic heterocyclyl groups may be 5-membered or 6-membered mono-cyclic aromatic ring systems.

Examples of 5-membered monocyclic heteroaryl groups include but are not limited to furanyl, thienyl, pyrrolyl, oxazolyl, oxadiazolyl (including 1,2,3 and 1,2,4 oxadiazolyls and furazanyl i.e. 1,2,5-oxadiazolyl), thiazolyl, isoxazolyl, isothiazolyl, pyrazolyl, imidazolyl, triazolyl (including 1,2,3, 1,2,4 and 1,3,4 triazolyls), oxatriazolyl, tetrazolyl, thiadiazolyl (including 1,2,3 and 1,3,4 thiadiazolyls) and the like.

Examples of 6-membered monocyclic heteroaryl groups include but are not limited to pyridinyl, pyrimidinyl, pyridazinyl, pyrazinyl, triazinyl, pyranyl, oxazinyl, dioxinyl, thiazinyl, thiadiazinyl and the like. Examples of 6-membered aromatic heterocyclyls containing nitrogen include pyridyl (1 nitrogen), pyrazinyl, pyrimidinyl and pyridazinyl (2 nitrogens).

Aromatic heterocyclyl groups may also be bicyclic or polycyclic heteroaromatic ring systems such as fused ring systems (including purine, pteridinyl, napthyridinyl, 1H thieno[2,3-c]pyrazolyl, thieno[2,3-b]furyl and the like) or linked ring systems (such as oligothiophene, polypyrrole and the like). Fused ring systems may also include aromatic 5-membered or 6-membered heterocyclyls fused to carbocyclic aromatic rings such as phenyl, napthyl, indenyl, azulenyl, fluorenyl, anthracenyl and the like, such as 5-membered aromatic heterocyclyls containing nitrogen fused to phenyl rings, 5-membered aromatic heterocyclyls containing 1 or 2 nitrogens fused to phenyl ring.

A bicyclic heteroaryl group may be, for example, a group selected from: a) a benzene ring fused to a 5- or 6-membered ring containing 1, 2 or 3 ring heteroatoms; b) a pyridine ring fused to a 5- or 6-membered ring containing 1, 2 or 3 ring heteroatoms; c) a pyrimidine ring fused to a 5- or 6-membered ring containing 1 or 2 ring heteroatoms; d) a pyrrole ring fused to a 5- or 6-membered ring containing 1, 2 or 3 ring heteroatoms; e) a pyrazole ring fused to a 5- or 6-membered ring containing 1 or 2 ring heteroatoms; f an imidazole ring fused to a 5- or 6-membered ring containing 1 or 2 ring heteroatoms; g) an oxazole ring fused to a 5- or 6-membered ring containing 1 or 2 ring heteroatoms; h) an isoxazole ring fused to a 5- or 6-membered ring containing 1 or 2 ring heteroatoms; i) a thiazole ring fused to a 5- or 6-membered ring containing 1 or 2 ring heteroatoms; j) an isothiazole ring fused to a 5- or 6-membered ring containing 1 or 2 ring heteroatoms; k) a thiophene ring fused to a 5- or 6-membered ring containing 1, 2 or 3 ring heteroatoms; l) a furan ring fused to a 5- or 6-membered ring containing 1, 2 or 3 ring heteroatoms; m) a cyclohexyl ring fused to a 5- or 6-membered ring containing 1, 2 or 3 ring heteroatoms; and n) a cyclopentyl ring fused to a 5- or 6-membered ring containing 1, 2 or 3 ring heteroatoms.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzofuran, benzothiophene, benzimidazole, benzoxazole, isobenzoxazole, benzisoxazole, benzothiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), benzodioxole and pyrazolopyridine (e.g. pyrazolo[1,5-a]pyridine) groups. A further example of a six membered ring fused to a five membered ring is a pyrrolopyridine group such as a pyrrolo[2,3-b]pyridine group.

Particular examples of bicyclic heteroaryl groups containing two fused six membered rings include but are not limited to quinoline, isoquinoline, chroman, thiochroman, chromene, isochromene, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydronaphthalene, tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoline, isoindoline and indane groups.

Examples of aromatic heterocyclyls fused to carbocyclic aromatic rings may therefore include but are not limited to benzothiophenyl, indolyl, isoindolyl, benzofuranyl, isobenzofuranyl, benzimidazolyl, indazolyl, benzoxazolyl, benzisoxazolyl, isobenzoxazoyl, benzothiazolyl, benzisothiazolyl, quinolinyl, isoquinolinyl, quinoxalinyl, quinazolinyl, cinnolinyl, benzotriazinyl, phthalazinyl, carbolinyl and the like.

The term “non-aromatic heterocyclyl” encompasses optionally substituted saturated and unsaturated rings which contain at least one heteroatom selected from the group consisting of N, S and O. The ring may contain 1, 2 or 3 heteroatoms. The ring may be a monocyclic ring or part of a polycyclic ring system. Polycyclic ring systems include fused rings and spirocycles. Not every ring in a non-aromatic heterocyclic polycyclic ring system must contain a heteroatom, provided at least one ring contains one or more heteroatoms.

Non-aromatic heterocyclyls may be 3-7 membered mono-cyclic rings.

Examples of 5-membered non-aromatic heterocyclyl rings include 2H-pyrrolyl, 1-pyrrolinyl, 2-pyrrolinyl, 3-pyrrolinyl, pyrrolidinyl, 1-pyrrolidinyl, 2-pyrrolidinyl, 3-pyrrolidinyl, tetrahydrofuranyl, tetrahydrothiophenyl, pyrazolinyl, 2-pyrazolinyl, 3-pyrazolinyl, pyrazolidinyl, 2-pyrazolidinyl, 3-pyrazolidinyl, imidazolidinyl, 3-dioxalanyl, thiazolidinyl, isoxazolidinyl, 2-imidazolinyl and the like.

Examples of 6-membered non-aromatic heterocyclyls include piperidinyl, piperidinonyl, pyranyl, dihyrdopyranyl, tetrahydropyranyl, 2H pyranyl, 4H pyranyl, thianyl, thianyl oxide, thianyl dioxide, piperazinyl, diozanyl, 1,4-dioxinyl, 1,4-dithianyl, 1,3,5-triozalanyl, 1,3,5-trithianyl, 1,4-morpholinyl, thiomorpholinyl, 1,4-oxathianyl, triazinyl, 1,4-thiazinyl and the like. Examples of 7-membered non-aromatic heterocyclyls include azepanyl, oxepanyl, thiepanyl and the like.

Non-aromatic heterocyclyl rings may also be bicyclic heterocyclyl rings such as linked ring systems (for example uridinyl and the like) or fused ring systems. Fused ring systems include non-aromatic 5-membered, 6-membered or 7-membered heterocyclyls fused to carbocyclic aromatic rings such as phenyl, napthyl, indenyl, azulenyl, fluorenyl, anthracenyl and the like. Examples of non-aromatic 5-membered, 6-membered or 7-membered heterocyclyls fused to carbocyclic aromatic rings include indolinyl, benzodiazepinyl, benzazepinyl, dihydrobenzofuranyl and the like.

The term “halo” refers to fluoro, chloro, bromo or iodo.

Unless otherwise defined, the term “optionally substituted” or “optional substituent” as used herein refers to a group which may or may not be further substituted with 1, 2, 3, 4 or more groups, preferably 1, 2 or 3, more preferably 1 or 2 groups selected from the group consisting of C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo (such as CF₃), C₁₋₆alkoxyhalo (such as OCF₃), carboxyl, esters, cyano, nitro, amino, substituted amino, disubstituted amino, acyl, ketones, substituted ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC₁₋₆alkyl, heterocyclyl and heteroaryl wherein each alkyl, alkenyl, alkynyl, cycloalkyl, aryl and heterocyclyl and groups containing them may be further optionally substituted. Optional substituents in the case of heterocycles containing N may also include but are not limited to C₁₋₆alkyl i.e. N—C₁₋₃alkyl, more preferably methyl particularly N-methyl.

For optionally substituted “C₁₋₆alkyl”, “C₂₋₆alkenyl” and “C₂₋₆alkynyl”, the optional substituent or substituents are preferably selected from halo, aryl, heterocyclyl, C₃₋₈cycloalkyl, C₁₋₆ alkoxy, hydroxyl, oxo, aryloxy, haloC₁₋₆alkyl, haloC₁₋₆alkoxyl and carboxyl. Each of these optional substituents may also be optionally substituted with any of the optional substituents referred to above, where nitro, amino, substituted amino, cyano, heterocyclyl (including non-aromatic heterocyclyl and heteroaryl), C₁₋₆alkyl, C₂₋₆alkenyl, C₂₋₆alkynyl, C₁₋₆alkoxyl, haloC₁₋₆ alkyl, haloC₁₋₆alkoxy, halo, hydroxyl and carboxyl are preferred.

It will be understood that suitable derivatives of aromatic heterocyclyls containing nitrogen include N-oxides thereof.

In the case of hybrid naming of substituent radicals, such as haloalkyl and alkylaryl, it is to be understood that no direction in the order of groups is intended so the point of attachment may be anywhere within the defined groups. For example, the terms “alkylaryl” and “arylalkyl” are intended to refer to the same group and the point of attachment may be via the alkyl or the aryl moiety (or both in the case of diradical species). The point of attachment of a hybrid named substituent radical may be denoted by “-”, for example, the term “-alkylaryl” indicates that the group is attached to rest of the molecule through the alkyl moiety, and similarly, “alkylaryl-” indicates that the group is attached to the rest of the molecule through the aryl moiety.

As used herein, except where the context requires otherwise, the term “comprise” and variations of the term, such as “comprising”, “comprises” and “comprised”, are not intended to exclude further additives, components, integers or steps.

It must be noted that as used herein and in the appended claims, the singular forms “a”, “an” and “the” include plural reference unless the context clearly dictates otherwise. Thus, for example, a reference to “a symptom” and/or “at least one symptom” may include one or more symptoms, and so forth.

The term “and/or” can mean “and” or “or”.

The term “(s)” following a noun contemplates the singular or plural form, or both.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±25%, 10%, 5%, 1% or ±0.1% of that value.

Further aspects of the present invention and further embodiments of the aspects described in the preceding paragraphs will become apparent from the following description, given by way of example and with reference to the accompanying drawings.

### DETAILED DESCRIPTION OF EMBODIMENTS

The invention provides a compound of Formula (I)

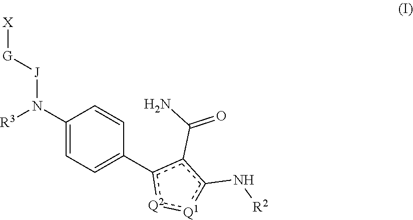

wherein
Q¹ and Q² are selected from N and NR¹, wherein when Q² is N, Q¹ is NR¹ and when Q¹ is N, Q² is NR¹;
R¹ and R³ are independently selected from H and an optionally substituted C₁₋₆-alkyl;
R² is H, an optionally substituted C₁-C₆-alkyl, an optionally substituted aryl or an optionally substituted heterocyclyl;
X is selected from optionally substituted C₁₋₆alkyl, optionally substituted haloC₁₋₆alkyl, optionally substituted —C₁₋₆alkylamino, optionally substituted C₂₋₆alkynyl, optionally substituted cycloalkyl, optionally substituted halocycloalkyl, optionally substituted aryl, optionally substituted alkylaryl, optionally substituted heterocyclyl, optionally substituted C₁₋₆ alkylheterocyclyl;
J is selected from carbonyl and

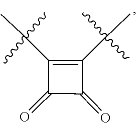 [SMILES: *c1c(*)c(=O)c1=O]

and
G is selected from a single bond, NR³, CR⁴R⁵ and optionally substituted heterocyclyl,
R⁴ and R⁵ are independently selected from H, optionally substituted C₁₋₆alkyl, optionally substituted aryl and optionally substituted amino;
or a pharmaceutically acceptable salt, solvate, tautomer, stereoisomer, N-oxide and/or prodrug thereof.

It will be appreciated that [symbol] denotes a single or a double bond. For example, the 5-membered heterocyclyl depicted in formula (I) with [symbol] is a pyrazole that may adopt one of two isomeric forms.

In some embodiments, Q² is N and Q¹ is NR¹. In these embodiments, the compound of formula (I) may be provided by a compound of formula (IA):

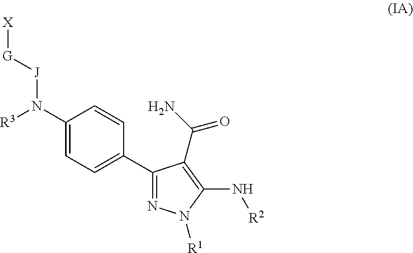

wherein X, G, J, R¹, R² and R³ are as defined for formula (I).

In some embodiments, Q¹ is N and Q² is NR¹. In these embodiments, the compound of formula (I) may be provided by a compound of formula (IB):

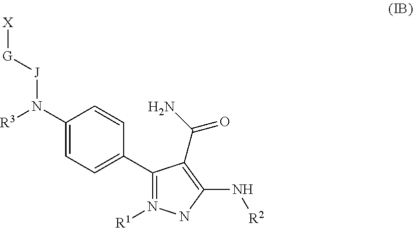

wherein X, G, J, R¹, R² and R³ are as defined for formula (I).

In some embodiments, X is selected from C₁₋₆alkyl, C₂₋₆alkynyl, C₃₋₆cycloalkyl, aryl, —C₁₋₂alkylaryl, —C₁₋₂alkylcycloalkyl, heterocyclyl, —C₁₋₂alkylheterocyclyl; wherein each alkyl and alkynyl is optionally substituted with one or more groups selected from halo, nitrile, aryl, R⁶, —OR⁶, —N(R⁷)R⁸;

R⁶, R⁷ and R⁸ are independently selected from H, aryl, C₁₋₆alkyl and haloC₁₋₆alkyl, and

wherein each aryl, heterocyclyl and cycloalkyl is optionally substituted with one or more groups that are independently selected from halo, nitrile, C₁₋₄alkyl, C₁₋₄alkoxy, haloC₁₋₄alkyl and haloC₁₋₄alkoxy.

In some embodiments, X is a bicyclic fused heterocyclyl or a spirocyclic heterocyclyl selected from any one of partial formulas X1-X3:

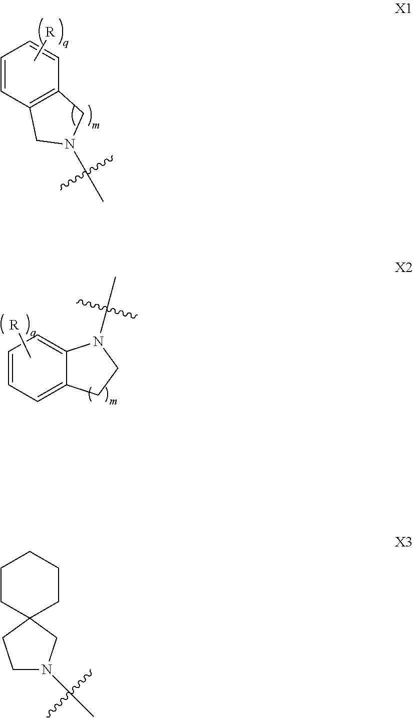

wherein R is selected from C₁₋₄alkyl, haloC₁₋₄alkyl, OC₁₋₄alkyl, OhaloC₁₋₄alkyl, and halo;
q is 0, 1 or 2; and
m is 1 or 2. When m is 2 in either of the partial formulas X1 and X2, the heterocyclyl is a 6,6-fused bicyclic system.

In some embodiments, R is trifluoromethyl.

In some embodiments, X is an optionally substituted heteroaryl, which may be selected from optionally substituted pyridyl, optionally substituted oxazolyl, optionally substituted diazolyl.

Preferred substituents include C₁₋₄alkyl and haloC₁₋₄alkyl.

In some embodiments, X is an optionally substituted —C₁₋₆alkylamino and G is a covalent bond.

In some embodiments, X is selected from any one of the following groups:

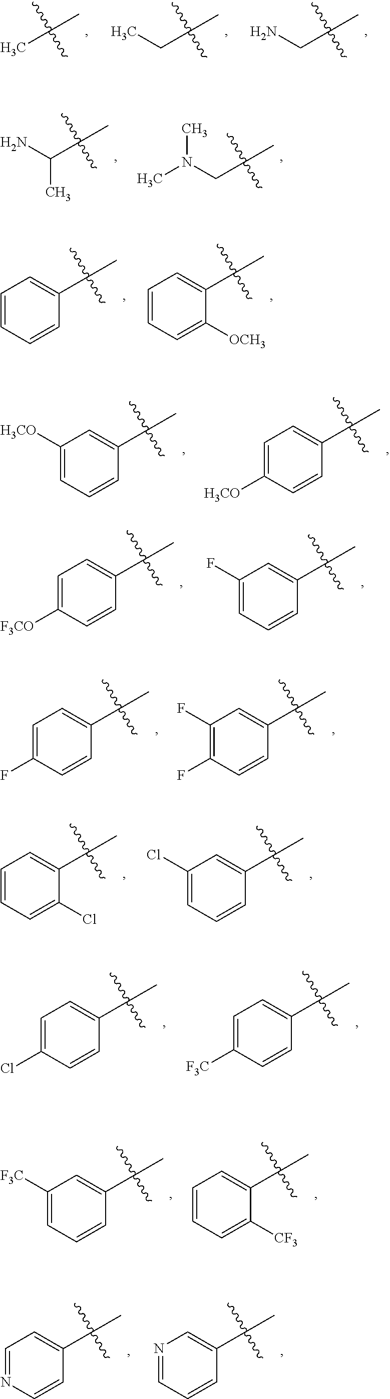

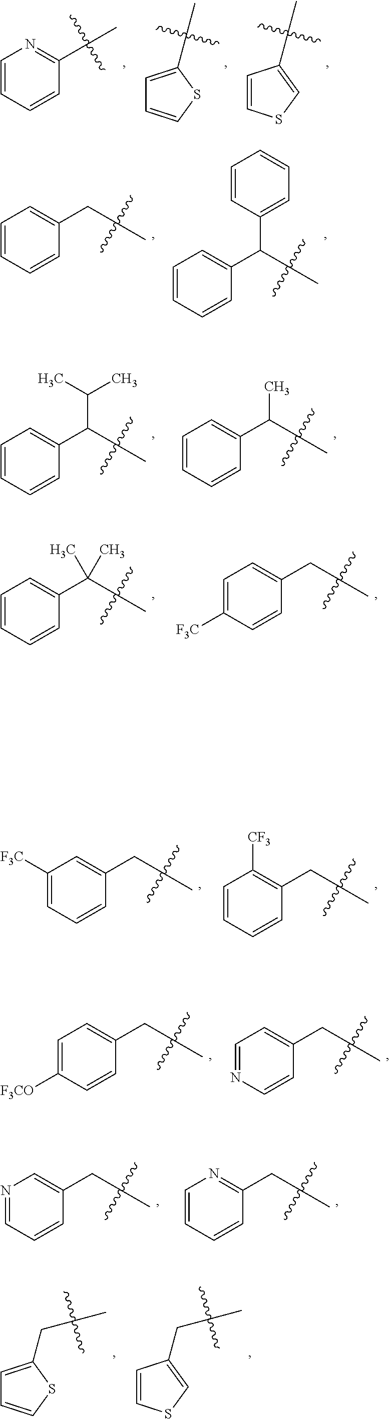

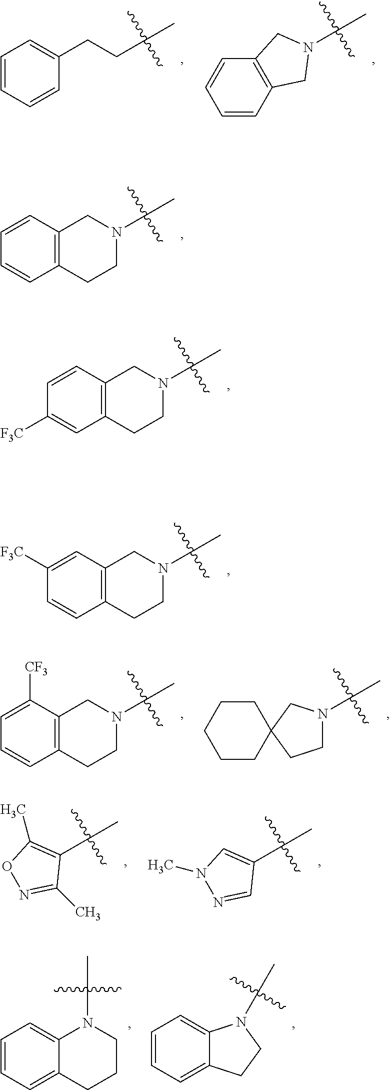

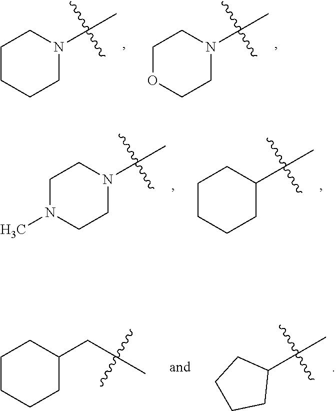

In some embodiments, R¹ and R³ are independently selected from H and optionally substituted C₁₋₄alkyl.

In some embodiments, each instance of R¹ and R³ is H.

In some embodiments, R² is represented by any one of partial formulas Ar1-Ar3:

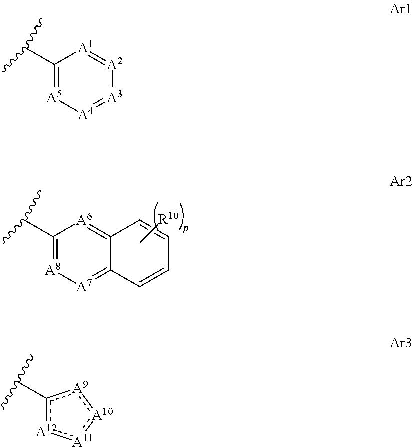

wherein
A¹, A², A³, A⁴, A⁵, A⁶, A⁷, A⁸, A⁹ and A¹⁰ are independently selected from CR¹ and N
wherein 0, 1 or 2 of A¹, A², A³, A⁴ and A⁵ are N
wherein 0, 1 or 2 of A⁶, A⁷ and A⁸ are N
A⁹, A¹⁰, A¹¹ and A¹² are independently selected from C(R¹¹)_{q}, O, S, N and NR¹²;
wherein at least 1 of A⁹, A¹⁰, A¹¹ and A¹² is selected from C(R¹¹)_{q}, O, S and NR¹²;
each R¹¹ is independently selected from H and R¹⁰;
each R¹⁰ is independently selected from halo, C₁₋₆alkyl, C₁₋₆alkoxy, C₃₋₁₀cycloalkyl, —OC₁₋₆alkylC₁₋₄alkoxy, haloC₁₋₆alkyl, haloC₁₋₆alkoxy, nitrile, amido, C₁₋₆alkylamido, (C₁₋₆alkyl)₂amido, haloC₁₋₆alkylamido, (haloC₁₋₆alkyl)₂amido, acyl, C₁₋₆alkylacyl, haloC₁₋₆ alkylacyl, arylacyl, heterocyclylacyl, C₃₋₁₀cycloalkylacyl, heterocyclyl, haloC₁₋₆alkoxy, C₃₋₁₀cycloalkyl, C₁₋₆alkylC₃₋₁₀cycloalkyl, C₁₋₆alkoxyC₃₋₁₀cycloalkyl, haloC₁₋₆alkylC₃₋₁₀cycloalkyl, haloC₁₋₆alkoxyC₃₋₁₀cycloalkyl, C₁₋₆alkylheterocyclyl, C₁₋₆alkoxyheterocyclyl, haloC₁₋₆alkylheterocyclyl, haloC₁₋₆alkoxyheterocyclyl, C₁₋₆alkylC₁₋₆ alkoxy, and —COOH;
each R¹² is independently selected from H, C₁₋₆alkyl, haloC₁₋₄alkyl, C₁₋₆alkylacyl and haloC₁₋₆alkylacyl;
or when two adjacent groups selected from A¹, A², A³, A⁴, A⁵, A⁷, A⁸ A⁹, A¹⁰, A¹¹ and A¹² (e.g. A¹ and A², A² and A³, A³ and A⁴, A⁴ and A⁵, A⁸ and A⁷, A⁹ and A¹⁰, A¹⁰ and A¹¹, A¹¹ and A¹²) are selected from CR¹ and NR¹², two R¹¹, two R¹² or one R¹¹ and one R¹² may together form an optionally substituted 5-10 membered ring selected from cycloalkyl, aryl and heterocyclyl;
p is an integer from 0 to 4; and
q is 1 or 2.

In some embodiments, R² is represented by partial formula Ar1.

In some embodiments, A¹ is N.

In some embodiments, A³ is N.

In some embodiments, A⁴ is N.

In some embodiments, A² is CR¹⁰.

In some embodiments, the compound of formula (I) is a compound of formula (II)

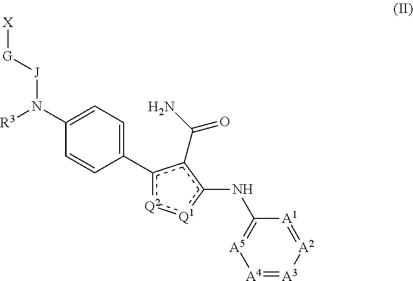

wherein Q¹, Q², X, G, J and R³ are as defined in formula (I) and A¹-A⁵ are as defined for partial formula Ar1.

In some embodiments, R² is represented by partial formula Ar3.

In some embodiments, A¹⁰ is NR¹² and A¹² is CR¹¹.

In some embodiments, A⁹ and A¹¹ may be independently selected from CR¹¹, N, O and S. In some embodiments, when A⁹ is CR¹¹, A¹¹ is N, O or S and when A⁹ is N, O or S, A¹¹ is CR¹¹.

In some embodiments, A⁹ and A¹¹ are each CR¹¹.

In some embodiments, A¹⁰ and A¹² are each CR¹¹.

In some embodiments, at least one of A⁹, A¹⁰, A¹¹ and A¹² is selected from O, S, N and NR¹².

In some embodiments, one of A⁹, A¹⁰, A¹¹ and A¹² is selected from O, S and NR¹².

In some embodiments, partial formula Ar3 is provided by any one of the partial formulas Ar3-I, Ar3-II, Ar3-III and Ar3-IV

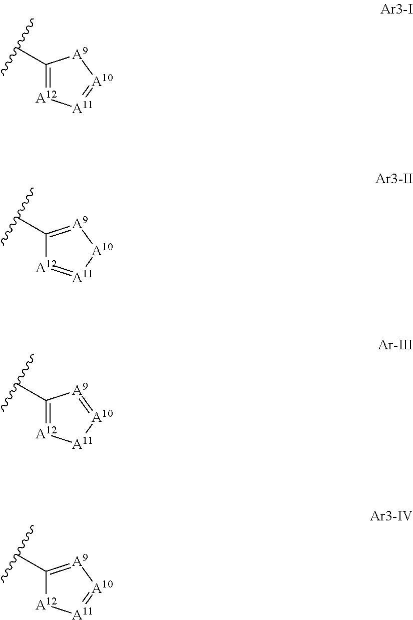

wherein
in Ar3-I, A⁹ is selected from C(R¹¹)₂, O, S and NR¹², preferably O, S and NR¹²;
in Ar3-II, A¹⁰ is selected from C(R¹¹)₂, O, S and NR¹², preferably O, S and NR¹²;
in Ar3-III, A¹¹ is selected from C(R¹¹)₂, O, S and NR¹², preferably O, S and NR¹²; and
in Ar3-IV, A¹² is selected from C(R¹¹)₂, O, S and NR¹², preferably O, S and NR¹².

In some embodiments, A¹⁰ and A¹¹ are independently selected from CR¹¹ and NR¹² such that two R¹¹, two R¹² or R¹¹ and R¹² together form a 5-10 membered cycloalkyl, aryl or heterocyclyl ring.

In some embodiments, A¹⁰ is CR¹¹ and A¹¹ is NR¹², and R¹¹ and R¹² together form a 5-10 membered cycloalkyl, aryl or heterocyclyl ring. In these embodiments, A¹² may be N and/or A⁹ may be CR¹¹. In some embodiments, when A¹⁰ is CR¹¹ and A¹¹ is NR¹², and R¹¹ and R¹² together form a 5-10 membered heterocyclyl ring, preferably a non-aromatic heterocyclyl ring. In some embodiments, when A¹⁰ is CR¹¹ and A¹¹ is NR¹², R¹¹ and R¹² together form a 5-8 membered cycloalkyl, aryl or heterocyclyl ring, preferably a 6 or 7 membered ring, more preferably a 6 or 7 membered heterocyclyl ring.

When two R¹¹, two R¹² or one R¹¹ and R¹² on adjacent ring atoms form a fused ring, the fused ring may be optionally substituted by 1-3 R¹⁰ groups. Any R¹⁰ group described herein may be suitable.

In some embodiments, R² is represented by any one of partial formula Ar3′:

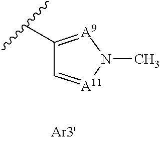

wherein
A⁹ and A¹¹ are independently selected from CR¹¹ and N,
wherein when A¹¹ is N, A⁹ is CR¹¹ and when A⁹ is N, A¹¹ is CR¹¹. R¹¹ may be as defined for any embodiment herein.

In some embodiments, each R¹⁰ is independently selected from halo, C₁₋₄alkyl, C₁₋₆ alkoxy, —OC₁₋₄alkylC₁₋₄alkoxy, haloC₁₋₄alkyl, haloC₁₋₄alkoxy, optionally substituted amido, nitrile, heterocyclyl and haloC₁₋₆alkoxy.

In some embodiments, R¹⁰ is an optionally substituted amido selected from —C(O)NR¹²R¹³ and —NR¹²C(O)R¹³, wherein R¹² and R¹³ are independently selected from H and optionally substituted C₁₋₄alkyl, preferably R¹³ is optionally substituted C₁₋₄alkyl.

In some embodiments, R¹⁰ is selected from fluoro, chloro, methyl, isopropyl, tert-butyl, difluoromethyl, trifluoromethyl, methoxy, ethoxy, difluoroethoxy, nitrile, amido, trifluoromethoxy, —OCH₂CH₂OCH₃, cyclopropyl and morpholino.

In some embodiments, the compound comprises not more than 1, 2, 3 or 4 instances of R¹⁰.

In some embodiments, the compound comprises not more than 1 or 2 instances of R¹⁰.

In some embodiments, p is 0, 1 or 2.

In some embodiments, p is 0 or 1.

In some embodiments, p is 0.

In some embodiments, R² is selected from any one of the following radicals:

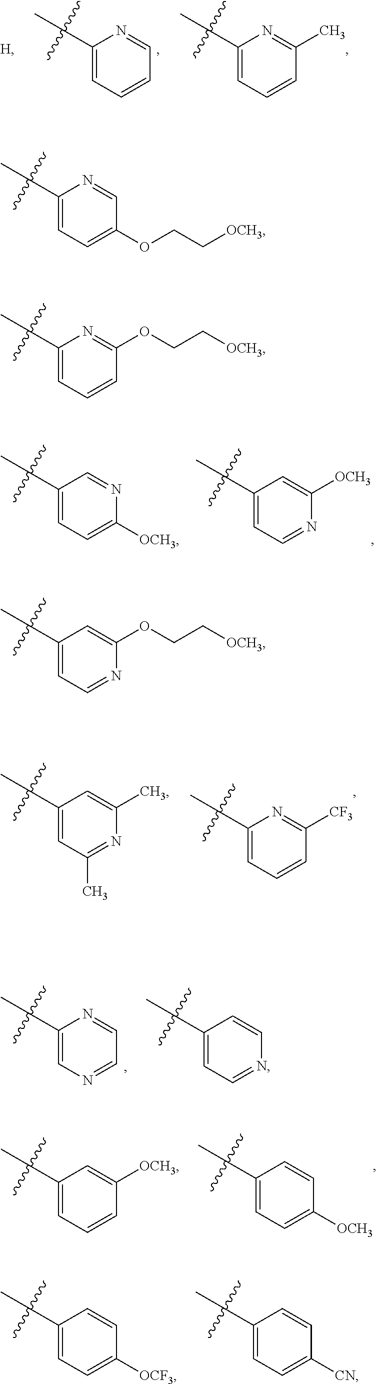

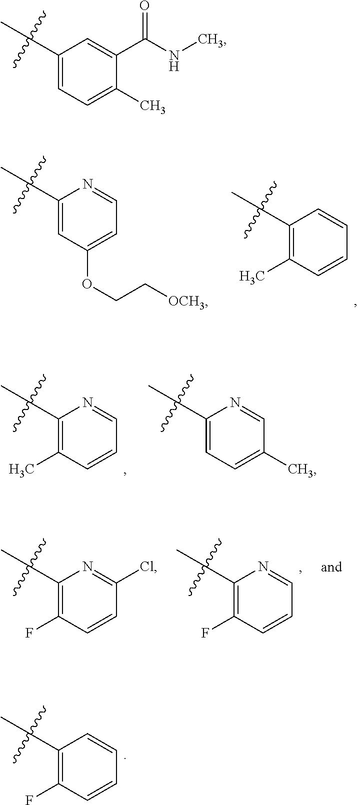

In some embodiments, G is a single bond.

In some embodiments, G is CR⁴R⁵. In some embodiments, R⁴ and R⁵ are independently selected from H, C₁₋₆alkyl, aryl and amino, wherein the alkyl may be substituted with one or more groups selected from halo, cycloalkyl, hetereocyclyl and aryl, the aryl may be substituted with one or more substitutents selected from halo, C₁₋₄alkyl, haloC₁₋₄alkyl, C₁₋₄alkoxyl and haloC₁₋₄alkoxyl and the amino may be substituted with a C₁₋₄alkyl.

In some embodiments, G is NR³. In some embodiments, R³ is selected from H and methyl.

In some embodiments, G is an optionally substituted heterocyclyl. In some embodiments, the optionally substituted heterocyclyl is a 4, 5 or 6-membered heterocyclyl. In some embodiments, the heterocyclyl comprises 1 or 2 N ring atoms. The heterocyclyl may be aromatic or non-aromatic, with 4, 5 and 6 membered non-aromatic and 5-membered heteroaromatics being preferred. Preferred substitutents for the hetereocyclyl groups include methyl and halo.

In some embodiments, G is an optionally substituted hetereocyclyl selected from:

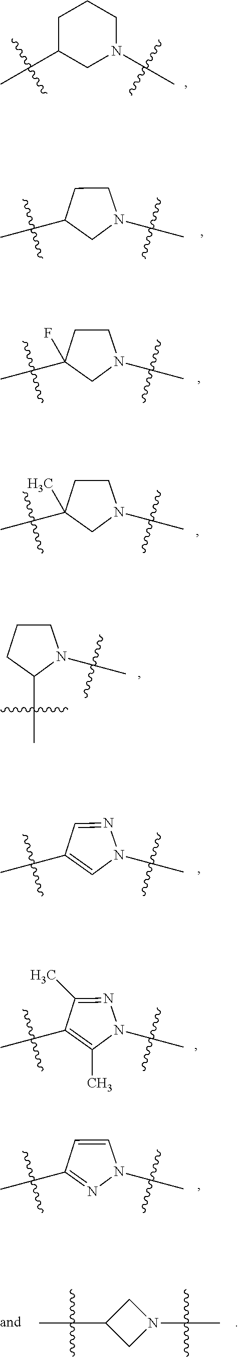

In some embodiments, J is carbonyl.

In some embodiments, J is

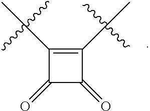 [SMILES: *c1c(*)c(=O)c1=O]

In some embodiments, J is carbonyl and G is NR³. In these embodiments, the compounds of the invention are substituted ureas.

In some embodiments, J is carbonyl and G is selected from a single bond, CR⁴R⁵ or an optionally substituted heterocyclyl.

Typically, the compounds of the invention may be prepared by techniques known in the art.

In another aspect, there is also provided a process for preparing a compound of formula (I) or a salt, solvate, tautomer, N-oxide, stereoisomer and/or prodrug thereof. In some embodiments, the process comprises any of the following steps:
converting a compound of formula (III) into a corresponding carbonate at the free anilino-nitrogen atom, followed by reaction with a compound of formula (IV), optionally followed by deprotection

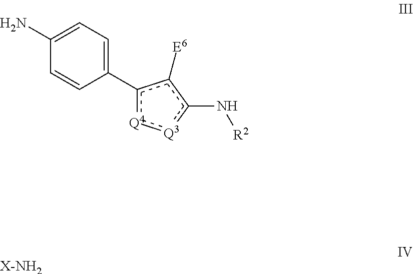

wherein X and R² are as defined for formula (I)
Q³ and Q⁴ are selected from N and N-PG¹, wherein when Q⁴ is N, Q³ is N-PG¹, and when Q³ is N, Q⁴ is N-PG¹;
PG¹ is R¹ or an amino protecting group, such as tert-butyl, benzyl, BOC and the like, and R¹ is as defined for formula (I); and
E⁶ is selected from —CN and —C(O)NH₂.
reacting a compound of formula (III) with a compound of formula V

X—N═C═O (V)
wherein X is as defined in formula (I)
reacting a compound of formula (III) with a compound of formula (VI)

X-G-CO₂H (VI)
wherein X and G are as defined in formula (I);
reacting a compound of formula (III) with a compound of formula (VII)

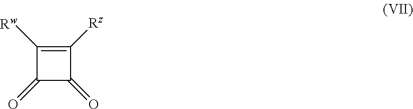

wherein R^{z} is C₁₋₄alkoxy and R^{w} is selected from C₁₋₄alkoxy and X-G-, wherein X and G are as defined in formula (I); optionally followed by a deprotection step;
converting a compound of formula (I) into one of its salts.

In some embodiments of the above process, wherein PG¹ is an amino protecting group, the process further comprises a deprotection step.

Embodiments of these steps are shown in Schemes 1-5 below with reference to compounds wherein R² is represented by partial formula Ar1.

The specific reagents and conditions for effecting each of these steps will depend on the specific substituents selected for each reaction partner. The skilled person would readily appreciate how to determine and/or optimise these reagents and conditions. Similarly, where a starting material is not commercially available, the skilled person would be able to design and implement its preparation based on techniques and reactions previously described. Embodiments of these steps are provided in the Examples with reference to specific compounds described herein.

### Methods

In another aspect, there is provided a method for inhibiting necroptosis in a subject in need thereof, the method comprising administering a therapeutically effective amount of a compound according to Formula (I) or a pharmaceutically acceptable salt, solvate, tautomer, stereoisomer, N-oxide and/or prodrug thereof.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

RIP1, RIP3 and MLKL are three proteins implicated in the necroptotic pathway. Upon necroptotic stimulus (e.g. using the combination of TNF, SMAC mimetic and QVD-OPh on suitable cell lines), RIP1 is auto-phosphorylated leading to association with RIP3, which in turn auto-phosphorylates itself. Activated RIP3 phosphorylates MLKL leading to a putative conformational change that triggers its necroptotic activity (Murphy, Immunity, 39, pp 443-453, 2013). MLKL acts downstream of RIP1 and RIP3. Compounds of this invention may bind to RIP1. Compounds of this invention may bind to MLKL. Some preferred compounds of this invention may bind to RIP1 and/or MLKL.

In some embodiments, administration of a compound according to Formula (I) modulates RIP1 activity. RIP1 is believed to be the switch between apoptosis and necroptosis. It has been shown that, in cases where apoptosis is inhibited (for example using a pan caspase inhibitor such as QVD-OPh), RIP1 mediates a necroptotic response. Moreover, it has also been shown that small molecule inhibitors of RIP1 can potently inhibit necroptosis (see for example Degterev et al, Nat Chem Biol, pp 112-119, 2005). Therefore, compounds of the invention may inhibit necroptosis by inhibiting RIP1.

In some embodiments, administration of a compound according to Formula (I) inhibits a conformational change of MLKL. In another embodiment, the conformational change of MLKL involves release of the four-helix bundle (4HB) domain of MLKL. In another embodiment, administration of the compound inhibits oligomerisation of MLKL. In yet another embodiment, administration of the compound inhibits translocation of MLKL to the cell membrane. In a further embodiment, administration of the compound inhibits a conformational change of MLKL, inhibits oligomerisation of MLKL and inhibits translocation of MLKL to the cell membrane.

It is envisaged that some compounds of the present disclosure can bind to MLKL in various species and inhibit necroptosis.

As used herein, the term “pseudokinase domain” as understood by a person skilled in the art, means a protein containing a catalytically-inactive or catalytically-defective kinase domain. “Pseudokinase domains” are often referred to as “protein kinase-like domains” as these domains lack conserved residues known to catalyse phosphoryl transfer. It would be understood by a person skilled in the art that although pseudokinase domains are predicted to function principally as catalysis independent protein-interaction modules, several pseudokinase domains have been attributed unexpected catalytic functions. Accordingly, in the present disclosure the term “pseudokinase domain” includes “pseudokinase domains” which lack kinase activity and “pseudokinase domains” which possess weak kinase activity.

As used herein, the term “ATP-binding site” as understood by a person skilled in the art, means a specific sequence of protein subunits that promotes the attachment of ATP to a target protein. An ATP binding site is a protein micro-environment where ATP is captured and hydrolyzed to ADP, thereby releasing energy that is utilized by the protein to work by changing the protein shape and/or making the enzyme catalytically active. In pseudokinase domains, the “ATP-binding site” is often referred to as the “pseudoactive site”. The term “ATP-binding site” may also be referred to as a “nucleotide-binding site” as binding at this site includes the binding of nucleotides other than ATP. It would be understood by a person skilled in the art that the term “nucleotide” includes any nucleotide. Exemplary nucleotides include, but are not limited to, AMP, ADP, ATP, AMPPNP, GTP, CTP and UTP. In some embodiments, the compounds of the invention may bind to the ATP-binding site of MLKL.

As described herein, inhibition of necroptosis includes both complete and partial inhibition of necroptosis. In one embodiment, inhibition of necroptosis is complete inhibition. In another embodiment, inhibition of necroptosis is partial inhibition.

Binding of the compound to the ATP-binding site of the pseudokinase domain of MLKL may inhibit phosphorylation of MLKL by an effector kinase or binding of the compound to the ATP-binding site of the pseudokinase domain of MLKL may not inhibit phosphorylation of MLKL by an effector kinase. The present disclosure demonstrates that compounds that bind to the ATP-binding site of the pseudokinase domain of the MLKL protein, as described herein, can inhibit necroptosis without inhibiting phosphorylation of MLKL by an effector kinase. In one embodiment, binding of the compound to the ATP-binding site of the pseudokinase domain of MLKL does not inhibit phosphorylation of MLKL by an effector kinase. In another embodiment, binding of the compound to the ATP-binding site of the pseudokinase domain of MLKL inhibits phosphorylation of MLKL by an effector kinase. Compounds that can simultaneously inhibit RIP1 auto-phosphorylation and MLKL activation may represent very powerful inhibitors of necroptosis due to the fact that they interfere with two key components of the pathway.

The compounds of the invention may be selective for RIP1 and/or MLKL. In some embodiments, the compounds of the invention may be selective for RIP1 over RIP3. In some embodiments, the compounds of the invention may be selective for MLKL over RIP3. In some embodiments, the compounds of the invention are selective for both RIP1 and MLKL over RIP3. A selective compound may have 5-fold, 10-fold, 50-fold, 100-fold, 500-fold, 1000-fold or greater selectivity for MLKL and/or RIP1 compared to RIP3 or other screening kinase or pseudokinase. Typically, the relative selectivity may be assessed by comparing K_{D} values for each respective compound binding to the relevant protein (ie MLKL and either or both of RIP1 and RIP3). Suitable assay conditions are described in the Examples below. Compounds selective for MLKL and/or RIP1 may avoid undesired side-effects associated with RIP3 loss of function.

In another aspect, there is provided use of a compound of Formula (I) a pharmaceutically acceptable salt, solvate, tautomer, stereoisomer, N-oxide and/or prodrug thereof in the preparation of a medicament for the inhibition of necroptosis in a subject.

In another aspect, there is provided use of a composition comprising a compound of Formula (I) or a pharmaceutically acceptable salt, solvate, tautomer, stereoisomer, N-oxide and/or prodrug thereof for the inhibition of necroptosis in a subject.

In another aspect, there is provided a compound of Formula (I) or a pharmaceutically acceptable salt, solvate, tautomer, stereoisomer, N-oxide and/or prodrug thereof for use as a medicament.

In another aspect, there is provided use of a compound of Formula (I) or a pharmaceutically acceptable salt, solvate, tautomer, stereoisomer, N-oxide and/or prodrug thereof for inhibiting necroptosis.

In another aspect, there is provided use of a composition comprising a compound of Formula (I) or a salt, solvate, or prodrug thereof for inhibiting necroptosis.

In yet another aspect, there is provided a compound according to Formula (I) or a salt, solvate, prodrug or polymorph thereof for use in inhibiting necroptosis.

In yet another aspect, there is provided a composition comprising a compound according to Formula (I) or a pharmaceutically acceptable salt, solvate, tautomer, stereoisomer, N-oxide and/or prodrug thereof for use in inhibiting necroptosis. In some embodiments, the composition is a pharmaceutical composition.

In yet another aspect, there is provided a compound according to Formula (I) or a pharmaceutically acceptable salt, solvate, tautomer, stereoisomer, N-oxide and/or prodrug thereof when used for inhibiting necroptosis.

In yet another aspect, there is provided a composition comprising a compound according to Formula (I) or a pharmaceutically acceptable salt, solvate, tautomer, stereoisomer, N-oxide and/or prodrug thereof when used for inhibiting necroptosis.

In another aspect, there is provided a method of inhibiting RIP1 and/or MLKL, comprising contacting a cell with an effective amount of a compound of formula (I) or a salt, solvate, tautomer, stereoisomer and/or prodrug thereof.

The salts of the compounds of Formula (I) are preferably pharmaceutically acceptable, but it will be appreciated that non-pharmaceutically acceptable salts also fall within the scope of the present disclosure, since these may be useful as intermediates in the preparation of pharmaceutically acceptable salts.

The term “pharmaceutically acceptable” may be used to describe any salt, solvate, tautomer, stereoisomer and/or prodrug thereof, or any other compound which upon administration to a subject, is capable of providing (directly or indirectly) a compound of Formula (I) or an active metabolite or residue thereof.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

Formula (I) is intended to cover, where applicable, solvated as well as unsolvated forms of the compounds. Thus, Formula (I) includes compounds having the indicated structures, including the hydrated or solvated forms, as well as the non-hydrated and non-solvated forms.

The compounds of Formula (I) or salts, tautomers, N-oxides, polymorphs or prodrugs thereof may be provided in the form of solvates. Solvates contain either stoichiometric or non-stoichiometric amounts of a solvent, and may be formed during the process of crystallization with pharmaceutically acceptable solvents such as water, alcohols such as methanol, ethanol or isopropyl alcohol, DMSO, acetonitrile, dimethyl formamide (DMF), acetic acid, and the like with the solvate forming part of the crystal lattice by either non-covalent binding or by occupying a hole in the crystal lattice. Hydrates are formed when the solvent is water, alcoholates are formed when the solvent is alcohol. Solvates of the compounds of the present invention can be conveniently prepared or formed during the processes described herein. In general, the solvated forms are considered equivalent to the unsolvated forms for the purposes of the compounds and methods provided herein.

Basic nitrogen-containing groups may be quarternised with such agents as C₁₋₆alkyl halide, such as methyl, ethyl, propyl, and butyl chlorides, bromides and iodides; dialkyl sulfates like dimethyl and diethyl sulfate; and others.

Nitrogen containing groups may also be oxidised to form an N-oxide.

The compound of Formula (I) or salts, tautomers, N-oxides, solvates and/or prodrugs thereof that form crystalline solids may demonstrate polymorphism. All polymorphic forms of the compounds, salts, tautomers, N-oxides, solvates and/or prodrugs are within the scope of this invention and may be used in the methods of the invention.

The compound of Formula (I) may demonstrate tautomerism. Tautomers are two interchangeable forms of a molecule that typically exist within an equilibrium. Any tautomers of the compounds of Formula (I) are to be understood as being within the scope of the invention and may be used in the methods of the invention. For example, when R¹ is H the compounds of formula (1A) and (1B) may exist as tautomers, eg in equilibrium with each other. The proportion of compounds of formula (1A) to (1B) in equilibrium may depend on the specific compound and conditions, such as solvent, temperature, concentration, etc. This equilibrium may be described as follows:

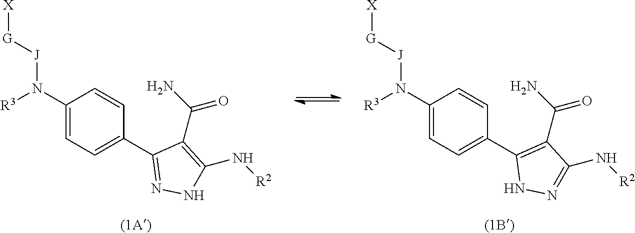

Similar tautomerism may occur for any pyrazole-containing compound described herein, including compounds of formula (II) and (Ill), various intermediate compounds (such as those depicted in Schemes 1-5) and compounds 1-130. All tautomers of these compounds are contemplated and considered within the scope of the present invention. In addition, further tautomeric forms may exist for the compounds described herein for example depending on various substituents selected.

The compound of Formula (I) may contain one or more stereocentres. All steoisomers of the compounds of formula (I) are within the scope of the invention. Stereoisomers include enantiomers, diastereomers, geometric isomers (E and Z olephinic forms and cis and trans substitution patterns) and atropisomers. In some embodiments, the compound is a stereoisomerically enriched form of the compound of formula (I) at any stereocentre. The compound may be enriched in one stereoisomer over another by about 60, 70, 80, 90, 95, 98 or 99%.

The compound of Formula (I) or its salts, tautomers, solvates, N-oxides, polymorphs and/or stereoisomers, may be isotopically enriched with one or more of the isotopes of the atoms present in the compound. For example, the compound may be enriched with one or more of the following minor isotopes: ²H, ³H, ¹³C, ¹⁴C, ¹⁵N and/or ¹⁷O. An isotope may be considered enriched when its abundance is greater than its natural abundance.

A “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of Formula (I). The amino acid residues include the naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula (I) through the carbonyl carbon prodrug sidechain.

Pharmaceutical compositions may be formulated from compounds according to Formula (I) for any appropriate route of administration including, for example, oral, rectal, nasal, vaginal, topical (including transdermal, buccal, ocular and sublingual), parenteral (including subcutaneous, intraperitoneal, intradermal, intravascular (for example, intravenous), intramuscular, spinal, intracranial, intrathecal, intraocular, periocular, intraorbital, intrasynovial and intraperitoneal injection, intracisternal injection as well as any other similar injection or infusion techniques), inhalation, insufflation, infusion or implantation techniques (e.g., as sterile injectable aqueous or non-aqueous solutions or suspensions).

In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile.

The formulations may be present in unit or multi-dose containers such as sealed ampoules or vials. Examples of components are described in Martindale—The Extra Pharmacopoeia (Pharmaceutical Press, London 1993), and Remington: The Science and Practice of Pharmacy, 21st Ed., 2005, Lippincott Williams & Wilkins. All methods include the step of bringing the active ingredient, for example a compound defined by Formula (I), or a pharmaceutically acceptable salt, solvate, tautomer, stereoisomer, N-oxide and/or prodrug thereof, into association with the carrier which constitutes one or more accessory ingredients. In general, the pharmaceutical compositions are prepared by uniformly and intimately bringing the active ingredient, for example a compound defined by Formula (I), or a pharmaceutically acceptable salt, solvate, tautomer, stereoisomer, N-oxide and/or prodrug thereof, into association with a liquid carrier or a finely divided solid carrier or both, and then, if necessary, shaping the product into the desired formulation. In the pharmaceutical composition the active object compound is included in an amount sufficient to produce the desired effect. In some embodiments, the method of the invention comprises administering a pharmaceutical comprising a compound of Formula (I) or a pharmaceutically acceptable salt, solvate, tautomer, stereoisomer, N-oxide and/or prodrug thereof and a pharmaceutically acceptable carrier, diluent and/or excipient.

In the context of this specification the term “administering” and variations of that term including “administer” and “administration”, includes contacting, applying, delivering or providing a compound or composition of the invention to an organism, or a surface by any appropriate means.

For the inhibition of necroptosis, the dose of the biologically active compound according to the invention may vary within wide limits and may be adjusted to individual requirements. Active compounds according to the present invention are generally administered in a therapeutically effective amount. The daily dose may be administered as a single dose or in a plurality of doses. The amount of active ingredient that may be combined with the carrier materials to produce a single dosage form will vary depending upon the subject treated and the particular mode of administration.

It will be understood, however, that the specific dose level for any particular subject will depend upon a variety of factors including the activity of the specific compound employed, the age, body weight, general health, sex and diet of the subject, time of administration, route of administration, and rate of excretion, drug combination (i.e. other drugs being used to treat the subject), and the severity of the particular disorder undergoing therapy. Such treatments may be administered as often as necessary and for the period of time judged necessary by the treating physician. A person skilled in the art will appreciate that the dosage regime or therapeutically effective amount of the compound of formula (I) to be administered may need to be optimized for each individual.

It will also be appreciated that different dosages may be required for treating different disorders. An effective amount of an agent is that amount which causes a statistically significant decrease in necroptosis.

For in vitro analysis, the necroptosis inhibition may be determined by assays used to measure TSQ-induced necroptosis, as described in the biological tests defined herein.

The terms “treating”, “treatment” and “therapy” are used herein to refer to curative therapy, prophylactic therapy and preventative therapy. Thus, in the context of the present disclosure the term “treating” encompasses curing, ameliorating or tempering the severity of necroptosis and/or associated diseases or their symptoms.

“Preventing” or “prevention” means preventing the occurrence of the necroptosis or tempering the severity of the necroptosis if it develops subsequent to the administration of the compounds or pharmaceutical compositions of the present invention.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

The term “inhibit” is used to describe any form of inhibition that results in prevention, reduction or otherwise amelioration of necroptosis, and/or RIP1 and/or MLKL function. The term “inhibit” includes complete and partial inhibition, eg a complete or partial reduction or otherwise amelioration of necroptosis, and/or RIP1 and/or MLKL function.

The compounds of the present invention may be administered along with a pharmaceutical carrier, diluent and/or excipient as described above.

The methods of the present disclosure can be used to prevent or treat the following disease(s), condition(s) and/or disorder(s) in a subject:
diseases of the bones, joints, connective tissue and of cartilage, such as osteoporosis, osteomyelitis including chronic recurrent multifocal osteomyelitis, arthritises including for example osteoarthritis, rheumatoid arthritis and psoriatic arthritis, avascular necrosis, progressive fibrodysplasia ossificans, rickets, Cushing's syndrome;
muscular diseases such as muscular dystrophy, such as for example Duchenne's muscular dystrophy, myotonic dystrophies, myopathies and myasthenias;
diseases of the skin, such as dermatitis, eczema, psoriasis, aging or even alterations of scarring;
cardiovascular diseases such as cardiac and/or vascular ischemia, myocardial infarction, ischemic cardiopathy, chronic or acute congestive heart failure, cardiac dysrythmia, atrial fibrillation, ventricular fibrillation, paroxystic tachycardia, congestive heart failure, hypertrophic cardiopathy, anoxia, hypoxia, secondary effects due to therapies with anti-cancer agents;
circulatory diseases such as atherosclerosis, arterial scleroses and peripheral vascular diseases, strokes including cerebrovascular strokes, aneurisms;
haematological and vascular diseases such as: anemia, aplastic anemia, vascular amyloidosis, haemorrhages, drepanocytosis, red cell fragmentation syndrome, neutropenia, leukopenia, medullar aplasia, pantocytopenia, thrombocytopenia, haemophilia;
lung diseases including pneumonia, asthma; obstructive chronic diseases of the lungs such as for example chronic obstructive pulmonary disease (COPD), chronic bronchitis and emphysema;
diseases of the gastro-intestinal tract, such as ulcers; inflammatory bowel diseases (IBD), including Crohn's disease, ulcerative colitis;
diseases of the liver such as for example hepatitis particularly hepatitis of viral origin or having as causative agent other infectious agents, auto-immune hepatitis, fulminating hepatitis, inflammatory hepatitis, certain hereditary metabolic disorders, Wilson's disease, cirrhoses, non-alcoholic fatty liver disease (NAFLD) including non-alcoholic hepatic steatosis and/or non-alcoholic steatohepatitis (NASH), diseases of the liver due to toxins and to drugs such as drug-induced liver injury, ethanol (or alcohol)-induced liver disease;
diseases of the pancreas such as for example acute or chronic pancreatitis;
metabolic diseases such as diabetes, including diabetes mellitus, pre-diabetes and insipid diabetes; thyroiditis;
diseases of the kidneys such as acute renal disorders (such as acute kidney injury (AKI), including ischaemic reperfusion injury (IRI)) or glomerulonephritis;
viral and bacterial infections such as septicemia;
severe intoxications by chemicals, toxins or drugs;
degenerative diseases associated with the Acquired Immune Deficiency Syndrome (AIDS);
disorders associated with aging such as the syndrome of accelerated aging;
inflammatory diseases such as Terminal ileitis including Crohn's disease, rheumatoid polyarthritis, TNF-induced systemic inflammatory syndrome;
auto-immune diseases such as erythematous lupus (including systemic lupus erythematosus), cleavage-resistant RIPK1-induced autoinflammatory (CRIA) syndrome;
dental disorders such as those resulting in degradation of tissues such as for example periodontitis;
ophthalmic diseases or disorders including diabetic retinopathies, glaucoma, macular degenerations, retinal degeneration, retinitis pigmentosa, retinal holes or tears, retinal detachment, retinal ischemia, acute retinopathies associated with trauma, inflammatory degenerations, post-surgical complications, medicinal retinopathies, cataract, cone cell degeneration;
disorders of the audition tracts, such as otosclerosis and deafness induced by antibiotics;
Ischemic reperfusion injury, including retinal ischaemic reperfusion injury;
Neuronal loss, including Alzheimer's disease and neurodegenerative diseases such as amyotrophic lateral sclerosis (ALS; also referred to as motor neuron disease (MND) and Charcot disease);
diseases associated with mitochondria (mitochondrial pathologies), such as Friedrich's ataxia, congenital muscular dystrophy with structural mitochondrial abnormality, certain myopathies (MELAS syndrome, MERFF syndrome, Pearson's syndrome), MIDD (mitochondrial diabetes and deafness) syndrome, Wolfram's syndrome, dystonia;
cancer and metastasis including but not limited to cancers of the lung and bronchus, including non-small cell lung cancer (NSCLC), squamous lung cancer, brochioloalveolar carcinoma (BAC), adenocarcinoma of the lung, and small cell lung cancer (SCLC); prostate cancer, including androgen-dependent and androgen-independent prostate cancer; breast cancer, including metastatic breast cancer; pancreatic cancer; cancers of the colon and rectum; thyroid cancer; cancers of the liver and intrahepatic bile duct; hepatocellular cancer; gastric cancer; endometrial cancer; melanoma; cancers of the kidney, renal pelvis, urinary bladder, uterine corpus and uterine cervix; ovarian cancer, including progressive epithelial or primary peritoneal cancer; multiple myeloma; oesophageal cancer, including squamous cell carcinoma and adenocarcinoma of the oesophagus; acute myelogenous leukemia (AML); chronic myelogenous leukemia (CML), including accelerated CML and CML blast phase (CML-BP); lymphocytic leukemia; myeloid leukemia; acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma, including diffuse large B-cell lymphoma (DLBCL); T-cell lymphoma; multiple myeloma (MM); amyloidosis; Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes; cancers of the brain, including glioma/glioblastoma, anaplastic oligodendroglioma, and adult anaplastic astrocytoma; neuroendocrine cancers, including metastatic neuroendocrine tumors; cancers of the head and neck, including, e.g., squamous cell carcinoma of the head and neck, and nasopharyngeal cancer; cancers of the oral cavity, pharynx and small intestine; bone cancer; soft tissue sarcoma; and villous colon adenoma; and
diseases of the central nervous system (CNS), such as multiple sclerosis (MS).

In some embodiments, the methods of the present disclosure may be for treating and/or preventing any one or more of the diseases, conditions and/or disorders disclosed herein. For example, in some embodiments, there is provided a method for treating and/or preventing any one or more of: retinal ischaemic reperfusion injury, chronic recurrent multifocal osteomyelitis, aplastic anaemia, CRIA, ethanol-induced liver disease, NASH, inflammatory hepatitis, acute kidney injury, IRI, multiple sclerosis, amyotrophic lateral sclerosis, Alzheimer's disease, stroke, systemic lupus erythematosus, myocardial infarction, diabetes, Crohn's disease, inflammatory bowel disease and COPD, comprising administering to a subject in need thereof an effective amount of a compound of the invention or a pharmaceutically acceptable salt, solvate, tautomer, N-oxide, stereoisomer and/or prodrug thereof.

The methods can also be used for protecting cells, tissues and/or transplanted organs, whether before, during (removal, transport and/or re-implantation) or after transplantation.

In some embodiments, the compound of the invention may be administered in combination with a further active pharmaceutical ingredient (API). The API may be any that is suitable for treating any of the diseases, conditions and/or disorders associated with necroptosis, such as those described herein. The compound of the invention may be co-formulated with the further API in any of the pharmaceutical compositions described herein, or the compound of the invention may be administered in a concurrent, sequential or separate manner. Concurrent administration includes administering the compound of the invention at the same time as the other API, whether coformulated or in separate dosage forms administered through the same or different route. Sequential administration includes administering, by the same or different route, the compound of the invention and the other API according to a resolved dosage regimen, such as within about 0.5, 1, 2, 3, 4, 5, or 6 hours of the other. When sequentially administered, the compound of the invention may be administered before or after administration of the other API. Separate administration includes administering the compound of the invention and the other API according to regimens that are independent of each other and by any route suitable for either active, which may be the same or different.

The methods may comprise administering the compound of Formula (I) in any pharmaceutically acceptable form. In some embodiments, the compound of Formula (I) is provided in the form of a pharmaceutically acceptable salt, solvate, N-oxide, polymorph, tautomer or prodrug thereof, or a combination of these forms in any ratio.

The methods may also comprise administering a pharmaceutical composition comprising the compound of formula (I) or a pharmaceutically acceptable salt, solvate, N-oxide, polymorph, tautomer or prodrug thereof to the subject in need thereof. The pharmaceutical composition may comprise any pharmaceutically acceptable carrier, diluent and/or excipient described herein.

The compounds of Formula (I), or a pharmaceutically acceptable salt or prodrug thereof, as defined herein, may be administered by any suitable means, for example, orally, rectally, nasally, vaginally, topically (including buccal and sub-lingual), parenterally, such as by subcutaneous, intraperitoneal, intravenous, intramuscular, or intracisternal injection, inhalation, insufflation, infusion or implantation techniques (e.g., as sterile injectable aqueous or non-aqueous solutions or suspensions).

The compounds of the invention may be provided as pharmaceutical compositions including those for oral, rectal, nasal, topical (including buccal and sub-lingual), parenteral administration (including intramuscular, intraperitoneal, sub-cutaneous and intravenous), or in a form suitable for administration by inhalation or insufflation. The compounds of Formula (I), or a pharmaceutically acceptable salt or prodrug thereof, together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, or in the form of sterile injectable solutions for parenteral (including subcutaneous) use.

Also provided is a kit of parts, comprising in separate parts:
a compound of Formula (I) or a pharmaceutically acceptable salt, solvate, N-oxide, polymorph, tautomer or prodrug thereof; and
instructions for its use in any of the methods of the invention.

The compounds, compositions, kits and methods described herein are described by the following illustrative and non-limiting examples.

### EXAMPLES

### Chemistry

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents, which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high-performance liquid chromatography (HPLC) or thin layer chromatography (TLC).

The expressions, “ambient temperature,” “room temperature,” and “RT”, as used herein, are understood in the art, and refer generally to a temperature, e.g. a reaction temperature, that is about the temperature of the room in which the reaction is carried out, for example, a temperature from about 20° C. to about 30° C.

Compounds of the invention can be prepared according to numerous preparatory routes known in the literature. Example synthetic methods for preparing compounds of the invention are provided in the Schemes below. In each of these schemes, A¹-A⁵, X and G have the meanings given above for any of the compounds of the invention, eg formulas (I) and (II).

### General Description of Chemistry

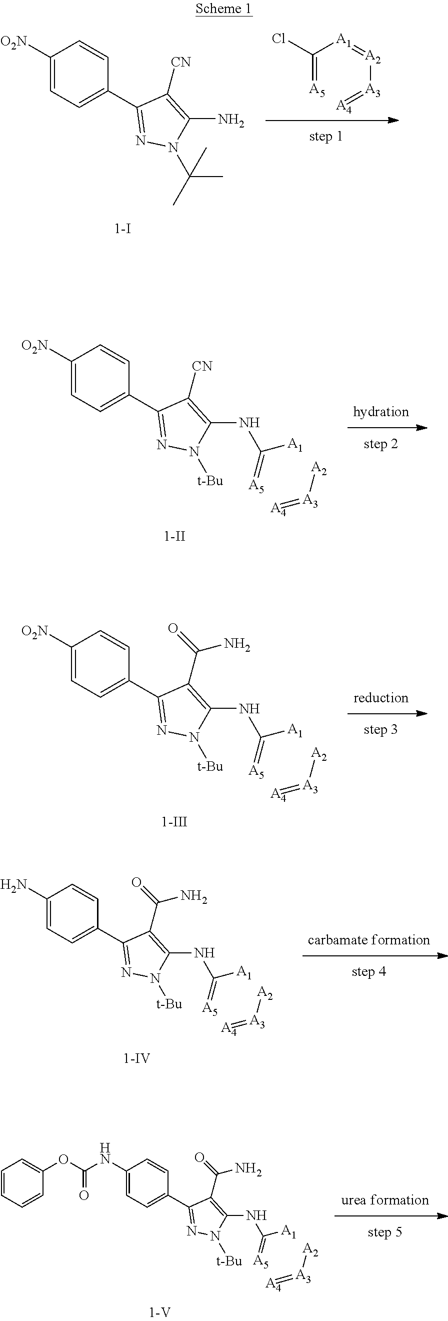

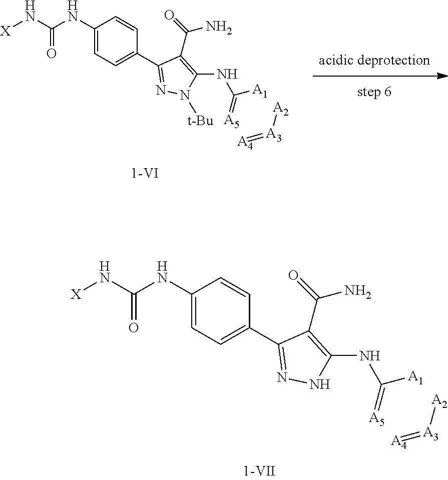

Scheme 1 shows a general synthesis of aminopyrazolocarboxamide compounds of the invention. Aminopyrazolonitrile (1-1), which can be prepared via routes known to one skilled in the art, can be converted to N-heteroaryl aminopyrazolonitriles 1-11 (step 1) by treatment with chloro heteroarenes or bromo heteroarenes in the presence of palladium sources such as Pd₂(dba)₃ or Pd(OAc)₂ and a ligand such as Xantphos with a base such as cesium carbonate in a solvent such as 1,4-dioxane or diglyme at elevated temperature such as 65° C. or under microwave reaction such as 150° C. The nitrile group can be converted to a primary amide (compound 1-III) in the presence of a reagent such as Ghaffar-Parkin's catalyst in a solvent such as dioxane and H₂O at elevated temperature such as 100° C., or with 30% hydrogen peroxide in water with an aqueous sodium hydroxide solution in a polar solvent such as DMSO and a protic solvent such as EtOH at elevated temperature such as 100° C. (step 2). The nitro substituent of compound 1-III can be reduced to give aniline 1-IV in the presence of an aqueous solution of ammonium chloride in a protic solvent such as methanol in the presence of Zn dust at room temperature (step 3). Aniline 1-IV can subsequently be converted to activated carbamate 1-V by reaction with phenyl chloroformate or 4-nitro phenyl chloroformate in the presence of a base such as pyridine and in a chlorinated solvent such as dichloromethane or chloroform at room temperature (step 4). The subsequent urea formation can be preformed by addition of an alkylamine in the presence of a tertiary amine base such as triethylamine or N,N-diisopropylethylamine in a polar solvent such as THF at room temperature (step 5). Compound 1-VIIs of invention (products of step 6) can be obtained via an acidic deprotection with an acid such as TFA in a solvent such as dichloromethane at room temperature (step 6).

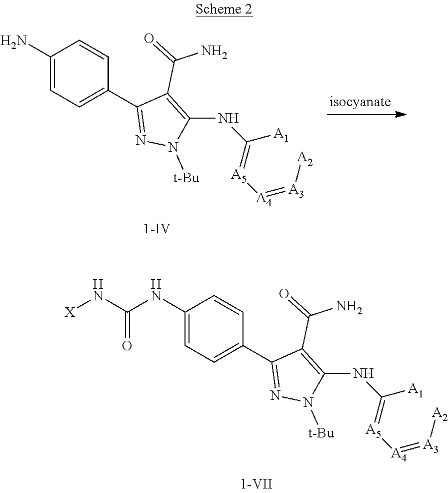

Alternatively, compound 1-VII can be directly prepared from the aniline 1-IV by treatment with an aryl/alkyl isocyanate in a solvent such as THF at room temperature.

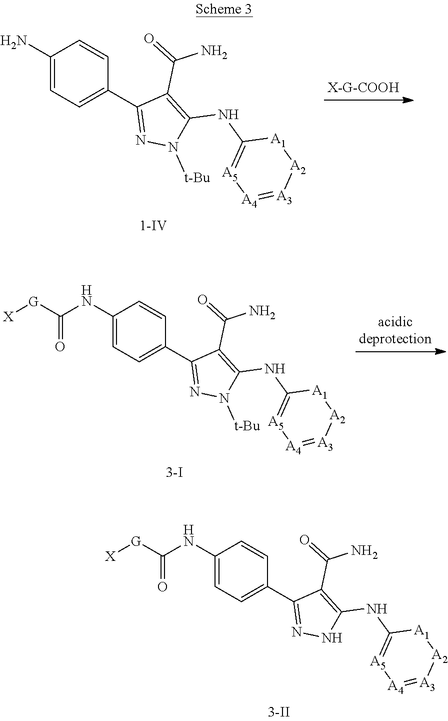

Scheme 3 shows that aniline I-IV can react with arylcarboxylic acid in the presence of a coupling reagent such as HATU with a tertiary amine base such as triethylamine or diisopropylethylamine in a polar solvent such as THF or DMF. Compounds 3-II can be obtained via an acidic deprotection with an acid such as TFA in a solvent such as dichloromethane at room temperature.

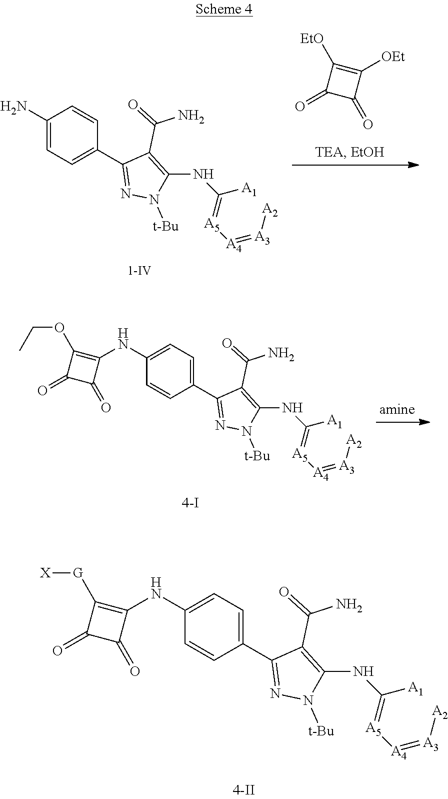

Scheme 4 describes a general synthesis of aminopyrazolocarboxamide squaramide compounds of the invention. 3,4-Diethoxycyclobut-3-ene-1,2-dione can be reacted with the aniline 1-IV to provide compound 4-I in a protic solvent such as ethanol in the presence of a tertiary amine base such as triethylamine at elevated temperature such as 60° C. (step 1). Displacement with an amine (eg a primary or secondary amine, such as an alkylamine or arylamine) in a protic solvent such as ethanol in the presence of a tertiary amine base such as triethylamine at elevated temperature such as 60° C. provide the squaramide compounds 4-II, wherein G is NR³ or a single bond and when G is a single bond, X is an optionally substituted heterocyclyl comprising a nitrogen atom at the point of attachment.

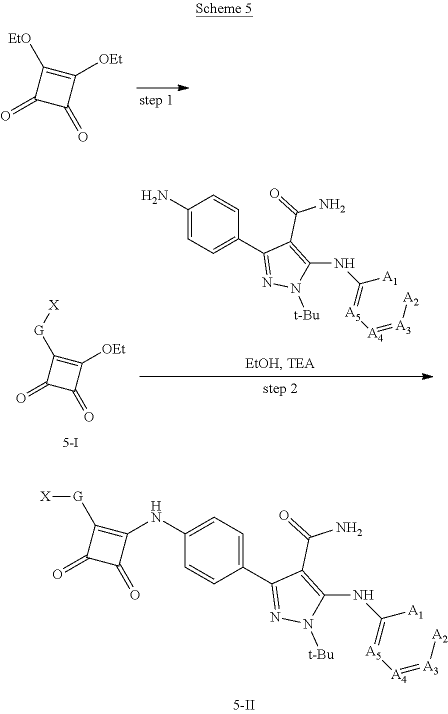

3,4-Diethoxycyclobut-3-ene-1,2-dione can be first reacted with a nucleophile, eg a substituted aniline, to provide the intermediate compounds 5-I. This reaction typically takes place in a protic solvent such as ethanol and in the presence of a tertiary amine base such as triethylamine at elevated temperature such as 60° C. (step 1). Aniline 1-IV can then be reacted with compound 5-I in a protic solvent such as ethanol in the presence of a tertiary amine base such as triethylamine at elevated temperature such as 60° C. (step 2) to give compound 5-II. Preferably, in compound 5-II, G is NR³ or a single bond and when G is a single bond, X is an optionally substituted heterocyclyl comprising a nitrogen atom at the point of attachment.

### General Chemistry Methods

### Definitions

AcCl (acetyl chloride);
ACN (acetonitrile);
Ac₂O (acetic anhydride);
AcOH (acetic acid);
atm (atmosphere);
B₂pin₂ (bis(pinacolato)diboron);
BINAP (2,2′-bis(diphenylphosphino)-1,1′-binaphthyl);
BnBr (benzyl bromide);
Boc₂O (di-tert-butyl dicarbonate);
CDCl₃ (deuterated chloroform);
CsF (cesium fluoride);
conc. (concentrated);
d (days)
DAST (diethylaminosulphur trifluoride);
dba (dibenzylideneacetone);
DCM (dichloromethane);
DIPEA (N,N-diisopropylethylamine);
DMAP (4-dimethylaminopyridine);
DME (1,2-dimethoxyethane);
DMF (N,N-dimethylformamide);
DMSO (dimethylsulfoxide);
DMSO-d₆ (deuterated dimethylsulfoxide);
EDCI (1-ethyl-3-(3-dimethylaminopropyl)carbodiimide);
eq (molar equivalent);
ES-API (electrospray atmospheric pressure ionization);
Et₃N (triethylamine);
Et₂O (diethyl ether);
EtOAc (ethyl acetate);
EtOH (ethanol);
g (gram);
h (hour);
HATU (1-[bis(dimethylamino)methylene]-1H-1,2,3-triazolo[4,5-b]pyridinium 3-oxide hexafluorophosphate);
HCl (hydrochloric acid/hydrogen chloride);
HOBt (hydroxybenzotriazole);
¹H NMR (proton nuclear magnetic resonance);
Hz (hertz);
L (litre);
LCMS (liquid chromatography-mass spectrometry);
LiAlH₄ (lithium aluminium hydride);
LiHMDS (lithium bis(trimethylsilyl)amide);
M (molar);
MeOH (methanol);
MeOD-d₄ (deuterated methanol);
mg (milligrams);
MHz (megahertz);
min (minutes);
mL (millilitres);
mmol (millimoles);
MsCl (methanesulfonyl chloride);
n-BuLi (n-butyllithium);
NaH (sodium hydride);
NaHCO₃ (sodium bicarbonate);
NaOEt (Sodium ethoxide);
NaOH (sodium hydroxide);
Na₂SO₄ (sodium sulphate);
NBS (N-bromosuccinamide)
Na₂CO₃ (sodium carbonate);
NH₄Cl (ammonium chloride);
NMP (N-methyl-2-pyrrolidone);
Pd/C (palladium on activated charcoal);
Pd₂(dba)₃ (tris(dibenzylideneacetone)dipalladium(0));
Pd(dppf)₂Cl₂·CH₂Cl₂ (1,1-Bis(diphenylphosphino)ferrocenedichloropalladium(II), complex with DCM)
Pd(t-Bu₃P)₂ (bis(tri-tert-butylphosphine)palladium(0))
PE (petroleum ether);
PhMe (toluene);
PhNHNH₂·HCl (phenylhydrazine hydrochloride)
prep-HPLC (preparative high-performance liquid chromatography);
prep-TLC (preparative thin layer chromatography);
ppm (parts per million);
psi (pounds per square inch);
p-TSA (p-toluenesulfonic acid);
[RhOH(cod)]₂ (hydroxy(1,5-cyclooctadiene)rhodium(I)dimer)
RT (room temperature);
SEMCl (2-(trimethylsilyl)ethoxymethyl chloride);
SPhos (2-dicyclohexylphosphino-2′,6′-dimethoxybiphenyl);
TBAF (tetra-n-butylammonium fluoride);
TFA (trifluoroacetic acid);
THF (tetrahydrofuran);
Ti(OEt)₄ (titanium(IV) ethoxide);
TLC (thin layer chromatography);
t-BuNHNH₂·HCl (tert-butylhydrazine hydrochloride)
v/v (volume/volume);
Xantphos (9,9-dimethyl-4,5-bis(di-tert-butylphosphino)xanthene).

### LCMS Methodology

Electrospray mass spectroscopy (MS) was carried out using one of the following methods:

Method A (5 minutes): LC model: Agilent 1200 (Pump type: Binary Pump, Detector type: DAD) MS model: Agilent G6110A Quadrupole. Column: Xbridge-C18, 2.5 μm, 2.1×30 mm. Column temperature: 30° C. Acquisition of wavelength: 214 nm, 254 nm. Mobile phase: A: 0.07% HCOOH aqueous solution, B: MeOH. Run time: 5 min. MS: Ion source: ES+ (or ES−). MS range: 50˜900 m/z. Fragmentor: 60. Drying gas flow: 10 L/min. Nebulizer pressure: 35 psi. Drying gas temperature: 350° C. Vcap: 3.5 kV.

Method B (3.5 minutes): LC model: Agilent 1200 (Pump type: Binary Pump, Detector type: DAD) MS model: Agilent G6110A Quadrupole. Column: Xbridge-C18, 2.5 μm, 2.1×30 mm. Column temperature: 30° C. Acquisition of wavelength: 214 nm, 254 nm. Mobile phase: A: 0.07% HCOOH aqueous solution, B: MeOH. Run time: 5 min. MS: Ion source: ES+ (or ES−). MS range: 50˜900 m/z. Fragmentor: 60. Drying gas flow: 10 L/min. Nebulizer pressure: 35 psi. Drying gas temperature: 350° C. Vcap: 3.5 kV.

Method C (4 minutes): Agilent LCMS system composed of an Agilent G6120B Mass Detector, 1260 Infinity G1312B Binary pump, 1260 Infinity G1367E HiPALS autosampler, and 1260 Infinity G4212B Diode Array Detector. Conditions for LCMS were as follows: column, Poroshell 120 EC-C18, 2.1×50 mm, 2.7 μm at 30° C.; injection volume, 2 μL; gradient, 5-100% B over 3 min (solvent A: water/0.1% formic acid; solvent B: AcCN/0.1% formic acid); flow rate, 1.0 mL/min; detection, 14 and 254 nm; acquisition time, 4.1 min; ion source: single quadrupole; ion mode: API-ES; drying gas temperature: 350° C.; capillary voltage: 4000; scan range 100-1000; step size: 0.1.

### Preparative HPLC

Instrument type: VARIAN 940 LC. Pump type: Binary Pump. Detector type: PDA.

### LC Conditions:

Column: Waters SunFire prep C18 OBD, 5 μm, 19×100 mm.
Acquisition wavelength: 214 nm, 254 nm.
Mobile Phase: A: 0.07% TFA aqueous solution, B: MeOH.

### NMR

Nuclear magnetic resonance spectra were recorded on Bruker Avance DRX 300 instrument at 300.13 MHz or Bruker 400 MHz for 1H nuclei as specified. Samples were recorded in deuterated solvent as specified, and data acquired at 25° C. Chemical shifts are reported in ppm on the 6 scale and referenced to the appropriate solvent peak. In reporting spectral data, the following abbreviations have been used: s, singlet; br s, broad singlet; d, doublet; t, triplet; q, quartet; m, multiplet

Synthesis of Common Intermediates

### Intermediate A1: 3-(4-aminophenyl)-1-(tert-butyl)-5-(pyridin-2-ylamino)-1H-pyrazole-4-carboxamide

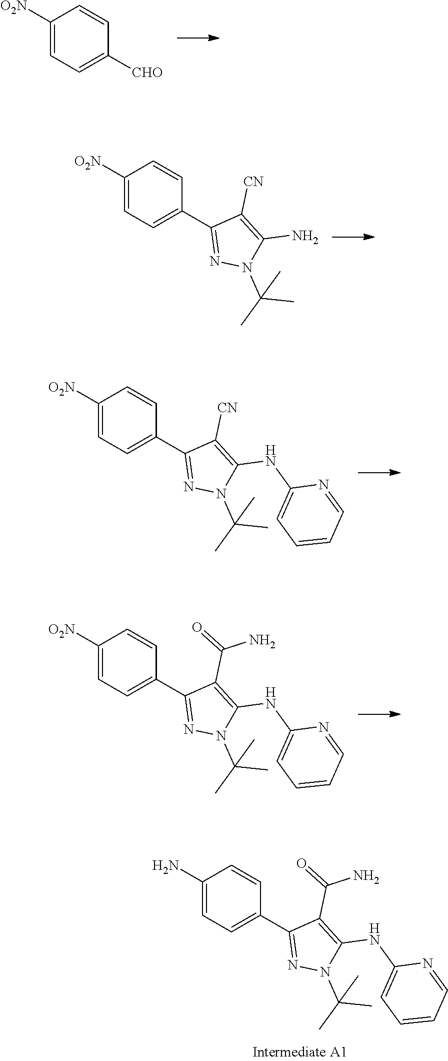 [SMILES: O=Cc1ccc([N+](=O)[O-])cc1 ; CC(C)(C)n1nc(-c2ccc([N+](=O)[O-])cc2)c(C#N)c1N ; CC(C)(C)n1nc(-c2ccc([N+](=O)[O-])cc2)c(C#N)c1Nc1ccccn1 ; CC(C)(C)n1nc(-c2ccc([N+](=O)[O-])cc2)c(C(N)=O)c1Nc1ccccn1 ; CC(C)(C)n1nc(-c2ccc(N)cc2)c(C(N)=O)c1Nc1ccccn1]

### Step 1: 5-amino-1-(tert-butyl)-3-(4-nitrophenyl)-1H-pyrazole-4-carbonitrile

A mixture of 4-nitrobenzaldehyde (100 g, 0.66 mol) and t-BuNHNH₂·HCl (90.7 g, 0.73 mol) in DMF (500 mL) was stirred at RT overnight. The reaction mixture was cooled to 0° C. and then NBS (129.6 g, 0.73 mol) was added slowly. The resultant mixture was stirred at 0° C. for 5 h and then a solution of malononitrile (52.5 g, 0.79 mol) and NaOEt (112.7 g, 1.66 mol) in EtOH (300 mL) was slowly added over a 30 min period at 0° C. The mixture was stirred at RT for 16 h and then partitioned between water (3 L) and EtOAc (3 L). The aqueous layer was extracted with EtOAc (2×3 L) and the combined organic layers were washed with brine, dried (Na₂SO₄) and concentrated under reduced pressure. The crude residue was purified by silica gel column chromatography (PE:EtOAc, 5:1) to afford the title product (58 g, 31%) as a yellow solid. LCMS (method A): 1.93 min; m/z=286.1 [M+H]⁺.

### Step 2: 1-(tert-butyl)-3-(4-nitrophenyl)-5-(pyridin-2-ylamino)-1H-pyrazole-4-carbonitrile

To a solution of 5-amino-1-(tert-butyl)-3-(4-nitrophenyl)-1H-pyrazole-4-carbonitrile (13 g, 45.6 mmol) in diglyme (200 mL), was added 2-bromopyridine (7.6 g, 47.8 mmol), Pd(OAc)₂ (614 mg, 2.73 mmol), Xantphos (1.6 g, 2.73 mmol) and Cs₂CO₃ (37.1 g, 114 mmol) and the mixture was stirred at 150° C. under N₂ for 8 h. The reaction mixture was filtered through Celite and the filter cake was washed with MeOH. The filtrate was concentrated under reduced pressure and the crude residue was purified by silica gel column chromatography (PE:EtOAc, 5:1) to afford the title product (7.0 g, 42%) as a yellow solid. LCMS (method A): 2.91 min; m/z=363.2 [M+H]⁺.

### Step 3: 1-(tert-butyl)-3-(4-nitrophenyl)-5-(pyridin-2-ylamino)-1H-pyrazole-4-carboxamide

To a solution of 1-(tert-butyl)-3-(4-nitrophenyl)-5-(pyridin-2-ylamino)-1H-pyrazole-4-carbonitrile (11 g, 30.3 mmol) in DMSO (35 mL) and EtOH (130 mL), was added 30% aq. H₂O₂ (35 mL) and 5% aq. NaOH (0.3 mL) and the mixture was stirred at 80° C. for 2 h. The reaction mixture was then concentrated under reduced pressure and the residue diluted with H₂O to form a yellow suspension. The precipitated solids were collected by filtration and dried under reduced pressure to give the title product (10.5 g, 90%) as a yellow solid. LCMS (method A): 2.65 min; m/z=381.1 [M+H]⁺.

### Step 4: 3-(4-aminophenyl)-1-(tert-butyl)-5-(pyridin-2-ylamino)-1H-pyrazole-4-carboxamide

To a solution of 1-(tert-butyl)-3-(4-nitrophenyl)-5-(pyridin-2-ylamino)-1H-pyrazole-4-carboxamide (10 g, 26.3 mmol), in MeOH (200 mL), was added sat. aq. NH₄Cl (100 mL) and Zn dust (8.6 g, 131.5 mmol) and the mixture was stirred at 40° C. for 2 h. The reaction mixture was filtered through Celite, and the filter cake was washed with MeOH. The filtrate was concentrated under reduced pressure, diluted with H₂O then and basified to pH 10 with sat. aq. Na₂CO₃. The mixture was extracted with DCM (3×100 mL) and the combined organics were washed with brine, dried (Na₂SO₄) and concentrated under reduced pressure to give the title product (8.0 g, 75%) as a yellow solid. LCMS (method A): 0.53 min; m/z=351.1 [M+H]⁺.

### Intermediate A2: 3-(4-aminophenyl)-1-(tert-butyl)-5-((6-(trifluoromethyl)pyridin-2-yl)amino)-1H-pyrazole-4-carboxamide

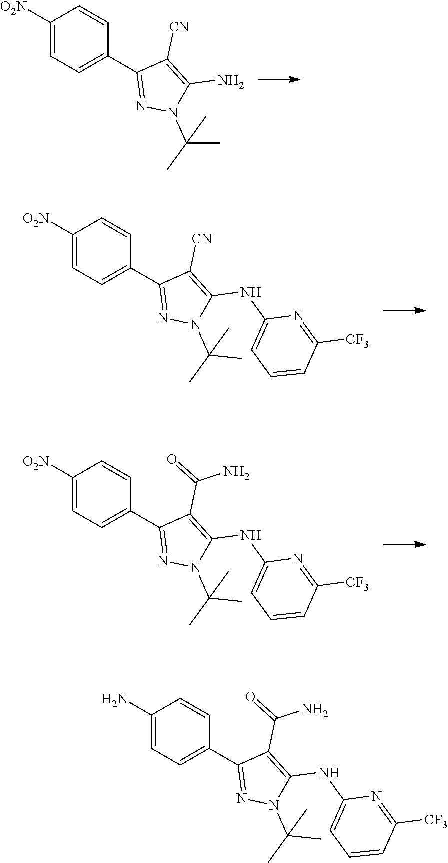 [SMILES: CC(C)(C)n1nc(-c2ccc([N+](=O)[O-])cc2)c(C#N)c1N ; CC(C)(C)n1nc(-c2ccc([N+](=O)[O-])cc2)c(C#N)c1Nc1cccc(C(F)(F)F)n1 ; CC(C)(C)n1nc(-c2ccc([N+](=O)[O-])cc2)c(C(N)=O)c1Nc1cccc(C(F)(F)F)n1 ; CC(C)(C)n1nc(-c2ccc(N)cc2)c(C(N)=O)c1Nc1cccc(C(F)(F)F)n1]

### Step 1: 1-tert-butyl-3-(4-nitrophenyl)-5-{[6-(trifluoromethyl)pyridin-2-yl]amino}-1H-pyrazole-4-carbonitrile

A mixture of 5-amino-1-tert-butyl-3-(4-nitrophenyl)-1H-pyrazole-4-carbonitrile (20 g, 70.0 mmol), Cs₂CO₃ (68.4 g, 210 mmol), Xantphos (8.10 g, 14.0 mmol), Pd₂(dba)₃ (6.41 g, 7.00 mmol) and 2-chloro-6-(trifluoromethyl)pyridine (12.7 g, 70.0 mmol) in degassed 1,4-dioxane (150 mL) was stirred at 65° C. under N₂ overnight. The reaction mixture was concentrated under reduced pressure and the crude residue was purified by silica gel column chromatography (DCM:MeOH, 25:1) to afford the desired product (28.5 g, 94%) as a yellow solid. LCMS (method A): 4.54 min; m/z=431.3 [M+H]⁺.

### Step 2: 1-tert-butyl-3-(4-nitrophenyl)-5-{[6-(trifluoromethyl)pyridin-2-yl]amino}-1H-pyrazole-4-carboxamide

To a solution of 1-tert-butyl-3-(4-nitrophenyl)-5-{[6-(trifluoromethyl)pyridin-2-yl]amino}-1H-pyrazole-4-carbonitrile (26.5 g, 61.5 mmol) in DMSO (240 mL), was added 30% aq. H₂O₂ (240 mL) and 1.0 M aq. NaOH (240 mL) in EtOH (960 mL), and the solution was stirred at 100° C. under N₂ overnight. The reaction mixture was concentrated under reduced pressure and the residue was partitioned between H₂O (2 L) and EtOAc (2 L). The organic layer was dried with Na₂SO₄ and concentrated under reduced pressure. The crude residue was purified by silica gel column chromatography (DCM:MeOH, 20:1) to afford the desired product (22 g, 80%) as a yellow solid. LCMS (method A): 4.22 min; m/z=449.1 [M+H]⁺.

### Step 3: 3-(4-aminophenyl)-1-(tert-butyl)-5-((6-(trifluoromethyl)pyridin-2-yl)amino)-1H-pyrazole-4-carboxamide

To a solution of 1-tert-butyl-3-(4-nitrophenyl)-5-{[6-(trifluoromethyl)pyridin-2-yl]amino}-1H-pyrazole-4-carboxamide (22 g, 49.0 mmol) in MeOH (450 mL), was added sat. aq.

NH₄Cl (150 mL) and Zn dust (16.0 g, 245 mmol) and the reaction mixture was stirred at 60° C. under N₂ overnight. The reaction mixture was filtered, then the filtrate was diluted with EtOAc (50 mL) and H₂O (50 mL) and the two layers were separated. The organic layer was washed with H₂O, dried (Na₂SO₄) and concentrated under reduced pressure. The crude residue was purified by silica gel column chromatography (DCM:MeOH, 15:1) to afford the title product (7.5 g, 36%) as a yellow solid. LCMS (method A): 3.44 min; m/z=419.1 [M+H]⁺. ¹H NMR (400 MHz, DMSO-d₆): 8.84 (s, 1H), 7.75 (d, J=16 Hz, 1H), 7.41 (t, J=8.4 Hz, 1H), 7.15 (br. s, 2H), 6.83 (s, 1H), 6.67 (s, 1H), 6.56 (t, J=8.4 Hz, 2H), 5.28 (s, 2H), 1.54 (s, 9H).

The following intermediate A3 was similarly prepared from 2-chloropyrazine and 5-amino-1-(tert-butyl)-3-(4-nitrophenyl)-1H-pyrazole-4-carbonitrile according to the method described for the synthesis of 3-(4-aminophenyl)-1-(tert-butyl)-5-(pyridin-2-ylamino)-1H-pyrazole-4-carboxamide (intermediate A1). Characterisation of intermediate A3 is provided in Table 1.

TABLE 1

| Compound ID | | | | Starting |
|---|---|---|---|---|
| and name | Structure | LCMS data | ¹HNMR data | material |
| Intermediate A3 3-(4- aminophenyl)-1- (tert-butyl)-5- (pyrazin-2- ylamino)-1H- pyrazole-4- carboxamide | 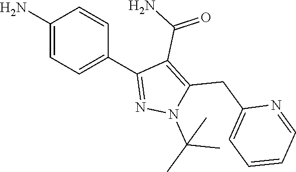 [SMILES: CC(C)(C)n1nc(-c2ccc(N)cc2)c(C(N)=O)c1Cc1ccccn1] | LCMS (method A): 1.53 min; m/z = 352.4 [M + H]⁺. | ¹H NMR (400 MHz, DMSO-de): 8.72 (s, 1H), 8.02 (d, J= 8.4 Hz, 2H), 7.92 (d, J = 2.4 Hz, 1H), 7.37 (d, J = 8.4 Hz, 2H), 7.05 (s, 1H), 6.73 (s, 1H), 6.55 (d, J= 8.4 Hz, 2H), 5.16 (s, 2H), 1.54 (s, 9H). | 2- chloro- pyrazine |

### Carbamate Intermediate A4

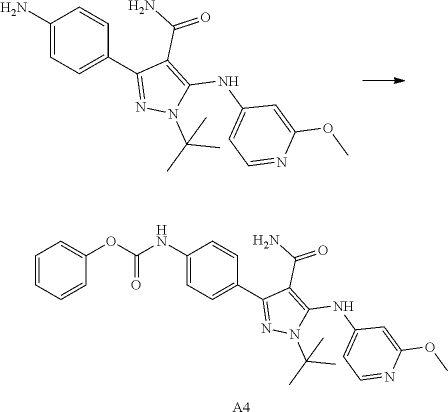 [SMILES: COc1cc(Nc2c(C(N)=O)c(-c3ccc(N)cc3)nn2C(C)(C)C)ccn1 ; COc1cc(Nc2c(C(N)=O)c(-c3ccc(NC(=O)Oc4ccccc4)cc3)nn2C(C)(C)C)ccn1 ; "A4"]

### Step 1: phenyl (4-(1-(tert-butyl)-4-carbamoyl-5-((2-methoxypyridin-4-yl)amino)-1H-pyrazol-3-yl)phenyl)carbamate

A solution of 3-(4-aminophenyl)-1-tert-butyl-5-[(2-methoxypyridin-4-yl)amino]-1H-pyrazole-4-carboxamide (1.2 g, 3.15 mmol), phenyl chloroformate (739 mg, 4.72 mmol) and pyridine (498 mg, 6.30 mmol) in DCM (20 mL) was stirred at RT under N₂ overnight. To the mixture, was added H₂O (300 mL) and the organics were extracted with EtOAc (3×100 mL). The combined organic layers were dried over Na₂SO₄ and concentrated under reduced pressure. The crude residue was purified by silica gel column chromatography (PE:EtOAc, 1:3) to afford the desired product (680 mg 43%) as a white solid. LCMS (method B): 0.72 min; m/z=501.1 [M+H]⁺. ¹H NMR (400 MHz, DMSO-d₆): 10.29 (s, 1H), 8.23 (s, 1H), 7.78 (d, J=5.6 Hz, 1H), 7.69 (d, J=8.4 Hz, 2H), 7.53 (d, J=8.4 Hz, 2H), 7.43 (m, 2H), 7.30-7.16 (m, 4H), 7.08 (s, 1H), 6.30 (br s, 1H), 5.80 (br s, 1H), 3.73 (s, 3H), 1.56 (s, 9H).

The following intermediates were similarly prepared from the appropriate starting material according to the method described for intermediate A4:

TABLE 2

| Intermediate ID and name | Structure | LCMS | SM |
|---|---|---|---|
| Intermediate A5: phenyl (4-(1-(tert- butyl)-4-carbamoyl-5-((6- (trifluoromethyl)pyridin-2-yl)amino)- 1H-pyrazol-3-yl)phenyl)carbamate | 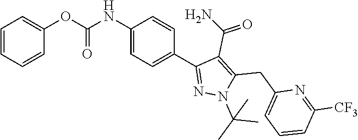 [SMILES: CC(C)(C)n1nc(-c2ccc(NC(=O)Oc3ccccc3)cc2)c(C(N)=O)c1Cc1cccc(C(F)(F)F)n1] | LCMS (method B): 2.50 min; m/z = 539.0 [M + H]⁺. | 3-(4-aminophenyl)-1-(tert-buyl)- 5-((6-(trifluoromethyl)pyridin-2- yl)amino)-1H-pyrazole-4- carboxamide |
| Intermediate A6: phenyl (4-(1-(tert- butyl)-4-carbamoyl-5-(pyridin-2- ylamino)-1H-pyrazol-3- yl)phenyl)carbamate | 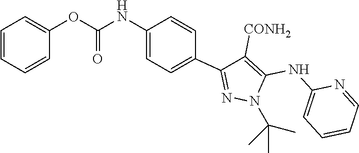 [SMILES: CC(C)(C)n1nc(-c2ccc(NC(=O)Oc3ccccc3)cc2)c(C(N)=O)c1Nc1ccccn1] | LCMS (method A): 2.32 min; m/z = 470.9 [M + H]⁺. | 3-(4-aminophenyl)-1-(tert- butyl)-5-(pyridin-2-ylamino)- 1H-pyrazole-4-carboxamide |
| Intermediate A7: phenyl (4-(1-(tert- butyl)-4-carbamoyl-5-((2,6- dimethylpyridin-4-yl)amino)-1H- pyrazol-3-yl)phenyl)carbamate | 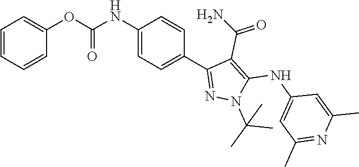 [SMILES: Cc1cc(Nc2c(C(N)=O)c(-c3ccc(NC(=O)Oc4ccccc4)cc3)nn2C(C)(C)C)cc(C)n1] | LCMS (method A): 2.82 min; m/z = 498.9 [M + H]⁺. | 3-(4-aminophenyl)-1-(tert- butyl)-5-((2,6-dimethylpyridin-4- yl)amino)-1H-pyrazole-4- carboxamide |
| Intermediate A8: phenyl N-(4-{1- tert-butyl-4-carbamoyl-5-[(pyrazin2- yl) amino]-1H-pyrazol-3- yl}phenyl)carbamate | 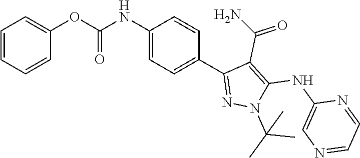 [SMILES: CC(C)(C)n1nc(-c2ccc(NC(=O)Oc3ccccc3)cc2)c(C(N)=O)c1Nc1cnccn1] | LCMS (method A): 3.59 min; m/z = 472.2 [M + H]⁺. | 3-(4-aminophenyl)-1-(tert- butyl)-5-(pyrazin-2-ylamino)- 1H-pyrazole-4-carboxamide |

### Intermediate A9

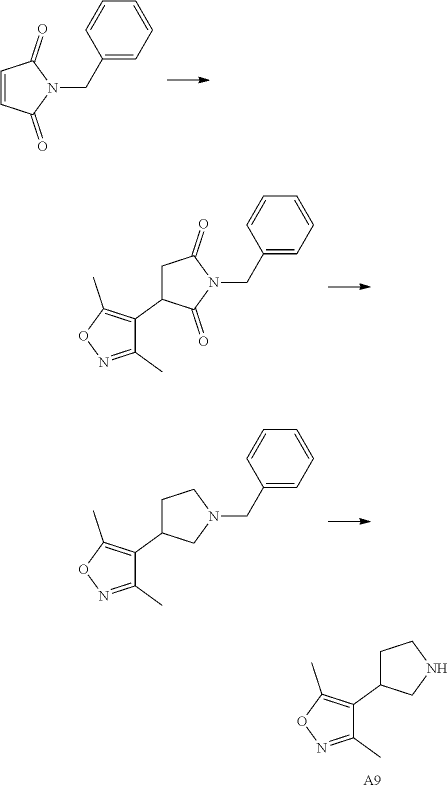

### Step 1: 1-benzyl-3-(3,5-dimethyl-1,2-oxazol-4-yl)pyrrolidine-2,5-dione

A mixture of [RhOH(cod)]₂ (121 mg, 0.267 mmol), (3,5-dimethylisoxazol-4-yl)boronic acid and Et₃N (54.0 mg, 0.534 mmol) in 1,4-dioxane (10 mL) and H₂O (1 mL) at 0° C. was stirred for 15 min. 1-Benzyl-2,5-dihydro-1H-pyrrole-2,5-dione (1 g, 5.34 mmol) was added and the mixture was stirred at RT overnight. Water was added and the mixture was extracted with EtOAc (2×20 mL). The combined organic phases were washed with H₂O and brine, dried (Na₂SO₄) and concentrated under reduced pressure. The crude residue was purified by silica gel column chromatography (PE:EtOAc, 3:1) to afford the desired product (1.2 g, 79%) as a yellow oil. LCMS (Method B): 2.64 min; m/z=284.9 [M+H]⁺.

### Step 2: 4-(1-benzylpyrrolidin-3-yl)-3,5-dimethyl-1,2-oxazole

A mixture of 1-benzyl-3-(3,5-dimethyl-1,2-oxazol-4-yl)pyrrolidine-2,5-dione (1.2 g, 4.22 mmol) and LiAlH₄ (705 mg, 16.8 mmol) in THF (30 mL) under N₂ was stirred at 70° C. for 4 h. Na₂SO₄·10H₂O (10 g) was added and the mixture was stirred for 10 min at RT, then filtered through Celite. The filtrate was concentrated, and the crude residue was purified by silica gel column chromatography (PE:EtOAc, 3:1) to afford the desired product (640 mg 59% yield) as a yellow oil. LCMS (Method A): 0.72 min; m/z=257.0 [M+H]⁺.

### Step 3: 3,5-dimethyl-4-(pyrrolidin-3-yl)isoxazole

A mixture of 4-(1-benzylpyrrolidin-3-yl)-3,5-dimethyl-1,2-oxazole (500 mg, 1.75 mmol), Pd/C (90 mg, 0.8457 mmol) and di-tert-butyl dicarbonate (571 mg, 2.62 mmol) in MeOH (20 mL) was stirred at 50° C. overnight. The mixture was filtered, and the filtrate was concentrated to give the desired product (480 mg, 92%) as a yellow oil. LCMS (method A): 3.01 min; m/z=267.2 [M+H]⁺

### Intermediate A10

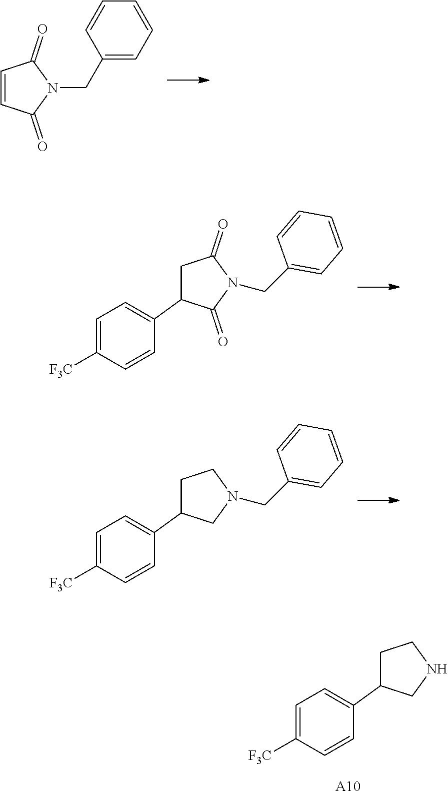

### Step 1: 1-benzyl-3-[4-(trifluoromethyl)phenyl]pyrrolidine-2,5-dione

A mixture of (4-(trifluoromethyl)phenyl)boronic acid (1.51 g, 8.00 mmol), [RhOH(cod)]₂ (145 mg, 0.32 mmol) and Et₃N (64.7 mg, 0.64 mmol) in 1,4-dioxane (10 mL) and H₂O (1 mL) was stirred at 0° C. After 15 min, 1-benzyl-2,5-dihydro-1H-pyrrole-2,5-dione (600 mg, 3.20 mmol) was added and the mixture stirred at RT overnight. The mixture was partitioned between EtOAc (20 mL) and H₂O (20 mL), and the organic layer was separated, dried (Na₂SO₄) and concentrated under reduced pressure. The crude residue was purified by silica gel column chromatography (PE:EtOAc, 5:1) to afford the title product (310 mg, 29%) as a yellow oil. LCMS (method A): 2.62 min; m/z=334.0 [M+H]⁺.

### Step 2: 1-benzyl-3-[4-(trifluoromethyl)phenyl]pyrrolidine

A mixture of 1-benzyl-3-[4-(trifluoromethyl)phenyl]pyrrolidine-2,5-dione (350 mg, 1.05 mmol) and LiAlH₄ (159 mg, 4.20 mmol) in THF (4 mL) was stirred at 70° C. for 4 h under N₂. Water (10 mL) was added followed by Na₂SO₄·10H₂O (2.4 g) and the mixture was filtered, and the filtrate was concentrated under reduced pressure. The crude residue was purified by silica gel column chromatography (PE:EtOAc, 5:1) to afford the desired product (140 mg, 44%) as yellow oil. LCMS (Method A): 4.16 min; m/z=306.2 [M+H]⁺.

### Step 3: 3-[4-(trifluoromethyl)phenyl]pyrrolidine

A mixture of 1-benzyl-3-[4-(trifluoromethyl)phenyl]pyrrolidine (140 mg, 0.46 mmol) and Pd/C (28 mg, 0.013 mmol) in MeOH (10 mL) was stirred at 50° C. overnight under an atmosphere of H₂. The reaction mixture was filtered, and the filtrate was concentrated under reduced pressure to give the desired product (84 mg, 85%) as yellow oil. LCMS (method A): 0.51 min; m/z=216.1 [M+H]⁺.

### Intermediate A11

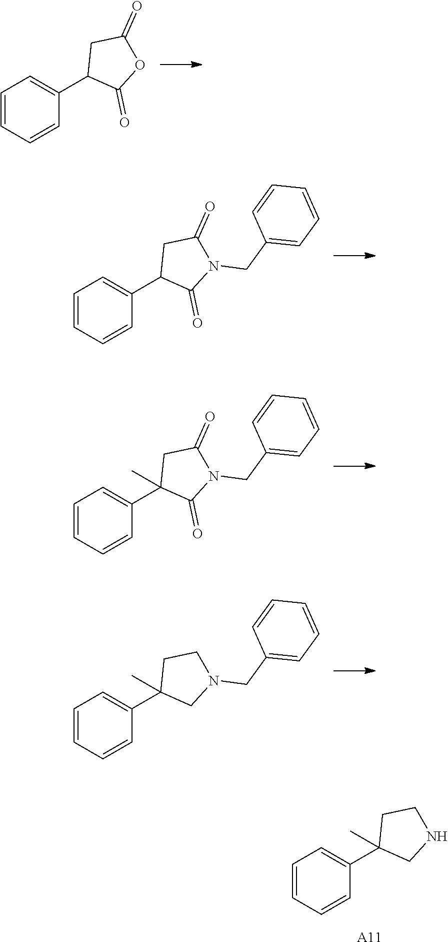

### Step 1: 1-benzyl-3-phenylpyrrolidine-2,5-dione

A solution of benzylamine (5.5 g, 51.3 mmol) in DCM (10 mL) was added dropwise to a stirred solution of 3-phenyloxolane-2,5-dione (10 g, 56.7 mmol) in DCM (20 mL) and the reaction mixture was stirred at RT for 1 h. The mixture was then concentrated under reduced pressure and the residue heated to 170° C. under N₂ for 1 h. The crude residue was purified by silica gel column chromatography (PE:EtOAc, 10:1 to 5:1) to afford the desired product (10 g, 67%) as a white solid. LCMS (Method A): 3.83 min; m/z=266.1 [M+H]⁺.

### Step 2: 1-benzyl-3-methyl-3-phenylpyrrolidine-2,5-dione

A solution of 1-benzyl-3-phenylpyrrolidine-2,5-dione (2 g, 7.53 mmol) in THF (80 mL) was cooled to −78° C. and LiHMDS (1.0 M in THF, 6.02 mL, 6.02 mmol) was added dropwise. The reaction mixture was stirred at RT for 30 min, then cooled to −78° C. and Mel (3.19 g, 22.5 mmol) was added and the mixture was stirred at RT overnight. The mixture was concentrated under reduced pressure and the crude residue was purified by silica gel column chromatography (PE:EtOAc, 10:1 to 5:1) to afford the desired product (1.5 g, 71%) as a white solid. LCMS (Method A): 4.07 min; m/z=280.1 [M+H]⁺.

### Step 3: 1-benzyl-3-methyl-3-phenylpyrrolidine

To a solution of LiAlH₄ (490 mg, 12.8 mmol) in THF (10 mL) was added a solution of 1-benzyl-3-methyl-3-phenylpyrrolidine-2,5-dione (700 mg, 2.50 mmol) in THF (6 mL). The mixture was stirred at 70° C. for 2 h under N₂, then cooled to RT and Na₂SO₄·10H₂O (7.5 g) was added and the mixture was stirred at RT for 30 min. The mixture was filtered (washing with EtOAc), and the filtrate was concentrated. The crude residue was purified by silica gel column chromatography (PE:EtOAc, 50:1 to 10:1) to afford the desired product (530 mg, 84%) as a colorless oil. LCMS (Method A): 0.85 min; m/z=253.1 [M+H]⁺.

### Step 4: 3-methyl-3-phenylpyrrolidine

A mixture of 1-benzyl-3-methyl-3-phenylpyrrolidine (530 mg, 2.10 mmol) and Pd/C (100 mg, 0.94 mmol) in MeOH (15 mL) was heated to 50° C. overnight under an atmosphere of H₂. The mixture was filtered, and the filtrate was concentrated under reduced pressure to afford the desired product (320 mg, 94.6%) as a colorless oil. LCMS (method A): 0.48 min; m/z=162.1 [M+H]+.

### Intermediate A12

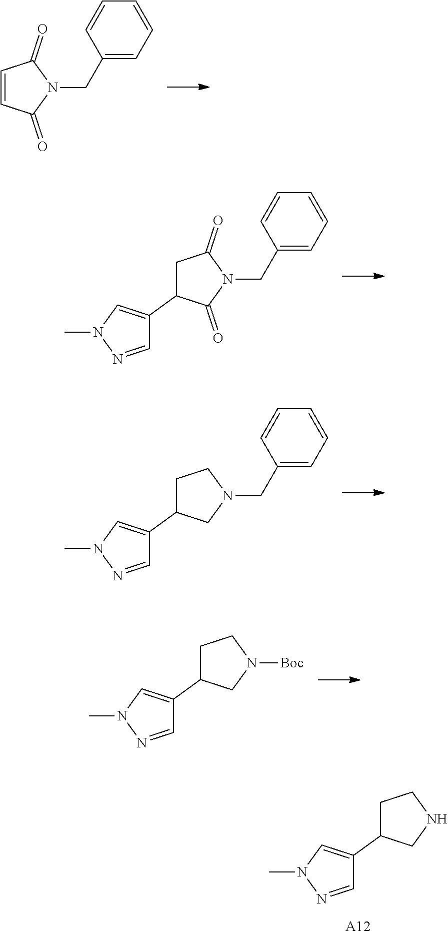

### Step 1: 1-benzyl-3-(3,5-dimethyl-1,2-oxazol-4-yl)pyrrolidine-2,5-dione

A mixture of [RhOH(cod)]₂ (241 mg, 0.5299 mmol) and Et₃N (1.07 g, 10.6 mmol) in 1,4-dioxane (50 mL) and H₂O (5 mL) was cooled to 0° C. and (1-methyl-1H-pyrazol-4-yl)boronic acid (3.33 g, 26.5 mmol) was then added. After addition, the mixture was stirred at 0° C. for an additional 15 min and 1-benzyl-2,5-dihydro-1H-pyrrole-2,5-dione (2 g, 10.6 mmol) was added. The mixture was stirred overnight at RT and then diluted with H₂O and extracted with EtOAc (2×20 mL). The combined organic phases were washed with brine, dried (Na₂SO₄) and then concentrated under reduced pressure. The crude residue was purified by silica gel column chromatography (PE:EtOAc, 2:1 to 1:2) and then triturated (EtOAc) to afford the desired product (270 mg, 9%) as a white solid. LCMS (Method B): 3.07 min; m/z=270.1 [M+H]⁺.

### Step 2: 4-(1-benzylpyrrolidin-3-yl)-1-methyl-1H-pyrazole

A mixture of 1-benzyl-3-(3,5-dimethyl-1,2-oxazol-4-yl)pyrrolidine-2,5-dione (270 mg, 1.0 mmol) and LiAlH₄ (151 mg, 4 mmol) in THF (15 mL) was stirred at 70° C. under N₂ for 4 h. Na₂SO₄·10H₂O (4 g) was added and the mixture was stirred at RT for 10 min. The mixture was filtered, and the filtrate was concentrated under reduced pressure. The crude residue was purified by silica gel column chromatography (PE:EtOAc, 35:1 to 15:1) to afford the desired product (200 mg, 83%) as a yellow oil. LCMS (Method A): 0.74 min; m/z=241.9 [M+H]⁺.

### Step 3: 1-methyl-4-(pyrrolidin-3-yl)-1H-pyrazole

A mixture of 4-(1-benzylpyrrolidin-3-yl)-1-methyl-1H-pyrazole (200 mg, 0.8287 mmol) and Pd/C (40 mg) in MeOH (5 mL) and AcOH (0.1 mL) was stirred at 50° C. overnight. The mixture was filtered, and the filtrate was concentrated under reduced pressure. The crude residue was purified by prep-TLC (DCM:MeOH, 15:1) to afford the desired product (120 mg, 58%) as a yellow oil. LCMS (method A): 0.32 min; m/z=152.1 [M+H]⁺.

### Intermediate A13

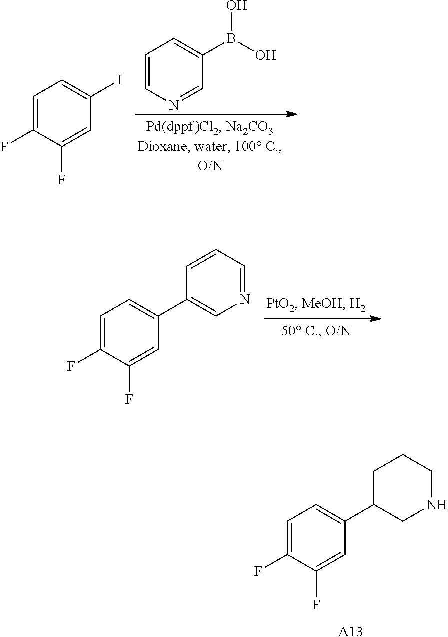

### Step 1: 3-(3,4-difluorophenyl)pyridine

A mixture of 1,2-difluoro-4-iodobenzene (2 g, 8.33 mmol), (pyridin-3-yl)boronic acid (1.02 g, 8.33 mmol), Na2CO3 (2.05 g, 16.6 mmol) and Pd(dppf)Cl₂ (609 mg, 833 μmol) in degassed 60% aq. 1,4-dioxane (8 mL) was stirred at 100° C. overnight. The mixture was poured into water (100 mL) and extracted with EtOAc (3×50 mL). The combined organic phases were dried over Na₂SO₄ and concentrated under reduced pressure. The crude residue was purified by silica gel column chromatography (petroleum ether (PE)/ethyl acetate (EtOAc)=10:1, v/v) to afford the desired product (900 mg, 57%) as a yellow solid. LC-MS (method A): Rₜ 2.78 min; [M+H]+ 192.1

### Step 2: 3-(3,4-difluorophenyl)piperidine

A mixture of 3-(3,4-difluorophenyl)pyridine (860 mg, 4.49 mmol) and platinum(IV) oxide (80 mg, 352 μmol) in 1.0 M aq. HCl (3 mL) and MeOH (10 mL) was stirred at 50° C. overnight. The mixture was concentrated under reduced pressure and the residue neutralized to pH 7 with sat. aq. Na₂CO₃ (5 mL). The mixture was diluted with water (100 mL) and the organics were extracted with EtOAc (2×50 mL). The combined organics were dried over Na₂SO₄ and concentrated under reduced pressure to give the title product (670 mg, 76%) as a white solid. LC-MS (method A): Rₜ 0.35 min; [M+H]+ 198.2.

The following compounds were similarly prepared from the appropriate iodoaryl and (pyridin-3-yl)boronic acid

TABLE 3

| Intermediate name | | | Starting |
|---|---|---|---|
| and ID | Structure | LCMS data | material |
| 3-(3,5- difluorophenyl)piperidine Intermediate A14 | 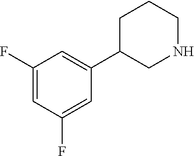 [SMILES: Fc1cc(F)cc(C2CCCNC2)c1] | LCMS (method A): Rt = 0.35 min, m/z = 198.1 [M + H]⁺. | 1,3-difluoro-5- iodobenzene |
| 3-(4- fluorophenyl)piperidine Intermediate A15 | 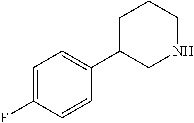 [SMILES: Fc1ccc(C2CCCNC2)cc1] | LCMS (method A): Rt = 0.40 min m/z = 180.1 [M + H]+ | 1-fluoro-4- iodobenzene |
| 3-(3- fluorophenyl)piperidine Intermediate A16 | 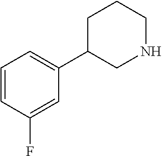 [SMILES: Fc1cccc(C2CCCNC2)c1] | LCMS (method A): Rt = 0.40 min; m/z =180.1 [M + H]+ | 1-fluoro-3- iodobenzene |

### Chiral Resolution of the Intermediates A14-A16:

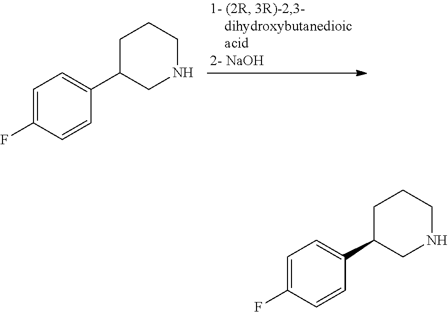 [SMILES: Fc1ccc(C2CCCNC2)cc1 ; Fc1ccc([C@H]2CCCNC2)cc1]

### (3R)-3-(4-fluorophenyl)piperidine

To a solution of 3-(4-fluorophenyl)piperidine (527 mg, 2.94 mmol) in EtOH (25 mL) was added (2S,3S)-2,3-dihydroxybutanedioic acid (441 mg, 2.94 mmol) in 75% aq. EtOH (8 mL). The mixture was slowly warmed to 70° C. for 10 min and then stirred at RT for 2 h. The precipitated solids were collected by filtration and washed with EtOH (10 mL).

The solids were treated with 1.0 M aq. NaOH (30 mL) and the aqeuous layer was extracted with EtOAc (3×50 mL). The combined organics were dried (Na₂SO₄) and concentrated under reduced pressure to give the desired product (450 mg, 86%) as a white solid.

The procedure was repeated four times to give the title product with an enantiomeric excess >98%). The ee was determined using an AD-H column with heptane/EtOH (0.1% DEA) as the mobile phase.

### Compound 1

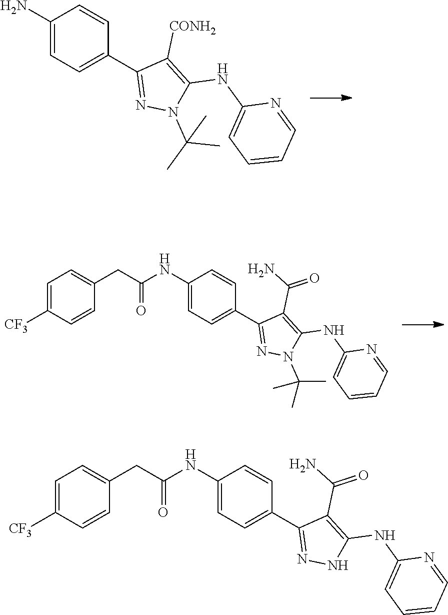 [SMILES: CC(C)(C)n1nc(-c2ccc(N)cc2)c(C(N)=O)c1Nc1ccccn1 ; CC(C)(C)n1nc(-c2ccc(NC(=O)Cc3ccc(C(F)(F)F)cc3)cc2)c(C(N)=O)c1Nc1ccccn1 ; NC(=O)c1c(-c2ccc(NC(=O)Cc3ccc(C(F)(F)F)cc3)cc2)n[nH]c1Nc1ccccn1]

### Step 1: 1-(tert-butyl)-5-(pyridin-2-ylamino)-3-(4-(2-(4-(trifluoromethyl)phenyl)acetamido)phenyl)-1H-pyrazole-4-carboxamide

A mixture of 3-(4-aminophenyl)-1-(tert-butyl)-5-(pyridin-2-ylamino)-1H-pyrazole-4-carboxamide (80 mg, 0.228 mmol), 2-(4-(trifluoromethyl)phenyl)acetic acid (47 mg, 0.228 mmol), HATU (130 mg, 0.343 mmol) and Et₃N (46 mg, 0.457 mmol) in THF (10 mL) was stirred at RT for 12 h. The mixture was concentrated under reduced pressure and the residue was diluted with EtOAc (15 mL) and washed with H₂O (2×10 mL). The organic layer was separated, dried (Na₂SO₄) and concentrated under reduced pressure. The crude residue was purified by prep-TLC (DCM:MeOH, 10:1) to afford the title product (30 mg, 25%) as a brown solid. LCMS (method A): 2.91 min; m/z=537.2 [M+H]⁺.

### Step 2: 3-(4-(2-(4-(trifluoromethyl)phenyl)acetamido)phenyl)-5-(pyridin-2-ylamino)-1H-pyrazole-4-carboxamide (Compound 1)

A solution of 3-(4-(2-(4-(trifluoromethyl)phenyl)acetamido)phenyl)-1-tert-butyl-5-(pyridin-2-ylamino)-1H-pyrazole-4-carboxamide (30 mg, 0.056 mmol) in TFA (5 mL) was stirred at 60° C. for 1 h. The mixture was concentrated under reduced pressure and the residue was triturated (Et₂O) to afford the title compound. mono TFA salt (5 mg, 19%) as a white solid. LCMS (method A): 2.53 min; m/z=481.1 [M+H]⁺. ¹H NMR (400 MHz, DMSO-d₆): 12.83 (s, 1H), 10.81 (s, 1H), 9.50 (s, 1H), 8.10 (s, 1H), 7.81 (d, J=7.6 Hz, 2H), 7.76-7.67 (m, 5H), 7.65-7.57 (m, 3H), 7.54 (d, J=7.6 Hz, 2H), 6.86 (s, 1H), 3.86 (s, 2H).

The following compounds were similarly prepared from the appropriate carboxylic acid and 3-(4-aminophenyl)-1-(tert-butyl)-5-(pyridin-2-ylamino)-1H-pyrazole-4-carboxamide (intermediate A1) following step 1 according to the method described for the synthesis of compound 1:

TABLE 4

| Compound | | | | | |
|---|---|---|---|---|---|
| No | Name | Structure | LCMS data | ¹HNMR data | SM |
| 2 | 3-(4-(3-methyl-2- phenyl- butanamido) phenyl)-5- (pyridin-2- ylamino)-1H- pyrazole-4- carboxamide | 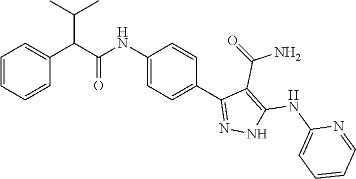 [SMILES: CC(C)C(C(=O)Nc1ccc(-c2n[nH]c(Nc3ccccn3)c2C(N)=O)cc1)c1ccccc1] | LCMS (method A): 2.51 min; m/z = 455.2 [M + H]⁺. | ¹H NMR (400 MHz, DMSO-d₆): 10.40 (br, s, 1H), 9.50 (br s, 1H), 8.31-8.17 (m, 1H), 7.99-7.95 (m, 1H), 7.76-7.48 (m, 4H), 7.50 (d, J = 8.4 Hz, 2H), 7.42 (d, J = 7.2 Hz, 2H), 7.35-7.32 (m, 2H), 7.26-7.00 (m, 1H), | 3-methyl-2- phenylbutanoic acid |
| | | | | 3.31-3.27 (m, 1H), | |
| | | | | 2.40-2.32 (m, 1H), | |
| | | | | 1.03 (d, J = 6.0 Hz, | |
| | | | | 3H), 0.69 (d, J = | |
| | | | | 6.8 Hz, 3H). | |
| 3 | 3-(4-(2- phenyl- acetamido) phenyl)-5- (pyridin-2- ylamino)-1H- pyrazole-4- carboxamide | 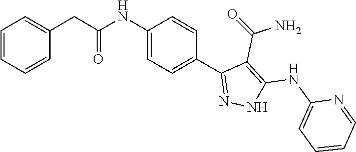 [SMILES: NC(=O)c1c(-c2ccc(NC(=O)Cc3ccccc3)cc2)n[nH]c1Nc1ccccn1] | LCMS (method A): 2.10 min; m/z = 413.1 [M + H]⁺. | ¹H NMR (400 MHz, DMSO-d₆): 13.7 (s, 1H), 12.80 (s 1H), 10.47 (s, 1H), 9.54 (s, 1H), 8.35 (s, 1H), 8.20 (s, 1H), 8.00 (m, 3H), 7.79 (m, 2H), 7.52 (m, 6H), 6.87 (s, 1H), 3.69 (s, 2H). | 2-phenylacetic acid |
| 4 | 5-(pyridin-2- ylamino)-3- (4-(2-(4- (trifluoro- methoxy) phenyl) acetamido) phenyl)-1H- | 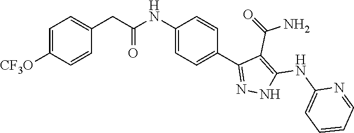 | LCMS (method A): 2.84 min; m/z = 497.1 [M + H]⁺. | ¹H NMR (400 MHz, MeOD-d₄): 8.30 (s, 1H), 7.83 (m, 3H), 7.61 (d, J = 8.4 Hz, 2H), 7.50 (d, J = 8.4 Hz, 2H), 7.09 (m, 4H), 3.79 (s, 2H). | 2-(4- (trifluoro- methoxy) phenyl)acetic acid |
| | pyrazole-4- | | | | |
| | carboxamide | | | | |
| 5 | 3-(4-(2,2- diphenyl- acetamido) phenyl)-5- (pyridin-2- ylamino)-1H- pyrazole-4- carboxamide | 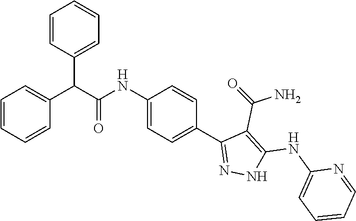 [SMILES: NC(=O)c1c(-c2ccc(NC(=O)C(c3ccccc3)c3ccccc3)cc2)n[nH]c1Nc1ccccn1] | LCMS (method A): 2.52 min; m/z = 489.2 [M + H]⁺. | ¹H NMR (400 MHz, DMSO-d₆): 10.66 (br s, 1H), 8.97 (d, J = 4.8 Hz, 1H), 8.35 (d, J = 8.4 Hz, 1H), 8.20-7.95 (m, 1H), 7.84-7.69 (m, 3H), 7.54-7.09 (m, 13H), 5.22 (s, 1H). | 2,2- diphenylacetic acid |
| 6 | 3-(4-(2- phenyl- propanamido) phenyl)-5- (pyridin-2- ylamino)-1H- pyrazole-4- carboxamide | 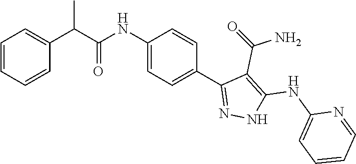 [SMILES: CC(C(=O)Nc1ccc(-c2n[nH]c(Nc3ccccn3)c2C(N)=O)cc1)c1ccccc1] | LCMS (method A): 2.36 min; m/z = 427.2 [M + H]⁺. | ¹H NMR (400 MHz, MeOD-d₄): 8.27- 8.25 (m, 1H), 7.85- 7.79 (m, 2H), 7.56- 7.54 (m, 1H), 7.40- 7.24 (m, 7H), 7.06- 6.79 (m, 2H), 3.87 (br s, 1H), 1.52 (d, J = 6.8 Hz, 3H). | 2- phenylpropanoic acid |
| 7 | 3-(4-(2-(2- (trifluoro- methyl) phenyl) acetamido) phenyl)-5- (pyridin-2- ylamino)-1H- pyrazole-4- carboxamide | 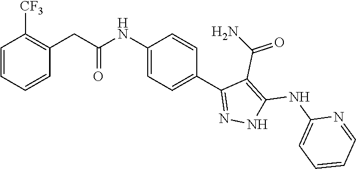 [SMILES: NC(=O)c1c(-c2ccc(NC(=O)Cc3ccccc3C(F)(F)F)cc2)n[nH]c1Nc1ccccn1] | LCMS (method A): 2.36 min; m/z = 481.2 [M + H]⁺. | ¹H NMR (400 MHz, DMSO-d₆): 12.79 (s, 1H), 10.48 (s, 1H), 9.49 (br s, 1H), 8.18 (s, 1H), 7.99- 7.54 (m, 12 H), 6.86 (s, 1H), 3.97 (s, 2H). | 2-(2- (trifluoromethyl) phenyl)acetic acid |
| 8 | 3-(4-(2- cyclohexyl- acetamido) phenyl)- 5-(pyridin-2- ylamino)-1H- pyrazole-4- carboxamide | 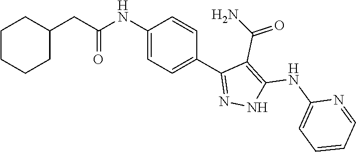 [SMILES: NC(=O)c1c(-c2ccc(NC(=O)CC3CCCCC3)cc2)n[nH]c1Nc1ccccn1] | LCMS (method A): 2.44 min; m/z = 419.2 [M + H]⁺. | ¹H NMR (400 MHz, DMSO-d₆): 10.12 (s, 1H), 9.58 (br s, 1H), 8.20 (d, J = 3.6 Hz, 1H), 7.76- 7.66 (m, 6H), 7.52- 7.50 (m, 3H), 6.87 (t, J = 5.6 Hz, 1H), 2.23 (d, J = 5.8 Hz, 2H), 1.78-1.66 (m, 11H). | 2- cyclohexylacetic acid |
| 9 | 3-(4-(3- phenyl- propanamido) phenyl)-5- (pyridin-2- ylamino)-1H- pyrazole-4- carboxamide | 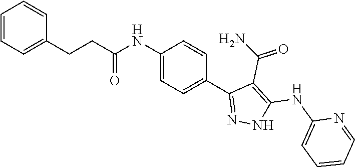 [SMILES: NC(=O)c1c(-c2ccc(NC(=O)CCc3ccccc3)cc2)n[nH]c1Nc1ccccn1] | LCMS (method A): 2.36 min; m/z = 427.1 [M + H]⁺. | ¹H NMR (400 MHz, DMSO-d₆): 13.69 (s, 1H), 10.07 (s, 1H), 8.98 (d, J = 7.2 Hz, 1H), 8.33 (d, J = 8.8 Hz, 1H), 7.90-7.65 (m, 3H), 7.60-7.45 (m, 1H), 7.40-7.05 (m, 7H), 6.98 (s, 1H), 2.96 | 3- phenylpropanoic acid |
| | | | | (t, J = 8.0 Hz, 2H), | |
| | | | | 2.69 (t, J = 8.0 Hz, | |
| | | | | 2H). | |
| 10 | 3-(4-(2-(pyridin- 2-yl)acetamido) phenyl)-5- (pyridin- 2-ylamino)-1H- pyrazole-4- carboxamide | 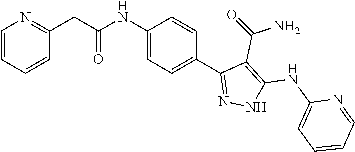 [SMILES: NC(=O)c1c(-c2ccc(NC(=O)Cc3ccccn3)cc2)n[nH]c1Nc1ccccn1] | LCMS (method A): 0.27 min; m/z = 414.2 [M + H]⁺. | ¹H NMR (400 MHz, CDCl₃): 10.26 (s, 1H), 9.99 (s, 1H), 8.67 (d, J = 4.0 Hz, 1H), 8.28 (d, J = 4.0 Hz, 1H), 7.77- 7.69 (m, 3H), 7.65- 7.55 (m, 3H), 7.37 (d, J = 8.0 Hz, 1H), | 2-(pyridin-2- yl)acetic acid |
| | | | | 7.32-7.29 (m, 2H), | |
| | | | | 6.61-6.87 (m, 2H), | |
| | | | | 5.48-5.44 (m, 2H), | |
| | | | | 3.94 (s, 2H). | |
| 11 | 5-(pyridin-2- ylamino)-3-(4- (2-(pyridin-3- yl)acetamido) phenyl)-1H- pyrazole- 4-carboxamide | 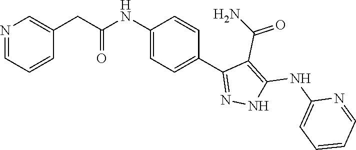 [SMILES: NC(=O)c1c(-c2ccc(NC(=O)Cc3cccnc3)cc2)n[nH]c1Nc1ccccn1] | LCMS (method A): 0.28 min; m/z = 414.2 [M + H]⁺. | ¹H NMR (400 MHz, DMSO-d₆): 13.71 (s, 1H), 10.47 (s, 1H), 8.97 (d, J = 6.8 Hz, 1H), 8.78 (s, 1H), 8.72 (d, J = 4.4 Hz, 1H), 8.36 (d, J = 7.2 Hz, 2H), 8.27 (d, J = 8.0 Hz, 1H), | 2-(pyridin-3- yl)acetic acid |
| | | | | 7.83-7.80 (m, 2H), | |
| | | | | 7.73 (d, J = 8.8 Hz, | |
| | | | | 2H), 7.54 (d, J = | |
| | | | | 8.8 Hz, 1H), 7.14- | |
| | | | | 7.08 (m, 1H), 3.94 | |
| | | | | (s, 2H). | |
| 12 | 5-(pyridin-2- ylamino)-3-(4- (pyridin-4- (2-yl)acetamido) phenyl)-1H- pyrazole- 4-carboxamide | 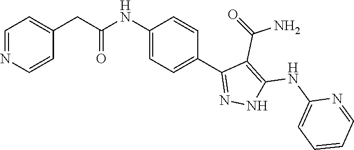 [SMILES: NC(=O)c1c(-c2ccc(NC(=O)Cc3ccncc3)cc2)n[nH]c1Nc1ccccn1] | LCMS (method A): 0.26 min; m/z = 414.2 [M + H]⁺. | ¹H NMR (400 MHz, DMSO-d₆): 12.80 (s, 1H), 10.68 (s, 1H), 9.47 (s, 1H), 8.53 (d, J = 5.6 Hz, 2H), 8.19 (s, 1H), 8.00-7.99 (m, 1H), 7.80-7.69 (m, 3H), 7.54 (d, J = 8.4Hz, | 2-(pyridin-4- yl)acetic acid hydrochloride |
| | | | | 2H), 7.38 (d, J = | |
| | | | | 5.6 Hz, 2H), 6.85 | |
| | | | | (br s, 1H), 5.92 (s, | |
| | | | | 1H), 3.78 (s, 2H). | |
| 13 | 5-(pyridin-2- ylamino)-3-(4- (2-(thiophen-3- yl)acetamido) phenyl)-1H- pyrazole- 4-carboxamide | 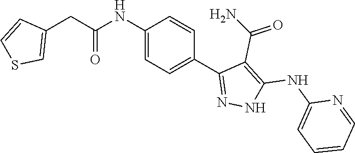 [SMILES: NC(=O)c1c(-c2ccc(NC(=O)Cc3ccsc3)cc2)n[nH]c1Nc1ccccn1] | LCMS (method A): 1.83 min; m/z = 419.1 [M + H]⁺. | ¹H NMR (400 MHz, DMSO-d₆): 12.80 (br s, 1H), 10.43 (br s, 1H), 9.51 (br s, 1H), 8.18 (br s, 1H), 7.99 (br s, 1H), 7.76-7.60 (m, 4H), 7.54-7.40 (m, 4H), 7.35 (br s, 1H), | 2-(thiophen-3- yl)acetic acid |
| | | | | 7.12 (d, J = 4.8 Hz, | |
| | | | | 1H), 6.85 (br s, | |
| | | | | 1H), 3.70 (s, 2H). | |
| 14 | 5-(pyridin-2- ylamino)-3-(4- (2-(thiophen-2- yl)acetamido) phenyl)-1H- pyrazole- 4-carboxamide | 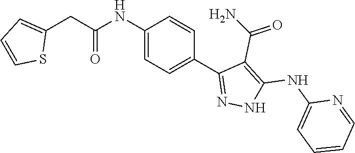 [SMILES: NC(=O)c1c(-c2ccc(NC(=O)Cc3cccs3)cc2)n[nH]c1Nc1ccccn1] | LCMS (method A): 1.90 min; m/z = 419.1 [M + H]⁺. | ¹H NMR (400 MHz, MeOD-d₄): 8.35- 8.29 (m, 2H), 8.08- 8.04 (m, 1H), 7.83 (d, J = 8.8 Hz, 2H), 7.62 (d, J = 8.0 Hz, 2H), 7.33 (d, J = 6.4 Hz, 1H), 7.17- 7.16 (m, 1H), 7.05- 7.00 (m, 2H), 3.95 (s, 2H). | 2-(thiophen-2- yl)acetic acid |
| 15 | 5-(pyridin-2- ylamino)- 3-(4-(4- (trifluoro- methoxy) benzamido) phenyl)- 1H-pyrazole-4- carboxamide | 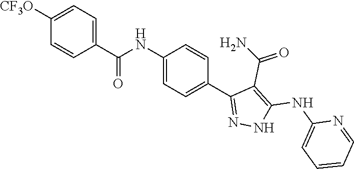 | LCMS (method A): 2.57 min; m/z = 483.1 [M + H]⁺. | ¹H NMR (400 MHz, DMSO-d₆): 12.85 (s, 1 H), 10.60 (s, 1H), 9.54 (s, 1H), 8.21 (s, 1H), 8.13 (d, J = 8.8 Hz, 2H), 8.07 (d, J = 8.8 Hz, 1H), 7.97 (d, J = 8.0 Hz, 2H), 7.71 (m, 1H), 7.59 (m, 5H), 6.87 (s, 1H), 5.92 (br, 1H). | 4- (trifluoromethoxy) benzoic acid |
| 128 | 3-(4-(2-(3- (trifluoro- methyl) phenyl) acetamido) phenyl)-5- (pyridin-2- ylamino)-1H- pyrazole-4- carboxamide | 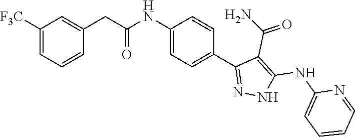 [SMILES: NC(=O)c1c(-c2ccc(NC(=O)Cc3cccc(C(F)(F)F)c3)cc2)n[nH]c1Nc1ccccn1] | LCMS (method A): 2.44 min; m/z = 481.2 [M + H]⁺. | ¹H NMR (400 MHz, DMSO-d₆): 12.78 (s, 1H), 10.55 (s, 1H), 9.50 (br s, 1H), 8.20 (s, 1H), 7.78- 7.52 (m, 12 H), 6.86 (s, 1H), 3.84 (s, 2H). | 2-(3- (trifluoromethyl) phenyl)acetic acid |

### Intermediate A17

### Step 1: 1-(tert-butyl)-3-((2-(2-methoxyethoxy)pyridin-4-yl)amino)-5-(4-nitrophenyl)-1H-pyrazole-4-carbonitrile

3-Amino-1-(tert-butyl)-5-(4-nitrophenyl)-1H-pyrazole-4-carbonitrile (103 mg, 0.360 mmol), Cs₂CO₃ (0.18 g, 0.54 mmol), Xantphos (36 mg, 0.070 mmol) and 4-bromo-2-(2-methoxyethoxy)pyridine (0.1 g, 0.43 mmol) were combined in a 10 mL RBF and 1,4-dioxane (3 mL) was added. The reaction mixture was flushed with N₂ for 10 min. Next, Pd(OAc)₂ (8.11 mg, 0.0400 mmol) was added and the reaction mixture was heated at 80° C. for 30 min and then at 100° C. for an additional 90 min. Water (50 mL) was added and the reaction mixture was extracted with EtOAc (3×40 mL). The combined organic layers were washed with brine, dried (Na₂SO₄) and concentrated. The crude residue was purified by silica gel column chromatography (PE:EtOAc, 1:0 to 0:1) to afford the title compound (105 mg, 67%) as a yellow oil. LCMS (method C): 2.37 min; m/z=437.2 [M+H]⁺.

### Step 2: 1-(tert-butyl)-3-((2-(2-methoxyethoxy)pyridin-4-yl)amino)-5-(4-nitrophenyl)-1H-pyrazole-4-carboxamide

To a mixture of 1-(tert-butyl)-3-((2-(2-methoxyethoxy)pyridin-4-yl)amino)-5-(4-nitrophenyl)-1H-pyrazole-4-carbonitrile (105 mg, 0.240 mmol) and K₂CO₃ (0.10 g, 0.72 mmol) in DMSO (5 mL), was added 30% aq. H₂O₂ (2 mL) and the reaction mixture was stirred at 60° C. for 1 h. An additional portion of H₂O₂ (2 mL) was added and the reaction mixture was continued stirring at 60° C. for 2 h. The reaction mixture was cooled to RT, H₂O (150 mL) was added and the reaction mixture was extracted with EtOAc (3×100 mL). The combined organic layers were washed with brine, dried (Na₂SO₄) and then concentrated under reduced pressure. The crude residue was purified by silica gel column chromatography (DCM:MeOH, 1:0 to 1:0) to afford the title product (25 mg, 23%) as a yellow oil. LCMS (method C): 2.00 min; m/z=455.2 [M+H]⁺.

### Step 3: 5-(4-aminophenyl)-1-(tert-butyl)-3-((2-(2-methoxyethoxy)pyridin-4-yl)amino)-1H-pyrazole-4-carboxamide (Intermediate A17)

A mixture of 1-(tert-butyl)-3-((2-(2-methoxyethoxy)pyridin-4-yl)amino)-5-(4-nitrophenyl)-1H-pyrazole-4-carboxamide (25 mg, 0.055 mmol) and Pd/C (5 mg) in MeOH (5 mL) was stirred under an atmosphere of H₂ overnight. The reaction mixture was filtered through Celite and the filtrate concentrated under reduced pressure to give the title product (21 mg, 90%) as a yellow oil, which solidified upon standing. LCMS (method C): 1.52 min; m/z=452.2 [M+H]⁺.

The following compounds were similarly prepared from the appropriate carboxylic acid and 5-(4-aminophenyl)-1-(tert-butyl)-3-((2-(2-methoxyethoxy)pyridin-4-yl)amino)-1H-pyrazole-4-carboxamide intermediate A17 according to the method described for the synthesis of compound 1:

TABLE 5

| Compound | | | | | |
|---|---|---|---|---|---|
| No | Name | Structure | LCMS data | ¹HNMR data | SM |
| 16 | 3-(4- acetamidophenyl)- 5-((2-(2- methoxyethoxy) pyridin-4- yl)amino)-1H- pyrazole-4- carboxamide | 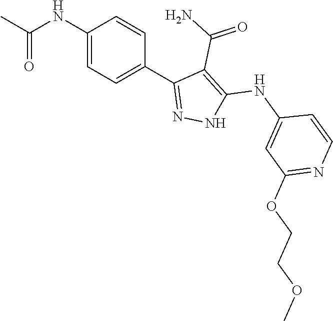 [SMILES: COCCOc1cc(Nc2[nH]nc(-c3ccc(NC(C)=O)cc3)c2C(N)=O)ccn1] | LCMS (method A): 0.32 min; m/z = 411.0 [M + H]⁺. | ¹H NMR (400 MHz, MeOD-d6): 7.84- 7.78 (m, 3H), 7.55 (d, J = 8.0 Hz, 2H), 7.22 (s, 1H), 6.92 (d, J = 4.0 Hz, 1H), 4.37 (s, 2H), 3.74 (s, 2H), 3.43 (s, 3H), 2.17 (s, 3H) ppm. | Acetic acid |
| 17 | 3-(4-(2- aminoacetamido) phenyl)-5-((2-(2- methoxyethoxy) pyridin-4- yl)amino)-1H- pyrazole-4- carboxamide | 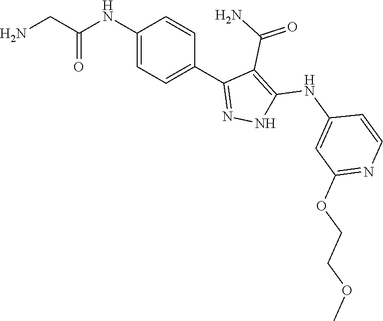 [SMILES: COCCOc1cc(Nc2[nH]nc(-c3ccc(NC(=O)CN)cc3)c2C(N)=O)ccn1] | LCMS (method A): 0.32 min; m/z = 426.0 [M + H]⁺. | ¹H NMR (400 MHz, MeOD-d4): 7.93 (d, J = 6.8 Hz, 1H), 7.83 (d, J = 8.4 Hz, 2H), 7.63-7.61 (d, 3H), 7.34 (br s, 1H), 4.49 (t, J = 4.4 Hz, 2H), 3.91 (s, 2H), 3.82 (t, J = 4.4 Hz, 2H ), 3.43 (s, 3H), 2.70 (s, 2H). | glycine |
| 18 | 3-(4-(2- aminopropan- amido)phenyl)-5- ((2-(2- methoxyethoxy) pyridin-4- yl)amino)-1H- pyrazole-4- carboxamide | 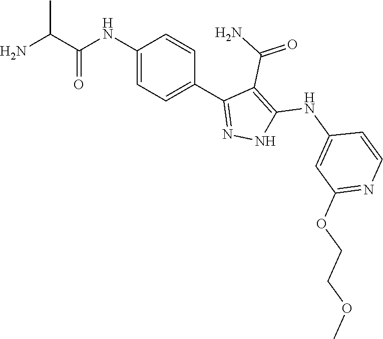 [SMILES: COCCOc1cc(Nc2[nH]nc(-c3ccc(NC(=O)C(C)N)cc3)c2C(N)=O)ccn1] | LCMS (method A): 0.39 min; m/z = 440.1 [M + H]⁺. | ¹H NMR (400 MHz, MeOD-da): 7.85- 7.81 (t, 3H), 7.58 (d, J = 8.8 Hz, 2H), 7.20 (d, J = 1.6 Hz, 1H), 6.91 (dd, J = 6.0, 2.0 Hz, 1H), 4.35 (t, J = 4.8 Hz, 2H), 3.76-3.74 (m, 3H), 3.42 (s, 3H), 1.46 (m, 3H). | alanine |
| 19 | 3-(4-(2- (dimethylamino) acetamido) phenyl)- 5-((2-(2- methoxyethoxy) pyridin- 4-yl)amino)-1H- pyrazole-4- carboxamide | 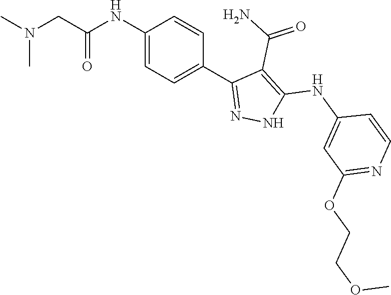 [SMILES: COCCOc1cc(Nc2[nH]nc(-c3ccc(NC(=O)CN(C)C)cc3)c2C(N)=O)ccn1] | LCMS (method A): 0.32 min; m/z = 454.1 [M + H]⁺. | ¹H NMR (400 MHz, MeOD-d₄): 7.85- 7.83 (m, 3H), 7.58 (d, J = 8.4 Hz, 2H), 7.22 (s, 1H), 6.92 (d, J = 5.6 Hz, 1H), 4.35 (t, J = 4.8 Hz, 2H), 3.75 (t, J = 4.8 Hz, 2H), 3.42 (s, 2H), 3.42 (s, 3H), 2.49 (s, 6H). | dimethylglycine |

### Compound 20

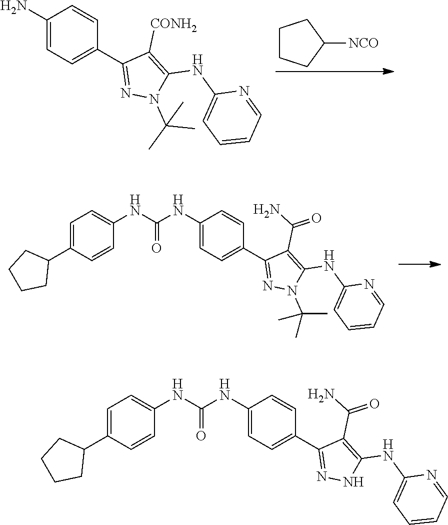

### Step 1: 1-(tert-butyl)-3-(4-(3-cyclopentylureido)phenyl)-5-(pyridin-2-ylamino)-1H-pyrazole-4-carboxamide

A mixture of 3-(4-aminophenyl)-1-(tert-butyl)-5-(pyridin-2-ylamino)-1H-pyrazole-4-carboxamide (50 mg, 0.14 mmol) and isocyanatocyclopentane (38 mg, 0.34 mmol) in THF (5 mL) was stirred at RT for 18 h. The mixture was concentrated under reduced pressure and the residue was diluted with EtOAc (15 mL) and washed with H₂O (2×10 mL). The organic layer was dried (Na₂SO₄) and then concentrated under reduced pressure. The crude residue was purified by prep-TLC (DCM:MeOH, 20:1) to afford the title product (12 mg, 25%) as a white solid. LCMS (method A): 2.48 min; m/z=462.1 [M+H]⁺.

### Step 2: 3-(4-(3-cyclopentylureido)phenyl)-5-(pyridin-2-ylamino)-1H-pyrazole-4-carboxamide (Compound 20)

A solution of 1-(tert-butyl)-3-(4-(3-cyclopentylureido)phenyl)-5-(pyridin-2-ylamino)-1H-pyrazole-4-carboxamide (12 mg, 0.026 mmol) in TFA (1 mL) was stirred at 60° C. for 1 h. The solution was concentrated under reduced pressure and the residue was triturated (Et₂O) to afford the title product·mono TFA salt (6 mg, 57%) as a white solid. LCMS (method A): 2.03 min; m/z=406.1 [M+H]⁺. ¹NMR (400 MHz, DMSO-d₆): 13.62 (s, 1H), 9.70 (s, 1H), 8.96 (d, J=7.3 Hz, 1H), 8.53 (s, 1H), 8.39 (m, 1H), 8.20 (s, 1H), 7.66 (m, 2H), 7.55 (m, 2H), 7.20 (m, 1H), 3.98 (m, 1H), 1.84 (d, J=6.4 Hz, 2H), 1.73 (m, 2H), 1.47 (m, 2H), 1.28 (br s, 2H).

### Compound 21

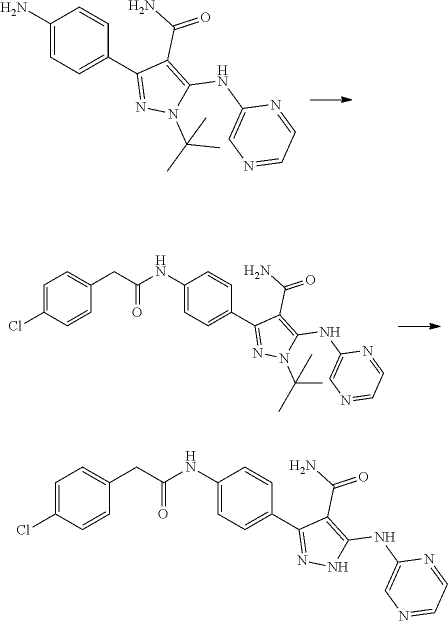 [SMILES: CC(C)(C)n1nc(-c2ccc(N)cc2)c(C(N)=O)c1Nc1cnccn1 ; CC(C)(C)n1nc(-c2ccc(NC(=O)Cc3ccc(Cl)cc3)cc2)c(C(N)=O)c1Nc1cnccn1 ; NC(=O)c1c(-c2ccc(NC(=O)Cc3ccc(Cl)cc3)cc2)n[nH]c1Nc1cnccn1]

### Step 1: 1-(tert-butyl)-3-(4-(2-(4-chlorophenyl)acetamido)phenyl)-5-(pyrazin-2-ylamino)-1H-pyrazole-4-carboxamide

To a solution of 3-(4-aminophenyl)-1-tert-butyl-5-[(pyrazin-2-yl)amino]-1H-pyrazole-4-carboxamide intermediate A3 (200 mg, 569 μmol) in DMF (8 mL) was added 2-(4-chlorophenyl)acetic acid (145 mg, 853 μmol), HATU (300 mg, 1.13 mmol) and Et₃N (229 mg, 2.27 mmol) and the resulting mixture was stirred at RT overnight. The mixture was diluted with PE:EtOAc (1:1, 50 mL) and then filtered through Celite. The filter cake was washed with PE:EtOAc (1:1, 3×20 mL) and then dried under reduced pressure to afford the title compound (200 mg, 70%) as a white solid. LCMS (method A): 3.65 min; m/z=504.2 [M+H]⁺.

### Step 2: 3-(4-(2-(4-chlorophenyl)acetamido)phenyl)-5-(pyrazin-2-ylamino)-1H-pyrazole-4-carboxamide (Compound 21)

A solution of 1-tert-butyl-3-{4-[2-(4-chlorophenyl)acetamido]phenyl}-5-[(pyrazin-2-yl)amino]-1H-pyrazole-4-carboxamide (100 mg, 198 μmol) in DCM (4 mL) and TFA (4 mL) was stirred at RT overnight. The reaction mixture was concentrated under reduced pressure and the residue was suspended in NH₄OH (5 mL) and then diluted with H₂O (10 mL). The precipitated solids were filtered, and the filter cake was dried under reduced pressure to give the desired product (15 mg, 17%) as a white solid. LCMS (method A): 3.42 min; m/z=448.1 [M+H]⁺. ¹H NMR (400 MHz, DMSO-d₆): 10.45 (s, 1H), 9.64 (s, 1H), 9.27 (s, 1H), 8.26-8.19 (s, 1H), 8.12 (d, J=2.8 Hz, 1H), 7.77 (d, J=8 Hz, 2H), 7.55 (d, J=8.4 Hz, 1H), 8.43-8.35 (m, 4H), 6.08 (br s, 1H), 3.70 (s, 2H).

The following compounds were similarly prepared from the appropriate carboxylic acid and 3-(4-aminophenyl)-1-tert-butyl-5-[(pyrazin-2-yl)amino]-1H-pyrazole-4-carboxamide intermediate A3 following step 1 according to the method described for the synthesis of compound 21:

TABLE 6

| Compound | | | | | |
|---|---|---|---|---|---|
| No | Name | Structure | LCMS data | ¹HNMR data | SM |
| 22 | 5-(pyrazin-2-ylamino)- 3-(4-(2-(4- (trifluoromethyl)phenyl) acetamido)phenyl)- 1H-pyrazole-4- carboxamide | 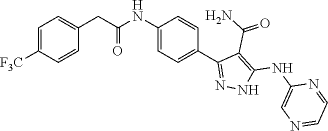 [SMILES: NC(=O)c1c(-c2ccc(NC(=O)Cc3ccc(C(F)(F)F)cc3)cc2)n[nH]c1Nc1cnccn1] | LCMS (method A): 3.59 min; m/z = 482.1 [M + H]⁺. | ¹H NMR (400 MHz, DMSO- d₆): 10.44 (br s, 1H), 9.71 (br s, 1H), 8.17-8.10 (m, 2H), 7.64 (d, J = 6.4 Hz, 8H), 3.81 (s, 3H). | 2-(4- (trifluoromethyl) phenyl)acetic acid |
| 23 | 5-(pyrazin-2-ylamino)- 3-(4-(2-(3- (trifluoromethoxy) phenyl) acetamido)phenyl)- 1H-pyrazole-4- carboxamide | 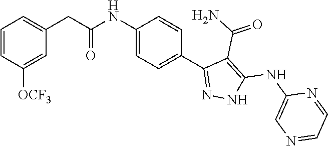 [SMILES: NC(=O)c1c(-c2ccc(NC(=O)Cc3cccc(OC(F)(F)F)c3)cc2)n[nH]c1Nc1cnccn1] | LCMS (method A): 3.64 min; m/z = 498.1 [M + H]⁺. | ¹H NMR (400 MHz, DMSO- d₆): 12.99 (s, 1H), 10.72 (s, 1H), 9.63 (s, 1H), 9.27 (s, 1H), 8.16 (d, J = 44.8 Hz, 2H), 7.79 (s, | 2-(3- (trifluoromethoxy) phenyl)acetic acid |
| | | | | 2H), 7.40 (m, | |
| | | | | 6H), 3.80 (s, | |
| | | | | 2H). | |
| 24 | 5-(pyrazin-2-ylamino)- 3-(4-(2-(3- (trifluoromethyl)phenyl) acetamido)phenyl)- 1H-pyrazole-4- carboxamide | 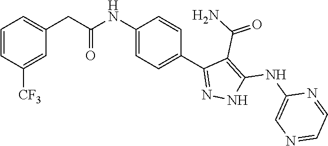 [SMILES: NC(=O)c1c(-c2ccc(NC(=O)Cc3cccc(C(F)(F)F)c3)cc2)n[nH]c1Nc1cnccn1] | LCMS (method A): 3.55 min; m/z = 482.1 [M + H]⁺. | ¹H NMR (400 MHz, DMSO- d₆): 12.95 (s, 1H), 10.49 (s, 1H), 9.63 (s, 1H), 9.27 (s, 1H), 8.22 (s, 1H), 8.11 (s, 1H), 7.74 (d, | 2-(3- (trifluoromethyl) phenyl)acetic acid |
| | | | | J = 15.6 Hz, | |
| | | | | 3H), 7.65-7.56 | |
| | | | | (m, 5H), 7.34 | |
| | | | | (s, 1H), 6.07 (s, | |
| | | | | 1H), 3.84 (s, | |
| | | | | 2H). | |
| 25 | 3-(4-(2-(3- methoxyphenyl) acetamido)phenyl)-5- (pyrazin-2-ylamino)- 1H-pyrazole-4- carboxamide | 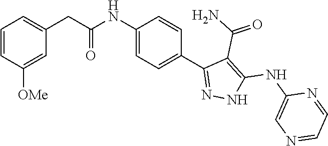 [SMILES: COc1cccc(CC(=O)Nc2ccc(-c3n[nH]c(Nc4cnccn4)c3C(N)=O)cc2)c1] | LCMS (method A): 3.23 min; m/z = 444.1 [M + H]⁺. | ¹H NMR (400 MHz, DMSO- d₆): 10.43 (s, 1H), 9.63 (s, 1H), 9.26 (s, 1H), 8.21 (d, J = 0.4 Hz, 1H), 8.10 (d, J = 2.4 Hz, 1H), 7.77 | 2-(3- methoxyphenyl) acetic acid |
| | | | | (d, J = 8.0 Hz, | |
| | | | | 2H), 7.53 (d, | |
| | | | | J = 8.0 Hz, | |
| | | | | 2H), 7.24 (t, | |
| | | | | J = 8.0 Hz, | |
| | | | | 1H), 6.92 (d, J = | |
| | | | | 7.2 Hz, 2H), | |
| | | | | 6.82 (t, J = 7.2 Hz, | |
| | | | | 1H), 3.75 (s, 3H), | |
| | | | | 3.65 (s, | |
| | | | | 2H). | |
| 26 | 3-(4-(2-(3- chlorophenyl) acetamido) phenyl)-5-(pyrazin- 2-ylamino)-1H- pyrazole-4- carboxamide | 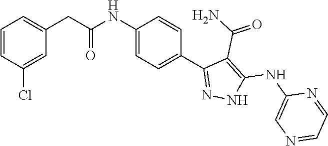 [SMILES: NC(=O)c1c(-c2ccc(NC(=O)Cc3cccc(Cl)c3)cc2)n[nH]c1Nc1cnccn1] | LCMS (method A): 3.43 min; m/z = 448.1 [M + H]⁺. | ¹H NMR (400 MHz, DMSO- d₆): 12.94 (s, 1H), 10.44 (s, 1H), 9.63 (s, 1H), 9.26 (d, J = 0.8 Hz, 1H), 8.22 (q, J = 2.4 Hz, 1H), 8.11 | 2-(3- chlorophenyl) acetic acid |
| | | | | (d, J = 2.4 Hz, | |
| | | | | 2H), 7.54 (d, | |
| | | | | J = 8.4 Hz, | |
| | | | | 2H), 7.42 (s, | |
| | | | | 1H), 7.37- | |
| | | | | 7.31 (m, 3H), | |
| | | | | 3.72 (s, 2H). | |
| 27 | 5-(pyrazin-2-ylamino)- 3-(4-(2-(2- (trifluoromethyl) phenyl) acetamido)phenyl)- 1H-pyrazole-4- carboxamide | 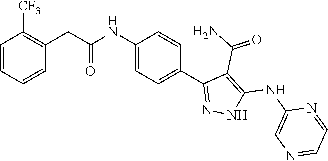 [SMILES: NC(=O)c1c(-c2ccc(NC(=O)Cc3ccccc3C(F)(F)F)cc2)n[nH]c1Nc1cnccn1] | LCMS (method A): 3.47 min; m/z = 482.1 [M + H]⁺. | ¹H NMR (400 MHz, DMSO- d₆): 13.05 (s, 1H), 10.88 (s, 1H), 9.63 (s, 1H), 9.26 (s, 1H), 8.21 (s, 1H), 8.10 (d, J = 2.4 Hz, 1H), 7.82 (d, J = 8.4 | 2-(2- (trifluoromethyl) phenyl)acetic acid |
| | | | | Hz, 2H), 7.71 | |
| | | | | (d, J = 7.6 Hz, | |
| | | | | 2H), 7.65 (t, | |
| | | | | J = 7.6 Hz, 1H), | |
| | | | | 7.54 (d, J = 8.4 | |
| | | | | Hz, 2H), 7.49 | |
| | | | | (t, J = 8 Hz, 3H), | |
| | | | | 4.01 (s, 2H). | |
| 28 | 3-(4-(2-(2- methoxyphenyl) acetamido)phenyl)-5- (pyrazin-2-ylamino)- 1H-pyrazole-4- carboxamide | 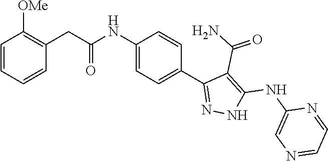 [SMILES: COc1ccccc1CC(=O)Nc1ccc(-c2n[nH]c(Nc3cnccn3)c2C(N)=O)cc1] | LCMS (method A): 3.25 min; m/z = 444.1 [M + H]⁺. | ¹H NMR (400 MHz, DMSO- d₆): 12.96 (s, 1H), 10.34 (s, 1H), 9.65 (s, 1H), 9.28 (s, 1H), 8.23 (s, 1H), 8.12 (s, 1H), 7.78 (s, 2H), 7.55 (s, | 2-(2- methoxyphenyl) acetic acid |
| | | | | 2H), 7.25 (s, | |
| | | | | 2H), 7.00 (s, | |
| | | | | 1H), 6.92 (s, | |
| | | | | 1H), 6.09 (br s, | |
| | | | | 1H), 3.78 (s, | |
| | | | | 3H), 3.68 (s, | |
| | | | | 2H). One active | |
| | | | | proton obscured. | |
| 29 | 3-(4-(2- phenylacetamido) phenyl)-5-(pyrazin- 2-ylamino)-1H- pyrazole- 4-carboxamide | 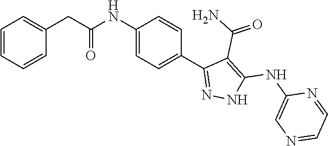 [SMILES: NC(=O)c1c(-c2ccc(NC(=O)Cc3ccccc3)cc2)n[nH]c1Nc1cnccn1] | LCMS (method A): 3.21 min; m/z = 414.1 [M + H]⁺. | ¹H NMR (400 MHz, DMSO- d₆): 12.94 (s, 1H), 10.42 (s, 1H), 9.64 (s, 1H), 9.27 (s, 1H), 8.22 (s, 1H), 8.11 (s, 1H), 7.78 (d, | Phenylacetic acid |
| | | | | J = 8.4 Hz, 2H), | |
| | | | | 7.54 (d, J = 8.4 | |
| | | | | Hz, 2H), 7.38- | |
| | | | | 7.22 (m, 6H), | |
| | | | | 3.69 (s, 2H). | |
| 30 | 5-(pyrazin-2-ylamino)- 3-(4-(2-(4- (trifluoromethoxy) phenyl)acetamido) phenyl)- 1H-pyrazole-4- carboxamide | 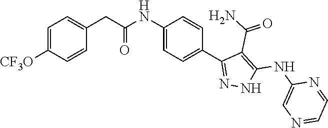 | LCMS (method A): 3.62 min; m/z = 498.1 [M + H]⁺. | ¹H NMR (400 MHz, MeOD- d₄): 12.95 (s, 1H), 10.51 (s, 1H), 9.63 (s, 1H), 9.26 (s, 1H), 7.77 (d, J = 8.0 Hz, | 2-(4- (trifluoromethoxy) phenyl)acetic acid |
| | | | | 1H), 7.54 (d, | |
| | | | | J = 8.8 Hz, 2H), | |
| | | | | 7.47 (d, J = 8.8 | |
| | | | | Hz, 2H), 7.33 | |
| | | | | (d, J = 8.0 Hz, | |
| | | | | 1H), 6.05 (s, | |
| | | | | 1H), 3.75 (s, | |
| | | | | 2H). | |
| 31 | 3-(4-(2-(4- methoxyphenyl) acetamido)phenyl)-5- (pyrazin-2-ylamino)- 1H-pyrazole-4- carboxamide | 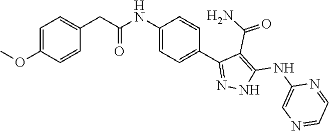 [SMILES: COc1ccc(CC(=O)Nc2ccc(-c3n[nH]c(Nc4cnccn4)c3C(N)=O)cc2)cc1] | LCMS (method A): 3.23 min; m/z = 444.2 [M + H]⁺. | ¹H NMR (400 MHz, DMSO- d₆): 12.94 (s, 1H), 10.37 (s, 1H), 9.64 (s, 1H), 9.27 (s, 1H), 8.22 (s, 1H), 8.21 (s. | 2-(4- methoxyphenyl) acetic acid |
| | | | | 1H), 7.77 (t, | |
| | | | | J = 2.0 Hz, 7H), | |
| | | | | 7.54 (d, J = 8.0 | |
| | | | | Hz, 2H), 7.26 | |
| | | | | (d, J = 8.0 Hz, | |
| | | | | 2H), 6.90 (d, J = | |
| | | | | 8.0 Hz, 2H), | |
| | | | | 6.02 (br s, 1H), | |
| | | | | 3.73 (s, 3H), | |
| | | | | 3.60 (s, 2H) | |
| 32 | 3-(4-(2-(2- chlorophenyl) acetamido) phenyl)-5-(pyrazin- 2-ylamino)-1H- pyrazole-4- carboxamide | 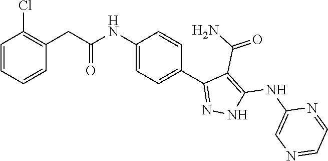 [SMILES: NC(=O)c1c(-c2ccc(NC(=O)Cc3ccccc3Cl)cc2)n[nH]c1Nc1cnccn1] | LCMS (Method A): 3.37 min; m/z = 448.1, 450.1 [M + H]⁺. | ¹H NMR (400 MHz, DMSO- d₆): 12.96 (br s, 1H), 10.48 (br s, 1H), 9.64 (br s, 1H), 9.27 (br s, 1H), 8.15 (d, J = 12.4 Hz, 2H), 7.76 (dd, J = | 2-(2- chlorophenyl) acetic acid |
| | | | | 2.0 Hz, 5.2 Hz, | |
| | | | | 2H), 7.56- | |
| | | | | 7.32 (m, 7H), | |
| | | | | 3.88 (s, | |
| | | | | 2H). One active | |
| | | | | proton | |
| | | | | obscured. | |

### Compound 33

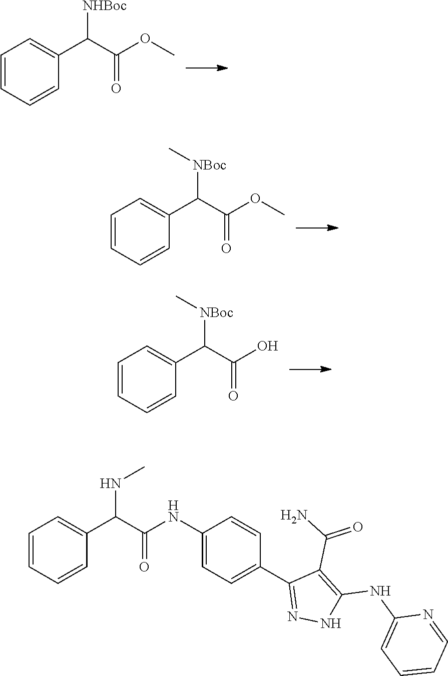 [SMILES: COC(=O)C(NC(=O)OC(C)(C)C)c1ccccc1 ; COC(=O)C(c1ccccc1)N(C)C(=O)OC(C)(C)C ; CN(C(=O)OC(C)(C)C)C(C(=O)O)c1ccccc1 ; CNC(C(=O)Nc1ccc(-c2n[nH]c(Nc3ccccn3)c2C(N)=O)cc1)c1ccccc1]

### Step 1: methyl 2-((tert-butoxycarbonyl)(methyl)amino)-2-phenylacetate

To a solution of methyl 2-((tert-butoxycarbonyl)amino)-2-phenylacetate (410 mg, 1.55 mmol) in DMF (5 mL), was added NaH (93 mg, 2.32 mmol) at −40° C. and the solution was then stirred at 0° C. for 20 min. Dimethyl sulfate (253 mg, 2.01 mmol) was added and the solution was stirred at RT for 2.5 h. The mixture was poured into a 3:1 mixture of sat. aq. NH₄Cl and 1.0 M aq. HCl and the resulting mixture was extracted with EtOAc. The organic layer was dried (Na₂SO₄), filtered and concentrated under reduced pressure. The crude residue was purified by silica gel column chromatography (PE:EtOAc, 50:1) to afford the title product (140 mg, 22%) as colorless oil. LCMS (method A): 2.72 min; m/z=280.1 [M+H]⁺.

### Step 2: 2-((tert-butoxycarbonyl)(methyl)amino)-2-phenylacetic acid

To a solution of methyl 2-((tert-butoxycarbonyl)(methyl)amino)-2-phenylacetate (85 mg, 0.30 mmol) in THF:MeOH:H₂O (3:2:1, 6 mL) at 0° C., was added LiOH·H₂O (38 mg, 0.9 mmol). The mixture was stirred at RT for 4 h, then diluted with H₂O (10 mL) and extracted with EtOAc (2×20 mL). The aqueous layer was acidified to pH 3-4 with 1.0 M aq. HCl and then extracted with EtOAc (2×20 mL). The combined organics were dried (Na₂SO₄) and concentrated under reduced pressure to afford the title product (103 mg, 66%) as an oil. LCMS (method A): 2.81 min; m/z=288.1 [M+Na]⁺.

### Step 3: 3-(4-(2-(methylamino)-2-phenylacetamido)phenyl)-5-(pyridin-2-yl amino)-1H-pyrazole-4-carboxamide (Compound 33)

2-((tert-Butoxycarbonyl)(methyl)amino)-2-phenylacetic acid (20 mg, 0.07 mmol), EDC.HCl (29 mg, 0.15 mmol) and DMAP (9 mg, 0.07 mmol) were dissolved in DMF (2 mL). The mixture was stirred at RT for 20 min and then 3-(4-aminophenyl)-1-(tert-butyl)-5-(pyridin-2-ylamino)-1H-pyrazole-4-carboxamide (26 mg, 0.07 mmol) was added in one portion. The resulting mixture was stirred at RT for 18 h under N₂. The mixture was diluted with H₂O and then extracted with EtOAc (2×5 mL). The combined organics were concentrated under reduced pressure and the crude residue was purified by prep-TLC (DCM:MeOH, 10:1) to give a mixture of tert-butyl (2-((4-(1-(tert-butyl)-4-carbamoyl-5-(pyridin-2-ylamino)-1H-pyrazol-3-yl)phenyl)amino)-2-oxo-1-phenylethyl)(methyl)carbamate (15 mg, 16%) (LCMS (method A): 2.90 min; m/z=598.4 [M+H]⁺) as a white solid and 1-(tert-butyl)-3-(4-(2-(methylamino)-2-phenylacetamido)phenyl)-5-(pyridin-2-ylamino)-1H-pyrazole-4-carboxamide (13 mg, 13%) (LCMS (method A): 2.17 min; m/z=498.3 [M+H]⁺) as a colorless oil. Both residues were dissolved in TFA (2 mL) and the reaction mixture was stirred at 60° C. for 4 h, then diluted with H₂O (10 mL) and extracted with EtOAc (2×10 mL). The aqueous phase was basified to pH 9-10 with aq. NaHCO₃ and then extracted with DCM (2×10 mL). The combined organic layers were dried (Na₂SO₄), concentrated under reduced pressure and the residue was triturated (Et₂O) to give the title product (13 mg, 63%) as a white solid. LCMS (method A): 2.57 min; m/z=442.2 [M+H]⁺. ¹H NMR (400 MHz, DMSO-d₆): 12.77 (br s, 1H), 10.30 (br s, 1H), 9.49 (br s, 1H), 8.19 (br s, 1H), 7.99-7.77 (m, 5H), 7.71-7.48 (m, 5H), 7.37-7.29 (m, 4H), 6.84 (br s, 1H), 4.27 (s, 1H), 2.31 (s, 3H).

### Compound 34

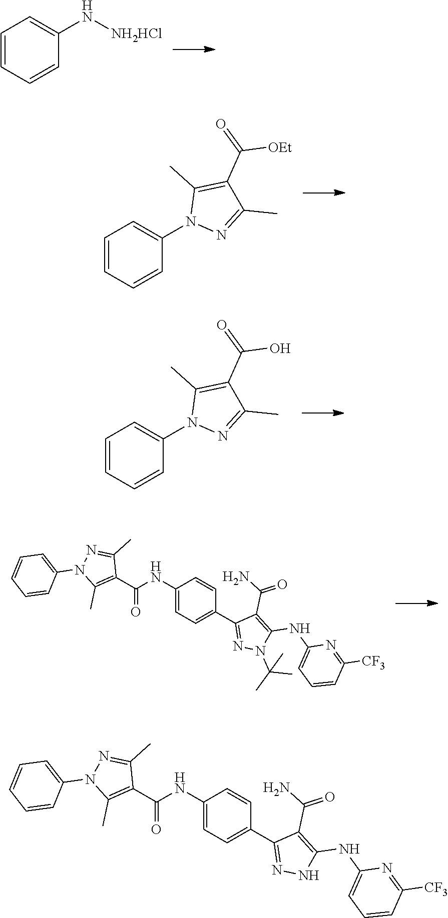

### Step 1: ethyl 3,5-dimethyl-1-phenyl-1H-pyrazole-4-carboxylate

To a solution of PhNHNH₂·HCl (1 g, 6.91 mmol) in EtOH (15 mL), was slowly added ethyl 2-acetyl-3-oxobutanoate (2.96 g, 17.2 mmol) in EtOH (30 mL) at 0° C. The mixture was stirred at 30° C. for 12 h and then diluted with H₂O (600 mL). The mixture was extracted with EtOAc (3×300 mL), and the combined organics were washed with brine (200 mL), dried (Na₂SO₄), and concentrated under reduced pressure. The crude residue was purified by silica gel column chromatography (PE:EtOAc, 50:1 to 30:1) to afford the desired product (270 mg, 16%) as a white solid. LCMS (method A): 2.90 min; m/z=245.4 [M+H]⁺.

### Step 2: 3,5-dimethyl-1-phenyl-1H-pyrazole-4-carboxylic acid

To a solution of ethyl 3,5-dimethyl-1-phenyl-1H-pyrazole-4-carboxylate (260 mg, 1.06 mmol) in MeOH (5 mL), was added 2.0 M aq. NaOH (5 mL) and the mixture was stirred at RT for 16 h. The mixture was acidified with 1.0 M aq. HCl and the precipitated solids were collected and dried under reduced pressure to provide the title product (170 mg, 74%) as a yellow solid. LCMS (method A): 0.98 min; m/z=216.9 [M+H]⁺. ¹H NMR (400 MHz, DMSO-d₆): 12.35 (s, 1H), 7.56-7.45 (m, 5H), 2.46 (s, 3H), 2.36 (s, 3H).

### Step 3: N-(4-(1-(tert-butyl)-4-carbamoyl-5-((6-(trifluoromethyl)pyridin-2-yl)amino)-1H-pyrazol-3-yl)phenyl)-3,5-dimethyl-1-phenyl-1H-pyrazole-4-carboxamide

A mixture of 3-(4-aminophenyl)-1-tert-butyl-5-{[6-(trifluoromethyl)pyridin-2-yl]amino}-1H-pyrazole-4-carboxamide (150 mg, 0.3584 mmol), 3,5-dimethyl-1-phenyl-1H-pyrazole-4-carboxylic acid (100 mg, 0.4659 mmol), HATU (272 mg, 0.7168 mmol) and Et₃N (144 mg, 1.43 mmol) in DMF (5 mL) was stirred at RT for 16 h. The mixture was partitioned between H₂O and EtOAc and the organic layer were washed with brine, dried (Na₂SO₄), and concentrated under reduced pressure. The crude residue was purified by prep-TLC (DCM:MeOH, 12:1) to afford the desired product (44 mg, 20%) as a brown solid. LCMS (method A): 3.09 min; m/z=617.2 [M+H]⁺.

### Step 4: N-(4-(4-carbamoyl-5-((6-(trifluoromethyl)pyridin-2-yl)amino)-1H-pyrazol-3-yl)phenyl)-3,5-dimethyl-1-phenyl-1H-pyrazole-4-carboxamide (Compound 34)

A mixture of 1-tert-butyl-3-[4-(3,5-dimethyl-1-phenyl-1H-pyrazole-4-amido)phenyl]-5-{[6-(trifluoromethyl)pyridin-2-yl]amino}-1H-pyrazole-4-carboxamide (43 mg, 0.06973 mmol) in TFA (2 mL) and DCM (2 mL) was stirred at RT for 16 h. The solution was concentrated under reduced pressure and the residue was diluted with H₂O and then basified to pH 10 with NH₄OH. The precipitated solids were collected by filtration to afford the desired product (27 mg, 69%) as a yellow solid. LCMS (method A): 0.83 min; m/z=561.1 [M+H]⁺. ¹H NMR (400 MHz, DMSO-d₆): 12.96 (s, 1H), 10.09 (s, 1H), 9.78 (s, 1H), 8.25 (d, J=6 Hz, 1H), 8.00 (t, J=8 Hz, 1H), 7.87 (d, J=6.8 Hz, 2H), 7.59-7.46 (m, 8H), 7.32 (d, J=6.8 Hz, 1H), 6.10 (br s, 1H), 2.43 (s, 3H), 2.38 (s, 3H).

### Intermediate Acid

### Step 1: ethyl 1-(4-chlorophenyl)-1H-pyrazole-4-carboxylate

To a solution of 4-chlorophenyl)hydrazine (1 g, 7.01 mmol) in EtOH (12 mL) cooled to 0° C., was added a solution of ethyl 2-formyl-3-oxopropanoate (3.02 g, 21.0 mmol) in EtOH (3 mL) and the reaction was stirred at RT overnight. The mixture was concentrated under reduced pressure and the residue was then partitioned between sat. aq. NaHCO₃ (20 mL) and EtOAc (20 mL). The aqueous layer was extracted with EtOAc (2×20 mL) and the combined organic layers were dried (Na₂SO₄) and concentrated under reduced pressure. The crude residue was purified by silica gel column chromatography (PE:EtOAc, 30:1) to afford the desired product (1.6 g, 91%) as a yellow solid. LCMS (method B): 2.53 min; m/z=251.7 [M+H]⁺.

### Step 2: 1-(4-chlorophenyl)-1H-pyrazole-4-carboxylic acid

To a solution of ethyl 1-(4-chlorophenyl)-1H-pyrazole-4-carboxylate (1.6 g, 6.38 mmol) in MeOH (64 mL) was added a solution of NaOH (764 mg, 19.1 mmol) in H₂O (64 mL) and the mixture was stirred at RT overnight. The mixture was concentrated under reduced pressure, and the residue was diluted with H₂O (30 mL) and acidified with 1.0 M aq. HCl (20 mL). The precipitated solids were collected by filtration and dried under reduced pressure to afford the desired product (784 mg, 55%) as a yellow solid. LCMS (method B): 2.53 min; m/z=223.6 [M+H]⁺.

The following compounds were similarly prepared from the appropriate phenylhydrazine and 1-tert-butyl-3-{4-[1-(4-chlorophenyl)-1H-pyrazole-4-amido]phenyl}-5-{[6-(trifluoromethyl)pyridine-2-yl]amino}-1H-pyrazole-4-carboxamide according to the method described for the synthesis of compound 34:

TABLE 7

| Compound No | Name | Structure | LCMS data | ¹H NMR data | SM |
|---|---|---|---|---|---|
| 35 | N-(4-(4-carbamoyl-5-((6- (trifluoromethyl)pyridin- 2-yl)amino)-1H-pyrazol- 3-yl)phenyl)-1-(4- trifluoromethoxy) phenyl)-1H-pyrazole-4- carboxamide | 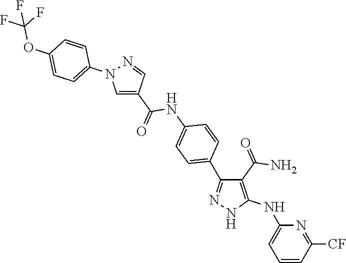 | LCMS (method A): 3.20 min; m/z = 616.8 [M + H]⁺. | ¹H NMR (400 MHz, DMSO-d₆): 12.98 (s, 1H), 10.25 (s, 1H), 9.77 (s, 1H), 9.18 (s, 1H), 8.40 (s, 1H), 8.25 (d, J = 8.8 Hz, 1H), 8.05- 7.92 (m, 6H), 7.62- 7.57 (m, 5H), 7.32 (d, J = 8.8 Hz, H). | (4- (trifluoro- methoxy) phenyl) hydrazine |
| 36 | N-(4-(4-carbamoyl-5-((6- (trifluoromethyl)pyridin- 2-yl)amino)-1H-pyrazol- 3-yl)phenyl)-1-(4- chlorophenyl)-1H- pyrazole-4-carboxamide | 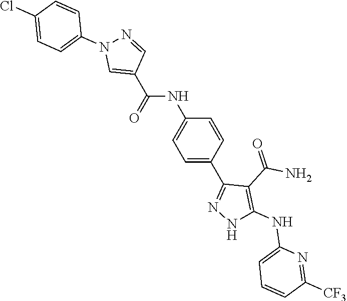 [SMILES: NC(=O)c1c(-c2ccc(NC(=O)c3cnn(-c4ccc(Cl)cc4)c3)cc2)n[nH]c1Nc1cccc(C(F)(F)F)n1] | LCMS (method B): 3.27 min; m/z = 567.9 [M + H]⁺. | ¹H NMR (400 MHz, DMSO-d₆): 13.09 (s, 1H), 10.84 (s, 1H), 9.775 (s, 1H), 9.68 (s, 1H), 8.46 (s, 1H), 8.25 (d, J = 7.2 Hz, 1H), 8.13 (d, J = 7.6 Hz, 1H), 7.63 (q, J = 8.4 Hz, 3H), 7.45 (s, 4H), 7.30 (d, J = 6.8 Hz, 1H). | (4- (chloro) phenyl) hydrazine |
| 37 | N-(4-(4-carbamoyl-5-((6- (trifluoromethyl)pyridin- 2-yl)amino)-1H-pyrazol- 3-yl)phenyl)-1-phenyl- 1H-pyrazole-3- carboxamide | 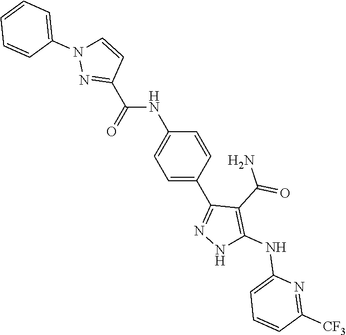 [SMILES: NC(=O)c1c(-c2ccc(NC(=O)c3ccn(-c4ccccc4)n3)cc2)n[nH]c1Nc1cccc(C(F)(F)F)n1] | LCMS (method A): 2.85 min; m/z = 533.1 [M + H]⁺. | ¹H NMR (400 MHz, DMSO-d₆): 12.95 (s, 1H), 10.78 (s, 1H), 9.74 (s, 1H), 8.23 (d, J = 8.8 Hz, 1H), 8.00 (t, J = 7.6 Hz, 1H), 7.84-7.81 (m, 3H), 7.59 (d, J = 8.8 Hz, 2H), 7.51-7.46 (m, 4H), 7.44-7.38 (m, 1H), 7.31 (d, J = 7.2 Hz, 1H), 7.10 (d, J = 2.0 Hz, 1H). Two active protons not detected. | Phenyl- hydrazine |
| 38 | N-(4-(4-carbamoyl-5-((2- methoxypyridin-4- yl)amino)-1H-pyrazol-3- yl)phenyl)-1-phenyl-1H- pyrazole-4-carboxamide | 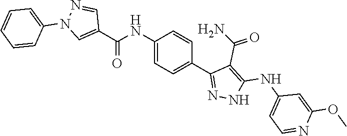 [SMILES: COc1cc(Nc2[nH]nc(-c3ccc(NC(=O)c4cnn(-c5ccccc5)c4)cc3)c2C(N)=O)ccn1] | LCMS (method A): 3.07 min; m/z = 495.0 [M + H]⁺. | ¹H NMR (400 MHz, DMSO-d₆): 12.88 (s, 1H), 10.20 (s, 1H), 9.26 (s, 1H), 9.14 (s, 1H), 8.35 (s, 1H), 7.93-7.87 (m, 5H), 7.61-7.55 (m, | Phenyl- hydrazine (step 1) And 3-(4- amino- phenyl)- 1-(tert- butyl)-5- |
| | | | | 4H), 7.42-7.12 | ((2- |
| | | | | (m, 1H), 7.11 (s, | methoxy- |
| | | | | 1H), 6.92 (d, J = | pyridin-4- |
| | | | | 4.4 Hz, 1H), 6.19 | yl)amino)- |
| | | | | (s, 1H), 3.81 (s, | 1H- |
| | | | | 3H). One active | pyrazole-4- |
| | | | | proton not detected. | carboxamide |
| | | | | | intermediate |
| | | | | | XX (step 4) |

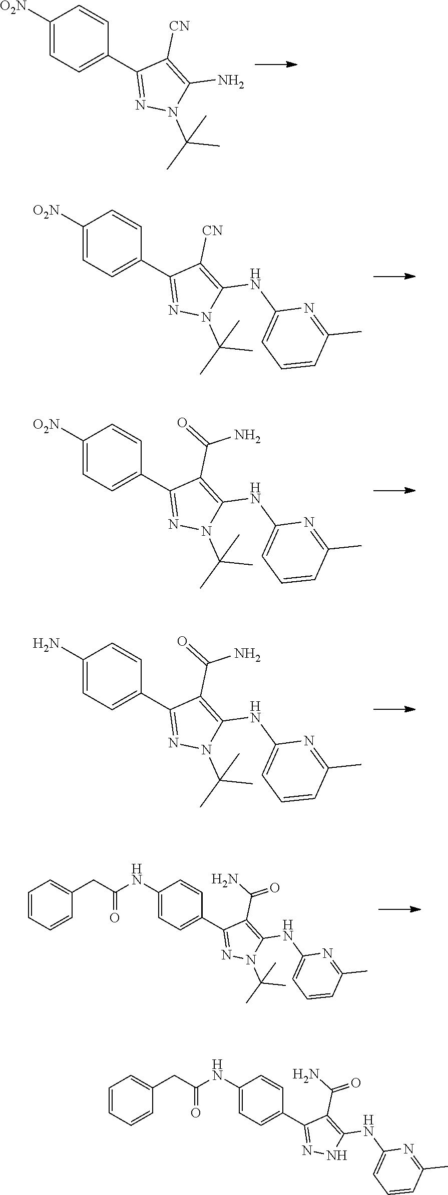 [SMILES: CC(C)(C)n1nc(-c2ccc([N+](=O)[O-])cc2)c(C#N)c1N ; Cc1cccc(Nc2c(C#N)c(-c3ccc([N+](=O)[O-])cc3)nn2C(C)(C)C)n1 ; Cc1cccc(Nc2c(C(N)=O)c(-c3ccc([N+](=O)[O-])cc3)nn2C(C)(C)C)n1 ; Cc1cccc(Nc2c(C(N)=O)c(-c3ccc(N)cc3)nn2C(C)(C)C)n1 ; Cc1cccc(Nc2c(C(N)=O)c(-c3ccc(NC(=O)Cc4ccccc4)cc3)nn2C(C)(C)C)n1 ; Cc1cccc(Nc2[nH]nc(-c3ccc(NC(=O)Cc4ccccc4)cc3)c2C(N)=O)n1]

### Compound 39

### Step 1: 5-(6-methylpyridin-2-ylamino)-1-tert-butyl-3-(4-nitrophenyl)-1H-pyrazole-4-carbonitrile

A mixture of 5-amino-1-(tert-butyl)-3-(4-nitrophenyl)-1H-pyrazole-4-carbonitrile (1 g, 3.5 mmol), 2-bromo-6-methylpyridine (602 mg, 3.5 mmol), Pd₂(dba)₃ (192 mg, 0.2 mmol), BINAP (130 mg, 0.2 mmol) and Cs₂CO₃ (5.1 g, 10.5 mmol) in diglyme (5 mL) was stirred at 150° C. under N₂ for 24 h. The solution was then diluted with EtOAc (50 mL) and H₂O (20 mL). The organic layer was separated, dried over Na₂SO₄ and concentrated under reduced pressure. The crude residue was purified by silica gel column chromatography (PE:EtOAc, 2:1) to afford the desired product (300 mg, 23%) as a yellow solid. LCMS (method A): 2.56 min; m/z=377.2 [M+H]⁺.

### Step 2: 5-(6-methylpyridin-2-ylamino)-1-tert-butyl-3-(4-nitrophenyl)-1H-pyrazole-4-carboxamide

To a solution of 5-(6-methylpyridin-2-ylamino)-1-tert-butyl-3-(4-nitrophenyl)-1H-pyrazole-4-carbonitrile (300 mg, 0.79 mmol) in DMSO (6 mL) and EtOH (30 mL), was added 30% aq. H₂O₂ (6 mL) and 5% aq. NaOH (2 drops), and the mixture was stirred at 80° C. for 2 h. The reaction mixture was concentrated under reduced pressure and the residue was diluted with EtOAc (50 mL) and H₂O (25 mL). The organic layer was separated, dried over Na₂SO₄ and concentrated under reduced pressure to give the desired product (300 mg, 95%) as a yellow solid. LCMS (method A): 2.52 min; m/z=395.2 [M+H]⁺.

### Step 3: 3-(4-(2-phenylacetamido)phenyl)-5-(6-methylpyridin-2-ylamino)-1-tert-butyl-1H-pyrazole-4-carboxamide

To a solution of 5-(6-methylpyridin-2-ylamino)-1-tert-butyl-3-(4-nitrophenyl)-1H-pyrazole-4-carboxamide (315 mg, 0.79 mmol) in MeOH (15 mL), was added sat. aq. NH₄Cl (5 mL) and Zn dust (208 mg, 3.2 mmol), and the mixture was stirred at 40° C. for 3 h. The reaction mixture was filtered and the filtrate concentrated under reduced pressure. The residue was diluted with EtOAc (25 mL) and washed with H₂O (15 mL). The organic layer was dried (Na₂SO₄) and concentrated under reduced pressure to afford the desired product (200 mg, 67%) as a yellow solid, which was used in the next step without further purification. LCMS (method A): 0.55 min; m/z=365.2 [M+H]⁺.

### Step 4: 3-(4-(2-phenylacetamido)phenyl)-5-(6-methylpyridin-2-ylamino)-1-tert-butyl-1H-pyrazole-4-carboxamide

A mixture of 3-(4-(2-phenylacetamido)phenyl)-5-(6-methylpyridin-2-ylamino)-1-tert-butyl-1H-pyrazole-4-carboxamide (30 mg, 0.08 mmol), HATU (30 mg, 0.08 mmol) and Et₃N (16 mg, 0.16 mmol) in THF (2 mL) was stirred at RT for 10 min, and then phenylacetic acid (11 mg, 0.08 mmol) was added. The resulting mixture was stirred at RT overnight and then diluted with EtOAc (15 mL) and H₂O (10 mL). The organic layer was dried (Na₂SO₄) and then concentrated under reduced pressure. The crude residue was purified by prep-TLC (DCM:MeOH, 15:1) to afford the desired product (17 mg, 43%) as a white solid. LCMS (method A): 2.51 min; m/z=483.2 [M+H]⁺.

### Step 5: 3-(4-(2-phenylacetamido)phenyl)-5-(6-methylpyridin-2-ylamino)-1H-pyrazole-4-carboxamide (Compound 39)

A solution of 3-(4-(2-phenylacetamido)phenyl)-5-(6-methylpyridin-2-ylamino)-1-tert-butyl-1H-pyrazole-4-carboxamide (57 mg, 0.12 mmol) in TFA (1 mL) was stirred at 60° C. for 4 h. The solution was concentrated under reduced pressure and the residue triturated (Et₂O) to afford the title product (40 mg, 80%) as a white solid. LCMS (method A): 2.20 min; m/z=427.2 [M+H]⁺. ¹H NMR (400 MHz, DMSO-d₆): 10.46 (s, 1H), 10.22 (s, 1H), 7.96 (br s, 2H), 7.79-6.97 (m, 10H), 3.69 (s, 2H), 2.50 (s, 3H). 3 active protons obscured.

The following compounds were similarly prepared from the appropriate chloropyridine and 5-amino-1-(tert-butyl)-3-(4-nitrophenyl)-1H-pyrazole-4-carbonitrile following step 1 according to the method described for the synthesis of compound 39:

TABLE 8

| Compound | | | | | |
|---|---|---|---|---|---|
| No | Name | Structure | LCMS data | ¹H NMR data | SM |
| 40 | 5-((3- methylpyridin- 2-yl)amino)-3- (4-(2- phenyl- acetamido) phenyl)-1H- pyrazole-4- carboxamide | 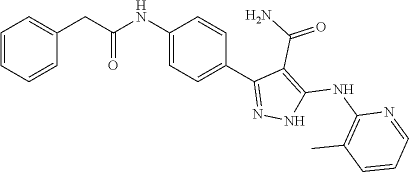 [SMILES: Cc1cccnc1Nc1[nH]nc(-c2ccc(NC(=O)Cc3ccccc3)cc2)c1C(N)=O] | LCMS (method A): 2.33 min; m/z = 427.2 [M + H]⁺. | ¹H NMR (400 MHz, DMSO-d₆): 12.2 (s, 1H), 10.83 (br s, 1H), 10.35 (br s, 2H), 8.16 (br s, 1H), 7.90-7.65 (m, 2H), 7.64-7.47 (m, 3H), 7.42-7.30 (m, 5H), 7.28-7.20 (m, 1H), 6.90 (br s, 1H), 3.67 (s, 2H), 2.27 (s, 3H). | 2-chloro-6- methyl- pyridine |
| 41 | 5-((6-chloro-3- fluoropyridin-2- yl)amino)-3-(4- (2- phenyl- acetamido) phenyl)-1H- pyrazole-4- carboxamide | 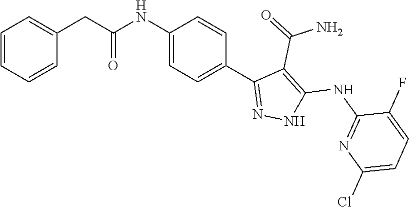 [SMILES: NC(=O)c1c(-c2ccc(NC(=O)Cc3ccccc3)cc2)n[nH]c1Nc1nc(Cl)ccc1F] | LCMS (method A): 2.83 min; m/z = 465.1 [M + H]⁺. | ¹H NMR (400 MHz, DMSO-d₆): 12.97- 12.86 (m, 1H), 10.35 (br s, 1H), 7.72- 7.55 (m, 5H), 7.35- 6.33 (m, 6H), 3.67 (s, 2H). | 2,6- dichloro-3- fluoro- pyridine |
| 42 | 5-((3- fluoropyridin-2- yl)amino)-3-(4- (2- phenyl- acetamido) phenyl)-1H- pyrazole-4- carboxamide | 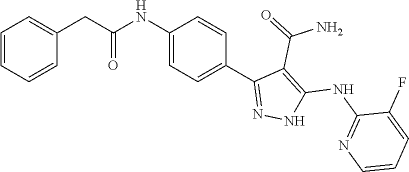 [SMILES: NC(=O)c1c(-c2ccc(NC(=O)Cc3ccccc3)cc2)n[nH]c1Nc1ncccc1F] | LCMS (method A): 2.59 min; m/z = 431.1 [M + H]⁺. | ¹H NMR (400 MHz, DMSO-d₆): 13.16 (s, 1H), 10.47-10.36 (m, 2H), 8.10 (br s, 1H), 7.95-7.65 (m, 3H), 7.64-7.45 (m, 2H), 7.44-7.15 (m, 5H), 6.99 (br s, 1H), 3.68 (s, 2H). | 2-chloro-3- fluoro- pyridine |
| 43 | 5-((6-(2- methoxyethoxy) pyridin-2- yl)amino)-3-(4- (2- phenyl- acetamido) phenyl)-1H- pyrazole-4- carboxamide | 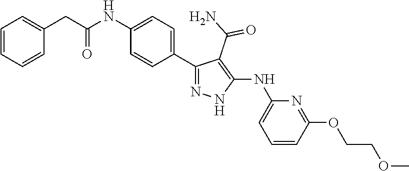 [SMILES: COCCOc1cccc(Nc2[nH]nc(-c3ccc(NC(=O)Cc4ccccc4)cc3)c2C(N)=O)n1] | LCMS (method C): 2.78 min; m/z = 487.2 [M + H]⁺. | ¹H NMR (300 MHz, DMSO-d₆): 12.78 (s, 1H), 10.43 (s, 1H), 9.32 (s, 1H), 7.77 (d, J = 8.7 Hz, 2H), 7.65-7.50 (m, 4H), 7.39-7.23 (m, 5H), 6.27 (d, J = 7.5 Hz, 2H), 5.96 (br s, 1H), 4.40-4.30 (m, 2H), 3.73-3.63 (m, 4H), 3.32 (s, 3H). | 2-chloro-6- (2- methoxy- ethoxy) pyridine |
| 44 | 5-((5-(2- methoxyethoxy) pyridin-2- yl)amino)-3-(4- (2- phenyl- acetamido) phenyl)-1H- pyrazole-4- carboxamide | 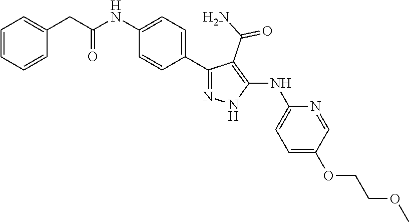 [SMILES: COCCOc1ccc(Nc2[nH]nc(-c3ccc(NC(=O)Cc4ccccc4)cc3)c2C(N)=O)nc1] | LCMS (method C): 2.38 min; m/z = 487.2 [M + H]⁺. | ¹H NMR (300 MHz, DMSO-d₆): 12.69 (s, 1H), 10.43 (s, 1H), 9.36 (s, 1H), 7.69-8.00 (m, 4H), 7.23-7.55 (m, 8H), 5.89 (bs, 2H), 4.11 (t, J = 4.5 Hz, 2H), 3.69 (s, 2H), 3.63- 3.67 (m, 2H), 3.31 (s, 3H). | 2-chloro-5- (2- methoxy- ethoxy) pyridine |
| 45 | 5-((4-(2- methoxyethoxy) pyridin-2- yl)amino)-3-(4- (2- phenyl- acetamido) phenyl)-1H- pyrazole-4- carboxamide | 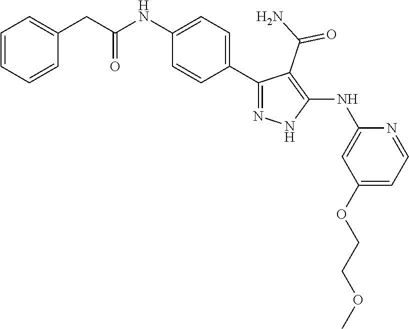 [SMILES: COCCOc1ccnc(Nc2[nH]nc(-c3ccc(NC(=O)Cc4ccccc4)cc3)c2C(N)=O)c1] | LCMS (method C): 1.90 min; m/z = 487.2 [M + H]⁺. | ¹H NMR (300 MHz, DMSO-d₆): 12.80 (s, 1H), 10.43 (s, 1H), 9.49 (s, 1H), 8.00 (d, J = 6.5 Hz, 1H), 7.78 (d, J = 8.5 Hz, 2H), 7.63 (s, 1H), 7.52 (d, J = 9.0 Hz, 2H), 7.23- 7.37 (m, 5H), 6.49 (ds, J = 4.3 Hz, 1H), 5.95 (br s, 2H), 4.16 (t, J = 4.5 Hz, 1H), 3.65-3.71 (m, 4H), 3.32 (s, 3H). | 2-bromo-4- (2- methoxy- ethoxy) pyridine |
| 129 | 5-((5- methylpyridin- 2-yl)amino)-3- (4-(2- phenyl- acetamido) phenyl)-1H- pyrazole-4- carboxamide | 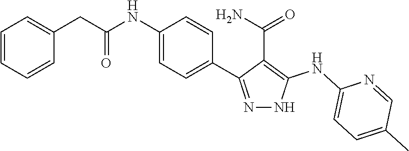 [SMILES: Cc1ccc(Nc2[nH]nc(-c3ccc(NC(=O)Cc4ccccc4)cc3)c2C(N)=O)nc1] | LCMS (method A): 2.24 min; m/z = 427.2 [M + H]⁺. | ¹H NMR (400 MHz, DMSO-d₆): 12.74 (s, 1H), 10.61 (s, 1H), 9.38 (br, 1H), 8.06-7.98 (br s, 1H), 7.96-7.88 (m, 1H), 7.83-7.76 (m, 2H), 7.58-7.48 (m, 3H), 7.40-7.30 (m, 5H), 7.28-7.22 (m, 2H), 3.69 (s, 2H), 2.20 (s, 3H). | 2-chloro-5- methyl- pyridine |

### Compound 46

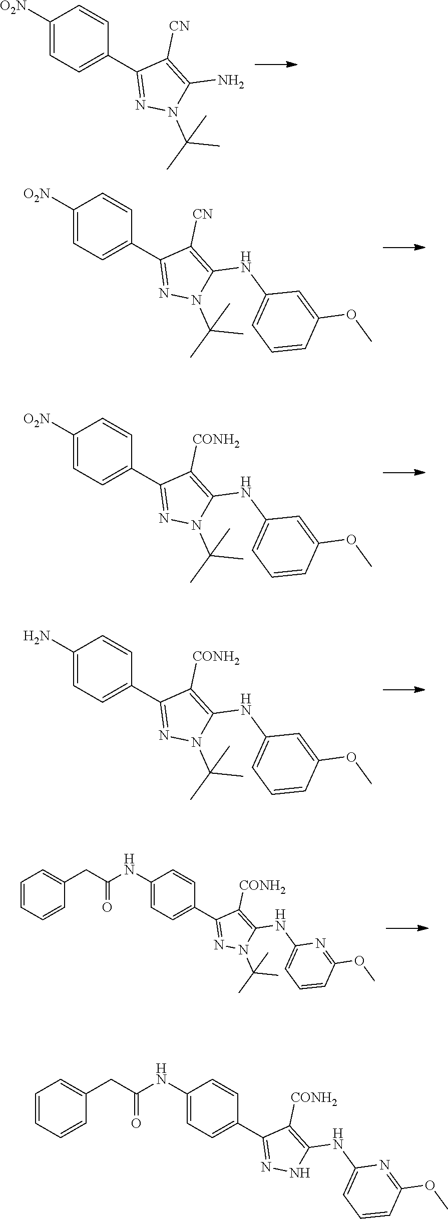 [SMILES: CC(C)(C)n1nc(-c2ccc([N+](=O)[O-])cc2)c(C#N)c1N ; COc1cccc(Nc2c(C#N)c(-c3ccc([N+](=O)[O-])cc3)nn2C(C)(C)C)c1 ; COc1cccc(Nc2c(C(N)=O)c(-c3ccc([N+](=O)[O-])cc3)nn2C(C)(C)C)c1 ; COc1cccc(Nc2c(C(N)=O)c(-c3ccc(N)cc3)nn2C(C)(C)C)c1 ; COc1cccc(Nc2c(C(N)=O)c(-c3ccc(NC(=O)Cc4ccccc4)cc3)nn2C(C)(C)C)n1 ; COc1cccc(Nc2[nH]nc(-c3ccc(NC(=O)Cc4ccccc4)cc3)c2C(N)=O)n1]

### Step 1: 1-(tert-butyl)-5-((3-methoxyphenyl)amino)-3-(4-nitrophenyl)-1H-pyrazole-4-carbonitrile

A mixture of 5-amino-1-(tert-butyl)-3-(4-nitrophenyl)-1H-pyrazole-4-carbonitrile (400 mg, 1.4 mmol), 1-bromo-3-methoxybenzene (262 mg, 1.4 mmol), Pd(OAc)₂ (15.7 mg, 0.07 mmol), Xantphos (48.6 mg, 0.084 mmol) and KOt-Bu (314 mg, 2.8 mmol) in 1,4-dioxane (8 mL) was heated to 110° C. under N₂ for 16 h. The reaction mixture was concentrated under reduced pressure and the crude residue was purified by silica gel column chromatography (PE:EtOAc, 2:1) to afford the title product (300 mg, 55%) as a yellow solid. LCMS (method A): 2.33 min; m/z=392.1 [M+H]⁺.

### Step 2: 1-(tert-butyl)-5-((3-methoxyphenyl)amino)-3-(4-nitrophenyl)-1H-pyrazole-4-carboxamide

To a solution of 1-(tert-butyl)-5-((3-methoxyphenyl)amino)-3-(4-nitrophenyl)-1H-pyrazole-4-carbonitrile (300 mg, 0.77 mmol) in DMSO (1 mL) and EtOH (4 mL), was added 30% aq. H₂O₂ (1 mL) followed by 5% aq. NaOH (2 drops). The mixture was heated to 80° C. for 2 h and then concentrated under reduced pressure. Water (2 mL) was added and the precipitated solids were collected by filtration and dried under reduced pressure to afford the title product (290 mg, 92%) as a yellow solid. LCMS (method A): 2.98 min; m/z=410.2 [M+H]⁺.

### Step 3: 3-(4-aminophenyl)-1-(tert-butyl)-5-((3-methoxyphenyl)amino)-1H-pyrazole-4-carboxamide

To a solution of 1-(tert-butyl)-5-((3-methoxyphenyl)amino)-3-(4-nitrophenyl)-1H-pyrazole-4-carboxamide (290 mg, 0.71 mmol) in MeOH (9 mL), was added sat. aq. NH₄Cl (3 mL) and Zn dust (232 mg, 3.54 mmol), and the mixture was stirred at 40° C. for 3 h. The reaction mixture was filtered through Celite and the filtrate concentrated under pressure. The residue was diluted with H₂O and the precipitated solids were collected by filtration and dried under reduced pressure to afford the title product (230 mg, 86%) as a yellow solid. LCMS (method A): 1.03 min; m/z=380.2 [M+H]⁺.

### Step 4: 1-(tert-butyl)-5-((3-methoxyphenyl)amino)-3-(4-(2-phenylacetamido)phenyl)-1H-pyrazole-4-carboxamide

To a solution of 3-(4-aminophenyl)-1-(tert-butyl)-5-((3-methoxyphenyl)amino)-1H-pyrazole-4-carboxamide (90 mg, 0.24 mmol) in THF (3 mL) was added 2-phenylacetic acid (33 mg, 0.24 mmol), HATU (108 mg, 0.28 mmol) and Et₃N (49 mg, 0.48 mmol). The mixture was stirred at RT for 16 h and then partitioned between H₂O (10 mL) and DCM (15 mL). The organic layer was separated, washed (brine), dried (Na₂SO₄) and then concentrated under reduced pressure. The crude residue was purified by prep-TLC (DCM:MeOH, 20:1) to afford the title product (13 mg, 11%) as white solid. LCMS (method A): 2.85 min; m/z=498.3 [M+H]⁺.

### Step 5: 5-((3-methoxyphenyl)amino)-3-(4-(2-phenylacetamido)phenyl)-1H-pyrazole-4-carboxamide (Compound 46)

A solution of 1-(tert-butyl)-5-((3-methoxyphenyl)amino)-3-(4-(2-phenyl acetamido)phenyl)-1H-pyrazole-4-carboxamide (13 mg, 0.026 mmol) in TFA (2 mL) was heated to 60° C. for 1 h and then concentrated under reduced pressure. The residue was triturated (Et₂O) to afford the title product·mono TFA salt (10 mg, 69%) as a white solid. LCMS (method A): 2.66 min; m/z=442.2 [M+H]⁺. ¹H NMR (400 MHz, DMSO-d₆): 12.63 (s, br, 1H), 10.41 (s, br, 1H), 9.00 (s, br, 1H), 7.76 (d, J=8.8 Hz, 2H), 7.51 (d, J=8.8 Hz, 2H), 7.34-7.32 (m, 4H), 7.27-7.23 (m, 2H), 7.14 (t, J=8.0 Hz, 1H), 6.97 (d, J=7.2 Hz, 1H), 6.40 (dd, J=8.0, 2.0 Hz, 1H), 3.73 (s, 3H), 3.68 (s, 2H).

### Compound 47

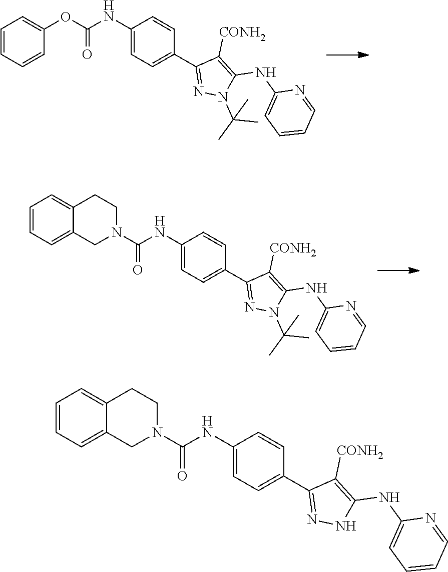 [SMILES: CC(C)(C)n1nc(-c2ccc(NC(=O)Oc3ccccc3)cc2)c(C(N)=O)c1Nc1ccccn1 ; CC(C)(C)n1nc(-c2ccc(NC(=O)N3CCc4ccccc4C3)cc2)c(C(N)=O)c1Nc1ccccn1 ; NC(=O)c1c(-c2ccc(NC(=O)N3CCc4ccccc4C3)cc2)n[nH]c1Nc1ccccn1]

### Step 1: N-(4-(1-(tert-butyl)-4-carbamoyl-5-(pyridin-2-ylamino)-1H-pyrazol-3-yl)phenyl)-3,4-dihydroisoquinoline-2(1H)-carboxamide

To a mixture of phenyl (4-(1-(tert-butyl)-4-carbamoyl-5-(pyridin-2-ylamino)-1H-pyrazol-3-yl)phenyl)carbamate (70 mg, 0.15 mmol) and DIPEA (387 mg, 3.0 mmol) in THF (6 mL), was added 1,2,3,4-tetrahydroisoquinoline (40 mg, 0.3 mmol) and the solution stirred at 85° C. for 4 h. The mixture was concentrated under reduced pressure and the residue was partitioned between EtOAc (50 mL) and H₂O (25 mL). The organic layer was separated, dried (Na₂SO₄) and then concentrated under reduced pressure. The crude residue was purified by prep-TLC (DCM:MeOH, 10:1) to afford the title product (50 mg, 47%) as a yellow solid. LCMS (method A): 2.68 min; m/z=510.3 [M+H]⁺.

### Step 2: N-(4-(4-carbamoyl-5-(pyridin-2-ylamino)-1H-pyrazol-3-yl)phenyl)-8-(trifluoromethyl)-3,4-dihydroisoquinoline-2(1H)-carboxamide (Compound 47)

A mixture of N-(4-(1-(tert-butyl)-4-carbamoyl-5-(pyridin-2-ylamino)-1H-pyrazol-3-yl)phenyl)-3,4-dihydroisoquinoline-2(1H)-carboxamide (50 mg, 0.098 mmol) in TFA (1 mL) was heated to 60° C. for 1 h, and then concentrated under reduced pressure. The crude residue was purified by prep-TLC (DCM:MeOH:NH₄OH 10:1:0.1) to afford the title product (8 mg, 18%) as a white solid. LCMS (method A): 2.56 min; m/z=454.2 [M+H]⁺. ¹H NMR (400 MHz, DMSO-d₆): 12.98 (s, 1H), 9.51 (s, 1H), 9.02 (s, 1H), 8.17 (s, 1H), 8.00 (s, 1H), 7.90-7.60 (m, 4H), 7.50-7.10 (m, 7H), 6.86-6.84 (m, 1H), 4.69 (s, 2H), 3.75 (br s, 2H), 2.87 (br s, 2H).

The following compounds were similarly prepared from the appropriate amine SM and phenyl (4-(1-(tert-butyl)-4-carbamoyl-5-(pyridin-2-ylamino)-1H-pyrazol-3-yl)phenyl)carbamate according to the method described for the synthesis of compound 47:

TABLE 9

| Compound | | | | | |
|---|---|---|---|---|---|
| No | Name | Structure | LCMS data | ¹H NMR data | SM |
| 48 | 3-(4-(3-methyl-3- phenylureido)phen yl)-5-(pyridin-2- ylamino)-1H- pyrazole-4- carboxamide | 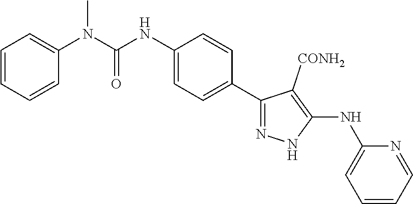 [SMILES: CN(C(=O)Nc1ccc(-c2n[nH]c(Nc3ccccn3)c2C(N)=O)cc1)c1ccccc1] | LCMS (method A): 2.07 min; m/z = 427.9 [M + H]⁺. | ¹H NMR (400 MHz, DMSO-d₆): 12.81 (br s, 1H), 9.53 (br s, 1H), 8.52 (br s, 1H), 8.18 (br s, 1H), 7.98 (s, 1H), 7.70- 758 (m, 3H) 7.46- 7.00 (m, 9H), 6.85 (br s, 1H), 3.31 (s, 3H). | N-methyl- aniline |

### Compound 49

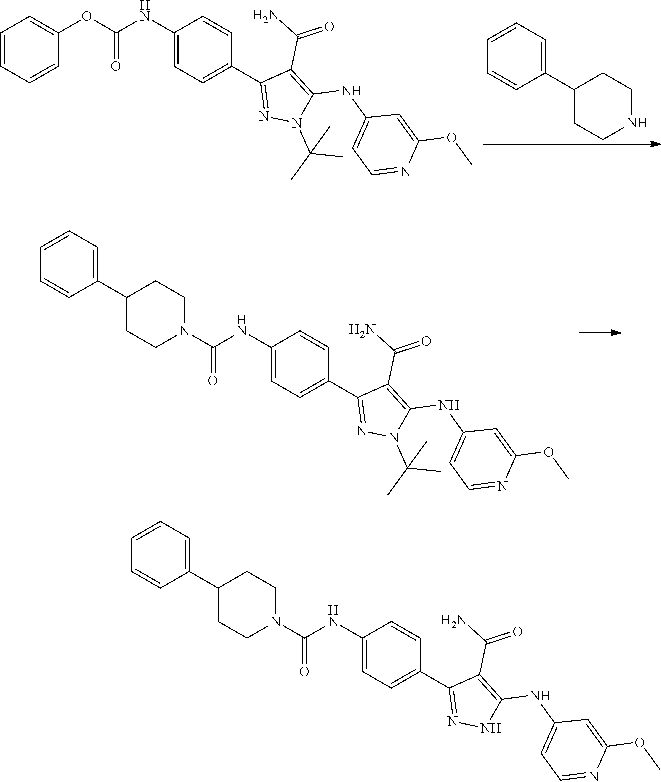 [SMILES: COc1cc(Nc2c(C(N)=O)c(-c3ccc(NC(=O)Oc4ccccc4)cc3)nn2C(C)(C)C)ccn1 ; c1ccc(C2CCNCC2)cc1 ; COc1cc(Nc2c(C(N)=O)c(-c3ccc(NC(=O)N4CCC(c5ccccc5)CC4)cc3)nn2C(C)(C)C)ccn1 ; COc1cc(Nc2[nH]nc(-c3ccc(NC(=O)N4CCC(c5ccccc5)CC4)cc3)c2C(N)=O)ccn1]

### Step 1: N-(4-(1-(tert-butyl)-4-carbamoyl-5-((2-methoxypyridin-4-yl)amino)-1H-pyrazol-3-yl)phenyl)-4-phenylpiperidine-1-carboxamide

A mixture of phenyl (4-(1-(tert-butyl)-4-carbamoyl-5-((2-methoxypyridin-4-yl)amino)-1H-pyrazol-3-yl)phenyl)carbamate (100 mg, 0.23 mmol), DIPEA (58.9 mg, 0.45 mmol) and 4-phenylpiperidine (44.1 mg, 0.27 mmol) in THF (10 mL) was stirred at RT overnight. The mixture was concentrated under reduced pressure and the residue was partitioned between H₂O (50 mL) and EtOAc (50 mL). The organic layer was dried over Na₂SO₄ and concentrated under reduced pressure. The crude residue was purified by silica gel column chromatography (PE:EtOAc, 5:1) to afford the desired product (65 mg, 50%) as a yellow solid. LCMS (method B): 1.36 min; m/z=568.1 [M+H]⁺.

### Step 2: N-(4-(4-carbamoyl-5-((2-methoxypyridin-4-yl)amino)-1H-pyrazol-3-yl)phenyl)-4-phenylpiperidine-1-carboxamide (Compound 49)

To a solution of N-(4-(1-(tert-butyl)-4-carbamoyl-5-((2-methoxypyridin-4-yl)amino)-1H-pyrazol-3-yl)phenyl)-4-phenylpiperidine-1-carboxamide (65 mg, 0.11 mmol), in DCM (3 mL), was added TFA (3 mL) and the mixture was stirred at RT overnight. Water (20 mL) was added, followed by NH₄OH (0.2 mL) and the organics were extracted with DCM (3×30 mL). The combined organic layers were dried over Na₂SO₄ and then concentrated under reduced pressure. The crude residue was purified by prep-TLC (DCM:MeOH, 12:1) to afford the title product (30 mg, 51%) as a white solid. LCMS (method B): 1.22 min; m/z=512.1 [M+H]⁺. ¹H NMR (400 MHz, MeOD-d₄): 7.88 (d, J=5.2 Hz, 1H), 7.63 (d, J=8.8 Hz, 2H), 7.52 (d, J=8.8 Hz, 2H), 7.48-7.45 (m, 1H), 7.37-7.36 (m, 1H), 7.35-7.34 (m, 1H), 7.32-7.30 (m, 1H), 7.28-7.26 (m, 1H), 7.20-7.17 (m, 1H), 7.04-7.02 (m, 1H), 4.58 (br s, 2H), 4.37-4.33 (m, 2H) 3.94 (s, 3H), 2.58-2.54 (m, 1H), 1.93-1.90 (m, 2H), 1.74-1.70 (m, 2H).

The following compounds were similarly prepared from the appropriate amine SM and phenyl (4-(1-(tert-butyl)-4-carbamoyl-5-((2-methoxypyridin-4-yl)amino)-1H-pyrazol-3-yl)phenyl)carbamate according to the method described for the synthesis of compound 49:

TABLE 10

| Compound | | | | | |
|---|---|---|---|---|---|
| No | Name | Structure | LCMS data | ¹H NMR data | SM |
| 50 | 5-((2- methoxypyridin-4- yl)amino)-3-(4-(3- phenylpyrrolidine- 1-carboxamido) phenyl)-1H- pyrazole-4- carboxamide | 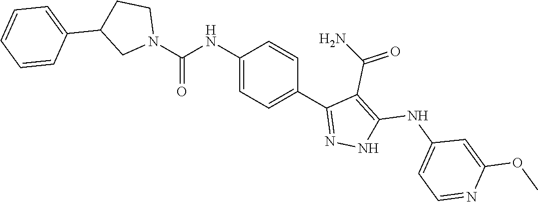 [SMILES: COc1cc(Nc2[nH]nc(-c3ccc(NC(=O)N4CCC(c5ccccc5)C4)cc3)c2C(N)=O)ccn1] | LCMS (method A): 3.07 min; m/z = 498.1 [M + H]⁺. | ¹H NMR (400 MHz, MeOD-d₄): 7.88 (d, J = 6.4 Hz, 1H), 7.70 (d, J = 8.8 Hz, 2H), 7.53 (d, J = 8.4 Hz, 2H), 7.34-7.33 (m, 5H), 7.25-7.24 (m, 1H), 7.04 (br s, 1H), 3.94 (s, 2H), 3.79- 3.74 (m, 1H), 3.64 (s, 3H), 3.62-3.56 (m, 2H), 3.52-3.34 (m, 2H). | 3- phenyl- pyrrolidine |
| 51 | N-(4-(4-carbamoyl- 5-((2- methoxypyridin-4- yl)amino)-1H- pyrazol-3- yl)phenyl)-3,4- dihydroisoquinoline- 2(1H)- carboxamide | 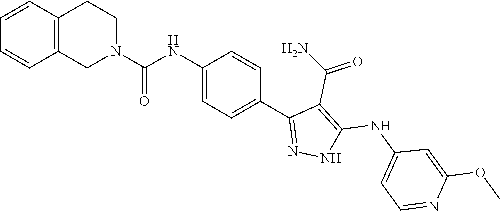 [SMILES: COc1cc(Nc2[nH]nc(-c3ccc(NC(=O)N4CCc5ccccc5C4)cc3)c2C(N)=O)ccn1] | LCMS (method A): 3.00 min; m/z = 484.2 [M + H]⁺. | ¹H NMR (400 MHz, MeOD-d₄): 7.86 (d, J = 6.4 Hz, 1H), 7.65 (d, J = 8.0 Hz, 2H), 7.51 (d, J = 8.4 Hz, 2H), 7.22-7.19 (m, 5H), 6.94 (d, J = 4.8 Hz, 1H), 4.58 (s, 2H), 3.88 (s, 3H), 3.79 (t, J = 5.6 Hz, 2H), 2.95 (t, J = 5.6 Hz, 2H). | 1,2,3,4- tetrahydro- isoquinoline |
| 52 | N-(4-(4-carbamoyl- 5-((2- methoxypyridin-4- yl)amino)-1H- pyrazol-3- yl)phenyl)-6- (trifluoromethyl)- 3,4- dihydroisoquinoline -2(1H)- carboxamide | 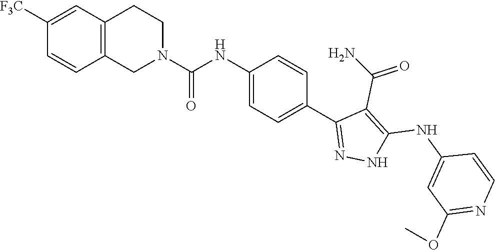 [SMILES: COc1cc(Nc2[nH]nc(-c3ccc(NC(=O)N4CCc5cc(C(F)(F)F)ccc5C4)cc3)c2C(N)=O)ccn1] | LCMS (method A): 2.00 min; m/z = 552.0 [M + H]⁺. | ¹H NMR (400 MHz, MeOD-d₄) δ (ppm): 7.86 (d, J = 6.4 Hz, 1H), 7.64 (d, J = 8.8 Hz, 2H), 7.53-7.50 (m, 4H), 7.39 (d, J = 8.0 Hz, 1H), 7.31 (s, 1H), 7.01 (d, J = 6.8 Hz , 1H), 4.80 (s, 2H), 3.93 (s, 3H), 3.83 (t, J = 6.4 Hz, 2H), 3.03 (t, J = 5.6 Hz, 2H) | 6- (trifluoro- methyl)- 1,2,3,4- tetrahydro- isoquinoline |
| 53 | 3-(4-(3- benzylazetidine-1- carboxamido) phenyl)-5-((2- methoxypyridin-4- yl)amino)-1H- pyrazole-4- carboxamide | 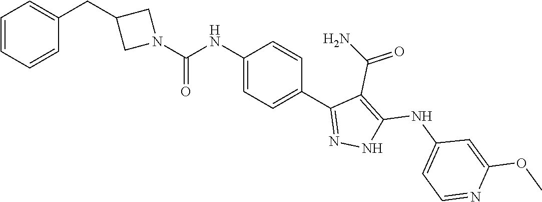 [SMILES: COc1cc(Nc2[nH]nc(-c3ccc(NC(=O)N4CC(Cc5ccccc5)C4)cc3)c2C(N)=O)ccn1] | LCMS (method B): 0.36 min; m/z = 498.0 [M + H]⁺. | ¹H NMR (400 MHz, MeOD-d₄): 7.98 (d, J = 6.4 Hz 1H), 7.68 (d, J = 8.8 Hz, 2H), 7.51 (d, J = 8.8 Hz, 2H), 7.44 (br s, 1H), 7.31-7.27 (m, 2H), 7.24-7.20 (m, 3H), 7.13 (br s, 1H), 4.16-4.12 (m, 2H), 4.00 (s, 3H), 3.83- 3.60 (m, 2H), 2.97 (br s, 3H). | 3- benzyl- azetidine |
| 54 | N-(4-(4-carbamoyl- 5-((2- methoxypyridin-4- yl)amino)-1H- pyrazol-3- yl)phenyl)-3,4- dihydroquinoline- 1(2H)-carboxamide | 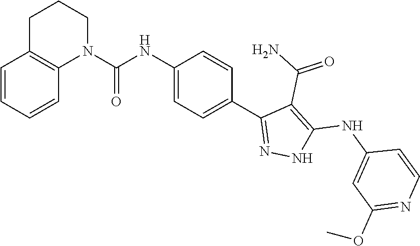 [SMILES: COc1cc(Nc2[nH]nc(-c3ccc(NC(=O)N4CCCc5ccccc54)cc3)c2C(N)=O)ccn1] | LCMS (method B): 0.55 min; m/z = 484.0 [M + H]⁺. | ¹H NMR (400 MHz, MeOD-d₄) δ (ppm): 7.86 (d, J = 6 Hz, 1H), 7.62 (d, J = 8.8 Hz, 2H), 7.52 ( d, J = 8.8 Hz, 2H), 7.36 (d, J = 8 Hz, 1H), 7.27 (s, 1H), 7.22-7.17 (m, 2H), 7.09-7.05 (m, 1H), 6.98 (d, J = 5.6 Hz, 1H), 3.91 (s, 3H), 3.79 (t, J = 6.4 Hz, 2H), 2.82 (t, J = 6.4 Hz, 2H), 2.05-1.98 (m, 2H). | 1,2,3,4- tetrahydro- quinoline |
| 55 | N-(4-(4-carbamoyl- 5-((2- methoxypyridin-4- yl)amino)-1H- pyrazol-3- yl)phenyl)indoline- 1-carboxamide | 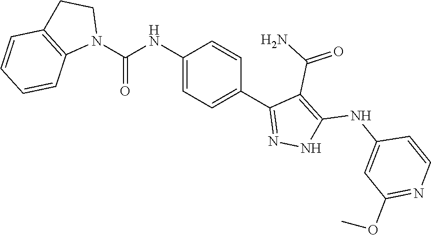 [SMILES: COc1cc(Nc2[nH]nc(-c3ccc(NC(=O)N4CCc5ccccc54)cc3)c2C(N)=O)ccn1] | LCMS (method B): 0.72 min; m/z = 470.1 [M + H]⁺. | ¹H NMR (400 MHz, DMSO-d₆): 12.96 (s, 1H), 9.55 (br s, 1H), 8.76 (s, 1H), 7.93 (d, J = 6.0 Hz, 1H), 7.90 (d, J = 8.0 Hz, 1H), 7.77 (d, J = 8.4 Hz, 2H), 7.55 (d, J = 8.4 Hz, 2H), 7.23-7.12 (m, 3H), 7.04 (br s,1H), 6.93 (t, J = 7.2 Hz, 1H), 4.17 (t, J = 8.4 Hz, 2H), 3.87 (s, 3H), 3.20 (t, J = 8.4 Hz, 2H). | indoline |
| 56 | 3-(4- (ethylsulfonamido) phenyl)-5-((6- (trifluoromethyl) pyridin-2-yl)amino)- 1H-pyrazole-4- carboxamide | 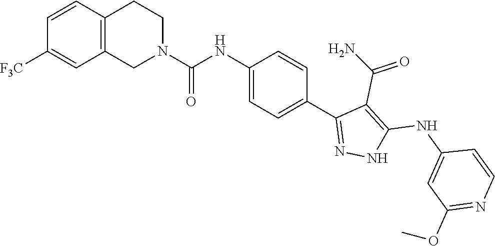 [SMILES: COc1cc(Nc2[nH]nc(-c3ccc(NC(=O)N4CCc5ccc(C(F)(F)F)cc5C4)cc3)c2C(N)=O)ccn1] | LCMS (method B): 1.95 min; m/z = 552.0 [M + H]⁺. | ¹H NMR (400 MHz, DMSO-d₆): 12.81 (s, 1H), 9.28 (s, 1H), 8.90 (s, 1H), 7.87 (d, J = 5.6 Hz, 1H), 7.68 (d, J = 8.4 Hz, 2H), 7.59 (s, 1H), 7.55 (d, J = 8.0 Hz, 1H), 7.48 (d, J = 8.4 Hz, 2H), 7.45 (d, J = 8 Hz, 1H), 7.11 (d, J = 1.2 Hz, 1H), 6.91 (dd, J = 1.6, 6 Hz, 1H), 4.75 (s, 2H), 3.80 (s, 3H), 3.76 (t, J = 6.0 Hz, 2H), 2.96 (t, J = 5.6 Hz, 2H). | 7- (trifluoro- methyl)- 1,2,3,4- tetrahydroiso quinoline |
| 57 | 5-((2- methoxypyridin-4- yl)amino)-3-(4-(3- methyl-3- phenylureido) phenyl)-1H- pyrazole-4- carboxamide | 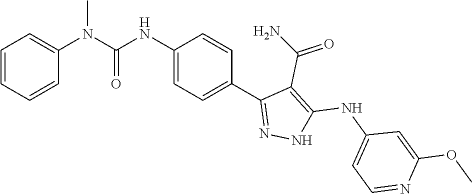 [SMILES: COc1cc(Nc2[nH]nc(-c3ccc(NC(=O)N(C)c4ccccc4)cc3)c2C(N)=O)ccn1] | LCMS (method B): 0.31 min; m/z = 458.0 [M + H]⁺. | ¹H NMR (400 MHz, DMSO-d₆): 12.80 (s, 1H), 9.28 (s, 1H), 8.44 (s, 1H), 7.88 (d, J = 5.6 Hz, 1H), 7.63 (d, J = 8.4 Hz, 2H), 7.46-7.41 (m, 4H), 7.35 (d, J = 8.0 Hz, 2H), 7.28-7.11 (m, 1H), 7.25 (s, 1H), 6.91 (d, J = 4.4 Hz, 1H), 6.07 (br s, 1H), 3.80 (s, 3H), 3.29 (s, 3H). | N- methylaniline |
| 58 | 5-((2- methoxypyridin-4- yl)amino)-3-(4-(2- phenylpyrrolidine- 1-carboxamido) phenyl)- 1H-pyrazole-4- carboxamide | 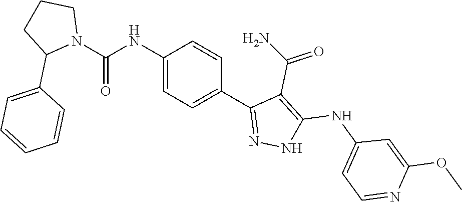 [SMILES: COc1cc(Nc2[nH]nc(-c3ccc(NC(=O)N4CCCC4c4ccccc4)cc3)c2C(N)=O)ccn1] | LCMS (method B): 0.66 min; m/z = 498.1 [M + H]⁺. | ¹H NMR (400 MHz, MeOD-d₄): 7.83 (d, J = 6.0 Hz, 1H), 7.53 (d, J = 8.8 Hz, 2H), 7.45 (d, J = 8.4 Hz, 2H), 7.35-7.21 (m, 6H), 6.03-6.01 (m, 1H), 5.18-5.15 (m, 1H), 3.87 (s, 3H), 3.85- 3.82 (m, 1H), 3.73-3.67 (m, 1H), 2.47-2.38 (m, 1H), 2.03-1.97 (m, 2H), 1.94-1.87 (m, 1H). | 2- phenyl- pyrrolidine |
| 59 | N-(4-(4-carbamoyl- 5-((2- methoxypyridin-4- yl)amino)-1H- pyrazol-3- yl)phenyl)-3- phenylpiperidine-1- carboxamide | 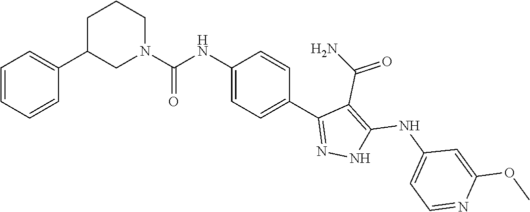 [SMILES: COc1cc(Nc2[nH]nc(-c3ccc(NC(=O)N4CCCC(c5ccccc5)C4)cc3)c2C(N)=O)ccn1] | LCMS (method B): 1.92 min; m/z = 512.3 [M + H]⁺. | ¹H NMR (400 MHz, DMSO-d₆): 12.81 (s, 1H), 9.31 (s, 1H), 8.80 (s, 1H), 7.87 (d, J = 4.6 Hz, 1H), 7.65 (d, J = 8.8 Hz, 2H), 7.45 (d, J = 8.4 Hz, 2H), 7.36-7.31 (m, 4H), 7.25-7.22 (m, 1H), 7.12 (d, J = 1.2 Hz, 1H), 6.91 (d, J = 6.8 Hz, 1H), 6.00 (br s, 1H), 4.24- 4.20 (m, 2H), 3.80 (s, 3H), 2.94-2.81 (m, 2H), 2.71-2.65 (m, 1H), 1.95-1.53 (m, 4H). | 3- phenyl- piperidine |
| 60 | (S)-N-(4-(4- carbamoyl-5-((2- methoxypyridin-4- yl)amino)-1H- pyrazol-3- yl)phenyl)-3- phenylpiperidine-1- carboxamide | 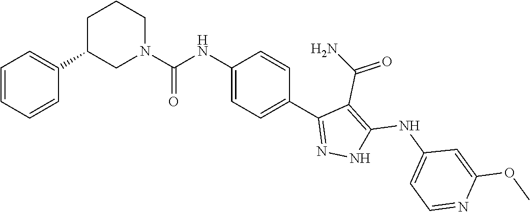 [SMILES: COc1cc(Nc2[nH]nc(-c3ccc(NC(=O)N4CCC[C@@H](c5ccccc5)C4)cc3)c2C(N)=O)ccn1] | LCMS (method A): 3.10 min; m/z = 512.2 [M + H]⁺. | ¹H NMR (400 MHz, DMSO-d₆): 12.83 (s, 1H), 9.33 (s, 1H), 8.83 (s, 1H), 7.88 (d, J = 6.0 Hz, 1H), 7.67 (d, J = 10.0 Hz, 2H), 7.46 (d, J = 8.8 Hz, 2H), 7.36-7.31 (m, 4H), 7.25-7.22 (m, 1H), 7.12 (d, J = 1.6 Hz, 1H), 6.92 (q, J = 5.6 Hz, 1H), 4.22 (t, J = 11.2 Hz, 2H), 3.80 (s, 3H), 2.94-2.65 (m, 3H), 1.95 (d, J = 12.8 Hz, 1H), 1.79- 1.67 (m, 2H), 1.57-1.53 (m, 1H), 1.23 (s, 1H). | (S)-3- phenyl- piperidine |
| 61 | (R)-N-(4-(4- carbamoyl-5-((2- methoxypyridin-4- yl)amino)-1H- pyrazol-3- yl)phenyl)-3- phenylpiperidine-1- carboxamide | 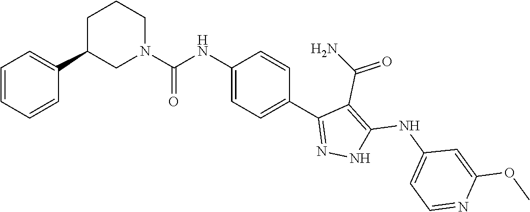 [SMILES: COc1cc(Nc2[nH]nc(-c3ccc(NC(=O)N4CCC[C@H](c5ccccc5)C4)cc3)c2C(N)=O)ccn1] | LCMS (method A): 3.16 min; m/z = 512.2 [M + H]⁺. | ¹H NMR (400 MHz, DMSO-d₆): 12.83 (s, 1H), 9.33 (s, 1H), 8.83 (s, 1H), 7.88 (d, J = 6.0 Hz, 1H), 7.67 (d, J = 10.0 Hz, 2H), 7.46 (d, J = 8.8 Hz, 2H), 7.36-7.31 (m, 4H), 7.25-7.22 (m, 1H), 7.12 (d, J = 1.6 Hz, 1H), 6.92 (q, J = 5.6 Hz, 1H), 4.22 (t, J = 11.2 Hz, 2H), 3.80 (s, 3H), 2.94-2.65 (m, 3H), 1.95 (d, J = 12.8 Hz, 1H), 1.79- 1.67 (m, 2H), 1.57-1.53 (m, 1H), 1.23 (s, 1H). | (R)-3- phenyl- piperidine |
| 62 | (S)-5-((2- methoxypyridin-4- yl)amino)-3-(4-(3- phenylpyrrolidine- 1-carboxamido) phenyl)- 1H-pyrazole-4- carboxamide | 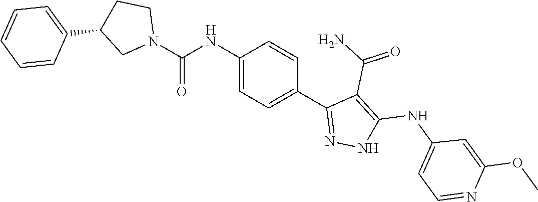 [SMILES: COc1cc(Nc2[nH]nc(-c3ccc(NC(=O)N4CC[C@@H](c5ccccc5)C4)cc3)c2C(N)=O)ccn1] | LCMS (method A): 3.60 min; m/z = 498.1 [M + H]⁺. | ¹H NMR (400 MHz, DMSO-d₆): 12.85 (s, 1H), 9.36 (s, 1H), 8.47 (s, 1H), 7.88 (d, J = 6.0 Hz, 1H), 7.73 (d, J = 8.4 Hz, 2H), 7.47 (d, J = 8.8 Hz, 2H), 7.35-7.34 (m, 4H), 7.27-7.21 (m, 1H), 7.13 (s, 1H), 6.94-6.93 (d, J = 5.2 Hz, 1H), 3.93-3.89 (m, 1H), 3.81 (s, 3H), 3.69-3.65 (m, 1H), 3.46-3.33 (m, 3H), 2.28-2.27 (m, 1H), 2.02-2.00 (m, 1H). Two active protons obscured. | (3S)-3- phenyl- pyrrolidine |
| 63 | (R)-5-((2- methoxypyridin-4- yl)amino)-3-(4-(3- phenylpyrrolidine- 1-carboxamido) phenyl)- 1H-pyrazole-4- carboxamide | 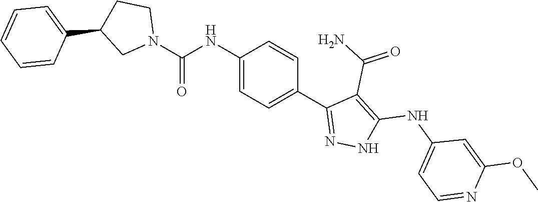 [SMILES: COc1cc(Nc2[nH]nc(-c3ccc(NC(=O)N4CC[C@H](c5ccccc5)C4)cc3)c2C(N)=O)ccn1] | LCMS (method A): 3.60 min; m/z = 498.1 [M + H]⁺. | ¹H NMR (400 MHz, DMSO-d₆): 12.85 (s, 1H), 9.36 (s, 1H), 8.47 (s, 1H), 7.88 (d, J = 6.0 Hz, 1H), 7.73 (d, J = 8.4 Hz, 2H), 7.47 (d, J = 8.8 Hz, 2H), 7.35-7.34 (m, 4H), 7.27-7.21 (m, 1H), 7.13 (s, 1H), 6.94-6.93 (d, J = 5.2 Hz, 1H), 3.93-3.89 (m, 1H), 3.81 (s, 3H), 3.69-3.65 (m, 1H), 3.46-3.33 (m, 3H), 2.28-2.27 (m, 1H), 2.02-2.00 (m, 1H). | (3R)-3- phenyl- pyrrolidine |
| 64 | 3-(4-(3-benzyl-3- methylureido) phenyl)-5-((2- methoxypyridin-4- yl)amino)-1H- pyrazole-4- carboxamide | 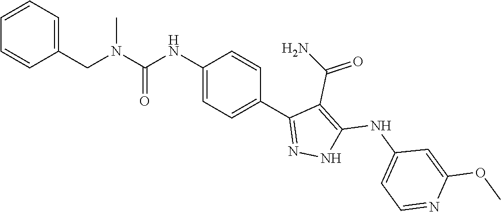 [SMILES: COc1cc(Nc2[nH]nc(-c3ccc(NC(=O)N(C)Cc4ccccc4)cc3)c2C(N)=O)ccn1] | LCMS (method B): 0.51 min; m/z = 472.0 [M + H]⁺. | ¹H NMR (400 MHz, DMSO-d₆): 12.83 (s, 1H), 9.35 (s, 1H), 8.68 (s, 1H), 7.89 (d, J = 6.0 Hz, 1H), 7.70 (d, J = 8.4 Hz, 2H), 7.48 (d, J = 8.8 Hz, 2H), 7.38-7.34 (m, 2H), 7.28-7.24 (m, 3H), 7.13-7.11 (m, 1H), 6.94 (br s, 1H), 4.58 (s, 2H), 3.81 (s, 3H), 3.16 (s, 2H), 2.94 (s, 3H). | benzyl(methyl) amine |
| 65 | N-(4-(4-carbamoyl- 5-((2- methoxypyridin-4- yl)amino)-1H- pyrazol-3-yl) phenyl) isoindoline-2- carboxamide | 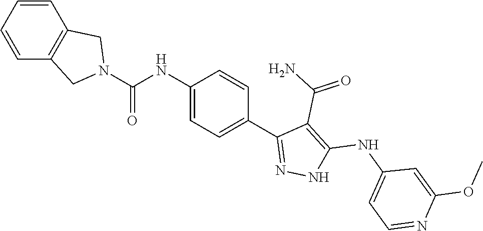 [SMILES: COc1cc(Nc2[nH]nc(-c3ccc(NC(=O)N4Cc5ccccc5C4)cc3)c2C(N)=O)ccn1] | LCMS (method B): 0.44 min; m/z = 470.0 [M + H]⁺. | ¹H NMR (400 MHz, DMSO-d₆): 12.96 (s, 1H), 9.61 (s, 1H), 8.65 (s, 1H), 7.93 (d, J = 6.0 Hz, 1H), 7.78 (d, J = 8.4 Hz, 2H), 7.51 (d, J = 8.4 Hz, 2H), 7.39-7.36 (m, 2H), 7.34-7.32 (m, 2H), 7.19 (s, 2H), 7.06 (s, 2H), 4.80 (s, 4H), 3.87 (s, 3H). | isoindoline |
| 66 | 5-[(2- methoxypyridin-4- yl)amino]-3-(4-{[(2- phenylethyl) carbamoyl]amino} phenyl)-1H- pyrazole-4- carboxamide | 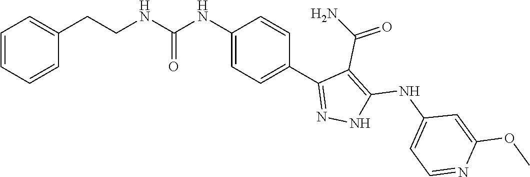 [SMILES: COc1cc(Nc2[nH]nc(-c3ccc(NC(=O)NCCc4ccccc4)cc3)c2C(N)=O)ccn1] | LCMS (method A): 2.95 min; m/z = 472.0 [M + H]⁺. | ¹H NMR (400 MHz, DMSO-d₆): 12.94 (s, 1H), 9.67 (s, 1H), 8.90 (s, 1H), 7.95 (d, J = 6.0 Hz, 1H), 7.56 (d, J = 8.0 Hz, 2H), 7.46 (d, J = 8.0 Hz, 2H), 7.36-7.03 (m, 4H), 7.33-7.28 (m, 3H), 7.08-7.03 (m, 2H), 6.35 (t, J = 5.6 Hz, 1H), 3.89 (s, 3H), 3.38-3.33 (m, 2H), 2.77 (t, J = 7.2 Hz, 2H). | 2- phenylethan- 1-amine |
| 67 | 5-((2- methoxypyridin-4- yl)amino)-3-(4-(3- methyl-3- phenethylureido) phenyl)-1H- pyrazole- 4-carboxamide | 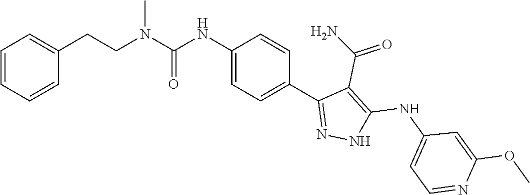 [SMILES: COc1cc(Nc2[nH]nc(-c3ccc(NC(=O)N(C)CCc4ccccc4)cc3)c2C(N)=O)ccn1] | LCMS (method A): 0.51 min; m/z = 486.1 [M + H]+. | ¹H NMR (400 MHz, DMSO-d₆): 12.88 (s, 1H), 9.45 (s, 1H), 8.51 (s, 1H), 7.90 (d, J = 6.0 Hz, 1H), 7.66 (d, J = 8.8 Hz, 2H), 7.46 (d, J = 8.8 Hz, 2H), 7.32-7.15 (m, 5H), 6.98 (d, J = 4.8 Hz, 1H), 6.10 (s, 1H), 3.84 (s, 3H), 3.57 (t, J = 7.6 Hz, 2H), 2.95 (s, 3H), 2.82 (t, J = 7.6 Hz, 2H). | N-methyl-2- phenylethan- 1-amine |
| 68 | 3-(4-(3- benzylureido) phenyl)-5-((2- methoxypyridin-4- yl)amino)-1H- pyrazole-4- carboxamide | 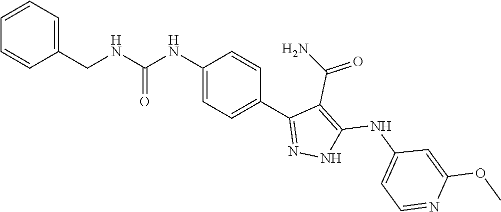 [SMILES: COc1cc(Nc2[nH]nc(-c3ccc(NC(=O)NCc4ccccc4)cc3)c2C(N)=O)ccn1] | LCMS (method A): 2.87 min; m/z = 458.0 [M + H]⁺. | ¹H NMR (400 MHz, MeOD-d₄): 7.86 (s, 1H), 7.63 (d, J = 8.0 Hz, 2H), 7.50-1.44 (m, 3H), 7.34-7.33 (m, 3H), 7.26-6.96 (m, 2H), 6.94 (d, J = 1.2 Hz, 1H), 4.42 (S, 2H), 3.89 (s, 3H). | phenyl- methanamine |
| 69 | 5-((2- methoxypyridin-4- yl)amino)-1- methyl-3-(4- ((phenylmethyl) sulfonamido) phenyl)- 1H-pyrazole-4- carboxamide | 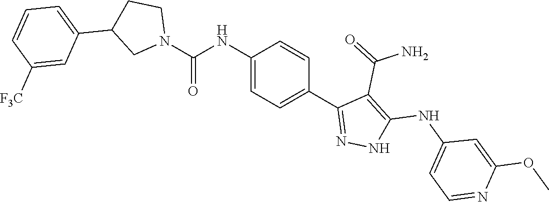 [SMILES: COc1cc(Nc2[nH]nc(-c3ccc(NC(=O)N4CCC(c5cccc(C(F)(F)F)c5)C4)cc3)c2C(N)=O)ccn1] | LCMS (method A): 2.08 min; m/z = 566.1 [M + H]⁺. | ¹H NMR (400 MHz, DMSO-d₆): 13.09 (br s, 1H), 9.97 (br s, 1H), 8.46 (br s, 1H), 8.00 (d, J = 6.4 Hz, 1H), 7.72- 7.58 (m, 6H), 7.50 (d, J = 8.8 Hz, 2H), 7.28-7.18 (m, 2H), 6.39 (br s, 1H), 3.96 (s, 3H), 3.72-3.68 (m, 2H), 3.42-3.37 (m, 2H), 2.36-2.30 (m, 1H), 2.15-1.99 (m, 2H). | 3-[3- (trifluoromethyl) phenyl] pyrrolidine |
| 70 | 5-((2- methoxypyridin-4- yl)amino)-3-(4-(3- (4- (trifluoromethyl) phenyl)pyrrolidine- 1-carboxamido) phenyl)-1H- pyrazole-4- carboxamide | 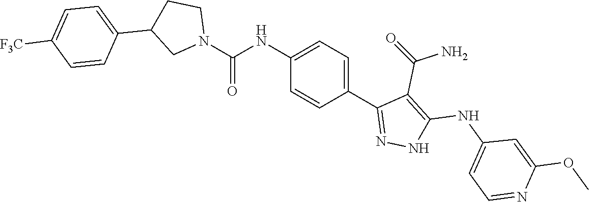 [SMILES: COc1cc(Nc2[nH]nc(-c3ccc(NC(=O)N4CCC(c5ccc(C(F)(F)F)cc5)C4)cc3)c2C(N)=O)ccn1] | LCMS (method A): 2.75 min; m/z = 566.2 [M + H]⁺. | ¹H NMR (400 MHz, DMSO-d₆): 12.82 (s, 1H), 9.29 (s, 1H), 8.49 (s, 1H), 7.87 (d, J = 6.0 Hz, 1H), 7.71 (d, J = 4.0 Hz, 4H), 7.58 (d, J = 8.0 Hz, 2H), 7.46 (d, J = 8.0 Hz, 2H), 7.27 (br s, 1H), 7.11 (s, 1H), 6.90 (d, J = 5.6 Hz, 1H), 6.01 (br s, 1H), 3.94 (t, J = 4.0 Hz, 1H), 3.80 (s, 3H), 3.68 (t, J = 8.0 Hz, 1H), 3.61-3.49 (m, 3H), 2.35- 2.32 (m, 1H), 2.10-2.00 (m, 1H). | 3-[4- (trifluoromethyl) phenyl] pyrrolidine |
| 71 | 5-((2- methoxypyridin-4- yl)amino)-3-(4-(3- (4- (trifluoromethoxy) phenyl)pyrrolidine- 1-carboxamido) phenyl)- 1H-pyrazole-4- carboxamide | 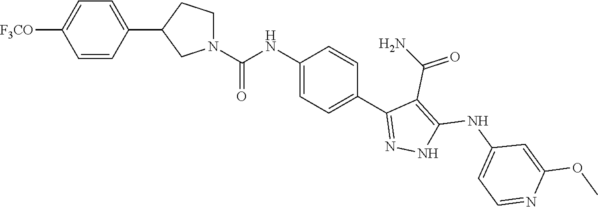 [SMILES: COc1cc(Nc2[nH]nc(-c3ccc(NC(=O)N4CCC(c5ccc(OC(F)(F)F)cc5)C4)cc3)c2C(N)=O)ccn1] | LCMS (method A): 3.04 min; m/z = 582.1 [M + H]⁺. | ¹H NMR (400 MHz, DMSO-d₆): 12.85 (s, 1H), 9.30 (s, 1H), 8.52 (s, 1H), 7.89 (d, J = 6.0 Hz, 1H), 7.76 (d, J = 8.4 Hz, 2H), 7.50-7.46 (m, 4H), 7.36 (d, J = 8.0 Hz, 2H), 7.10 (s, 1H), 6.91-6.90 (m, 1H), 6.05 (br s , 1H), 5.33 (t, J = 6.0 Hz, 1H), 3.94 (t, J = 6.0 Hz, 1H), 3.81 (s, 3H), 3.69 (t, J = 8.8 Hz, 1H), 3.53-3.47 (m, 3H), 2.34- 2.30 (m, 1H), 2.06-2.00 (m, 1H). | 3-(4- (trifluoromethoxy) phenyl) pyrrolidine |
| 72 | 5-((2- methoxypyridin-4- yl)amino)-3-(4-(3- methyl-3- phenylpyrrolidine- 1-carboxamido) phenyl)- 1H-pyrazole-4- carboxamide | 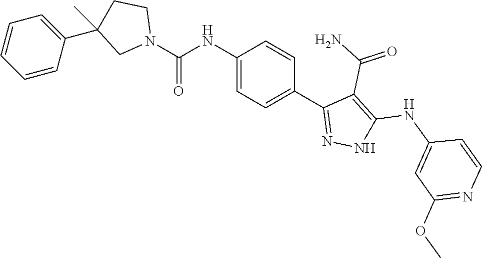 | LCMS (method A): 2.98 min; m/z = 568.3 [M + H]⁺. | ¹H NMR (400 MHz, DMSO-d₆): 12.85 (br s, 1H), 9.43 (br s, 1H), 7.90 (d, J = 5.6 Hz, 1H), 7.73 (d, J = 8.4 Hz, 2H), 7.48 (d, J = 8.4 Hz, 2H), 7.37- 7.35 (m, 4H), 7.25-7.22 (m, 1H), 7.15 (s, 1H), 6.98 (d, J = 8.0 Hz, 1H), 3.83 (s, 3H), 3.73-3.70 (m, 1H), 3.56-3.52 (m, 3H), 2.21-2.13 (m, 2H), 1.33 (s, 3H). | 3-methyl-3- phenyl- pyrrolidine |
| 73 | 3-(4-(3-(3,5- dimethylisoxazol-4- yl)pyrrolidine-1- carboxamido) phenyl)-5-((2- methoxypyridin-4- yl)amino)-1H- pyrazole-4- carboxamide | 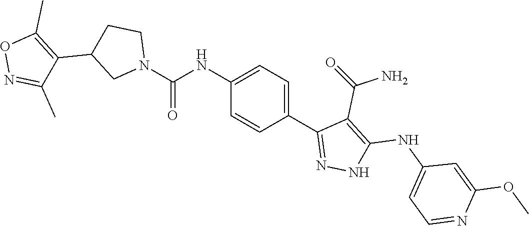 [SMILES: COc1cc(Nc2[nH]nc(-c3ccc(NC(=O)N4CCC(c5c(C)noc5C)C4)cc3)c2C(N)=O)ccn1] | LCMS (method A): 0.78 min; m/z = 517.1 [M + H]⁺. | ¹H NMR (400 MHz, DMSO-d₆): 12.80 (s, 1H), 9.28 (s, 1H), 8.44 (s, 1H), 7.87 (d, J = 6.0 Hz, 1H), 7.70 (d, J = 8.4 Hz, 2H), 7.46 (d, J = 8.4 Hz, 2H), 7.30 (br s, 1H), 7.11 (s, 1H), 6.90 (d, J = 4.4 Hz, 1H), 6.04 (br s, 1H), 3.80 (m, 4H), 3.63 (t, J = 9.2 Hz, 1H), 3.46-3.40 (m, 2H), 2.37 (s, 3H), 2.24 (s, 3H), 2.19-2.14 (m, 1H), 2.10-1.97 (m, 2H). | 3,5-dimethyl- 4-(pyrrolidin- 3- yl)isoxazole |
| 74 | 5-((2- methoxypyridin-4- yl)amino)-3-(4-(3- (1-methyl-1H- pyrazol-4- yl)pyrrolidine-1- carboxamido) phenyl)-1H- pyrazole-4- carboxamide | 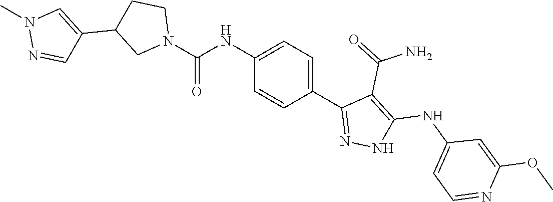 | LCMS (method A): 0.79 min; m/z = 501.8 [M + H]⁺. | ¹H NMR (400 MHz, DMSO-d₆): 12.8 (s, 1H), 9.32 (s, 1H), 8.40 (s, 1H), 7.88-7.86 (d, J = 5.6 Hz, 1H), 7.72- 7.70 (d, J = 8.0 Hz, 2H), 7.59 (s, 1H), 7.46-7.44 (d, J = 8.4 Hz, 1H), 7.36 (s, 1H), 7.12 (s, 1H), 6.92-6.91 (d, J = 5.2 Hz, 1H), 3.81 (s, 3H), 3.79 (s, 3H), 3.61-3.56 (t, J = 9.2 Hz, 1H), 3.47-3.40 (m, 2H), 3.28-3.24 (m, 2H), 2.22 (s, 1H), 1.92-1.87 (m, 1H). | 1-methyl-4- (pyrrolidin-3- yl)-1H- pyrazole |

The following compounds were similarly prepared from the appropriate amine SM and phenyl (4-(1-(tert-butyl)-4-carbamoyl-5-((6-(trifluoromethyl)pyridin-2-yl)amino)-1H-pyrazol-3-yl)phenyl)carbamate according to the method described for the synthesis of compound 49:

TABLE 11

| Compound | | | | | |
|---|---|---|---|---|---|
| No | Name | Structure | LCMS data | ¹H NMR data | SM |
| 75 | N-(4-(4- carbamoyl-5- ((6- (trifluoromethyl) pyridin-2- yl)amino)-1H- pyrazol-3- yl)phenyl)-3,4- dihydroiso- quinoline-2(1H)- carboxamide | 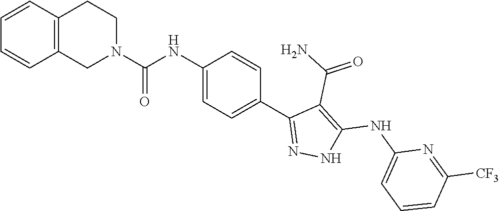 [SMILES: NC(=O)c1c(-c2ccc(NC(=O)N3CCc4ccccc4C3)cc2)n[nH]c1Nc1cccc(C(F)(F)F)n1] | LCMS (method A): 2.93 min; m/z = 522.1 [M + H]⁺. | ¹H NMR (400 MHz, DMSO-d₆): 12.90 (s, 1H), 9.80 (s, 1H), 8.87 (s, 1H), 8.27 (d, J = 8.4 Hz, 1H), 7.99 (t, J = 8.0 Hz, 1H), 7.71 (d, J = 8.4 Hz, 2H), 7.50 (d, J = 8.8 Hz, 2H), 7.32 (d, J = 7.6 Hz, 1H), 7.20 (s, 4H), 4.68 (s, 2H) 3.74 (t, J = 6.0 Hz, 2H), 2.88 (t, J = 6.0 Hz, 2H). One active proton not detected. | 1,2,3,4- tetrahydroisoquinoline |
| 76 | N-(4-(4- carbamoyl-5- ((6- (trifluoromethyl) pyridin-2- yl)amino)-1H- pyrazol-3- yl)phenyl)-6- (trifluoromethyl)- 3,4- dihydroiso- quinoline-2(1H)- carboxamide | 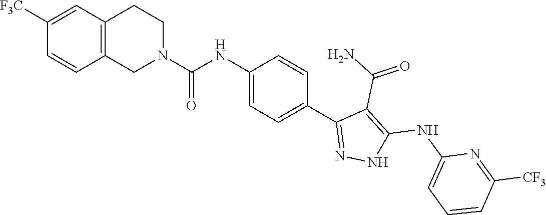 [SMILES: NC(=O)c1c(-c2ccc(NC(=O)N3CCc4cc(C(F)(F)F)ccc4C3)cc2)n[nH]c1Nc1cccc(C(F)(F)F)n1] | LCMS (method A): 3.10 min; m/z = 590.1 [M + H]⁺. | ¹H NMR (400 MHz, DMSO-d₆): 12.90 (s, 1H), 9.79 (s, 1H), 8.91 (s, 1H), 8.26 (d, J = 8.4 Hz, 1H), 7.99 (t, J = 8.0 Hz, 1H) 7.70 (d, J = 8.4 Hz, 2H), 7.60 (s, 1H), 7.57 (d, J = 8.0 Hz, 1H), 7.51 (d, J = 8.8 Hz, 2H), 7.45 (d, J = 8.0 Hz, 1H), 7.32 (d, J = 8.0 Hz, 1H), 4.76 (s, 2H), 3.77 (t, J = 6.0 Hz, 2H), 2.98 (t, J = 6.6 Hz, 2H). Two active protons not detected. | 6- (trifluoromethyl)- 1,2,3,4- tetrahydroisoquinoline |
| 77 | N-(4-(4- carbamoyl-5- ((6- (trifluoromethyl) pyridin-2- yl)amino)-1H- pyrazol-3- yl)phenyl)-7- (trifluoromethyl)- 3,4- dihydroiso- quinoline-2(1H)- carboxamide | 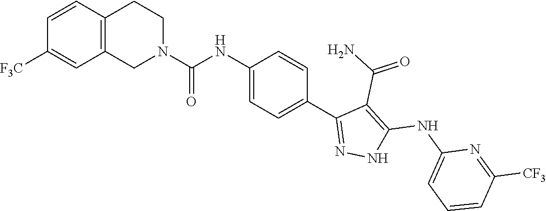 [SMILES: NC(=O)c1c(-c2ccc(NC(=O)N3CCc4ccc(C(F)(F)F)cc4C3)cc2)n[nH]c1Nc1cccc(C(F)(F)F)n1] | LCMS (method A): 3.10 min; m/z = 590.1 [M + H]⁺. | ¹H NMR (400 MHz, DMSO-d₆): 12.90 (s, 1H), 9.79 (s, 1H), 8.91 (s, 1H), 8.26 (d, J = 8.8 Hz, 1H), 7.98 (t, J = 8.0 Hz, 1H), 7.70 (d, J = 8.4 Hz, 2H), 7.59 (s, 1H), 7.57 (d, J = 8.4 Hz, 1H), 7.50 (d, J = 8.8 Hz, 2H), 7.45 (d, J = 8 Hz, 1H), 7.31 (d, J = 7.2 Hz, 1H), 4.75 (s, 2H), 3.76 (t, J = 5.6 Hz, 2H), 2.97 (t, J = 5.6 Hz, 2H). Two active protons not detected. | 7- (trifluoromethyl)- 1,2,3,4- tetrahydroisoquinoline |
| 78 | (R)-N-(4-(4- carbamoyl-5- ((6- (trifluoromethyl) pyridin-2- yl)amino)-1H- pyrazol-3- yl)phenyl)-3-(4- fluorophenyl) piperidine-1- carboxamide | 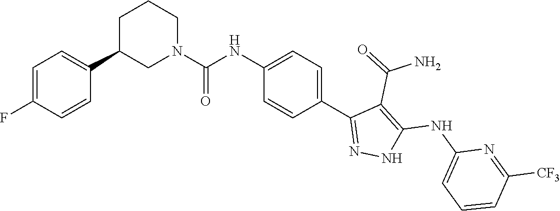 [SMILES: NC(=O)c1c(-c2ccc(NC(=O)N3CCC[C@H](c4ccc(F)cc4)C3)cc2)n[nH]c1Nc1cccc(C(F)(F)F)n1] | LCMS: (method A), 4.106 min, m/z = 568.2 [M + H]⁺ | ¹H NMR (400 MHz, DMSO-d₆) δ (ppm): 12.90 (s, 1H), 9.81 (s, 1H), 8.81 (s, 1H), 8.26 (d, J = 8.8 Hz, 1H), 7.99 (t, J = 7.6 Hz, 1H), 7.67 (d, J = 8.4 Hz, 2H), 7.47 (d, J = 8.8 Hz, 2H), 7.39-7.35 (m, 2H), 7.31 (d, J = 7.6 Hz, 1H), 7.16 (t, J = 8.8 Hz, 2H), 5.97 (br. s, 1H), 4.24-4.17(m, 2H), 2.93-2.82 (m, 2H), 2.74-2.67 (m, 1H), 1.95-1.91 (m, 1H), 1.80-1.75 (m, 1H), 1.69-1.50 (m, 2H). | (R)-3-(4- fluorophenyl) piperidine |
| 79 | (S)-N-(4-(4- carbamoyl-5- ((6- (trifluoromethyl) pyridin-2- yl)amino)-1H- pyrazol-3- yl)phenyl)-3-(4- fluorophenyl) piperidine-1- carboxamide | 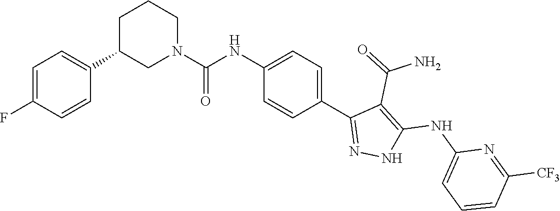 [SMILES: NC(=O)c1c(-c2ccc(NC(=O)N3CCC[C@@H](c4ccc(F)cc4)C3)cc2)n[nH]c1Nc1cccc(C(F)(F)F)n1] | LCMS: (method A), 4.108 min, m/z = 568.2 [M + H]⁺ | ¹H NMR (400 MHz, DMSO-d₆) δ (ppm): 12.90 (s, 1H), 9.81 (s, 1H), 8.81 (s, 1H), 8.26 (d, J = 8.4 Hz, 1H), 7.99 (t, J = 7.6 Hz, 1H), 7.66 (d, J = 8.8 Hz, 2H), 7.47 (d, J = 8.8 Hz, 2H), 7.38-7.35 (m, 2H), 7.31 (d, J = 7.2 Hz, 1H), 7.16 (t, J = 9.2 Hz, 2H), 5.97 (br. s, 1H), 4.24-4.17(m, 2H), 2.93-2.82 (m, 2H), 2.74-2.67 (m, 1H), 1.95-1.91 (m, 1H), 1.80-1.75 (m, 1H), 1.69-1.50 (m, 2H). | (S)-3-(4- fluorophenyl) piperidine |
| 80 | (R)-N-(4-(4- carbamoyl-5- ((6- (trifluoromethyl) pyridin-2- yl)amino)-1H- pyrazol-3- yl)phenyl)-3-(3- fluorophenyl) piperidine-1- carboxamide | 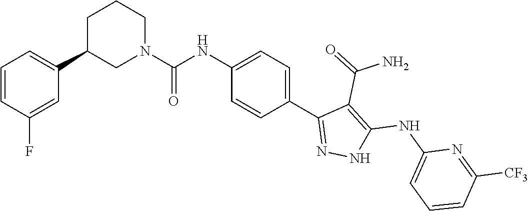 [SMILES: NC(=O)c1c(-c2ccc(NC(=O)N3CCC[C@H](c4cccc(F)c4)C3)cc2)n[nH]c1Nc1cccc(C(F)(F)F)n1] | LCMS: (method A) 4.158 min, m/z = 568.2 [M + H]⁺ | ¹H NMR (400 MHz, DMSO-d₆) δ (ppm): 12.90 (s, 1H), 9.81 (s, 1H), 8.81 (s, 1H), 8.24 (br. s, 1H), 7.99 (t, J = 8.0 Hz, 1H), 7.67 (d, J = 8.4 Hz, 2H), 7.47 (d, J = 8.4 Hz, 2H), 7.37 (q, J = 8.4 Hz, 2H), 7.31 (d, J = 7.2 Hz, 2H), 7.21-7.17 (m, 2H), 7.08 (t, J = 8.4 Hz, 1H), 5.98 (br. s, 1H), 4.24-4.17 (m, 2H), 2.98-2.83 (m, 2H), 2.77-2.69 (m, 1H), 1.96-1.91 (m, 1H), 1.79-1.64 (m, 2H), 1.57-1.49 (m, 1H). | (R)-3-(3- fluorophenyl) piperidine |
| 81 | N-(4-(4- carbamoyl-5- ((6- (trifluoromethyl) pyridin-2- yl)amino)-1H- pyrazol-3- yl)phenyl)-3- (3,5- difluorophenyl) piperidine-1- carboxamide | 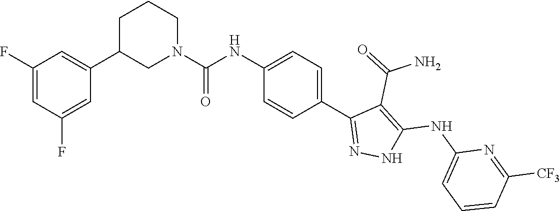 [SMILES: NC(=O)c1c(-c2ccc(NC(=O)N3CCCC(c4cc(F)cc(F)c4)C3)cc2)n[nH]c1Nc1cccc(C(F)(F)F)n1] | LCMS: (method A), 4.2 min, m/z = 586.2 [M + H]⁺ | ¹H NMR (400 MHz, DMSO-d₆) δ 12.98 (br s, 1H), 9.80 (br s, 1H), 9.00 (br s, 1H), 8.25 (d, J = 8.4 Hz, 1H), 7.98 (t, J = 7.6 Hz, 1 H), 7.71 (d, J = 8.4 Hz, 2H), 7.46 (d, J = 8.4 Hz, 2H), 7.30 (d, J = 7.6 Hz, 1H), 7.12-7.06 (m, 3H), 4.26-4.20 (m, 2H), 2.99-2.74 (m, 3 H), 1.95-1.50 (m, 4H). | 3-(3,5- difluorophenyl) piperidine |
| 82 | (R)-N-(4-(4- carbamoyl-5- ((6- (trifluoromethyl) pyridin-2- yl)amino)-1H- pyrazol-3- yl)phenyl)-3- phenyl- piperidine- 1-carboxamide | 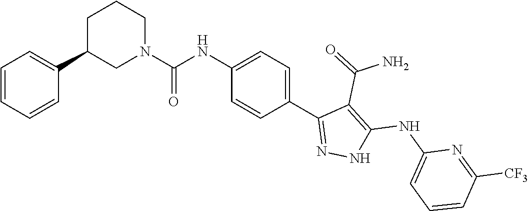 [SMILES: NC(=O)c1c(-c2ccc(NC(=O)N3CCC[C@H](c4ccccc4)C3)cc2)n[nH]c1Nc1cccc(C(F)(F)F)n1] | LCMS: (method A), 4.1 min, m/z = 550.2 [M + H]⁺ | ¹H NMR (400 MHz, DMSO-d₆) δ 12.89 (brs, 1H), 9.80 (brs, 1H), 8.80 (brs, 1H), 8.25 (d, J = 7.6 Hz, 1H), 7.98 (t, J = 8.0 Hz, 1H), 7.66 (d, J = 8.4 Hz, 2H), 7.47 (d, J = 8.4 Hz, 2H), 7.36-7.29 (m, 5H), 7.26-7.22 (m, 1H), 5.98 (brs, 1H), 4.23-4.20 (m, 2H), 2.95-2.82 (m, 2H), 2.72-2.65 (m, 1H), 1.95- 1.92 (m, 1H), 1.79-1.52 (m, 3H). | (R)-3- phenylpiperidine |
| 83 | N-(4-(4- carbamoyl-5- ((6- (trifluoromethyl) pyridin-2- yl)amino)-1H- pyrazol-3- yl)phenyl)-3- (3,4- difluorophenyl) piperidine-1- carboxamide | 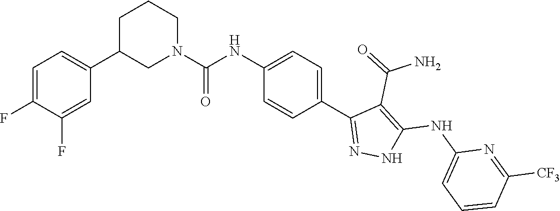 [SMILES: NC(=O)c1c(-c2ccc(NC(=O)N3CCCC(c4ccc(F)c(F)c4)C3)cc2)n[nH]c1Nc1cccc(C(F)(F)F)n1] | LCMS: (method A), 4.23 min, m/z = 586.5 [M + H]⁺ | ¹H NMR (400 MHz, DMSO-d₆) δ 12.89 (s, 1H), 9.79 (s, 1H), 8.79 (s, 1H), 8.25 (d, J = 5.6 Hz, 1H), 7.98 (t, J = 8.4 Hz, 1H), 7.66 (d, J = 8.4 Hz, 2H), 7.47-7.39 (m, 5H), 7.31 (d, J = 7.6 Hz, 1H), 7.19 (s, 1H), 5.94 (s, 1H), 4.19 (t, J = 11.6, 2H), 2.96-2.81 (m, 2H), 2.76-2.67 (m, 1H), 1.93 (d, J = 8 Hz, 1H), 1.77-1.62 (m, 2H), 1.56-1.51 (m, 1H) | 3-(3,4- difluorophenyl) piperidine |

The following compounds were similarly prepared from the appropriate amine SM and phenyl (4-(1-(tert-butyl)-4-carbamoyl-5-((2,6-dimethylpyridin-4-yl)amino)-1H-pyrazol-3-yl)phenyl)carbamate according to the method described for the synthesis of compound 49:

TABLE 12

| Compound | | | | | |
|---|---|---|---|---|---|
| No | Name | Structure | LCMS data | ¹H NMRdata | SM |
| 84 | 5-((2,6- dimethylpyridin-4- yl)amino)-3-(4-(3- phenylpyrrolidine- 1-carboxamido)phenyl)- 1H-pyrazole-4- carboxamide | 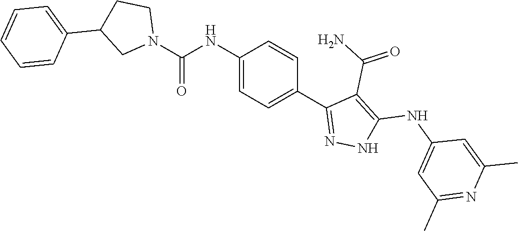 [SMILES: Cc1cc(Nc2[nH]nc(-c3ccc(NC(=O)N4CCC(c5ccccc5)C4)cc3)c2C(N)=O)cc(C)n1] | LCMS (method A): 1.74 min; m/z = 496.1 [M + H]⁺. | ¹H NMR (400 MHz, MeOD-d₄): 7.69 (d, J = 8.0 Hz, 3H), 7.52 (d, J = 8.0 Hz, 3H), 7.34 (d, J = 4.0 Hz, 4H), 7.25 (m, 1H), 3.97 (m, 1H), 3.77 (m, 1H), 3.56 (m, 3H), 2.56 (s, 6H), 2.39 (m, 1H), 2.14 (m, 1H). | 3-phenylpyrrolidine |
| 85 | N-(4-(4-carbamoyl- 5-((2,6- dimethylpyridin-4- yl)amino)-1H- pyrazol-3- yl)phenyl)-3,4- dihydroisoquinoline- 2(1H)- carboxamide | 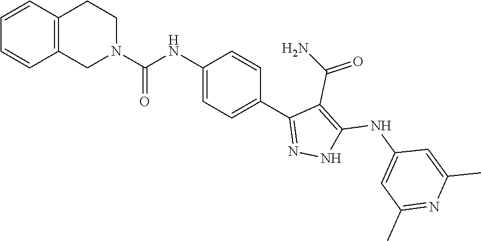 [SMILES: Cc1cc(Nc2[nH]nc(-c3ccc(NC(=O)N4CCc5ccccc5C4)cc3)c2C(N)=O)cc(C)n1] | LCMS (method A): 0.82 min; m/z = 482.1 [M + H]⁺. | ¹H NMR (400 MHz, MeOD-d₄): 7.66- 7.64 (m, 3H), 7.53-7.51 (m, 3H), 7.20 (s, 4H), 4.73 (s, 2H), 3.81 (t, J = 6.0 Hz, 2H), 2.96 (t, J = 6.0 Hz, 2H), 2.56 (s, 6H). | 1,2,3,4- tetrahydroisoquinoline |
| 86 | 5-((2,6- dimethylpyridin-4- yl)amino)-3-(4-(2- phenylpyrrolidine- 1- carboxamido)phenyl)- 1H-pyrazole-4- carboxamide | 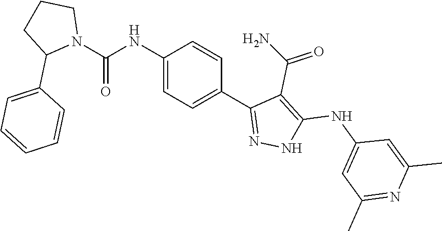 [SMILES: Cc1cc(Nc2[nH]nc(-c3ccc(NC(=O)N4CCCC4c4ccccc4)cc3)c2C(N)=O)cc(C)n1] | LCMS (method B): 0.87 min; m/z = 496.1 [M + H]⁺. | ¹H NMR (400 MHz, MeOD-d₄): 8.53 (d, J = 7.6 Hz, 1H), 8.47 (d, J = 5.2 Hz, 1H), 7.59 (d, J = 6.8 Hz, 2H), 7.45 (d, J = 8.8 Hz, 2H), 3.21 (q, J = 7.2 Hz, 2H), 2.63 (s, 3H), 1.33 (q, J = 7.2 Hz, 3H). | 2-phenylpyrrolidine |
| 87 | N-(4-(4-carbamoyl- 5-((2,6- dimethylpyridin-4- yl)amino)-1H- pyrazol-3- yl)phenyl)-3- phenylpiperidine-1- carboxamide | 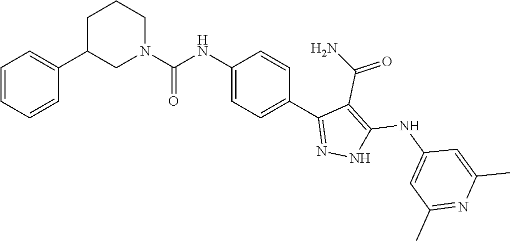 [SMILES: Cc1cc(Nc2[nH]nc(-c3ccc(NC(=O)N4CCCC(c5ccccc5)C4)cc3)c2C(N)=O)cc(C)n1] | LCMS (method B): 1.90 min; m/z = 510.1 [M + H]⁺. | ¹H NMR (400 MHz, MeOD-d₄): 7.61 (d, J = 8.8 Hz, 3H), 7.51 (d, J = 8.8 Hz, 3H), 7.32 (t, J = 5.2 Hz, 4H), 7.24-7.21 (m, 1H), 4.29-4.24 (m, 2H), 3.02-2.95 (m, 2H), 2.80-2.74 (m, 1H), 2.56 (s, 6H), 2.05 (d, J = 12.8 Hz, 3H), 1.90-1.79(m, 2H), 1.71-1.63 (m, 1H). | 3-phenylpiperidine |

### Compound 88

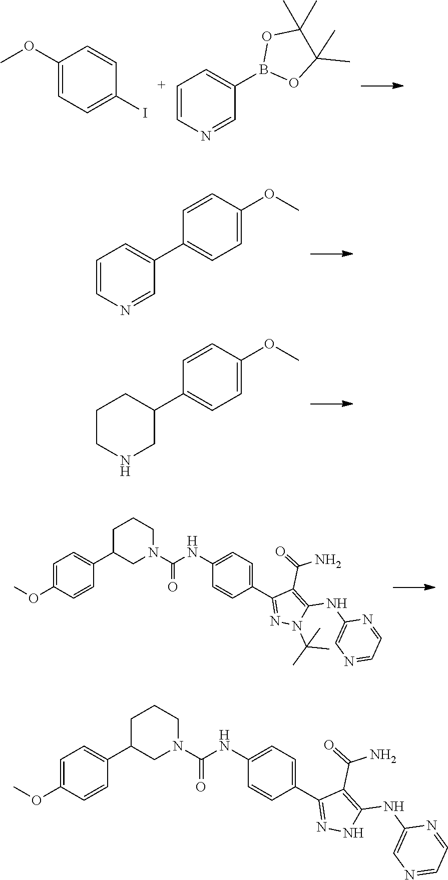

### Step 1: 3-(4-methoxyphenyl)pyridine

To a solution of 3-(4,4,5,5-tetramethyl-1,3,2-dioxaborolan-2-yl)pyridine (1 g, 4.87 mmol) in degassed 1,4-dioxane (44 mL) and H₂O (11 mL), was added 1-iodo-4-methoxybenzene (1.25 g, 5.35 mmol), Pd(dppf)Cl₂ (445 mg, 487 μmol) and Na₂CO₃ (1.03 g, 9.71 mmol). The reaction mixture was stirred at 100° C. under N₂ overnight. The mixture was then concentrated under reduced pressure and the crude residue purified by silica gel column chromatography (PE:EtOAc, 1:1) to afford the title product (730 mg, 80%) as a white solid. LCMS (method A): 2.05 min; m/z=186.1 [M+Na]⁺.

### Step 2: 3-(4-methoxyphenyl)piperidine

A solution of 3-(4-methoxyphenyl)pyridine (550 mg, 2.96 mmol), conc. HCl (1 mL) and PtO₂ (67.2 mg) in MeOH (20 mL) was stirred at RT overnight under a H₂ atmosphere. The mixture was filtered through Celite, and the filtrate concentrated to afford the title compound·HCl salt (760 mg, >100%) as a colorless oil. LCMS (method A): 0.39 min; m/z=192.1 [M+H]⁺.

### Step 3: N-(4-{1-tert-butyl-4-carbamoyl-5-[(pyrazin-2-yl)amino]-1H-pyrazol-3-yl}phenyl)-3-(4-methoxyphenyl)piperidine-1-carboxamide

A solution of 3-(4-methoxyphenyl)piperidine (91.2 mg, 477 μmol), DIPEA (204 mg, 1.58 mmol) and phenyl N-(4-{1-tert-butyl-4-carbamoyl-5-[(pyrazin2-yl)amino]-1H-pyrazol-3-yl}phenyl)carbamate (150 mg, 318 μmol) in DMF (10 mL) was stirred at 60° C. under N₂ for 4 h. The mixture was diluted with H₂O (50 mL) and then filtered. The filter cake was washed with H₂O and dried under reduced pressure to afford the title product (150 mg, 83%) as a white solid. LCMS (method A): 3.76 min; m/z=569.3 [M+Na]⁺.

### Step 4: N-(4-(4-carbamoyl-5-(pyrazin-2-ylamino)-1H-pyrazol-3-yl)phenyl)-3-(4-methoxyphenyl)piperidine-1-carboxamide (Compound 88)

A solution of N-(4-{1-tert-butyl-4-carbamoyl-5-[(pyrazin-2-yl)amino]-1H-pyrazol-3-yl}phenyl)-3-(4-methoxyphenyl)piperidine-1-carboxamide (150 mg, 263 μmol) in DCM (2 mL) and TFA (2 mL) was stirred at RT overnight under N₂. The mixture was concentrated under reduced pressure and the residue was then partitioned between DCM (5 mL) and sat. aq. NaHCO₃ (5 mL). The organic layer was concentrated under reduced pressure and the crude residue was purified by prep-TLC (DCM:MeOH:NH₄OH, 10:1:0.1) to afford the title product (25 mg, 18%) as a yellow solid. LCMS (method A): 3.55 min; m/z=513.2 [M+H]⁺. ¹H NMR (400 MHz, DMSO-d₆): 9.73 (s, 1H), 9.22 (s, 1H), 8.78 (s, 1H), 8.23 (s, 1H), 8 8.12 (d, J=2 Hz, 1H), 7.66 (d, J=8.4 Hz, 2H), 7.46 (d, J=8.4 Hz, 2H), 7.23 (d, J=8.4 Hz, 2H), 6.90 (d, J=8.4 Hz, 2H), 4.19 (t, J=13.2 Hz, 2H), 3.73 (s, 3H), 2.88-2.80 (m, 2H), 2.66-2.60 (m, 1H), 1.91 (d, J=10.8 Hz, 1H), 1.76 (d, J=12 Hz, 1H), 1.68-1.61 (m, 1H), 1.58-1.51 (m, 1H).

The following compounds were similarly prepared from the appropriate amine SM prepared following step 1 and 2 of Compound 11 and phenyl N-(4-{1-tert-butyl-4-carbamoyl-5-[(pyrazin2-yl)amino]-1H-pyrazol-3-yl}phenyl)carbamate also prepared according to the method described for the synthesis of Compound 88:

TABLE 13

| Compound | | | | | |
|---|---|---|---|---|---|
| No | Name | Structure | LCMS data | ¹H NMR data | SM |
| 89 | N-(4-(4-carbamoyl- 5-(pyrazin-2- ylamino)-1H- pyrazol-3- yl)phenyl)-3- phenylpiperidine-1- carboxamide | 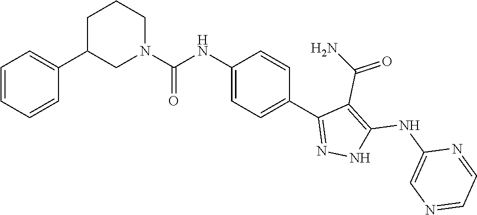 [SMILES: NC(=O)c1c(-c2ccc(NC(=O)N3CCCC(c4ccccc4)C3)cc2)n[nH]c1Nc1cnccn1] | LCMS (method A): 2.50 min m/z = 483.2 [M + H]⁺. | ¹H NMR (400 MHz, DMSO-d₆): 9.67 (s, 1H), 9.29 (s, 1H), 8.81 (s, 1H), 8.22 (t, J = 2.4 Hz, 1H), 8.11 (d, J = 2.8 Hz, 1H), 7.66 (d, J = 8.4 Hz, 2H), 7.46 (d, J = 8.4 Hz, 2H), 7.33 (t, J = 3.2 Hz, 4H), 7.24 (t, J = 2.8 Hz, 1H), 4.23- 4.09 (m, 1H), 3.16 (d, J = 5.2 Hz, 1H), 2.92-2.89 (m, 2H), 2.69-2.66 (m, 1H), 1.93 (d, J = 13.2 Hz, 1H), 1.75-1.68 (m, 4H). | 3-phenylpiperidine |
| 90 | (3R)-N-(4-{4- carbamoyl-5- [(pyrazin-2- yl)amino]-1H- pyrazol-3- yl}phenyl)-3- phenylpiperidine-1- carboxamide | 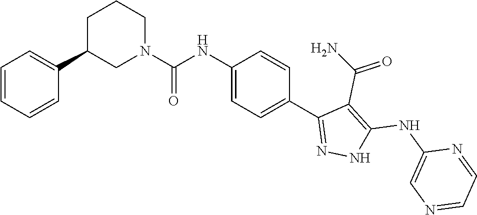 [SMILES: NC(=O)c1c(-c2ccc(NC(=O)N3CCC[C@H](c4ccccc4)C3)cc2)n[nH]c1Nc1cnccn1] | LCMS (method A): 2.50 min; m/z = 483.2 [M + H]⁺. | ¹H NMR (400 MHz, DMSO-d₆) δ 9.70 (s, 1H), 9.20 (brs, 1H), 8.81 (s, 1H), 8.23-8.22 (m, 1H), 8.11 (d, J = 2.4 Hz, 1H), 7.64 (d, J = 8.8 Hz, 2H), 7.45 (d, J = 8.8 Hz, 2H), 7.35-7.30 (m, 4H), 7.25-7.21 (m, 1H), 4.20 (d, J = 12.8 Hz, 2H), 2.94-2.82 (m, 2H), 2.71-2.64 (m, 1H), 1.92 (d, J = 12.4 Hz, 1H), 1.79-1.64 (m, 2H), 1.59-1.52 (m, 1H) | |
| 91 | (3S)-N-(4-{4- carbamoyl-5- [(pyrazin-2- yl)amino]-1H- pyrazol-3- yl}phenyl)-3- phenylpiperidine-1- carboxamide | 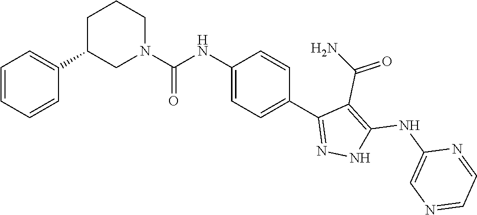 [SMILES: NC(=O)c1c(-c2ccc(NC(=O)N3CCC[C@@H](c4ccccc4)C3)cc2)n[nH]c1Nc1cnccn1] | LCMS (method A): 2.51 min; m/z = 483.2 [M + H]⁺. | ¹H NMR (400 MHz, DMSO-d₆) δ 12.92 (s, 1H), 9.65 (s, 1H), 9.27 (s, 1H), 8.82 (s, 1H), 8.23-8.22 (m, 1H), 8.11 (d, J = 2.8 Hz, 1H), 7.65 (d, J = 8.4 Hz, 2H), 7.45 (d, J = 8.4 Hz, 2H), 7.36-7.31 (m, 4H), 7.26-7.21 (m, 1H), 4.23- 4.19 (m, 2H), 2.94-2.82 (m, 2H), 2.72-2.65 (m, 1H), 1.92 (d, J = 12.4 Hz, 1H), 1.79-1.65 (m, 2H), 1.59-1.52 (m, 1H) | |
| 92 | N-(4-(4-carbamoyl- 5-(pyrazin-2- ylamino)-1H- pyrazol-3- yl)phenyl)-3-(4- chlorophenyl)piperidine- 1-carboxamide | 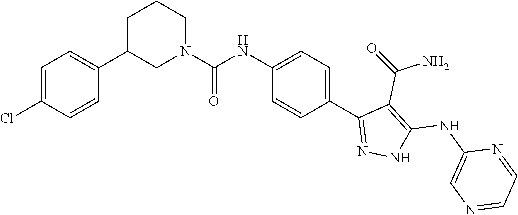 [SMILES: NC(=O)c1c(-c2ccc(NC(=O)N3CCCC(c4ccc(Cl)cc4)C3)cc2)n[nH]c1Nc1cnccn1] | LCMS (method A): 3.83 min; m/z = 517.2 [M + H]⁺. | ¹H NMR (400 MHz, DMSO-d₆): 9.72 (br s, 1H), 9.22 (br s, 1H), 8.81 (s, 1H), 8.23 (s, 1H), 8.11 (d, J = 2.4 Hz, 1H), 7.65 (d, J = 8.8 Hz, 2H), 7.46 (d, J = 8.8 Hz, 2H), 7.41-7.35 (m, 5H), 4.23- 4.17 (m, 2H), 2.95-2.82 (m, 2H), 2.74-2.68 (m, 1H), 1.95-1.91 (m, 1H), 1.79-1.76 (m, 1H), 1.69- 1.66 (m, 1H), 1.57 (s, 1H) | 3-(4- chlorophenyl) piperidine |
| 93 | N-(4-(4-carbamoyl- 5-(pyrazin-2- ylamino)-1H- pyrazol-3- yl)phenyl)-3-(4- (trifluoromethyl) phenyl)piperidine-1- carboxamide | 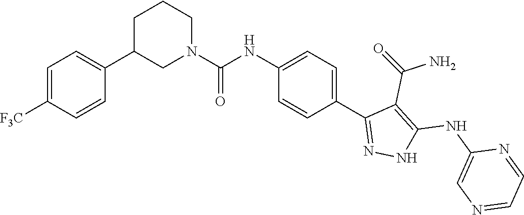 [SMILES: NC(=O)c1c(-c2ccc(NC(=O)N3CCCC(c4ccc(C(F)(F)F)cc4)C3)cc2)n[nH]c1Nc1cnccn1] | LCMS (method A): 3.74 min; m/z = 551.2 [M + H]⁺. | ¹H NMR (400 MHz, DMSO-d₆): 12.92 (s, 1H), 9.67 (s, 1H), 9.29 (s, 1H), 8.84 (s, 1H), 8.22 (q, J = 1.6 Hz, 1H), 8.11 (d, J = 2.4 Hz, 1H), 7.71 (d, J = 8.4 Hz, 2H), 7.67 (d, J = 8.4 Hz, 2H), 7.57 (d, J = 8.0 Hz, 2H), 7.47 (d, J = 8.8 Hz, 2H), 7.34-7.09 (m, 1H), 5.97 (s, 1H), 4.23 (d, J = 12.8 Hz, 2H), 2.98 (t, J = 12.0 Hz, 2H), 2.91-2.79 (m, 2H), 1.96 (d, J = 12.0 Hz, 1H), 1.80-1.72 (m, 2 H), 1.61-1.55 (m, 1 H). | 3-(4- (trifluoromethyl) phenyl)piperidine |
| 94 | N-(4-(4-carbamoyl- 5-(pyrazin-2- ylamino)-1H- pyrazol-3- yl)phenyl)-3-(4- fluorophenyl)piperidine- 1-carboxamide | 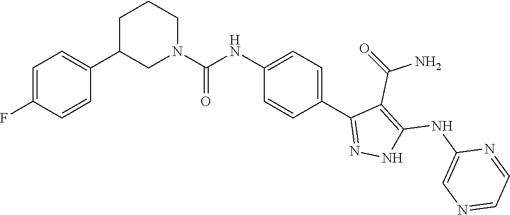 [SMILES: NC(=O)c1c(-c2ccc(NC(=O)N3CCCC(c4ccc(F)cc4)C3)cc2)n[nH]c1Nc1cnccn1] | LCMS (method A): 3.63 min; m/z = 501.2 [M + H]⁺. | ¹H NMR (400 MHz, DMSO-d₆): 12.91 (s, 1H), 9.67 (s, 1H), 9.29 (s, 1H), 8.81 (s, 1H), 8.23 (s, 1H), 8.11 (d, J = 2.4 Hz, 1H), 7.67 (d, J = 8.4 Hz, 2H), 7.47 (d, J = 8.4 Hz, 2H), 7.37 (q, J = 8.4, 5.6 Hz, 2H), 7.16 (t, J = 8.4 Hz, 2H), 5.98 (br s, 1H), 4.24-4.17 (m, 2H), 2.93-2.82 (m, 2H), 2.75-2.67 (m, 1H), 1.95-1.90 (m, 1H), 1.79-1.64 (m, 2H), 1.59- 1.52 (m, 1H) | 3-(4- fluorophenyl) piperidine |
| 95 | N-(4-(4-carbamoyl- 5-(pyrazin-2- ylamino)-1H- pyrazol-3- yl)phenyl)-3-(3- fluorophenyl)piperidine- 1-carboxamide | 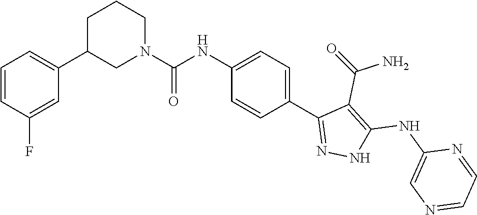 [SMILES: NC(=O)c1c(-c2ccc(NC(=O)N3CCCC(c4cccc(F)c4)C3)cc2)n[nH]c1Nc1cnccn1] | LCMS (method A): 3.63 min; m/z = 501.2 [M + H]⁺. | ¹H NMR (400 MHz, DMSO-d₆): 12.92 (s, 1H), 9.66 (s, 1H), 9.28 (s, 1H), 8.84 (s, 1H), 8.22 (s, 1H), 8.11 (d, J = 2.4 Hz, 2H), 7.46 (d, J = 6.8 Hz, 2H), 7.37 (q, J = 6.4 Hz, 1H), 7.18 (q, J = 4.4 Hz, 1H), 7.06 (d, J = 15.2 Hz, 1H), 4.22-4.19 (m, 2H), 2.97- 2.82 (m, 2H), 2.76-2.70 (m, 1H), 1.95 (q, J = 10.8 Hz, 1H), 1.78- 1.65 (m, 2H), 1.56-1.52 (m, 1H). | 3-(3- fluorophenyl) piperidine |
| 96 | N-(4-(4-carbamoyl- 5-(pyrazin-2- ylamino)-1H- pyrazol-3- yl)phenyl)-3-(3- chlorophenyl)piperidine- 1-carboxamide | 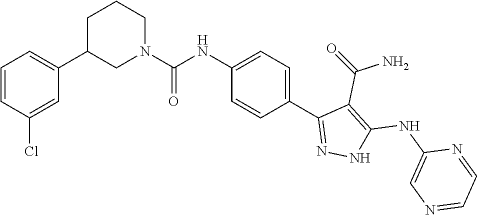 [SMILES: NC(=O)c1c(-c2ccc(NC(=O)N3CCCC(c4cccc(Cl)c4)C3)cc2)n[nH]c1Nc1cnccn1] | LCMS (method A): 3.72 min; m/z = 517.3 [M + H]⁺. | ¹H NMR (400 MHz, DMSO-d₆): 9.72 (s, 1H), 9.21 (s, 1H), 8.78 (d, J = 4.8 Hz, 1H), 8.22 (s, 1H), 8.11 (d, J = 2.4 Hz, 1H), 7.65 (d, J = 8.8 Hz, 2H), 7.48-7.30 (m, 7H), 4.23-4.18 (m, 2H), 2.99- 2.83 (m, 2H), 2.76-2.70 (m, 1H), 1.95-1.92 (m, 1H), 1.78-1.69 (m, 2H), 1.56 (s, 1H). | 3-(3- chlorophenyl) piperidine |
| 97 | N-(4-(4-carbamoyl- 5-(pyrazin-2- ylamino)-1H- pyrazol-3- yl)phenyl)-3-(3- methoxyphenyl) piperidine-1- carboxamide | 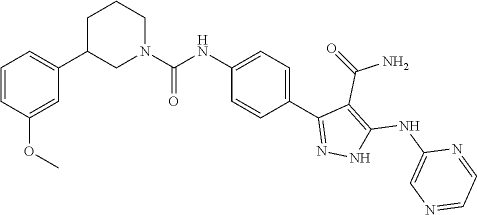 [SMILES: COc1cccc(C2CCCN(C(=O)Nc3ccc(-c4n[nH]c(Nc5cnccn5)c4C(N)=O)cc3)C2)c1] | LCMS (method A): 3.53 min; m/z = 513.2 [M + H]⁺. | ¹H NMR (400 MHz, DMSO-d₆): 12.88 (s, 1H), 9.67 (s, 1H), 9.29 (s, 1H), 8.80 (s, 1H), 8.23-8.22 (m, 1H), 8.11 (d, J = 2.8 Hz, 1H), 7.67 (d, J = 8.4 Hz, 2H), 7.46 (d, J = 8.4 Hz, 2H), 7.25 (t, J = 12.0 Hz, 2H), 6.90-6.88 (m, 2H), 6.82-6.80 (m, 1H), 5.97 (s, 1H ), 4.21 (br s, 2H), 3.76 (s, 3H), 2.96-2.82 (m, 2H), 2.73- 2.60 (m, 1H), 1.95-1.91 (m, 1H), 1.78-1.68 (m, 2H), 1.58-1.50 (m, 1H). | 3-(3- methoxyphenyl) piperidine |
| 98 | N-(4-(4-carbamoyl- 5-(pyrazin-2- ylamino)-1H- pyrazol-3- yl)phenyl)-3-(3- (trifluoromethyl)phenyl) piperidine-1- carboxamide | 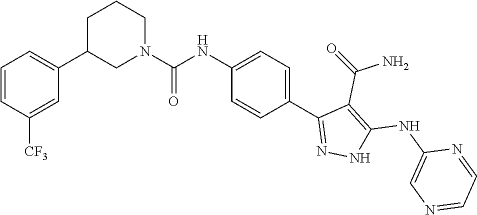 [SMILES: NC(=O)c1c(-c2ccc(NC(=O)N3CCCC(c4cccc(C(F)(F)F)c4)C3)cc2)n[nH]c1Nc1cnccn1] | LCMS (method A): 3.83 min; m/z = 551.2 [M + H]⁺. | ¹H NMR (400 MHz, DMSO-d₆): 12.91 (s, 1H), 9.68 (s, 1H), 9.29 (br s, 1H), 8.82 (s, 1H), 8.23 (s, 1H), 8.12 (s, 1H), 7.75-7.55 (m, 7H), 7.47 (d, J = 8.4 Hz, 2H), 5.97 (br s, 1H), 4.29-4.19 (m, 2H), 3.02 (t, J = 7.6 Hz, 1H), 2.94-2.81 (m, 2H), 2.05-1.92 (m, 1H), 1.82-1.71 (m, 2H), 1.62- 1.52 (m, 1H). | 3-(3- (trifluoromethyl) phenyl)piperidine |
| 99 | N-(4-(4-carbamoyl- 5-(pyrazin-2- ylamino)-1H- pyrazol-3- yl)phenyl)-7- (trifluoromethyl)- 3.4- dihydroisoquinoline- 2(1H)- carboxamide | 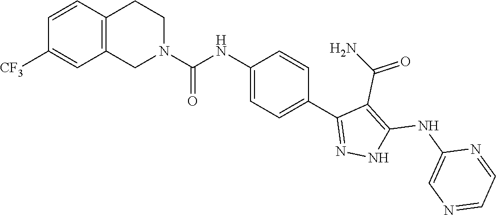 [SMILES: NC(=O)c1c(-c2ccc(NC(=O)N3CCc4ccc(C(F)(F)F)cc4C3)cc2)n[nH]c1Nc1cnccn1] | LCMS (method A): 3.65 min; m/z = 523.2 [M + H]⁺. | ¹H NMR (400 MHz, DMSO-d₆): 12.92 (br s, 1H), 9.66 (br s, 1H), 9.28 (br s, 1H), 8.93 (s, 1H), 8.22 (s, 1H), 8.11 (s, 1H), 7.69- 7.67 (m, 2H), 7.59 (s, 1H), 7.55- 7.42 (m, 5H), 4.76 (s, 2H), 3.77 (t, J = 5.6 Hz, 2H), 2.96 (t, J = 4.0 Hz, 2H). | 7- (trifluoromethyl)- 1,2,3,4- tetrahydroiso quinoline |
| 100 | N-(4-(4-carbamoyl- 5-(pyrazin-2- ylamino)-1H- pyrazol-3- yl)phenyl)-6- (trifluoromethyl)-3.4- dihydroisoquinoline- 2(1H)- carboxamide | 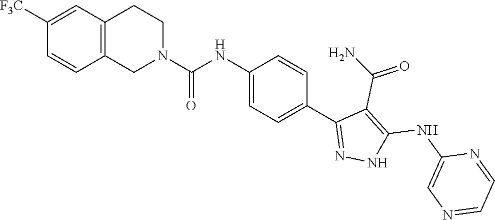 [SMILES: NC(=O)c1c(-c2ccc(NC(=O)N3CCc4cc(C(F)(F)F)ccc4C3)cc2)n[nH]c1Nc1cnccn1] | LCMS (method A): 3.71 min; m/z = 523.1 [M + H]⁺. | ¹H NMR (400 MHz, MeOD-d₄): 8.26 (s, 1H), 8.08 (s, 4H), 7.63- 7.50 (m, 7H), 7.40 (d, J = 8.4 Hz, 1H), 4.80 (s, 2H), 3.83 (t, J = 11.6 Hz, 2H). | 6- (trifluoromethyl)- 1,2,3,4- tetrahydroiso quinoline |
| 101 | N-(4-{4- carbamoyl-5- [(pyrazin-2- yl)amino]-1H- pyrazol-3- yl}phenyl)-1,2,3,4- tetrahydroisoquinoline- 2-carboxamide | 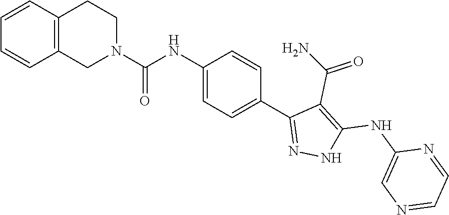 [SMILES: NC(=O)c1c(-c2ccc(NC(=O)N3CCc4ccccc4C3)cc2)n[nH]c1Nc1cnccn1] | LCMS (Method A): 3.28 min; m/z = 455.1 [M + H]⁺. | ¹H NMR (400 MHz, DMSO-d₆): 12.92 (s, 1H), 9.67 (s, 1H), 9.28 (s, 1H), 8.85 (s, 1H), 8.22 (t, J = 2.0 Hz, 1H), 8.11 (d, J = 2.4 Hz, 1H), 7.69 (d, J = 8.4 Hz, 2H), 7.48 (d, J = 8.8 Hz, 2H), 7.19 (s, 5H), 4.67 (s, 2H), 3.73 (t, J = 6.0 Hz, 2H), 2.87 (t, J = 6.0 Hz, 2H). | 1,2,3,4- tetrahydroiso quinoline |
| 102 | N-(4-(4-carbamoyl- 5-(pyrazin-2- ylamino)-1H- pyrazol-3- yl)phenyl)isoindoline- 2-carboxamide | 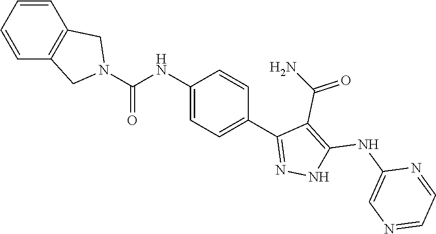 [SMILES: NC(=O)c1c(-c2ccc(NC(=O)N3Cc4ccccc4C3)cc2)n[nH]c1Nc1cnccn1] | LCMS (method A): 1.07 min; m/z = 467.8 [M + H]⁺. | ¹H NMR (400 MHz, DMSO-d₆): 12.92 (s, 1H), 9.67 (s, 1H), 9.29 (s, 1H), 8.64 (s, 1H), 8.22 (s, 1H), 8.12 (t, J = 2.8 Hz, 1H), 7.79 (t, J = 8.8, 2H), 7.51 (t, J = 8.8, 2H), 7.38 (br s, 2H), 7.32 (br s, 2H), 4.81 (s, 4H). | isoindoline |

### Compound 103

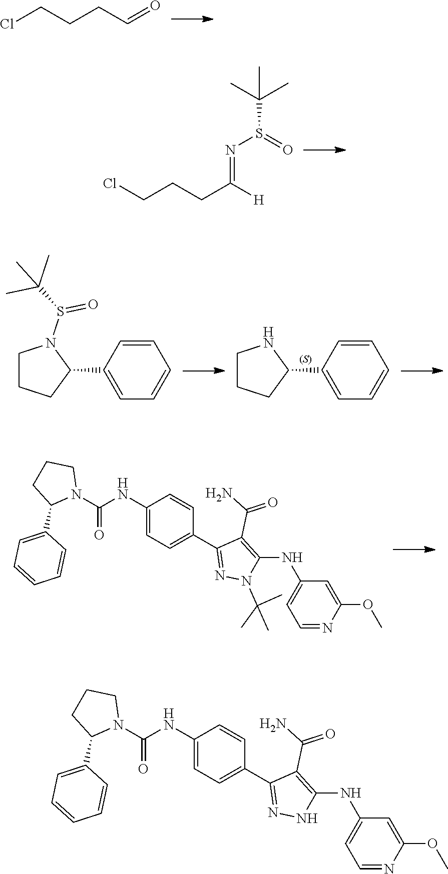

### Step 1: (S,E)-N-(4-chlorobutylidene)-2-methylpropane-2-sulfinamide

To a solution of 4-chlorobutanal (2 g, 18.7 mmol) in THF (40 mL), was added Ti(OEt)₄ (5.10 g, 22.4 mmol) and (S)-2-methylpropane-2-sulfinamide (2.26 g, 18.7 mmol) and the mixture was stirred at RT under N₂ for 16 h. The mixture was partitioned between brine (100 mL) and EtOAc (100 mL) and then stirred for a further 1 h at RT. The solids were removed by filtration and the filtrate was washed with H₂O (2×30 mL). The organic layer was concentrated under reduced pressure and the crude residue was purified by silica gel column chromatography (PE:EtOAc, 10:1) to afford the title product (660 mg, 11%) as yellow oil. LCMS (method A): 2.48 min; m/z=210.1 [M+H]⁺.

### Step 2: (S)-1-((S)-tert-butylsulfinyl)-2-phenylpyrrolidine

To a solution of (S)-N-[(1E)-4-chlorobutylidene]-2-methylpropane-2-sulfinamide (900 mg, 4.29 mmol) in DCM (40 mL) at −78° C. under N₂, was added 3.0 M PhMgBr in THF (931 mg, 5.14 mmol, 1.7 mL). After stirring for 5 h at −78° C., the reaction mixture was quenched with sat. NH₄Cl (10 mL) and the organic layer was separated, washed with H₂O, and concentrated under reduced pressure. The residue was dissolved in THF (30 mL) at RT under N₂ and then a solution of 3.0 M LiHMDS in THF (1.07 g, 6.43 mmol, 2.1 mL) was added and the solution was stirred at RT for 1 h. The reaction mixture was quenched with sat. NH₄Cl (10 mL) and then extracted with EtOAc (50 mL). The organic layer was washed with water (50 mL), dried (Na₂SO₄), and concentrated under reduced pressure. The crude residue was purified by prep-TLC (PE:EtOAc, 4:1) to afford the title compound (130 mg, 13%) as a white solid. LCMS (method A): 2.94 min; m/z=252.2 [M+H]⁺. ¹H NMR (400 MHz, DMSO-d₆): 7.34-7.19 (m, 5H), 4.911 (dd, J=8.0, 3.2 Hz, 1H), 3.64-3.58 (m, 1H), 3.46-3.40 (m, 1H), 2.07-1.98 (m, 1H), 1.82-1.59 (m, 3H), 0.96 (s, 9H).

### Step 3: (S)-2-phenylpyrrolidine

To a solution of (2S)-1-[(S)-2-methylpropane-2-sulfinyl]-2-phenylpyrrolidine (75 mg, 0.298 mmol) in MeOH (10 mL), was added 4.0 M HCl in MeOH (1 mL). The mixture was stirred at RT for 30 min and then concentrated under reduced pressure to give the title product (60 mg, >100%) as a colorless oil. LCMS (method A): 0.62 min; m/z=147.9 [M+H]⁺.

### Step 4: (S)-1-(tert-butyl)-5-((2-methoxypyridin-4-yl)amino)-3-(4-(2-phenyl pyrrolidine-1-carboxamido)phenyl)-1H-pyrazole-4-carboxamide

To a solution of phenyl N-(4-{1-tert-butyl-4-carbamoyl-5-[(2-methoxypyridin-4-yl)amino]-1H-pyrazol-3-yl}phenyl)carbamate (90 mg, 0.179 mmol) in THF (10 mL) was added DIPEA (229 mg, 1.78 mmol) and (2S)-2-phenylpyrrolidine (26.3 mg, 179 μmol). The mixture was stirred at 60° C. under N₂ overnight. The mixture was then partitioned between H₂O (50 mL) and EtOAc (50 mL). The organic layer was separated, dried (Na₂SO₄), and then concentrated under reduced pressure. The crude residue was purified by prep-TLC (DCM:MeOH, 20:1) to afford the title product (60 mg, 60%) as a yellow solid. LCMS (method A): 2.83 min; m/z=544.2 [M+H]⁺.

### Step 5: (S)-5-((2-methoxypyridin-4-yl)amino)-3-(4-(2-phenylpyrrolidine-1-carboxamido)phenyl)-1H-pyrazole-4-carboxamide (Compound 103)

A solution of 1-tert-butyl-5-[(2-methoxypyridin-4-yl)amino]-3-(4-{[(2S)-2-phenyl pyrrolidine-1-carbonyl]amino}phenyl)-1H-pyrazole-4-carboxamide (60 mg, 0.108 mmol) in TFA (2 mL) was stirred at 60° C. for 1 h. The reaction mixture was concentrated under reduced pressure and the residue was triturated (Et₂O), then dried under reduced pressure to afford the title compound·mono TFA salt (30 mg, 55%) as a white solid. LCMS (method A): 3.15 min; m/z=498.2 [M+H]⁺. ¹H NMR (400 MHz, DMSO-d₆): 12.79 (s, 1H), 9.30 (s, 1H), 8.42 (s, 1H), 7.86 (d, J=6.0 Hz, 1H), 7.63 (d, J=8.4 Hz, 2H), 7.41 (d, J=8.8 Hz, 2H), 7.31-7.29 (m, 2H), 7.22-7.18 (m, 3H), 7.10 (d, J=1.6 Hz, 1H), 6.90 (dd, J=6.4, 1.6 Hz, 1H), 5.98 (br s, 1H), 5.15 (dd, J=7.6, 2.4 Hz, 1H), 3.83-3.82 (m, 1H), 3.79 (s, 3H), 3.62-3.56 (m, 1H), 2.35-2.26 (m, 1H), 1.97-1.82 (m, 2H), 1.80-1.73 (m, 1H).

The following compound was similarly prepared from the appropriate amine (R)-2-phenylpyrrolidine and phenyl N-(4-{1-tert-butyl-4-carbamoyl-5-[(2-methoxypyridin-4-yl)amino]-1H-pyrazol-3-yl}phenyl)carbamate according to the method described for the synthesis of compound 103:

TABLE 14

| Compound | | | | | |
|---|---|---|---|---|---|
| No | Name | Structure | LCMS data | ¹H NMR data | SM |
| 104 | (R)-5-((2- methoxypyridin- 4-yl)amino)-3-(4- (2-phenyl- pyrrolidine- 1-carboxamido) phenyl)-1H- pyrazole-4- carboxamide (mono TFA salt) | 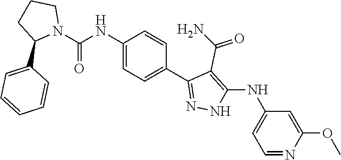 [SMILES: COc1cc(Nc2[nH]nc(-c3ccc(NC(=O)N4CCC[C@@H]4c4ccccc4)cc3)c2C(N)=O)ccn1] | LCMS (method A): 3.14 min; m/z = 498.2 [M + H]⁺. | ¹H NMR (400 MHz, DMSO-d₆): 12.79 (s, 1H), 9.30 (s, 1H), 8.42 (s, 1H), 7.86 (d, J = 6.0 Hz, 1H), 7.63 (d, J = 8.8 Hz, 2H), 7.41 (d, J = 8.8 Hz, 2H), 7.31-7.29 (m, 2H), 7.22- 7.18 (m, 3H), 7.11 (d, J = 1.6 Hz, 1H), 6.90 (dd, J = 6.0, 1.6 Hz, 1H), 5.98 (br s, 1H), 5.14 (dd, J = 7.6, 2.4 Hz, 1H), 3.83-3.82 (m, 1H), 3.80 (s, 3H), 3.62-3.56 (m, 1H), 2.35-2.26 (m, 1H), 1.98-1.83 (m, 2H), 1.80-1.73 (m, 1H). | (R)-2- phenyl- pyrro- lidine |

### Compound 105

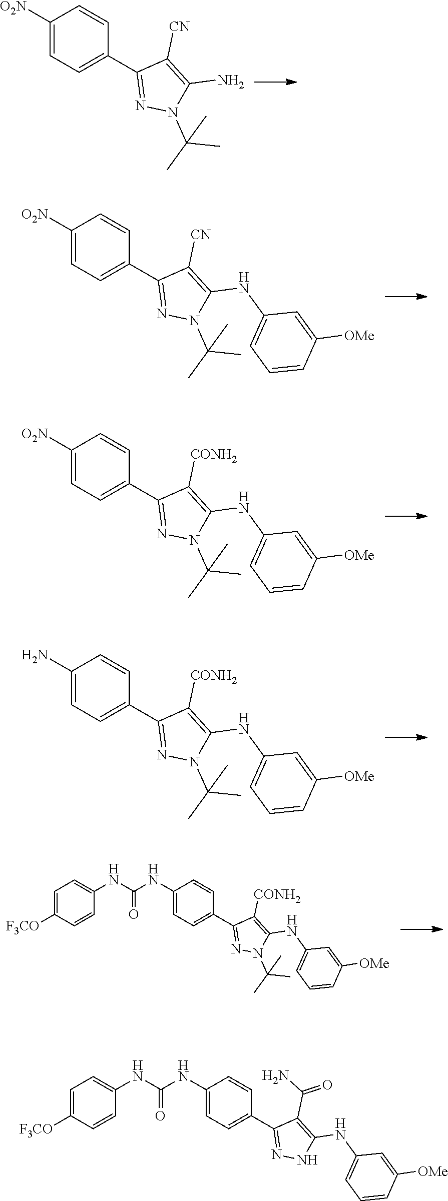 [SMILES: CC(C)(C)n1nc(-c2ccc([N+](=O)[O-])cc2)c(C#N)c1N ; COc1cccc(Nc2c(C#N)c(-c3ccc([N+](=O)[O-])cc3)nn2C(C)(C)C)c1 ; COc1cccc(Nc2c(C(N)=O)c(-c3ccc([N+](=O)[O-])cc3)nn2C(C)(C)C)c1 ; COc1cccc(Nc2c(C(N)=O)c(-c3ccc(N)cc3)nn2C(C)(C)C)c1 ; COc1cccc(Nc2c(C(N)=O)c(-c3ccc(NC(=O)Nc4ccc(OC(F)(F)F)cc4)cc3)nn2C(C)(C)C)c1 ; COc1cccc(Nc2[nH]nc(-c3ccc(NC(=O)Nc4ccc(OC(F)(F)F)cc4)cc3)c2C(N)=O)c1]

### Step 1: 1-(tert-butyl)-5-((3-methoxyphenyl)amino)-3-(4-nitrophenyl)-1H-pyrazole-4-carbonitrile

A mixture of 5-amino-1-(tert-butyl)-3-(4-nitrophenyl)-1H-pyrazole-4-carbonitrile (1.5 g, 5.2 mmol), 1-bromo-3-methoxybenzene (0.60 g, 6.3 mmol), Pd₂(dba)₃ (0.3 g, 0.32 mmol), BINAP (90 mg, 0.32 mmol) and Cs₂CO₃ (1.6 g, 11.0 mmol) in diglyme (30 mL) was stirred at 150° C. under N₂ for 24 h. The residue was diluted with EtOAc (50 mL), and the mixture was washed with H₂O (25 mL). The organic layer was dried over Na₂SO₄ and then concentrated under reduced pressure. The crude residue was purified by silica gel column chromatography (PE:EtOAc, 10:1) to afford the desired product (200 mg, 10%) as grey oil. LCMS (method A): 3.35 min; m/z=392.1 [M+H]⁺.

### Step 2: 1-(tert-butyl)-5-((3-methoxyphenyl)amino)-3-(4-nitrophenyl)-1H-pyrazole-4-carboxamide

To a solution of 1-(tert-butyl)-5-((3-methoxyphenyl)amino)-3-(4-nitrophenyl)-1H-pyrazole-4-carbonitrile (200 mg, 0.51 mmol) and K₂CO₃ (212 mg, 1.53 mmol) in DMSO (5 mL), was added 30% aq. H₂O₂ (1.4 mL) and the mixture was stirred at 120° C. for 24 h. The mixture was diluted with EtOAc (50 mL) and washed with H₂O (25 mL). The organic layer was dried over Na₂SO₄ and concentrated under reduced pressure. The crude residue was purified by silica gel column chromatography (PE:EtOAc, 20:1) to afford the desired product (20 mg, 10%) as a grey solid. LCMS (method A): 2.96 min; m/z=410.1 [M+H]⁺.

### Step 3: 3-(4-aminophenyl)-1-(tert-butyl)-5-((3-methoxyphenyl)amino)-1H-pyrazole-4-carboxamide

To a solution of 1-(tert-butyl)-5-((3-methoxyphenyl)amino)-3-(4-nitrophenyl)-1H-pyrazole-4-carboxamide (50 mg, 0.12 mmol) in MeOH (6 mL), was added aq. NH₄Cl solution (2 mL) and Zn dust (40 mg, 0.61 mmol), and the mixture was stirred at 60° C. for 3 h. The mixture was filtered through Celite, rinsed with MeOH (5 mL) and the combined filtrates were concentrated under reduced pressure. The residue was diluted with EtOAc (25 mL) and H₂O (15 mL). The organic layer was separated, dried (Na₂SO₄), and concentrated under reduced pressure to afford the desired product (22 mg, 49%) as a grey solid, which was used in the next step without further purification. LCMS (method A): 2.34 min; m/z=380.1 [M+H]⁺.

### Step 4: 1-(tert-butyl)-5-((3-methoxyphenyl)amino)-3-(4-(3-(4-(trifluoromethoxy)phenyl)ureido)phenyl)-1H-pyrazole-4-carboxamide

A mixture of 3-(4-aminophenyl)-1-(tert-butyl)-5-((3-methoxyphenyl)amino)-1H-pyrazole-4-carboxamide (22 mg, 0.054 mmol) and 1-isocyanato-4-(trifluoromethoxy)benzene (24 mg, 0.11 mmol) in THF (3 mL) was stirred at RT for 1 h, then concentrated. The residue was diluted with EtOAc (15 mL), and the organic phase was washed with H₂O (10 mL), dried (Na₂SO₄) and then concentrated under reduced pressure. The crude residue was purified by prep-TLC (DCM:MeOH, 20:1) to afford the desired product (15 mg, 46%) as a grey solid. LCMS (method A): 3.22 min; m/z=583.1 [M+H]⁺.

### Step 5: 5-((3-methoxyphenyl)amino)-3-(4-(3-(4-(trifluoromethoxy)phenyl)ureido)phenyl)-1H-pyrazole-4-carboxamide (Compound 105)

A solution of 1-(tert-butyl)-5-((3-methoxyphenyl)amino)-3-(4-(3-(4-(trifluoromethoxy)phenyl)ureido)phenyl)-1H-pyrazole-4-carboxamide (15 mg, 0.026 mmol) in TFA (1 mL) was stirred at 60° C. for 1 h, then concentrated under reduced pressure. The crude residue was purified by prep-HPLC (Agilent 10 prep-C18, 10 μm, 250×21.2 mm column, eluting with a gradient of MeOH in water with 0.1% TFA, at a flow rate of 20 mL/min) to afford the desired product as mono TFA salt (5 mg, 37%) as a white solid. LCMS (method A): 2.92 min; m/z=527.2 [M+H]⁺. ¹NMR (400 MHz, DMSO-d₆): 12.63 (s, 1H), 9.08-9.06 (m, 3H), 7.65-7.50 (m, 8H), 7.32-7.28 (m, 3H), 7.13 (t, J=8.1 Hz, 1H), 6.98 (d, J=7.1 Hz, 1H), 6.40 (d, J=6.4 Hz, 1H), 3.74 (s, 3H).

### Compound 106

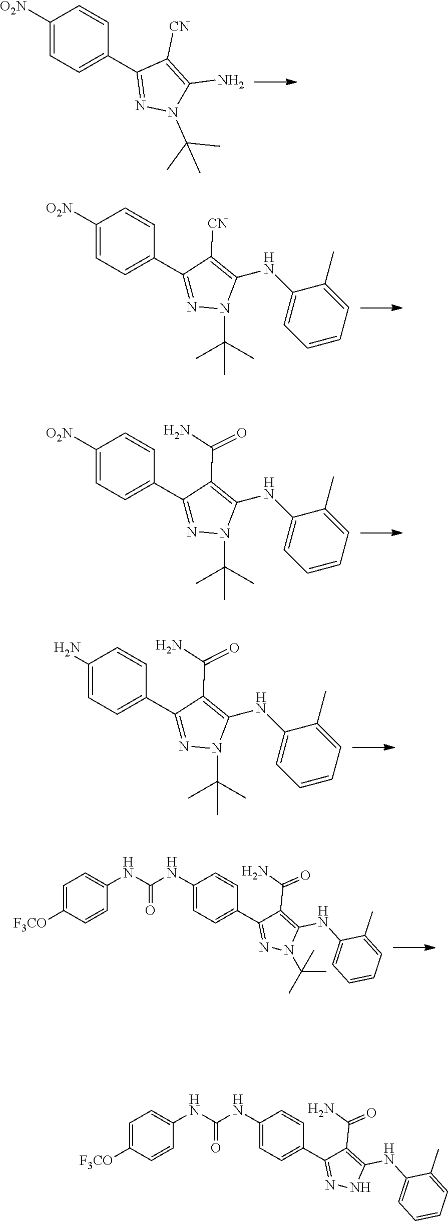 [SMILES: CC(C)(C)n1nc(-c2ccc([N+](=O)[O-])cc2)c(C#N)c1N ; Cc1ccccc1Nc1c(C#N)c(-c2ccc([N+](=O)[O-])cc2)nn1C(C)(C)C ; Cc1ccccc1Nc1c(C(N)=O)c(-c2ccc([N+](=O)[O-])cc2)nn1C(C)(C)C ; Cc1ccccc1Nc1c(C(N)=O)c(-c2ccc(N)cc2)nn1C(C)(C)C ; Cc1ccccc1Nc1c(C(N)=O)c(-c2ccc(NC(=O)Nc3ccc(OC(F)(F)F)cc3)cc2)nn1C(C)(C)C ; Cc1ccccc1Nc1[nH]nc(-c2ccc(NC(=O)Nc3ccc(OC(F)(F)F)cc3)cc2)c1C(N)=O]

### Step 1: 5-(o-tolylamino)-1-tert-butyl-3-(4-nitrophenyl)-1H-pyrazole-4-carbonitrile

A mixture of 1-tert-butyl-5-amino-3-(4-nitrophenyl)-1H-pyrazole-4-carbonitrile (5.01 g, 17.5 mmol), 1-bromo-2-methylbenzene (3.59 g, 21 mmol), Pd(t-Bu₃P)₂ (0.90 g, 1.75 mmol) and Cs₂CO₃ (11.40 g, 35 mmol) in degassed PhMe (50 mL) was stirred at 110° C. under N₂ for 8 h. The reaction mixture was concentrated under reduced pressure and then diluted with EtOAc (100 mL) and H₂O (50 mL). The organic layer was separated, dried (Na₂SO₄) and concentrated under reduced pressure. The crude residue was purified by silica gel column chromatography (PE:EtOAc, 1:1) to afford the desired product (4.99 g, 76%) as a grey solid. LCMS (method A): 2.42 min; m/z=376.2 [M+H]⁺.

### Step 2: 5-(o-tolylamino)-1-tert-butyl-3-(4-nitrophenyl)-1H-pyrazole-4-carboxamide

To a solution of 5-(o-tolylamino)-1-tert-butyl-3-(4-nitrophenyl)-1H-pyrazole-4-carbonitrile (300 mg, 0.77 mmol) in DMSO (3 mL) and EtOH (5 mL), was added 30% aq. H₂O₂ (2 mL) and 5% aq. NaOH (0.3 mL). The mixture was stirred at 80° C. for 2 h, then diluted with EtOAc (50 mL) and H₂O (25 mL). The organic layer was separated, dried (Na₂SO₄) and concentrated under reduced pressure. The crude residue was purified by silica gel column chromatography (PE:EtOAc, 5:1) to afford the desired product (20 mg, 10%) as a grey solid. LCMS (method A): 2.75 min; m/z=394.2 [M+H]⁺.

### Step 3: 5-(o-tolylamino)-1-tert-butyl-3-(4-aminophenyl)-1H-pyrazole-4-carboxamide

To a solution of 5-(o-tolylamino)-1-tert-butyl-3-(4-nitrophenyl)-1H-pyrazole-4-carboxamide (220 mg, 0.56 mmol) in MeOH (6 mL), was added sat. aq. NH₄Cl (2 mL) and Zn dust (181 mg, 2.8 mmol), and the mixture was stirred at 60° C. for 3 h. The mixture was filtered, concentrated under reduced pressure and the residue was diluted with EtOAc (25 mL) and H₂O (15 mL). The organic layer was separated, dried (Na₂SO₄) and concentrated to give the desired product (200 mg, 97%) as a yellow solid, which was used in the next step without further purification. LCMS (method A): 2.02 min; m/z=364.1 [M+H]+.

### Step 4: 1-(tert-butyl)-5-(o-tolylamino)-3-(4-(3-(4-(trifluoromethoxy)phenyl)ureido)phenyl)-1H-pyrazole-4-carboxamide

A mixture of 5-(o-tolylamino)-1-tert-butyl-3-(4-aminophenyl)-1H-pyrazole-4-carboxamide (180 mg, 0.49 mmol) and 1-isocyanato-4-(trifluoromethoxy)benzene (151 mg, 0.74 mmol) in THF (8 mL) was stirred at RT for 1 h. The mixture was concentrated under reduced pressure and the residue was diluted with EtOAc (15 mL), and H₂O (10 mL). The organic layer was separated, dried (Na₂SO₄) and concentrated under reduced pressure. The crude residue was purified by prep-TLC (DCM:MeOH, 20:1) to afford the desired product (170 mg, 61%) as a grey solid. LCMS (method A): 3.86 min; m/z=567.2 [M+H]⁺.

### Step 5: 5-(o-tolylamino)-3-(4-(3-(4-(trifluoromethoxy)phenyl)ureido)phenyl)-1H-pyrazole-4-carboxamide (Compound 106)

A solution of 1-(tert-butyl)-5-(o-tolylamino)-3-(4-(3-(4-(trifluoromethoxy)phenyl)ureido)phenyl)-1H-pyrazole-4-carboxamide (170 mg, 0.30 mmol) in TFA (1 mL) was stirred at 60° C. for 1 h. The solution was concentrated under reduced pressure and the residue was purified by prep-TLC (DCM/MeOH=10:1) to afford the title compound (80 mg, 54%) as a white solid. LCMS (method A): 3.03 min; m/z=511.2 [M+H]⁺. ¹H NMR (400 MHz, DMSO-d₆): 12.63 (s, 1H), 9.96 (d, J=1.2 Hz, 2H), 9.21 (s, 1H), 8.27 (d, J=8.0 Hz, 1H), 7.67 (d, J=8.4 Hz, 2H), 7.60 (d, J=8.8 Hz, 2H), 7.52 (d, J=8.8 Hz, 2H), 7.31 (d, J=8.8 Hz, 2H), 7.17-7.13 (m, 2H), 6.77 (t, J=7.2 Hz, 1H), 2.27 (s, 3H).

The following compounds were similarly prepared from the appropriate haloaryl SM and 1-tert-butyl-5-amino-3-(4-nitrophenyl)-1H-pyrazole-4-carbonitrile following step 1 of the synthesis of compound 106:

TABLE 15

| Compound | | | | | |
|---|---|---|---|---|---|
| No | Name | Structure | LCMS data | ¹H NMR data | SM |
| 107 | 5-(pyrazin-2- ylamino)-3-(4-(3- (4- (trifluoromethoxy) phenyl)ureido)phenyl)- 1H-pyrazole-4- carboxamide | 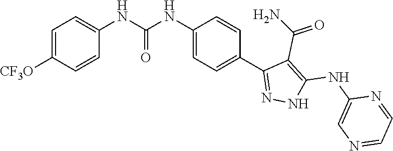 | LCMS (method A): 3.75 min; m/z = 499.1 [M + H]⁺. | ¹H NMR (400 MHz, DMSO-d₆): 12.93 (s, 1H), 9.66 (s, 1H), 9.28 (s, 1H), 9.02 (s, 1H), 8.99 (s, 1H), 8.23 (s, 1H), 8.12 (s, 1H), 7.65 (t, J = | 2- bromo- pyrazine |
| | | | | 8.4 Hz, 2H), | |
| | | | | 7.55 (q, J = 3.2 | |
| | | | | Hz, 5H), 7.30 | |
| | | | | (t, J = 8.4 Hz, 3H). | |
| 108 | 5-(pyridin-2- ylamino)-3-(4-(3- (4- (trifluoromethoxy) phenyl)ureido)phenyl)- 1H-pyrazole-4- carboxamide | 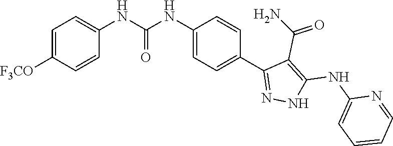 [SMILES: NC(=O)c1c(-c2ccc(NC(=O)Nc3ccc(OC(F)(F)F)cc3)cc2)n[nH]c1Nc1ccccn1] | LCMS (method A): 2.56 min; m/z = 498.1 [M + H]⁺. | ¹H NMR (400 MHz, MeOD-d₄): 8.31 (d, J = 6.0 Hz, 1H), 8.24- 8.20 (m, 1H), 7.74-7.71 (m, 3H), 7.61-7.56 (m, 4H), 7.28- 7.22 (m, 3H). | 2- chloro- pyridine |
| 109 | 5-(pyridin-4- ylamino)-3-(4-(3- (4- (trifluoromethoxy) phenyl)ureido)phenyl)- 1H-pyrazole-4- carboxamide | 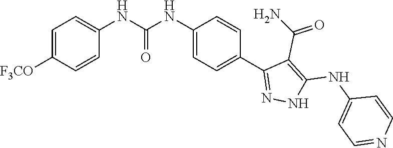 [SMILES: NC(=O)c1c(-c2ccc(NC(=O)Nc3ccc(OC(F)(F)F)cc3)cc2)n[nH]c1Nc1ccncc1] | LCMS (method A): 2.47 min; m/z = 498.2 [M + H]⁺. | ¹H NMR (400 MHz, DMSO-d₆): 12.23 (s, 1H), 10.19 (s, 1H), 10.10-9.90 (m, 2H), 8.37 (d, J = 6.8 Hz, 2H), 7.75-7.35 (m, 9H), 7.29 (d, | 4- chloro- pyridin |
| | | | | J = 8.8 Hz, 2H), | |
| | | | | 6.64 (br s, 1H). | |
| 110 | 5-(pyridin-3- ylamino)-3-(4-(3- (4- (trifluoromethoxy) phenyl)ureido)phenyl)- 1H-pyrazole-4- carboxamide | 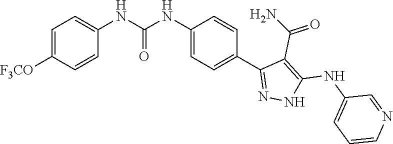 [SMILES: NC(=O)c1c(-c2ccc(NC(=O)Nc3ccc(OC(F)(F)F)cc3)cc2)n[nH]c1Nc1cccnc1] | LCMS (method A): 2.46 min; m/z = 498.2 [M + H]⁺. | ¹H NMR (400 MHz, DMSO-d₆): 12.80 (s, 1H), 9.33 (s, 1H), 9.13 (d, J = 7.6 Hz, 2H), 8.93 (d, J = 1.6 Hz, 1H), 8.33-8.31 (m, 1H), 8.18 | 3- chloro- pyridine |
| | | | | (d, J = 4.4 Hz, | |
| | | | | 1H), 7.65-7.52 | |
| | | | | (m, 7H), 7.31 | |
| | | | | (d, J = 8.4 Hz, | |
| | | | | 1H), 7.22 (s, | |
| | | | | 1H), 7.09 (s, | |
| | | | | 1H), 6.97 (s, | |
| | | | | 1H). | |
| 111 | 5-((4- methoxyphenyl) amino)-3-(4-(3-(4- (trifluoromethoxy) phenyl)ureido)phenyl)- 1H-pyrazole-4- carboxamide | 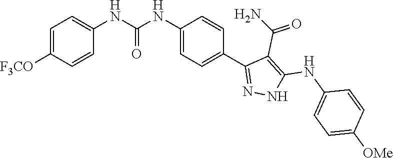 [SMILES: COc1ccc(Nc2[nH]nc(-c3ccc(NC(=O)Nc4ccc(OC(F)(F)F)cc4)cc3)c2C(N)=O)cc1] | LCMS (method A): 2.96 min; m/z = 527.0 [M + H]⁺. | ¹H NMR (400 MHz, DMSO-d₆): 12.51 (s, 1H), 9.01 (d, J = 8.8 Hz, 2H), 8.81 (s, 1H), 7.67- 7.42 (m, 9H), 7.31 (d, J = 8.0 Hz, 2H), 6.86 (d, J = 8.0 Hz, | 1-bromo- 4- methoxy benzene |
| | | | | 2H), 5.80 (br s, | |
| | | | | 1H), 3.71 (s, | |
| | | | | 3H). | |
| 112 | 5-((2- fluorophenyl)amino)- 3-(4-(3-(4- (trifluoromethoxy) phenyl)ureido)phenyl)- 1H-pyrazole-4- carboxamide | 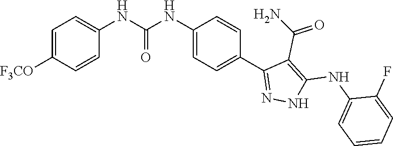 [SMILES: NC(=O)c1c(-c2ccc(NC(=O)Nc3ccc(OC(F)(F)F)cc3)cc2)n[nH]c1Nc1ccccc1F] | LCMS (method A): 3.02 min; m/z = 515.1 [M + H]⁺. | ¹H NMR (400 MHz, DMSO-d₆): 12.73 (s, 1H), 9.39 (s, 1H), 9.32-9.29 (m, 2H), 8.37 (t, J = 8.0 Hz, 1H), 7.68 (d, J = 8.0 Hz, 2H), 7.60 | 1-bromo- 2- fluoro benzene |
| | | | | (d, J = 8.0 Hz, | |
| | | | | 2H), 7.52 (d, | |
| | | | | J = 8.0 Hz, 2H), | |
| | | | | 7.31 (d, J = 8.0 | |
| | | | | Hz, 2H), 7.23- | |
| | | | | 7.12 (m, 2H), | |
| | | | | 6.85-6.81 (m, | |
| | | | | 1H). | |
| 113 | 5-((4- cyanophenyl)amino)- 3-(4-(3-(4- (trifluoromethoxy) phenyl)ureido)phenyl)- 1H-pyrazole-4- carboxamide | 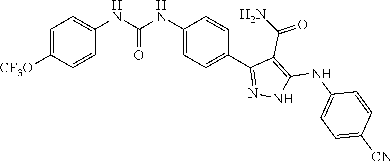 | LCMS (method A): 2.96 min; m/z = 521.9 [M + H]⁺. | ¹H NMR (400 MHz, DMSO-d₆): 9.07 (s, 1H), 9.02 (s, 1H), 8.97 (s, 1H), 7.80- 7.70 (m, 3H), 7.67-7.42 (m, 8H), 7.31 (d, J = 8.4 Hz, 2H). | 4- bromo- benzo nitrile |

### Compound 114

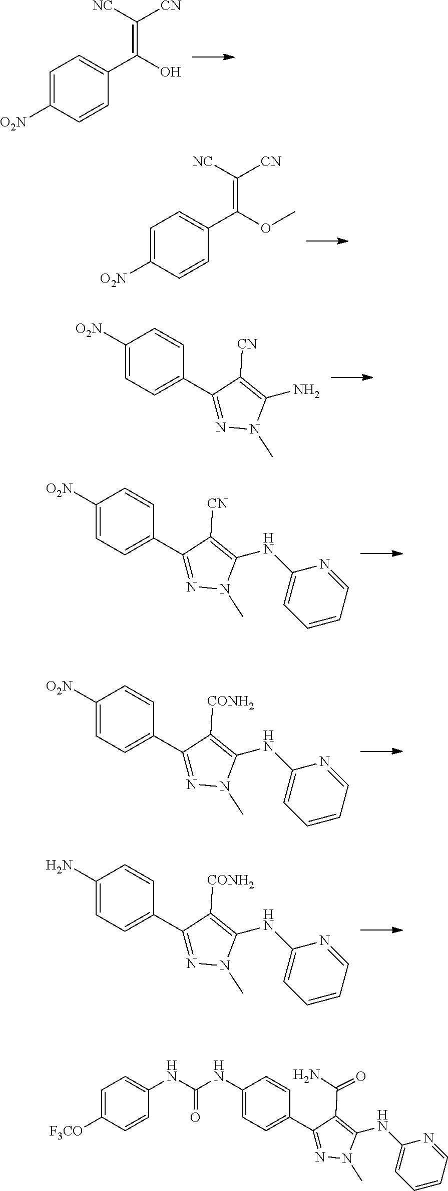

### Step 1: 2-(methoxy(4-nitrophenyl)methylene)malononitrile

To a solution of 2-(hydroxy(4-nitrophenyl)methylene)malononitrile (3 g, 13.9 mmol) in dioxane (20 mL) and H₂O (2 mL), was added NaHCO₃ (4.5 g, 53.6 mmol) and dimethyl sulfate (4.5 mL, 47.6 mmol) at RT. The reaction mixture was stirred at 85° C. for 1 h, and then diluted with EtOAc (100 mL) and H₂O (100 mL). The organic layer was separated, dried (Na₂SO₄) and concentrated under reduced pressure. The crude residue was purified by silica gel column chromatography (PE:EtOAc, 5:1) to afford the desired product (2.0 g, 63%) as a yellow solid. LCMS (method A): 1.75 min; m/z=252.0 [M+Na]⁺.

### Step 2: 5-amino-1-methyl-3-(4-nitrophenyl)-1H-pyrazole-4-carbonitrile

A mixture of 2-(methoxy(4-nitrophenyl)methylene)malononitrile (2 g, 8.73 mmol), methylhydrazinium sulphate (1.38 g, 9.60 mmol) and Et₃N (968 mg, 9.60 mmol) in EtOH (10 mL) was stirred at 80° C. for 1 h. The mixture was concentrated to dryness and then diluted with EtOAc (100 mL) and H₂O (100 mL). The organic layer was separated, dried (Na₂SO₄) and concentrated under reduced pressure. The crude residue was purified by silica gel column chromatography (DCM:MeOH, 20:1) to afford the desired product (1.2 g, 71%) as a yellow solid. LCMS (method A): 2.37 min; m/z=244.1 [M+H]⁺.

### Step 3: 1-methyl-3-(4-nitrophenyl)-5-(pyridin-2-ylamino)-1H-pyrazole-4-carbonitrile

A mixture of 5-amino-1-methyl-3-(4-nitrophenyl)-1H-pyrazole-4-carbonitrile (1.58 g, 6.50 mmol), 2-bromopyridine (1.13 g, 7.15 mmol), Pd(t-Bu₃P)₂ (331 mg, 0.65 mmol) and Cs₂CO₃ (1.6 g, 11.0 mmol) in PhMe (10 mL) was stirred at 110° C. under N₂ for 8 h. The mixture was concentrated under reduced pressure and the residue was then diluted with EtOAc (100 mL) and H₂O (100 mL). The organic layer was separated, dried (Na₂SO₄) and concentrated under reduced pressure. The crude residue was purified by silica gel column chromatography (PE:EtOAc, 1:1) to afford the title product (240 mg, 12%) as a green powder. LCMS (method A): 3.35 min; m/z=321.1 [M+H]⁺.

### Step 4: 1-methyl-3-(4-nitrophenyl)-5-(pyridin-2-ylamino)-1H-pyrazole-4-carboxamide

To a solution of 1-methyl-3-(4-nitrophenyl)-5-(pyridin-2-ylamino)-1H-pyrazole-4-carbonitrile (240 mg, 0.75 mmol) in DMSO (5 mL) and EtOH (30 mL), was added 30% aq. H₂O₂ (5 mL) and 5 M aq. NaOH (1 drop). The resulting mixture was stirred at 120° C. for 1 h, and then diluted with EtOAc (50 mL) and H₂O (25 mL). The organic layer was separated, dried (Na₂SO₄) and concentrated under reduced pressure. The residue (300 mg, >100%) was used in the next reaction without further purification. LCMS (method A): 0.48 min; m/z=339.1 [M+H]⁺.

### Step 5: 3-(4-aminophenyl)-1-methyl-5-(pyridin-2-ylamino)-1H-pyrazole-4-carboxamide

To a solution of 1-methyl-3-(4-nitrophenyl)-5-(pyridin-2-ylamino)-1H-pyrazole-4-carboxamide (300 mg, 0.888 mmol) in MeOH (15 mL), was added aq. NH₄Cl (5 mL) and Zn dust (231 mg, 3.550 mmol). The mixture was stirred at RT overnight, and then filtered. The filtrate was concentrated under reduced pressure and the residue was diluted with EtOAc (25 mL) and H₂O (15 mL). The organic layer was separated, dried (Na₂SO₄) and concentrated under reduced pressure. The crude residue was purified by prep-TLC (DCM:MeOH, 20:1) to afford the title product (40 mg, 15%) as a brown solid. LCMS (method A): 0.46 min; m/z=309.1 [M+H]⁺.

### Step 6: 1-methyl-5-(pyridin-2-ylamino)-3-(4-(3-(4-(trifluoromethoxy)phenyl)ureido)phenyl)-1H-pyrazole-4-carboxamide (Compound 114)

A mixture of 3-(4-aminophenyl)-1-methyl-5-(pyridin-2-ylamino)-1H-pyrazole-4-carboxamide (40 mg, 0.130 mmol) and 1-isocyanato-4-(trifluoromethoxy)benzene (26 mg, 0.130 mmol) in THF (10 mL) was stirred at RT for 1 h. The mixture was concentrated under reduced pressure and the residue was diluted with EtOAc (15 mL), and H₂O (10 mL). The organic layer was separated, dried (Na₂SO₄) and concentrated under reduced pressure. The crude residue was purified by prep-TLC (DCM:MeOH, 10:1) to afford the title product (11 mg, 17%) as a white solid. LCMS (method A): 2.59 min; m/z=512.2 [M+H]⁺. ¹H NMR (400 MHz, DMSO-d₆): 10.10 (s, 1H), 10.00 (s, 1H), 9.70 (s, 1H), 8.18 (d, J=3.6 Hz, 1H), 8.03 (d, J=8.4 Hz, 1H), 7.72-7.69 (m, 3H), 7.58 (d, J=9.2 Hz, 2H), 7.43 (d, J=8.4 Hz, 2H), 7.28 (d, J=8.8 Hz, 2H), 7.00-6.80 (m, 1H), 3.56 (s, 3H).

### Compound 115

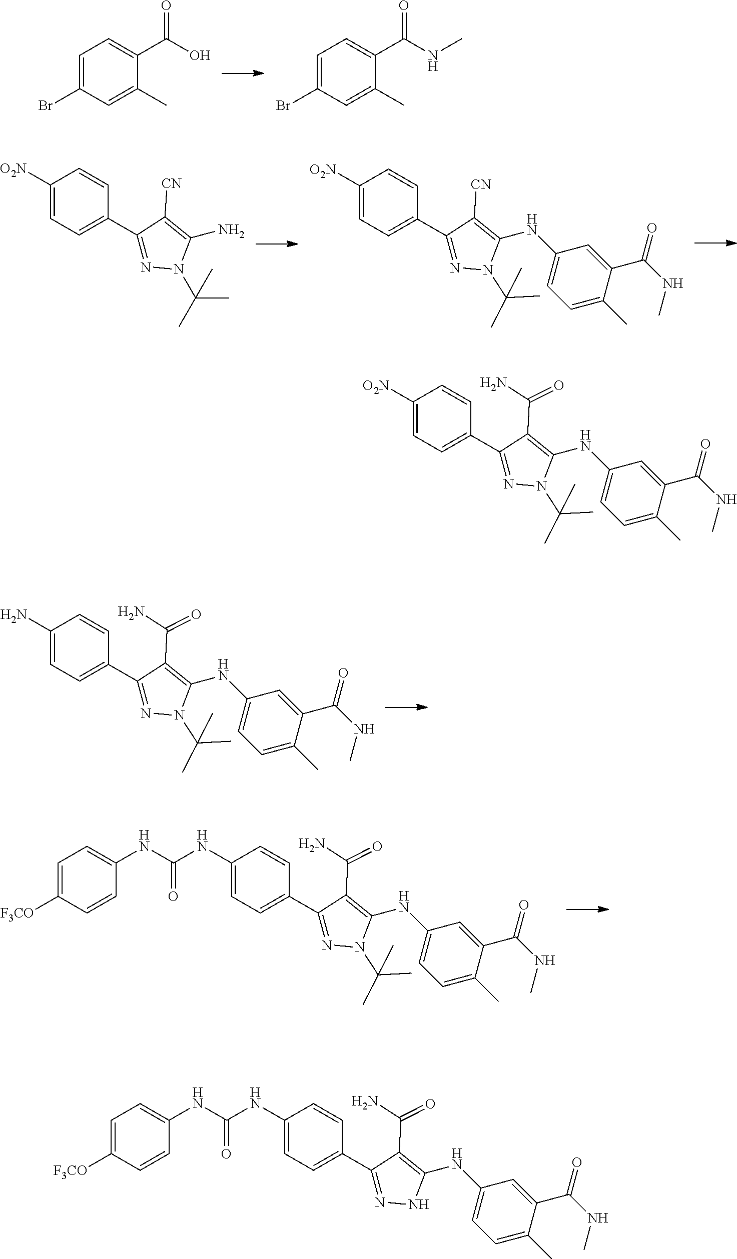

### Step 1: 5-bromo-N,2-dimethylbenzamide

A mixture of 5-bromo-2-methylbenzoic acid (2.1 g, 10.0 mmol), methanamine hydrochloride (1.35 g, 20.0 mmol), HOBt (2.7 g, 20.0 mmol), EDC.HCl (3.8 g, 20.0 mmol) and Et₃N (2.0 g, 20.0 mmol) in DCM (25 mL) was stirred at RT under N₂ for 18 h, and then diluted with H₂O (25 mL). The organic layer was separated, dried (Na₂SO₄) and concentrated under reduced pressure. The crude residue was purified by silica gel column chromatography (DCM:MeOH, 50:1) to afford the desired product (1.5 g, 48%) as a white solid. LCMS (method A): 2.01 min; m/z=228.1, 230.1 [M+H]⁺.

### Step 2: 5-((1-(tert-butyl)-4-cyano-3-(4-nitrophenyl)-1H-pyrazol-5-yl)amino)-N,2-dimethylbenzamide

5-Amino-1-(tert-butyl)-3-(4-nitrophenyl)-1H-pyrazole-4-carbonitrile (2.0 g, 7.0 mmol), 5-bromo-N,2-dimethylbenzamide (0.96 g, 4.2 mmol), Pd(t-Bu₃P)₂ (0.18 g, 0.35 mmol) and Cs₂CO₃ (2.3 g, 7.0 mmol) in PhMe (10 mL) were stirred at 110° C. under N₂ for 24 h. The reaction mixture was concentrated under reduced pressure and the residue was diluted with EtOAc (50 mL) and H₂O (25 mL). The organic layer was separated, dried (Na₂SO₄) and concentrated under reduced pressure. The crude residue was purified by silica gel column chromatography (PE:EtOAc, 10:1) to afford the desired product (0.6 g, 40%) as a yellow solid. LCMS (method A): 3.17 min; m/z=433.1 [M+H]⁺.

### Step 3: 1-(tert-butyl)-5-((4-methyl-3-(methylcarbamoyl)phenyl)amino)-3-(4-nitrophenyl)-1H-pyrazole-4-carboxamide

To a solution of 5-((1-(tert-butyl)-4-cyano-3-(4-nitrophenyl)-1H-pyrazol-5-yl)amino)-N,2-dimethylbenzamide (0.6 g, 1.4 mmol) in DMSO (10 mL), was added 30% aq. H₂O₂ (5.0 g) and K₂CO₃ (0.6 g, 4.2 mmol) at 0° C., and then the mixture was allowed to stir at 120° C. for 24 h. The resulting mixture was then diluted with EtOAc (50 mL) and H₂O (25 mL). The organic layer was separated, dried (Na₂SO₄) and concentrated under reduced pressure. The crude residue was purified by silica gel column chromatography (DCM:MeOH, 50:1) to afford the desired product (300 mg, 48%) as a yellow solid. LCMS (method A): 2.25 min; m/z=451.1 [M+H]⁺.

### Step 4: 3-(4-aminophenyl)-1-(tert-butyl)-5-((4-methyl-3-(methylcarbamoyl)phenyl)amino)-1H-pyrazole-4-carboxamide

To a solution of 1-(tert-butyl)-5-((4-methyl-3-(methylcarbamoyl)phenyl)amino)-3-(4-nitrophenyl)-1H-pyrazole-4-carboxamide (300 mg, 0.67 mmol) in MeOH (30 mL), was added aq. NH₄Cl (10 mL) and Zn dust (220 mg, 3.33 mmol) at 0° C., and then the mixture was allowed to stir at 60° C. for 3 h. The mixture was filtered, and the filter cake was washed with MeOH. The combined filtrates were evaporated, and the residue was diluted with Et₂O (25 mL) and H₂O (15 mL). The organic layer was separated, dried (Na₂SO₄) and concentrated under reduced pressure. The crude residue was purified by silica gel column chromatography (DCM:MeOH, 50:1) to afford the desired product (120 mg, 43%) as a yellow solid. LCMS (method A): 1.95 min; m/z=421.1 [M+H]⁺.

### Step 5: 1-(tert-butyl)-5-((4-methyl-3-(methylcarbamoyl)phenyl)amino)-3-(4-(3-(4-(trifluoromethoxy)phenyl)ureido)phenyl)-1H-pyrazole-4-carboxamide

3-(4-Aminophenyl)-1-(tert-butyl)-5-((4-methyl-3-(methylcarbamoyl)phenyl)amino)-1H-pyrazole-4-carboxamide (120 mg, 0.29 mmol) and 1-isocyanato-4-(trifluoromethoxy)benzene (46 mg, 0.23 mmol) in THF (5 mL) were stirred at RT for 1 h, and then evaporated. The residue was diluted with EtOAc (15 mL) and H₂O (10 mL) and the organic layer was separated, dried (Na₂SO₄) and concentrated under reduced pressure. The crude residue was purified by prep-TLC (DCM:MeOH, 10:1) to afford the desired product (80 mg, 44%) as a white solid. LCMS (method A): 3.07 min; m/z=624.1 [M+H]⁺.

### Step 6: 5-((4-methyl-3-(methylcarbamoyl)phenyl)amino)-3-(4-(3-(4-(trifluoromethoxy)phenyl)ureido)phenyl)-1H-pyrazole-4-carboxamide (Compound 115)

1-(tert-Butyl)-5-((4-methyl-3-(methylcarbamoyl)phenyl)amino)-3-(4-(3-(4-(trifluoromethoxy)phenyl)ureido)phenyl)-1H-pyrazole-4-carboxamide (80 mg, 0.13 mmol) was dissolved in TFA (1 mL) and the mixture was stirred at 60° C. for 1 h. The mixture was concentrated under reduced pressure and the residue was triturated (MeOH) to give the desired product (38 mg, 52%) as a yellow solid as a mono TFA salt. LCMS (method A): 2.78 min; m/z=568.2 [M+H]⁺. ¹H NMR (400 MHz, DMSO-d₆): 12.62 (s, 1H), 9.05 (s, 1H), 9.02 (s, 1H), 8.13 (q, J=4.4 Hz, 1H), 7.65 (d, J=8.4 Hz, 2H), 7.59 (d, J=9.2 Hz, 2H), 7.51 (d, J=8.4 Hz, 2H), 7.43 (dd, J=8.0, 2.0 Hz, 1H), 7.32 (d, J=8.8 Hz, 2H), 7.10 (d, J=8.0 Hz, 1H), 2.75 (d, J=4.4 Hz, 3H), 2.23 (s, 3H).

### Compound 116

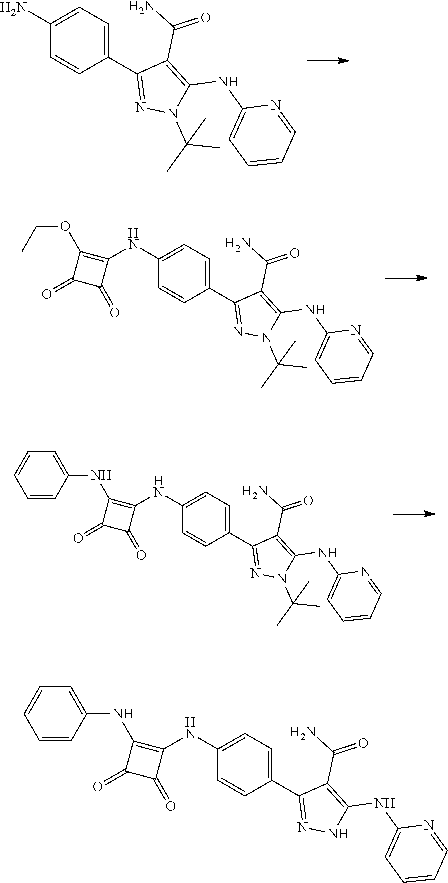 [SMILES: CC(C)(C)n1nc(-c2ccc(N)cc2)c(C(N)=O)c1Nc1ccccn1 ; CCOc1c(Nc2ccc(-c3nn(C(C)(C)C)c(Nc4ccccn4)c3C(N)=O)cc2)c(=O)c1=O ; CC(C)(C)n1nc(-c2ccc(Nc3c(Nc4ccccc4)c(=O)c3=O)cc2)c(C(N)=O)c1Nc1ccccn1 ; NC(=O)c1c(-c2ccc(Nc3c(Nc4ccccc4)c(=O)c3=O)cc2)n[nH]c1Nc1ccccn1]

### Step 1: 1-(tert-butyl)-3-(4-((2-ethoxy-3,4-dioxocyclobut-1-en-1-yl)amino)phenyl)-5-(pyridin-2-ylamino)-1H-pyrazole-4-carboxamide

A solution of 3-(4-aminophenyl)-1-(tert-butyl)-5-(pyridin-2-ylamino)-1H-pyrazole-4-carboxamide (150 mg, 0.43 mmol), 3,4-diethoxy cyclobut-3-ene-1,2-dione (87 mg, 0.52 mmol) and Et₃N (43 mg, 0.43 mmol) in EtOH (20 mL) was stirred at 60° C. for 2 h. The solvent was evaporated under reduced pressure, and the residue was triturated (Et₂O) to afford the desired product (150 mg, 74%) as a yellow solid. LCMS (method A): 2.43 min; m/z=474.9 [M+H]⁺. ¹H NMR (400 MHz, MeOD-d₄): 7.94 (d, J=4.0 Hz, 1H), 7.65 (d, J=8.8 Hz, 2H), 7.49 (m, 1H), 7.34 (m, 2H), 6.68 (t, J=6.0 Hz, 1H), 6.55 (d, J=8.4 Hz, 1H), 3.20 (m, 2H), 1.55 (s, 9H), 1.41 (t, J=7.2 Hz, 3H).

### Step 2: 1-(tert-butyl)-3-(4-((3,4-dioxo-2-(phenylamino)cyclobut-1-en-1-yl)amino)phenyl)-5-(pyridin-2-ylamino)-1H-pyrazole-4-carboxamide

A solution of 1-(tert-butyl)-3-(4-((2-ethoxy-3,4-dioxocyclobut-1-en-1-yl)amino)phenyl)-5-(pyridin-2-ylamino)-1H-pyrazole-4-carboxamide (90 mg, 0.19 mmol), aniline (21 mg, 0.23 mmol) and Et₃N (19 mg, 0.19 mmol) in EtOH (15 mL) was stirred at 80° C. for 2 d. The solvent was evaporated under reduced pressure, and the residue was triturated (Et₂O) to afford the title product (80 mg, 81%) as a yellow solid. LCMS (method A): 2.57 min; m/z=521.9 [M+H]⁺. ¹H NMR (400 MHz, DMSO-d₆): 11.34 (br s, 2H), 8.38 (s, 1H), 8.06 (d, J=4.0 Hz, 1H), 7.74 (d, J=8.8 Hz, 2H), 7.64 (m, 4H), 7.55 (t, J=9.6 Hz, 1H), 7.37 (t, J=8.0 Hz, 2H), 7.16 (s, 1H), 7.07 (t, J=7.6 Hz, 1H), 7.00 (s, 1H), 6.72 (t, J=5.6 Hz, 1H), 6.57 (d, J=8.0 Hz, 1H), 1.57 (s, 9H).

### Step 3: 3-(4-((3,4-dioxo-2-(phenylamino)cyclobut-1-en-1-yl)amino)phenyl)-5-(pyridin-2-ylamino)-1H-pyrazole-4-carboxamide (Compound 116)

A mixture of 1-(tert-butyl)-3-(4-((3,4-dioxo-2-(phenylamino)cyclobut-1-en-1-yl)amino)phenyl)-5-(pyridin-2-ylamino)-1H-pyrazole-4-carboxamide (80 mg, 0.15 mmol) in TFA (2 mL) was stirred at 60° C. for 1 h. The solvent was evaporated under reduced pressure and the residue was triturated (Et₂O) to give the title product (50 mg, 71%) as a yellow solid. LCMS (method A): 2.19 min; m/z=465.9 [M+H]⁺. ¹H NMR (400 MHz, DMSO-d₆): 13.75 (br s, 1H), 11.70 (br s, 1H), 11.60-11.40 (m, 2H), 9.65 (br s, 1H), 8.99 (br s, 1H), 8.40 (br s, 1H), 8.20 (br s, 1H), 8.10-6.80 (m, 11H).

Following the full synthesis of compound 116, starting from 1-(tert-butyl)-3-(4-((2-ethoxy-3,4-dioxocyclobut-1-en-1-yl)amino)phenyl)-5-(pyridin-2-ylamino)-1H-pyrazole-4-carboxamide with the mentioned reagents as described in step 2, the following compounds were prepared:

TABLE 16

| Compound | | | | | |
|---|---|---|---|---|---|
| No | Name | Structure | LCMS data | ¹H NMR data | SM |
| 117 | 3-(4-((2- (dimethylamino)-3,4- dioxocyclobut-1-en-1- yl)amino)phenyl)-5- (pyridin-2-ylamino)- 1H-pyrazole-4- carboxamide | 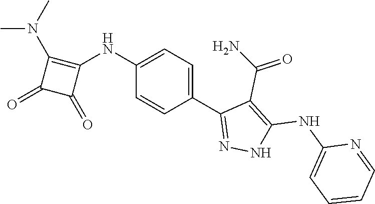 [SMILES: CN(C)c1c(Nc2ccc(-c3n[nH]c(Nc4ccccn4)c3C(N)=O)cc2)c(=O)c1=O] | LCMS (method A): 0.34 min; m/z = 418.0 [M + H]⁺. | ¹H NMR (400 MHz, MeOD-d₄): 12.8 (br s, 1H), 9.5 (br s, 1H), 8.20-8.00 (m, 2H), 7.52-6.39 (m, 8H), 6.90 (br s, 1H), 3.32 (s, 6H). | dimethyl- amine |
| 118 | 3-(4-((2- (methylamino)-3,4- dioxocyclobut-1-en-1- yl)amino)phenyl)-5- (pyridin-2-ylamino)- 1H-pyrazole-4- carboxamide | 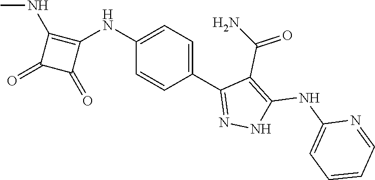 [SMILES: CNc1c(Nc2ccc(-c3n[nH]c(Nc4ccccn4)c3C(N)=O)cc2)c(=O)c1=O] | LCMS (method A): 2.95 min; m/z = 404.2 [M + H]⁺. | ¹H NMR (400 MHz, DMSO-d₆): 13.73 (s, 1H), 10.45-9.90 (m, 2H), 8.95 (d, J = 7.2 Hz, 1H), 8.38 (d, J = 8.0 Hz, 1H), 7.84- 7.82 (m, 2H), 7.57-7.55 (m, 3H), 7.27 (s, | methyl- amine |
| | | | | 1H), 7.15 (s, 1H), | |
| | | | | 7.02-7.00 (m, | |
| | | | | 1H), 3.25 (d, J = | |
| | | | | 4.0 Hz, 3H). | |
| 119 | 3-(4-((3,4-dioxo-2- (piperidin-1- yl)cyclobut-1-en-1- yl)amino)phenyl)-5- (pyridin-2-ylamino)- 1H-pyrazole-4- carboxamide | 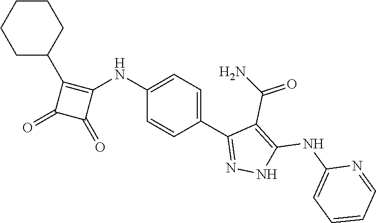 [SMILES: NC(=O)c1c(-c2ccc(Nc3c(C4CCCCC4)c(=O)c3=O)cc2)n[nH]c1Nc1ccccn1] | LCMS (method A): 1.96 min; m/z = 45821 [M + H]⁺. | ¹H NMR (400 MHz, DMSO-d₆): 13.75 (br s, 1H), 9.68-9.64 (m, 2H), 8.98 (d, J = 6.8 Hz, 1H), 8.35-8.21 (m, 2H), 7.84-7.75 (m, 2H), 7.54 (d, J = 8.4 Hz, 2H), 7.30-6,90 (m, 4H), 3.75 (br s, 4H), 1.65 (br s, 6H). | piperidine |
| 120 | 3-(4-((2- (benzylamino)-3,4- dioxocyclobut-1-en-1- yl)amino)phenyl)-5- (pyridin-2-ylamino)- 1H-pyrazole-4- carboxamide | 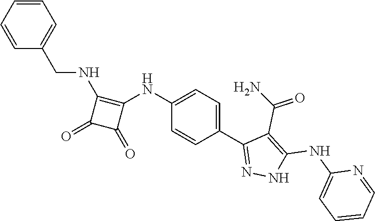 [SMILES: NC(=O)c1c(-c2ccc(Nc3c(NCc4ccccc4)c(=O)c3=O)cc2)n[nH]c1Nc1ccccn1] | LCMS (method A): 2.53 min; m/z = 480.2 [M + H]⁺. | ¹H NMR (400 MHz, DMSO-d₆): 13.75 (br s, 1H), 9.92 (s,1H), 8.97 (s, 1H), 8.39 (d, J = 8.4 Hz, 1H), 8.23 (br s, 1H), 7.84- 7.82 (m, 1H), 7.58-7.54 (m, 4H), 7.42-7.40 (m, 4H), 7.34- 7.33 (m, 2H), 7.23-7.10 (m, | benzyl- amine |
| | | | | 2H), 6.97 (s, | |
| | | | | 1H), 4.85-4.82 | |
| | | | | (m, 2H). | |
| 121 | 3-(4-((2-((4- methoxyphenyl) amino)- 3,4-dioxocyclobut-1- en-1- yl)amino)phenyl)-5- (pyridin-2-ylamino)- 1H-pyrazole-4- carboxamide | 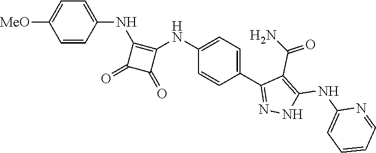 [SMILES: COc1ccc(Nc2c(Nc3ccc(-c4n[nH]c(Nc5ccccn5)c4C(N)=O)cc3)c(=O)c2=O)cc1] | LCMS (method A): 2.21 min; m/z = 496.2 [M + H]⁺. | ¹H NMR (400 MHz, DMSO-d₆): 13.73 (br s, 1H), 10.50-9.50 (m, 3H), 8.97 (br s, 1H), 8.42 (br s, 1H), 8.22 (br s, 1H), 8.00-6.60 (m, 11H), 3.75 (s, 3H). | 4- methoxy- aniline |
| 122 | 3-(4-((2-((4- fluorophenyl)amino)- 3,4-dioxocyclobut-1- en-1- yl)amino)phenyl)-5- (pyridin-2-ylamino)- 1H-pyrazole-4- carboxamide | 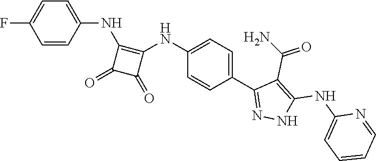 [SMILES: NC(=O)c1c(-c2ccc(Nc3c(Nc4ccc(F)cc4)c(=O)c3=O)cc2)n[nH]c1Nc1ccccn1] | LCMS (method A): 2.28 min; m/z = 484.2 [M + H]⁺. | ¹H NMR (400 MHz, DMSO-d₆): 13.74 (br, s, 1H), 10.26 (s, 1H), 10.15 (s, 1H), 10.09 (s, 1H), 9.91 (s, 1H), 8.98 (d, J = 6.8 Hz, 1H), 8.43 (d, J = 8.8 | 4- fluoroaniline |
| | | | | Hz, 1H), 8.24 | |
| | | | | (d, J = 5.2 Hz, | |
| | | | | 1H), 7.84 (s, | |
| | | | | 1H), 7.65-7.51 | |
| | | | | (m, 4H), 7.27- | |
| | | | | 7.13 (m, 4H), | |
| | | | | 7.00-6.80 (m, | |
| | | | | 2H). | |
| 123 | 3-(4-((2-morpholino- 3,4-dioxocyclobut-1- en-1- yl)amino)phenyl)-5- (pyridin-2-ylamino)- 1H-pyrazole-4- carboxamide | 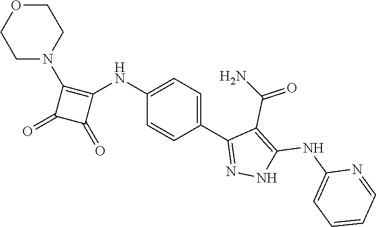 [SMILES: NC(=O)c1c(-c2ccc(Nc3c(N4CCOCC4)c(=O)c3=O)cc2)n[nH]c1Nc1ccccn1] | LCMS (method A): 2.28 min; m/z = 460.2 [M + H]⁺. | ¹H NMR (400 MHz, DMSO-d₆): 13.71 (br s, 1H), 9.70 (br s, 1H), 8.98 (d, J = 6.8 Hz, 1H), 8.37 (d, J = 8.4 Hz, 2H), 7.83- 7.81 (m, 1H), 7.54 (d, J = 8.8 Hz, 1H), 7.35- 7.00 (m, 6H), 4.00-3.60 (m, 8H). | morpholine |
| 124 | 3-(4-((2-(4- methylpiperazin-1-yl)- 3,4-dioxocyclobut-1- en-1- yl)amino)phenyl)-5- (pyridin-2-ylamino)- 1H-pyrazole-4- carboxamide | 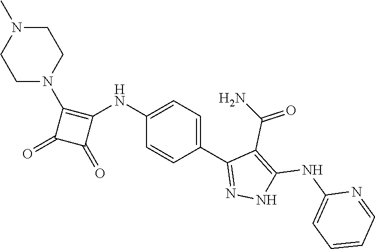 [SMILES: CN1CCN(c2c(Nc3ccc(-c4n[nH]c(Nc5ccccn5)c4C(N)=O)cc3)c(=O)c2=O)CC1] | LCMS (method A): 2.03 min; m/z = 473.2 [M + H]⁺. | ¹H NMR (400 MHz, DMSO-d₆): 12.81 (br s, 1H), 9.51 (br s, 1H), 8.19 (br s, 1H), 7.98 (br s, 1H), 7.72-7.69 (m, 1H), 7.52 (d, J = 8.0 Hz, 2H), 7.36 (d, J = 8.0 Hz, 2H), 7.30-7.20 (m, 1H), 7.02-7.00 (m, 2H), 7.00-6.67 (m, 1H), 5.95 | 1- methyl- piperazine |
| | | | | (br s, 1H), 3.79 | |
| | | | | (br s, 4H), 2.50- | |
| | | | | 2.48 (m, 4H), | |
| | | | | 2.23 (s, 3H). | |

### Compound 125

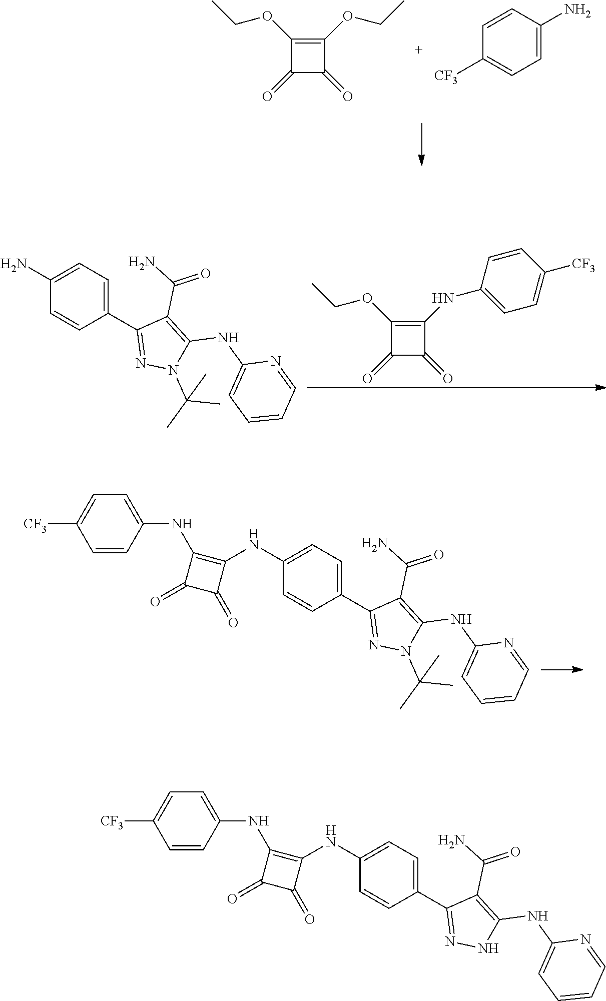 [SMILES: CCOc1c(OCC)c(=O)c1=O ; Nc1ccc(C(F)(F)F)cc1 ; CC(C)(C)n1nc(-c2ccc(N)cc2)c(C(N)=O)c1Nc1ccccn1 ; CCOc1c(Nc2ccc(C(F)(F)F)cc2)c(=O)c1=O ; CC(C)(C)n1nc(-c2ccc(Nc3c(Nc4ccc(C(F)(F)F)cc4)c(=O)c3=O)cc2)c(C(N)=O)c1Nc1ccccn1 ; NC(=O)c1c(-c2ccc(Nc3c(Nc4ccc(C(F)(F)F)cc4)c(=O)c3=O)cc2)n[nH]c1Nc1ccccn1]

### Step 1: 3-ethoxy-4-((4-(trifluoromethyl)phenyl)amino)cyclobut-3-ene-1,2-dione

A mixture of 3,4-diethoxycyclobut-3-ene-1,2-dione (500 mg, 2.9 mmol), 4-(trifluoromethyl)aniline (710 mg, 4.4 mmol) and Et₃N (293 mg, 2.9 mmol) in EtOH (10 mL) was stirred at 80° C. for 2 h. The mixture was concentrated under reduced pressure and the crude residue was purified by silica gel column chromatography (DCM:MeOH, 70:1) to afford the title product (400 mg, 48%) as a yellow oil. LCMS (method A): 3.12 min; m/z=286.1 [M+H]⁺.

### Step 2: 1-(tert-butyl)-3-(4-((3,4-dioxo-2-((4-(trifluoromethyl)phenyl)amino)cyclobut-1-en-1-yl)amino)phenyl)-5-(pyridin-2-ylamino)-1H-pyrazole-4-carboxamide

A mixture of 3-(4-aminophenyl)-1-(tert-butyl)-5-(pyridin-2-ylamino)-1H-pyrazole-4-carboxamide (50 mg, 0.14 mmol), 3-ethoxy-4-((4-(trifluoromethyl)phenyl)amino)cyclobut-3-ene-1,2-dione (61 mg, 0.21 mmol) and Et₃N (14 mg, 0.14 mmol) in EtOH (5 mL) was stirred at 60° C. for 3 h. The solution was concentrated under reduced pressure and the residue was triturated (Et₂O) and dried under reduced pressure to give the title product (65 mg, 78%) as a yellow solid. LCMS (method A): 2.93 min; m/z=590.2 [M+H]⁺.

### Step 3: 3-(4-((3,4-dioxo-2-((4-(trifluoromethyl)phenyl)amino)cyclobut-1-en-1-yl)amino)phenyl)-5-(pyridin-2-ylamino)-1H-pyrazole-4-carboxamide (Compound 125)

A mixture of 1-(tert-butyl)-3-(4-((3,4-dioxo-2-((4-(trifluoromethyl)phenyl)amino)cyclobut-1-en-1-yl)amino)phenyl)-5-(pyridin-2-ylamino)-1H-pyrazole-4-carboxamide (65 mg, 0.11 mmol) in TFA (2 mL) was stirred at 60° C. for 1 h. The solvent was evaporated under reduced pressure, and the residue was diluted with EtOAc (50 mL) and washed with sat. Na₂CO₃ (20 mL). The organic layer was separated, dried (Na₂SO₄), and concentrated under reduced pressure. The crude residue was purified by prep-TLC (DCM:MeOH, 1:0 to 9:1) to afford the title product (20 mg, 34%) as a yellow solid. LCMS (method A): 2.62 min; m/z=534.0 [M+H]⁺. ¹H NMR (400 MHz, DMSO-d₆): 12.84 (br s, 1H), 10.21 (br s, 2H), 9.48 (br s, 1H), 8.19 (br s, 1H), 7.99 (br s, 1H), 7.74-7.63 (m, 10H), 6.87 (br s, 1H), 6.07 (br s, 1H).

The following compounds were similarly prepared from the appropriate arylamine and 1-(tert-butyl)-3-(4-((2-ethoxy-3,4-dioxocyclobut-1-en-1-yl)amino)phenyl)-5-(pyridin-2-ylamino)-1H-pyrazole-4-carboxamide according to the method described for the synthesis of compound 125:

TABLE 17

| Compound | | | | | |
|---|---|---|---|---|---|
| No | Name | Structure | LCMS data | ¹H NMR data | SM |
| 126 | 3-(4-((2- (methyl(phenyl) amino)-3,4- dioxocyclobut- 1-en-1- yl)amino)phenyl)-5- (pyridin-2-ylamino)- 1H-pyrazole-4- carboxamide | 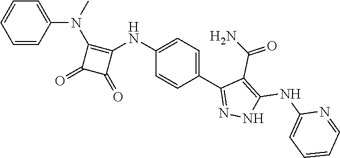 [SMILES: CN(c1ccccc1)c1c(Nc2ccc(-c3n[nH]c(Nc4ccccn4)c3C(N)=O)cc2)c(=O)c1=O] | LCMS (method A): 2.46 min; m/z = 480.1 [M + H]⁺. | ¹H NMR (400 MHz, DMSO-d₆): 12.70 (br s, 1H), 9.66 (s, 1H), 8.25-8.20 (m, 1H), 7.76-7.74 (m, 1H), 7.39-7.08 (m, 13H), 6.95- 6.88 (m, 1H), 6.90 (s, 1H), 3.76 (s, 3H). | N- methyl- aniline |
| 130 | 3-(4-((3,4-dioxo-2- ((4- (trifluoromethoxy) phenyl)amino) cyclobut-1-en-1- yl)amino)phenyl)-5- (pyridin-2-ylamino)- 1H-pyrazole-4- carboxamide | 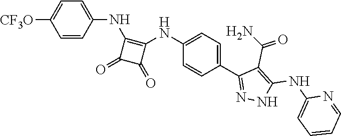 | LCMS (method A): 2.55 min; m/z = 550.0 [M + H]⁺. | ¹H NMR (400 MHz, DMSO-d₆): 13.71 (s, 1H), 10.20-10.10 (m, 2H), 9.77 (br s, 1H), 8.99 (br s, 1H), 8.45(d, J = 8.4 Hz, 1H), 8.23 (d, J = 4.4 Hz, 1H), 7.64 (br s, 1H), 7.61 (br s, 3H), 7.60 (br s, 1H), 7.59 (br s, 2H), 7.23 (br s, 2H), 7.10-6.98 (m, 3H). | 4- (trifluoro- methoxy) aniline |

### Compound 127

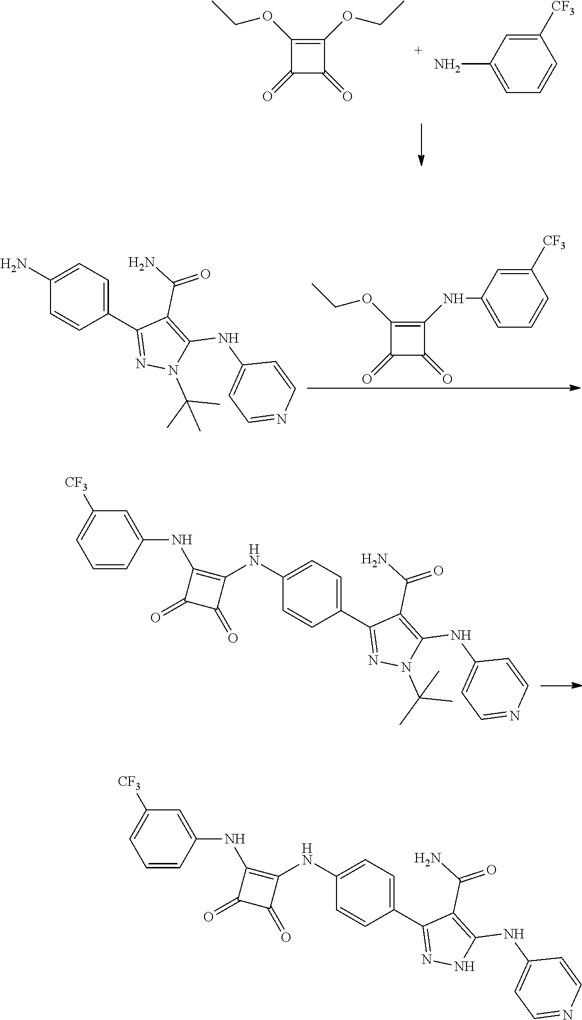 [SMILES: CCOc1c(OCC)c(=O)c1=O ; Nc1cccc(C(F)(F)F)c1 ; CC(C)(C)n1nc(-c2ccc(N)cc2)c(C(N)=O)c1Nc1ccncc1 ; CCOc1c(Nc2cccc(C(F)(F)F)c2)c(=O)c1=O ; CC(C)(C)n1nc(-c2ccc(Nc3c(Nc4cccc(C(F)(F)F)c4)c(=O)c3=O)cc2)c(C(N)=O)c1Nc1ccncc1 ; NC(=O)c1c(-c2ccc(Nc3c(Nc4cccc(C(F)(F)F)c4)c(=O)c3=O)cc2)n[nH]c1Nc1ccncc1]

### Step 1: 3-ethoxy-4-((3-(trifluoromethyl)phenyl)amino)cyclobut-3-ene-1,2-dione

A mixture of 3,4-diethoxycyclobut-3-ene-1,2-dione (500 mg, 2.9 mmol) and 3-(trifluoromethyl)aniline (710 mg, 4.4 mmol) in EtOH (15 mL) was stirred at 60° C. overnight. The mixture was concentrated under reduced pressure and the crude residue was purified by silica gel column chromatography (DCM:MeOH, 70:1) to afford the title product (295 mg, 35%) as a yellow solid. LCMS (method A): 3.58 min; m/z=286.0 [M+H]⁺.

### Step 2: 1-(tert-butyl)-3-(4-((3,4-dioxo-2-((3-(trifluoromethyl)phenyl)amino)cyclobut-1-en-1-yl)amino)phenyl)-5-(pyridin-4-ylamino)-1H-pyrazole-4-carboxamide

A mixture of 3-(4-aminophenyl)-1-(tert-butyl)-5-(pyridin-4-ylamino)-1H-pyrazole-4-carboxamide (from compound 123, 100 mg, 0.29 mmol), 3-ethoxy-4-((3-(trifluoromethyl)phenyl)amino)cyclobut-3-ene-1,2-dione (100 mg, 0.35 mmol), and Et₃N (30 mg, 0.29 mmol, 1.0 eq) in EtOH (8 mL) was stirred at 70° C. for 5 d. The mixture was concentrated under reduced pressure and the crude residue was purified by prep-TLC (DCM:MeOH, 10:1) to afford the title product (72 mg, 42%) as a yellow solid. LCMS (method A): 2.12 min; m/z=590.1 [M+H]⁺.

### Step 3: 3-(4-((3,4-dioxo-2-((3-(trifluoromethyl)phenyl)amino)cyclobut-1-en-1-yl)amino)phenyl)-5-(pyridin-4-ylamino)-1H-pyrazole-4-carboxamide (Compound 127)

A mixture of 1-(tert-butyl)-3-(4-((3,4-dioxo-2-((3-(trifluoromethyl)phenyl)amino)cyclobut-1-en-1-yl)amino)phenyl)-5-(pyridin-4-ylamino)-1H-pyrazole-4-carboxamide (72 mg, 0.12 mmol) in TFA (5 mL) was stirred at 85° C. for 24 h. The mixture was concentrated under reduced pressure and the crude residue was purified by Prep-TLC (DCM:MeOH, 95:5) to afford the desired product (3.6 mg, 6%) as a yellow solid. LCMS (method A): 2.98 min; m/z=534.0 [M+H]⁺. ¹H NMR (400 MHz, DMSO-d₆): 10.39 (br s, 1H), 10.27 (br s, 1H), 8.54 (d, J=6.8 Hz, 2H), 7.99 (s, 1H), 7.76 (d, J=6.4 Hz, 2H), 7.72 (d, J=8.0 Hz, 1H), 7.64-7.58 (m, 3H), 7.54 (d, J=8.4 Hz, 1H), 7.43 (d, J=7.6 Hz, 1H).

### Biology

### In Vitro Assay

Binding affinity of the test compounds for MLKL (full length), RIP1 and RIP3 was determined using the KINOMEscan™ technology developed by DiscoverX (USA; http://www.discoverx.com). The assay was conducted according to manufacturer instructions

### Protocol Description

Kinase assays. For most assays, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions to screen test compounds for kinase binding activity were assembled by combining kinases, liganded affinity beads, and test compounds in 1× binding buffer (20% SeaBlock, 0.17×PBS, 0.05% Tween 20, 6 mM DTT). All reactions were performed in polypropylene 384-well plates in a final volume of 20 μL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

### Compound Handling

An 11-point 3-fold serial dilution of each test compound was prepared in 100% DMSO at 100× final test concentration and subsequently diluted to 1× in the assay (final DMSO concentration=1%). Most K_{D}s were determined using a compound top concentration=30,000 nM. If the initial K_{D} determined was <0.5 nM (the lowest concentration tested in the initial serial dilution), the measurement was repeated with a further 11 point 3-fold serial dilution starting at 3,000 nM.

### Binding Constants (K_{D}s)

K_{D} for each test compound was calculated with a standard dose-response curve using the Hill equation (equation (1)):

$$Response = Background + \frac{Signal - Background}{1 + ( {K}_{D}^{Hill Slope} / {Dose}^{Hill Slope}} \qquad ( 1 )$$

The Hill Slope was set to −1.

Curves were fitted using a non-linear least square fit with the Levenberg-Marquardt algorithm.

### Refs:

Fabian, M. A. et al. A small molecule-kinase interaction map for clinical kinase inhibitors. Nat. Biotechnol. 23, 329-336 (2005).
Karaman, M. W. et al. A quantitative analysis of kinase inhibitor selectivity. Nat. Biotechnol. 26, 127-132 (2008).
Hill, A. V. The possible effects of the aggregation of the molecules of hemoglobin on its dissociation curves. J. Physiol. (Lond.). 40, iv-vii (1910).
Levenberg, K. A method for the solution of certain non-linear problems in least squares. Q. Appl. Math. 2, 164-168 (1944).

TABLE 18
Results of binding assay for compounds of the invention against MLKL,
RIPK1 and RIPK3. Binding affinity is measured by the equilibrium
dissociation constant (K_{D}). The smaller the K_{D} value, the
greater the binding affinity of the ligand for its target.
Activity is provided as follows:
+++ = K_{D} < 100 nM
++ = 100 nM < K_{D} < 1,000 nM
+ = K_{D} > 1,000 nM
Blank = not determined

| Compound | MLKL | RIP1 | RIP3 |
|---|---|---|---|
| ID | (K_{D}) | (K_{D}) | (K_{D}) |
| 1 | + | + | + |
| 2 | + | + | + |
| 3 | +++ | | + |
| 4 | ++ | | + |
| 5 | + | + | + |
| 6 | ++ | + | + |
| 7 | ++ | ++ | + |
| 8 | ++ | + | + |
| 9 | + | | + |
| 10 | ++ | + | + |
| 11 | + | + | + |
| 12 | + | + | + |
| 13 | +++ | ++ | + |
| 14 | + | + | + |
| 16 | ++ | + | + |
| 17 | ++ | + | + |
| 18 | +++ | + | + |
| 19 | ++ | + | + |
| 20 | ++ | | + |
| 21 | +++ | + | + |
| 22 | +++ | + | + |
| 23 | +++ | ++ | + |
| 24 | +++ | ++ | + |
| 25 | ++ | + | + |
| 26 | +++ | + | + |
| 27 | +++ | + | + |
| 28 | ++ | + | + |
| 29 | +++ | + | + |
| 30 | +++ | + | + |
| 31 | +++ | + | + |
| 32 | +++ | ++ | + |
| 33 | ++ | + | + |
| 34 | ++ | + | + |
| 35 | + | + | + |
| 36 | + | + | + |
| 37 | ++ | + | + |
| 38 | +++ | + | + |
| 39 | ++ | + | + |
| 40 | + | + | + |
| 41 | + | + | + |
| 42 | ++ | + | + |
| 43 | ++ | + | ++ |
| 44 | +++ | ++ | ++ |
| 45 | ++ | + | + |
| 46 | ++ | + | + |
| 47 | ++ | + | + |
| 48 | ++ | + | + |
| 49 | ++ | + | + |
| 50 | +++ | + | + |
| 51 | +++ | + | + |
| 52 | +++ | + | + |
| 53 | +++ | + | + |
| 54 | +++ | + | + |
| 55 | +++ | + | + |
| 56 | +++ | ++ | + |
| 57 | +++ | + | + |
| 58 | ++ | + | + |
| 59 | +++ | ++ | + |
| 60 | +++ | ++ | + |
| 61 | +++ | +++ | + |
| 62 | +++ | + | + |
| 63 | +++ | + | + |
| 64 | +++ | ++ | + |
| 65 | +++ | + | + |
| 66 | +++ | ++ | + |
| 67 | +++ | + | + |
| 68 | +++ | + | + |
| 69 | +++ | + | + |
| 70 | +++ | + | + |
| 71 | +++ | + | + |
| 72 | +++ | + | + |
| 73 | ++ | + | + |
| 74 | ++ | + | + |
| 75 | +++ | + | + |
| 76 | +++ | + | + |
| 77 | +++ | + | + |
| 78 | +++ | +++ | + |
| 79 | + | + | + |
| 80 | +++ | +++ | + |
| 81 | ++ | +++ | + |
| 82 | ++ | + | + |
| 83 | ++ | +++ | + |
| 84 | +++ | + | + |
| 85 | +++ | + | + |
| 86 | ++ | + | + |
| 87 | +++ | ++ | + |
| 88 | ++ | ++ | + |
| 89 | +++ | +++ | + |
| 90 | +++ | +++ | + |
| 91 | +++ | ++ | + |
| 92 | +++ | ++ | + |
| 93 | ++ | ++ | + |
| 94 | +++ | +++ | + |
| 95 | +++ | +++ | ++ |
| 96 | +++ | +++ | + |
| 97 | +++ | +++ | + |
| 98 | +++ | +++ | + |
| 99 | +++ | ++ | + |
| 100 | +++ | + | + |
| 101 | +++ | + | + |
| 102 | +++ | + | + |
| 103 | +++ | ++ | + |
| 104 | +++ | ++ | + |
| 105 | ++ | +++ | + |
| 106 | + | | + |
| 107 | +++ | + | + |
| 108 | ++ | + | + |
| 109 | ++ | ++ | + |
| 110 | ++ | | + |
| 111 | ++ | +++ | + |
| 112 | + | | + |
| 113 | + | + | + |
| 114 | ++ | | + |
| 115 | ++ | ++ | + |
| 116 | +++ | + | + |
| 117 | ++ | + | + |
| 118 | + | + | + |
| 119 | + | + | + |
| 120 | +++ | + | + |
| 121 | + | + | + |
| 122 | ++ | + | + |
| 123 | + | + | -+ |
| 124 | + | | + |
| 124 | + | + | + |
| 125 | ++ | + | + |
| 126 | +++ | + | + |
| 127 | + | + | + |

### Cellular Assay: Screening Compounds for Inhibition of TSQ Induced Necroptosis, 384 Well Plate Format.

Cell Line ID: U937 human histiocytic leukemia cell line.

Cell Concentration (cells/well): Final cell density is 20000 cells per well.

Cell growth medium: HT-RPMI medium+7.4% Fetal Bovine Serum (FBS). Cells are cultured in Corning 150 cm² tissue culture flasks with vented caps at 37° C./5% CO₂.

Incubation: Plates were incubated at 37° C./5% CO₂ in a humidified incubator for 48 hours following addition of compounds and death stimuli (TSQ cocktail).

Compound concentration: 36 μM starting concentration, 1:3 dilution, 10 point

DMSO final concentration (% v/v): 0.3%.

Compounds in TSQ cocktail (T: TNF; S: Smac mimetic; Q: Q-VD-OPh) and their final concentrations:

hTNF-Fc (100 ng/ml)—produced by standard procedures as shown in Bossen et al., J Biol Chem, 2006, 281(20), 13964-13971.

Compound A (500 nM)—Smac mimetic, Tetralogic and SYNthesis med chem Q-VD-OPh (10 μM)—MP Biomedicals

### Assay Experimental Outline

The cellular assay was carried out according to the following steps:
1. Each well was prepared by sequential addition of:
   a. DMSO (control; columns 1-2 and 23-24) or compound in DMSO—addition was performed using acoustic transfer of nl volumes of stock compound to give final test concentrations of 36, 12, 4, 1.3, 0.44, 0.148, 0.049, 0.016, 0.005 and 0.002 μM. All wells were backfilled with DMSO to a final total volume in the well of 100 nl.
   b. Following compound/DMSO addition in step (a), 40 μL of cell suspension (5×10⁵ cells/mL) was added to provide a final cell concentration of 20,000 cells per well, and
   c. Following cell addition in step (b), 10 μL of 5×TSQ cocktail (except to positive controls; columns 1 and 23) was added to each well.
2. After 48 hours, plate was removed from the 37° C. incubator and equilibrated to room temperature for 45 minutes.
3. 15 μL of room temperature CellTitre-Glo2™ (Promega™) was added to each well.
4. Shook plates for 2 minutes (˜600 rpm) and incubated at room temperature for 15 minutes to allow signal to stabilise.
5. Read luminescence readout on a plate reader.

### Analysis:

Percent viability was calculated for each compound according to equation (2):

% viability=100×((RawData−NSA)/(TA−NSA) (2)

wherein
RawData is the readout of any cell containing a compound of the invention
TA is the total activity provided by the luminescence readout from DMSO only wells (columns 2 and 24)=100% viability
NSA is the non-specific activity provided by DMSO+TSQ wells (columns 1 and 23)=0% viability

Curve fitting: 10-point titration curves are fitted with the 4-parameter logistic nonlinear regression model and the IC₅₀ reported is the inflection point of the curve

Analysis: Data was loaded into Dotmatics™ and visualised using the Tibco® Spotfire™ software. points titration curve were fitted with the 4 parameter logistic nonlinear regression model and the IC₅₀ reported reflect the inflection point of the curve for curve fitting.

### Interpretation of Results:

Assay involving the TSQ cocktail (T: TNF; S: Smac mimetic; Q: Q-VD-OPh): TSQ treatment ensures that cells specifically undergo necroptotic cell death. TNF activates the TNF receptor, Smac mimetic directs the signal away from proinflammatory signaling and toward the RIP1/RIP3-mediated cell death pathways, and Q-VD-OPh ensures that the apoptotic response is blocked leaving only the programmed necrosis response. The compounds' activity (solution in DMSO) tested in this TSQ-induced assay was evaluated by determining the number of viable cells in culture by measuring the amount of ATP present as measured by CelltiterGlo.

Counter screen: In parallel, all compounds were tested for their ability to affect cell viability. The same U937 cells were treated with compound in DMSO without the TSQ cocktail. This counter screen enabled evaluation of off-target effects. In this case, cell viability was measured by CelltiterGlo.

The results of the screening of the compounds described above are shown below in the Table 19.

TABLE 19
Table showing the results of cell-based assays performed, analysed as the
half maximal inhibitory concentration (IC₅₀), being the concentration
of the test compound needed to inhibit TSQ induced necroptosis
by 50%. The lower the IC₅₀, the more potent the compound.
Activity is provided as follows:
+++ = IC₅₀ < 100 nM
++ = 100 nM < IC₅₀ < 1,000 nM
+ = IC₅₀ > 1,000 nM
Blank = not determined

| | Inhibition of | |
|---|---|---|
| Compound | TSQ-induced necroptosis | Off-target effect |
| No | (IC₅₀ cell-based assay) | (IC₅₀ cell-based assay) |
| 1 | ++ | |
| 3 | + | + |
| 7 | ++ | |
| 8 | + | |
| 13 | ++ | + |
| 21 | + | + |
| 22 | + | + |
| 23 | +++ | + |
| 24 | ++ | + |
| 25 | + | + |
| 27 | + | + |
| 30 | ++ | + |
| 32 | ++ | + |
| 39 | ++ | |
| 44 | ++ | + |
| 47 | + | |
| 48 | + | |
| 58 | + | + |
| 59 | ++ | + |
| 60 | ++ | + |
| 61 | +++ | + |
| 64 | ++ | + |
| 66 | ++ | + |
| 67 | + | + |
| 78 | +++ | + |
| 80 | +++ | + |
| 81 | +++ | + |
| 82 | + | + |
| 83 | +++ | + |
| 87 | ++ | + |
| 88 | ++ | + |
| 89 | ++ | + |
| 90 | +++ | + |
| 91 | + | + |
| 92 | ++ | + |
| 93 | ++ | + |
| 94 | +++ | + |
| 95 | +++ | + |
| 96 | ++ | + |
| 97 | +++ | + |
| 98 | +++ | + |
| 103 | + | + |
| 105 | ++ | |
| 107 | +++ | + |
| 109 | ++ | + |
| 110 | ++ | |
| 111 | +++ | |
| 115 | +++ | |

It will be appreciated by persons skilled in the art that numerous variations and/or modifications may be made to the above-described embodiments, without departing from the broad general spirit and scope of the present disclosure. The present embodiments are, therefore, to be considered in all respects as illustrative and not restrictive.

1. A compound of formula (I) 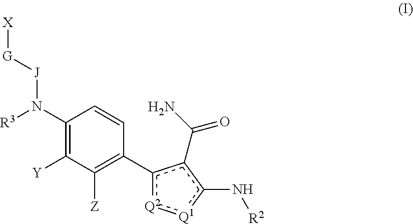
wherein
Q¹ and Q² are selected from N and NR¹, wherein when Q² is N, Q¹ is NR¹ and when Q¹ is N, Q² is NR¹;
R¹ and R³ are independently selected from H and an optionally substituted C₁₋₆-alkyl;
R² is H, an optionally substituted C₁-C₆-alkyl, an optionally substituted aryl or an optionally substituted heterocyclyl;
X is selected from optionally substituted C₁₋₆alkyl, optionally substituted haloC₁₋₆alkyl, optionally substituted —C₁₋₆alkylamino, optionally substituted C₂₋₆alkynyl, optionally substituted cycloalkyl, optionally substituted halocycloalkyl, optionally substituted aryl, optionally substituted alkylaryl, optionally substituted heterocyclyl, optionally substituted C₁₋₆alkylheterocyclyl;
J is selected from carbonyl and 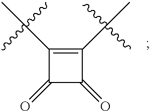 [SMILES: *c1c(*)c(=O)c1=O]
G is selected from a single bond, NR³, CR⁴R⁵ and optionally substituted heterocyclyl; and
R⁴ and R⁵ are independently selected from H, optionally substituted C₁₋₆alkyl, optionally substituted aryl and optionally substituted amino;
or a pharmaceutically acceptable salt, solvate, tautomer, stereoisomer, N-oxide and/or prodrug thereof.

2. The compound of claim 1, or pharmaceutically acceptable salt, solvate, tautomer, stereoisomer, N-oxide and/or prodrug thereof,
wherein X is selected from C₁₋₆alkyl, C₂₋₆alkynyl, C₃₋₆cycloalkyl, aryl, —C₁₋₂alkylaryl, —C₁₋₂alkylcycloalkyl, heterocyclyl, —C₁₋₂alkylheterocyclyl;
wherein each alkyl and alkynyl is optionally substituted with one or more groups selected from halo, nitrile, aryl, R⁶, —OR⁶, —N(R⁷)R⁸;
R⁶, R⁷ and R⁸ are independently selected from H, aryl, C₁₋₆alkyl and haloC₁₋₆alkyl, and
wherein each aryl, heterocyclyl and cycloalkyl is optionally substituted with one or more groups that are independently selected from halo, nitrile, C₁₋₄alkyl, C₁₋₄alkoxy, haloC₁₋₄alkyl and haloC₁₋₄alkoxy.

3. The compound of claim 1 or 2, or pharmaceutically acceptable salt, solvate, tautomer, stereoisomer, N-oxide and/or prodrug thereof, wherein X is a bicyclic fused heterocyclyl or a spirocyclic heterocyclyl selected from any one of partial formulas X1-X3: 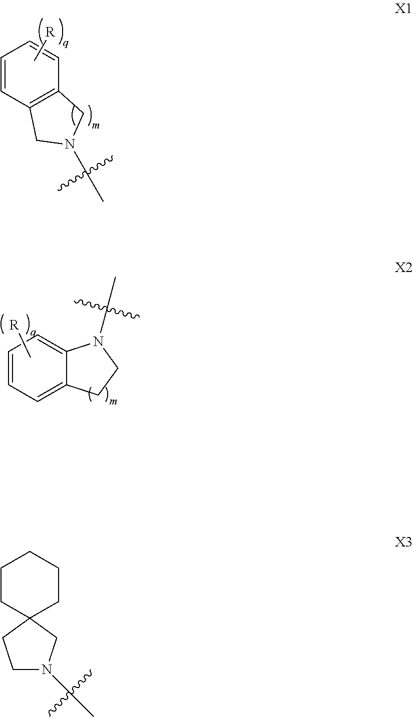
wherein R is selected from C₁₋₄alkyl, haloC₁₋₄alkyl, OC₁₋₄alkyl, OhaloC₁₋₄alkyl, and halo;
q is 0, 1 or 2; and
m is 1 or 2.

4. The compound of any one of claims 1-3, or a pharmaceutically acceptable salt, solvate, tautomer, stereoisomer, N-oxide and/or prodrug thereof, wherein
R⁴ is selected from C₁₋₆alkyl, aryl, and —(CH₂)ₘR¹¹, and
R¹¹ is selected from C₃₋₁₀cycloalkyl, aryl, heterocyclyl,
wherein each cycloalkyl, aryl and heterocyclyl are optionally substituted with one or more groups independently selected from halo, C₁₋₄alkyl, C₁₋₄alkoxy, haloC₁₋₄alkyl and haloC₁₋₄alkoxy.

5. The compound of any one of claims 1-4, or a pharmaceutically acceptable salt, solvate, tautomer, stereoisomer, N-oxide and/or prodrug thereof, wherein R² is selected from an optionally substituted phenyl, an optionally substituted 5-membered monocyclic heteroaryl, an optionally substituted 6-membered monocyclic heteroaryl or an optionally substituted 10-membered bicyclic heteroaryl.

6. The compound of any one of claims 1-5, or a pharmaceutically acceptable salt, solvate, tautomer, stereoisomer, N-oxide and/or prodrug thereof, wherein R² is represented by any one of partial formulas Ar1, Ar2 and Ar3: 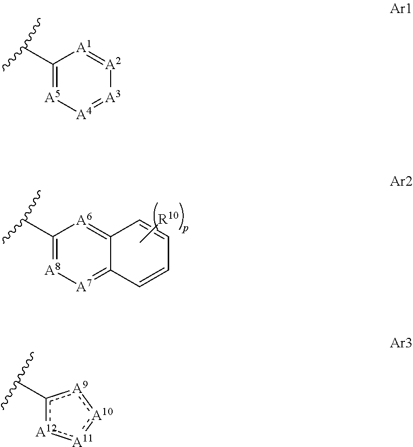
wherein
A¹, A², A³, A⁴, A⁵, A⁶, A⁷, and A⁸ are independently selected from CR¹ and N
A⁹, A¹⁰, A¹¹ and A¹² are independently selected from C(R¹¹)_{q}, O, S, N and NR¹²;
wherein not more than 2 of A¹, A², A³, A⁴ and A⁵ are N;
wherein not more than 2 of A⁶, A⁷ and A⁸ are N;
wherein at least 1 of A⁹, A¹⁰, A¹¹ and A¹² is selected from C(R¹¹)_{q}, O, S and NR¹²;
each R¹¹ is independently selected from H and R¹⁰;
each R¹⁰ is independently selected from halo, C₁₋₆alkyl, C₁₋₆alkoxy, C₃₋₆cycloalkyl, —OC₁₋₆alkylC₁₋₆alkoxy, haloC₁₋₆alkyl, haloC₁₋₆alkoxy, nitrile, amido, C₁₋₆alkylamido, (C₁₋₆alkyl)₂amido, haloC₁₋₆alkylamido, (haloC₁₋₆alkyl)₂amido, acyl, C₁₋₆alkylacyl, haloC₁₋₆alkylacyl, arylacyl, heterocyclylacyl, cycloalkylacyl, heterocyclyl, haloC₁₋₆alkoxy, C₃₋₁₀cycloalkyl, C₁₋₆alkylC₃₋₁₀cycloalkyl, C₁₋₆alkoxyC₃₋₁₀cycloalkyl, haloC₁₋₆alkylC₃₋₁₀cycloalkyl, haloC₁₋₆alkoxyC₃₋₁₀cycloalkyl, C₁₋₆alkylheterocyclyl, C₁₋₆alkoxyheterocyclyl, haloC₁₋₆alkylheterocyclyl, haloC₁₋₆alkoxyheterocyclyl, C₁₋₆alkylC₁₋₆alkoxy, and —COOH;
each R¹² is independently selected from H, C₁₋₆alkyl, haloC₁₋₄alkyl, C₁₋₆alkylacyl and haloC₁₋₆alkylacyl;
or when two adjacent groups selected from A¹, A², A³, A⁴, A⁵, A⁷, A⁸ A⁹, A¹⁰, A¹¹ and A¹² are selected from CR¹¹ and NR¹², two R¹¹, two R¹² or one R¹¹ and one R¹² may together form an optionally substituted 5-10 membered ring selected from cycloalkyl, aryl and heterocyclyl;
p is an integer from 0 to 4; and
q is 1 or 2.

7. The compound of claim 6, or a pharmaceutically acceptable salt, solvate, tautomer, stereoisomer, N-oxide and/or prodrug thereof, wherein R² is represented by partial formula Ar1.

8. The compound of claim 6 or 7, or a pharmaceutically acceptable salt, solvate, tautomer, stereoisomer, N-oxide and/or prodrug thereof, wherein A¹ is N.

9. The compound of any one of claims 6-8, or a pharmaceutically acceptable salt, solvate, tautomer, stereoisomer, N-oxide and/or prodrug thereof, wherein A⁴ is N.

10. The compound of any one of claims 6-9, or a pharmaceutically acceptable salt, solvate, tautomer, stereoisomer, N-oxide and/or prodrug thereof, wherein A² is CR¹⁰.

11. The compound of any one of claims 1-10, or a pharmaceutically acceptable salt, solvate, tautomer, stereoisomer, N-oxide and/or prodrug thereof, wherein R¹ and R³ are H.

12. The compound of claim 1, or a pharmaceutically acceptable salt, solvate, tautomer, stereoisomer, N-oxide and/or prodrug thereof, selected from:
3-((3-methoxyphenyl)amino)-5-(4-(3-(4-(trifluoromethoxy)phenyl)ureido)phenyl)-1H-pyrazole-4-carboxamide
5-((4-methoxyphenyl)amino)-3-(4-(3-(4-(trifluoromethoxy)phenyl)ureido)phenyl)-1H-pyrazole-4-carboxamide
5-((4-methyl-3-(methylcarbamoyl)phenyl)amino)-3-(4-(3-(4-(trifluoromethoxy)phenyl)ureido)phenyl)-1H-pyrazole-4-carboxamide
3-(4-(2-phenylacetamido)phenyl)-5-(pyridin-2-ylamino)-1H-pyrazole-4-carboxamide
5-(pyridin-3-ylamino)-3-(4-(3-(4-(trifluoromethoxy)phenyl)ureido)phenyl)-1H-pyrazole-4-carboxamide
5-(pyridin-4-ylamino)-3-(4-(3-(4-(trifluoromethoxy)phenyl)ureido)phenyl)-1H-pyrazole-4-carboxamide
5-((6-methylpyridin-2-yl)amino)-3-(4-(2-phenylacetamido)phenyl)-1H-pyrazole-4-carboxamide
3-(4-(2-cyclohexylacetamido)phenyl)-5-(pyridin-2-ylamino)-1H-pyrazole-4-carboxamide
5-(pyridin-2-ylamino)-3-(4-(2-(2-(trifluoromethyl)phenyl)acetamido)phenyl)-1H-pyrazole-4-carboxamide
5-(pyridin-2-ylamino)-3-(4-(2-(4-(trifluoromethyl)phenyl)acetamido)phenyl)-1H-pyrazole-4-carboxamide
N-(4-(4-carbamoyl-5-(pyridin-2-ylamino)-1H-pyrazol-3-yl)phenyl)-3,4-dihydroisoquinoline-2(1H)-carboxamide
3-(4-(3-methyl-3-phenylureido)phenyl)-5-(pyridin-2-ylamino)-1H-pyrazole-4-carboxamide
5-(pyridin-2-ylamino)-3-(4-(2-(thiophen-3-yl)acetamido)phenyl)-1H-pyrazole-4-carboxamide
5-((5-(2-methoxyethoxy)pyridin-2-yl)amino)-3-(4-(2-phenylacetamido)phenyl)-1H-pyrazole-4-carboxamide
5-((2-methoxypyridin-4-yl)amino)-3-(4-(2-phenylpyrrolidine-1-carboxamido)phenyl)-1H-pyrazole-4-carboxamide
N-(4-(4-carbamoyl-5-((2-methoxypyridin-4-yl)amino)-1H-pyrazol-3-yl)phenyl)-3-phenylpiperidine-1-carboxamide
3-(4-(3-benzyl-3-methylureido)phenyl)-5-((2-methoxypyridin-4-yl)amino)-1H-pyrazole-4-carboxamide
5-((2-methoxypyridin-4-yl)amino)-3-(4-(3-phenethylureido)phenyl)-1H-pyrazole-4-carboxamide
5-((2-methoxypyridin-4-yl)amino)-3-(4-(3-methyl-3-phenethylureido)phenyl)-1H-pyrazole-4-carboxamide
N-(4-(4-carbamoyl-5-((2,6-dimethylpyridin-4-yl)amino)-1H-pyrazol-3-yl)phenyl)-3-phenylpiperidine-1-carboxamide
rac-(R)-5-((2-methoxypyridin-4-yl)amino)-3-(4-(2-phenylpyrrolidine-1-carboxamido)phenyl)-1H-pyrazole-4-carboxamide
rac-(R)-N-(4-(4-carbamoyl-5-((2-methoxypyridin-4-yl)amino)-1H-pyrazol-3-yl)phenyl)-3-phenylpiperidine-1-carboxamide
(S)-N-(4-(4-carbamoyl-5-((2-methoxypyridin-4-yl)amino)-1H-pyrazol-3-yl)phenyl)-3-phenylpiperidine-1-carboxamide
N-(4-(4-carbamoyl-5-(pyrazin-2-ylamino)-1H-pyrazol-3-yl)phenyl)-3-phenylpiperidine-1-carboxamide
5-(pyrazin-2-ylamino)-3-(4-(2-(4-(trifluoromethoxy)phenyl)acetamido)phenyl)-1H-pyrazole-4-carboxamide
5-(pyrazin-2-ylamino)-3-(4-(3-(4-(trifluoromethoxy)phenyl)ureido)phenyl)-1H-pyrazole-4-carboxamide
N-(4-(4-carbamoyl-5-(pyrazin-2-ylamino)-1H-pyrazol-3-yl)phenyl)-3-(3-fluorophenyl)piperidine-1-carboxamide
3-(4-(2-(4-chlorophenyl)acetamido)phenyl)-5-(pyrazin-2-ylamino)-1H-pyrazole-4-carboxamide
5-(pyrazin-2-ylamino)-3-(4-(2-(2-(trifluoromethyl)phenyl)acetamido)phenyl)-1H-pyrazole-4-carboxamide
N-(4-(4-carbamoyl-5-(pyrazin-2-ylamino)-1H-pyrazol-3-yl)phenyl)-3-(4-fluorophenyl)piperidine-1-carboxamide
N-(4-(4-carbamoyl-5-(pyrazin-2-ylamino)-1H-pyrazol-3-yl)phenyl)-3-(3-(trifluoromethyl)phenyl)piperidine-1-carboxamide
5-(pyrazin-2-ylamino)-3-(4-(2-(3-(trifluoromethoxy)phenyl)acetamido)phenyl)-1H-pyrazole-4-carboxamide
3-(4-(2-(3-methoxyphenyl)acetamido)phenyl)-5-(pyrazin-2-ylamino)-1H-pyrazole-4-carboxamide
N-(4-(4-carbamoyl-5-(pyrazin-2-ylamino)-1H-pyrazol-3-yl)phenyl)-3-(4-(trifluoromethyl)phenyl)piperidine-1-carboxamide
5-(pyrazin-2-ylamino)-3-(4-(2-(3-(trifluoromethyl)phenyl)acetamido)phenyl)-1H-pyrazole-4-carboxamide
5-(pyrazin-2-ylamino)-3-(4-(2-(4-(trifluoromethyl)phenyl)acetamido)phenyl)-1H-pyrazole-4-carboxamide
3-(4-(2-(2-chlorophenyl)acetamido)phenyl)-5-(pyrazin-2-ylamino)-1H-pyrazole-4-carboxamide
N-(4-(4-carbamoyl-5-(pyrazin-2-ylamino)-1H-pyrazol-3-yl)phenyl)-3-(3-methoxyphenyl)piperidine-1-carboxamide
N-(4-(4-carbamoyl-5-(pyrazin-2-ylamino)-1H-pyrazol-3-yl)phenyl)-3-(3-chlorophenyl)piperidine-1-carboxamide
N-(4-(4-carbamoyl-5-(pyrazin-2-ylamino)-1H-pyrazol-3-yl)phenyl)-3-(4-chlorophenyl)piperidine-1-carboxamide
N-(4-(4-carbamoyl-5-(pyrazin-2-ylamino)-1H-pyrazol-3-yl)phenyl)-3-(4-methoxyphenyl)piperidine-1-carboxamide
(S)-N-(4-(4-carbamoyl-5-(pyrazin-2-ylamino)-1H-pyrazol-3-yl)phenyl)-3-phenylpiperidine-1-carboxamide
(R)-N-(4-(4-carbamoyl-5-(pyrazin-2-ylamino)-1H-pyrazol-3-yl)phenyl)-3-phenylpiperidine-1-carboxamide
N-(4-(4-carbamoyl-5-((6-(trifluoromethyl)pyridin-2-yl)amino)-1H-pyrazol-3-yl)phenyl)-3-(3,4-difluorophenyl)piperidine-1-carboxamide
N-(4-(4-carbamoyl-5-((6-(trifluoromethyl)pyridin-2-yl)amino)-1H-pyrazol-3-yl)phenyl)-3-(3,5-difluorophenyl)piperidine-1-carboxamide
(R)-N-(4-(4-carbamoyl-5-((6-(trifluoromethyl)pyridin-2-yl)amino)-1H-pyrazol-3-yl)phenyl)-3-phenylpiperidine-1-carboxamide
(R)-N-(4-(4-carbamoyl-5-((6-(trifluoromethyl)pyridin-2-yl)amino)-1H-pyrazol-3-yl)phenyl)-3-(4-fluorophenyl)piperidine-1-carboxamide
(R)-N-(4-(4-carbamoyl-5-((6-(trifluoromethyl)pyridin-2-yl)amino)-1H-pyrazol-3-yl)phenyl)-3-(3-fluorophenyl)piperidine-1-carboxamide

13. A medicament comprising a compound of any one of claims 1-12, or a pharmaceutically acceptable salt, solvate, tautomer, stereoisomer, N-oxide and/or prodrug thereof.

14. A pharmaceutical composition comprising a compound of any one of claims 1-12, or a pharmaceutically acceptable salt, solvate, tautomer, stereoisomer, N-oxide and/or prodrug thereof, and a pharmaceutically acceptable excipient.

15. A method of treating necroptosis, comprising administering to a subject in need thereof an effective amount of a compound of any one of claims 1-12, or a pharmaceutically acceptable salt, solvate, tautomer, stereoisomer, N-oxide and/or prodrug thereof, the medicament of claim 13, or the pharmaceutical composition of claim 14.

16. The method of claim 15, wherein the subject has a disease selected from the group consisting of diseases of the bones, joints, connective tissue and cartilage, muscular diseases, skin diseases, cardiovascular diseases, circulatory diseases, hematological and vascular diseases, diseases of the lung, diseases of the gastro-intestinal tract, diseases of the liver, diseases of the pancreas, metabolic diseases, diseases of the kidneys, viral and bacterial infections, severe intoxications, degenerative diseases associated with the Acquired Immune Deficiency Syndrome (AIDS), disorders associated with aging, inflammatory diseases, auto-immune diseases, dental disorders, ophthalmic diseases or disorders, diseases of the audition tracts, diseases associated with mitochondria, neuronal loss, ischemic reperfusion injury, cancer and metastatic cancer.

17. Use of a compound of any one of claims 1-12, or a pharmaceutically acceptable salt, solvate, tautomer, stereoisomer, N-oxide and/or prodrug thereof, in the manufacture of a medicament for treating necroptosis.

18. A compound of any one of claims 1-12, or a pharmaceutically acceptable salt, solvate, tautomer, stereoisomer, N-oxide and/or prodrug thereof, for use in the treatment of necroptosis.

19. A method of inhibiting receptor-interacting serine/threonine protein kinase 1 (RIP1) and/or mixed lineage kinase domain-like protein (MLKL), comprising contacting a cell with a compound of any one of claims 1-12, or a pharmaceutically acceptable salt, solvate, tautomer, stereoisomer, N-oxide and/or prodrug thereof.

20. A RIP1 and/or MLKL inhibitor comprising a compound of any one of claims 1-12, or a pharmaceutically acceptable salt, solvate, tautomer, stereoisomer, N-oxide and/or prodrug thereof.
